(19) United States
(12) Patent Application Publication
(10) Pub. No.: US 2024/0011005 A1
(43) Pub. Date: Jan. 11, 2024

(54) NOVEL CRISPR-CAS12F SYSTEMS AND USES THEREOF

(71) Applicant: HuidaGene Therapeutics Co., Ltd., Shanghai (CN)

(72) Inventors: Xiangfeng Kong, Shanghai (CN); Lecong Wang, Shanghai (CN); Xuqiang Kong, Shanghai (CN)

(21) Appl. No.: 18/331,431

(22) Filed: Jun. 8, 2023

Related U.S. Application Data
(63) Continuation of application No. PCT/CN23/90685, filed on Apr. 25, 2023.

(30) Foreign Application Priority Data
Apr. 25, 2022 (WO) PCT/CN2022/089053
Dec. 27, 2022 (WO) PCT/CN2022/142467

(51) Int. Cl. C12N 9/22; C12N 15/86

(52) U.S. Cl. CPC C12N 9/22; C12N 15/86; C12N 2310/20; C12N 2750/00043

(57) ABSTRACT

The disclosure provides Cas12f polypeptides, fusion proteins comprising such Cas12f polypeptides, CRISPR-Cas12f systems comprising such Cas12f polypeptides or fusion proteins, and methods of using the same.

### CROSS-REFERENCE TO RELATED APPLICATIONS

This application is a continuation of PCT Patent Application No. PCT/CN2023/090685, filed on Apr. 25, 2023, which claims the benefits of and priorities to PCT Patent Application No. PCT/CN2022/089053, filed on Apr. 25, 2022, entitled “NOVEL CRISPR-CAS SYSTEMS AND USES THEREOF”, and PCT Patent Application No. PCT/CN2022/142467, filed on Dec. 27, 2022, entitled “NOVEL CRISPR-CAS SYSTEMS AND USES THEREOF”. The entire contents of each of the foregoing applications, including any sequence listing and drawings, are incorporated herein by reference in their entireties.

### REFERENCE TO AN ELECTRONIC SEQUENCE LISTING

The instant application contains a Sequence Listing XML which has been submitted electronically in XML format and is hereby incorporated by reference in its entirety. Said XML copy, created on Jun. 7, 2023, is named 132045-10301_SL.xml and is 539,016 bytes in size.

According to WIPO Standard ST.26, symbol “t” is used to denote both T in DNA and U in RNA (See “Table 1: List of nucleotides symbols”, the definition of symbol “t” is “thymine in DNA/uracil in RNA (t/u)”). Thus, in a sequence listing prepared according to ST.26, wherever a sequence is an RNA, the T in the sequence shall be deemed as U.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

Citation or identification of any document in the disclosure is not an admission that such a document is available as prior art to the disclosure. Each of the references mentioned or cited in the disclosure is incorporated by reference in its entirety.

### SUMMARY

It is against the above background that the disclosure provides certain advantages and advancements over the prior art. Although the disclosure is not limited to specific advantages or functionalities, in one aspect, the disclosure provides a Cas12f polypeptide comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 1-34 (optionally any one of SEQ ID NOs: 1, 2, 4, 5, 15, 28, 29, 31, and 32).

In another aspect, the disclosure provides a system comprising:
(1) a Cas12f polypeptide comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 1-34 (optionally any one of SEQ ID NOs: 1, 2, 4, 5, 15, 28, 29, 31, and 32), or a polynucleotide encoding the Cas12f polypeptide; and
(2) a guide nucleic acid or a polynucleotide encoding the guide nucleic acid, the guide nucleic acid comprising:
(i) a scaffold sequence capable of forming a complex with the Cas12f polypeptide; and
(ii) a guide sequence capable of hybridizing to a target sequence of a target DNA, thereby guiding the complex to the target DNA.

In yet another aspect, the disclosure provides polynucleotide encoding the Cas12f polypeptide of the disclosure.

In yet another aspect, the disclosure provides delivery system comprising (1) the Cas12f polypeptide of the disclosure, the polynucleotide of the disclosure, or the system of the disclosure; and (2) a delivery vehicle.

In yet another aspect, the disclosure provides vector comprising the polynucleotide of the disclosure; optionally wherein the vector encodes a guide nucleic acid as defined in the disclosure; optionally wherein the vector is a plasmid vector, a recombinant AAV (rAAV) vector, or a recombinant lentivirus vector.

In yet another aspect, the disclosure provides ribonucleoprotein (RNP) comprising the Cas12f polypeptide of the disclosure and a guide nucleic acid optionally as defined in the disclosure.

In yet another aspect, the disclosure provides lipid nanoparticle (LNP) comprising the Cas12f polypeptide of the disclosure 9 or the system of the disclosure.

In yet another aspect, the disclosure provides method for modifying a target DNA, comprising contacting the target DNA with the system of the disclosure, the vector of the disclosure, the ribonucleoprotein of the disclosure, or the lipid nanoparticle of the disclosure, wherein the spacer sequence is capable of hybridizing to a target sequence of the target DNA, wherein the target DNA is modified by the complex.

In yet another aspect, the disclosure provides cell modified by the method of the disclosure.

In yet another aspect, the disclosure provides pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient.

In yet another aspect, the disclosure provides method for diagnosing, preventing, or treating a disease in a subject in need thereof, comprising administering to the subject the system of the disclosure, the vector of claim 29, the ribonucleoprotein of the disclosure, the lipid nanoparticle of the disclosure, the cell of the disclosure, or the pharmaceutical composition of the disclosure, wherein the disease is associated with a target DNA, wherein the spacer sequence is capable of hybridizing to a target sequence of the target DNA, wherein the target DNA is modified by the complex, and wherein the modification of the target DNA diagnose, prevents, or treats the disease.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other aspect or embodiment of the disclosure to constitute another embodiment explicitly or implicitly disclosed herein unless otherwise indicated.

### Overview

Cas12f, as a subtype of Class 2, Type V CRISPR associated protein (Cas12), is capable of binding to or function on a target nucleic acid (e.g., a dsDNA) as guided by a guide nucleic acid (e.g., a guide RNA (gRNA, used interchangeably with single guide RNA or sgRNA in the disclosure)) comprising a guide sequence targeting the target nucleic acid. In some embodiments, the target nucleic acid is eukaryotic.

Without wishing to be bound by theory, in some embodiments, the guide nucleic acid comprises a scaffold sequence responsible for forming a complex with the Cas12f, and a guide sequence (used interchangeable with a spacer sequence in the disclosure) that is intentionally designed to be responsible for hybridizing to a target sequence of the target nucleic acid, thereby guiding the complex comprising the Cas12f and the guide nucleic acid to the target nucleic acid.

Referring to FIG. 24, an exemplary target dsDNA (e.g., a target gene) is depicted to comprise a 5′ to 3′ single DNA strand and a 3′ to 5′ single DNA strand.

An exemplary guide nucleic acid is depicted to comprise a guide sequence and a scaffold sequence. The guide sequence is designed to hybridize to a part of the 3′ to 5′ single DNA strand, and so the guide sequence “targets” that part. And thus, the 3′ to 5′ single DNA strand is referred to as a “target strand (TS)” of the target dsDNA, while the opposite 5′ to 3′ single DNA strand is referred to as a “nontarget strand (NTS)” of the target dsDNA. That part of the target strand based on which the guide sequence is designed and to which the guide sequence may hybridize is referred to as a “target sequence”, while the opposite part on the nontarget strand corresponding to that part is referred to as the “protospacer sequence”, which is 100% (fully) reversely complementary to the target sequence.

Generally, a nucleic acid sequence (e.g., a DNA sequence, an RNA sequence) is written in 5′ to 3′ direction/orientation.

For example, for a DNA sequence of ATGC, it is usually understood as 5′-ATGC-3′ unless otherwise indicated. Its reverse sequence is 5′-CGTA-3′, its fully complement sequence is 5′-TACG-3′, and its fully reverse complement sequence is 5′-GCAT-3′.

Generally, the double-strand sequence of a dsDNA may be represented with the sequence of its 5′ to 3′ single DNA strand conventionally written in 5′ to 3′ direction/orientation unless otherwise indicated.

For example, for a dsDNA having a 5′ to 3′ single DNA strand of 5′-ATGC-3′ and a 3′ to 5′ single DNA strand of 3′-TACG-5′, the dsDNA may be simply represented as 5′-ATGC-3′.

5′ ----- ATGC ----- 3′
3′ ----- TACG ----- 5′

It should be noted that either the 5′ to 3′ single DNA strand or the 3′ to 5′ single DNA strand of a dsDNA can be a nontarget strand from which a protospacer sequence is selected or a target strand to which the guide sequence is designed to hybridize.

Generally, for a gene as a dsDNA, the 5′ to 3′ single DNA strand is the sense strand of the gene, and the 3′ to 5′ single DNA strand is the antisense strand of the gene. But it should be noted that either the sense strand or the antisense strand of a gene can be a nontarget strand from which a protospacer sequence is selected or a target strand to which the guide sequence is designed to hybridize.

To hybridize to a target dsDNA, in one embodiment, the guide sequence of a guide nucleic acid (e.g., a guide RNA) is designed to have a RNA sequence of 5′-AUGC-3′ that is fully reversely complementary to the 3′ to 5′ strand of the target dsRNA, which would be set forth in ATGC in the electric sequence listing but annotated as RNA; and in another embodiment, the guide sequence of a guide nucleic acid (e.g., a guide RNA) is designed to have a RNA sequence of 5′-GCAU-3′ that is fully reversely complementary to the 5′ to 3′ strand of the target dsRNA, which would be set forth in GCAT in the electric sequence listing but annotated as RNA.

In the case that the guide sequence of a guide nucleic acid is fully reversely complementary to the target sequence and the target sequence is fully reversely complementary to the protospacer sequence, the guide sequence is identical to the protospacer sequence except for the U in the guide sequence if it is an RNA sequence and correspondingly the T in the protospacer sequence. According to WIPO standard ST.26, symbol “t” is used to denote both T in DNA and U in RNA (See “Table 1: List of nucleotides symbols”, the definition of symbol “t” is “thymine in DNA/uracil in RNA (t/u)”). Thus, in the sequence listing of the disclosure prepared according to ST.26, such a guide sequence could be set forth in the same sequence as a corresponding protospacer sequence. For convenience, a single SEQ ID NO in the sequence listing can be used to denote both such guide sequence and protospacer sequence, although such a single SEQ ID NO may be marked as either DNA or RNA in the sequence listing. When a reference is made to such a SEQ ID NO that sets forth a protospacer/guide sequence, it refers to either a protospacer sequence that is a DNA sequence or a guide sequence that may be an RNA sequence depending on the context, no matter whether it is marked as DNA or RNA in the sequence listing.

### Term

Unless otherwise specified, all technical and scientific terms used in the disclosure have the meaning commonly understood by one of ordinary skill in the art to which the disclosure belongs. Throughout the specification, several terms are employed that are defined in the following paragraphs. Other definitions are also found within the body of the specification.

As used herein, the terms “nucleic acid”, “nucleic acid molecule”, or “polynucleotide” are used interchangeably. They refer to a polymer of deoxyribonucleotides or ribonucleotides or their mixtures in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “polypeptide” and “protein” are used interchangeably to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component.

As used herein, a “fusion protein” refers to a protein created through the joining of two or more originally separate proteins, or portions thereof. In some embodiments, a linker may be present between each protein.

As used herein, the term “heterologous,” in reference to polypeptide domains, refers to the fact that the polypeptide domains do not naturally occur together (e.g., in the same polypeptide). For example, in fusion proteins generated by the hand of man, a polypeptide domain from one polypeptide may be fused to a polypeptide domain from a different polypeptide. The two polypeptide domains would be considered “heterologous” with respect to each other, as they do not naturally occur together.

As used herein, the term “nuclease” refers to a polypeptide capable of cleaving the phosphodiester bonds between the nucleotide subunits of nucleic acids; the term “endonuclease” refers to a polypeptide capable of cleaving the phosphodiester bond within a polynucleotide chain.

As used herein, the term “Cas12f” is used interchangeably with Cas12f protein or Cas12f polypeptide in the disclosure and used in its broadest sense and includes parental or reference Cas12f proteins (e.g., Cas12f protein comprising any of SEQ ID NOs: 1-34), derivatives or variants thereof, and functional fragments such as nucleic acid-binding fragments thereof, including endonuclease deficient (dead) Cas12f polypeptides, and Cas12f nickases.

As used herein, the term “guide nucleic acid” refers to a nucleic acid-based molecule capable of forming a complex with a CRISPR-Cas protein (e.g., a Cas12f of the disclosure) (e.g., via a scaffold sequence of the guide nucleic acid), and comprises a sequence (e.g., guide sequences) that are sufficiently complementary to a target nucleic acid to hybridize to the target nucleic acid and guide the complex to the target nucleic acid, which include but are not limited to RNA-based molecules, e.g., guide RNA. As used herein, the term “single guide RNA (sgRNA)” is used interchangeably with guide RNA (gRNA) or RNA guide. As used in the disclosure, the term “guide sequence” is used interchangeably with the term “spacer sequence”. The guide nucleic acid may be a DNA molecule, an RNA molecule, or a DNA/RNA mixture molecule. By “DNA/RNA mixture molecule” it refers to a nucleic acid comprising both one or more modified or unmodified ribonucleotides and one or more modified or unmodified deoxyribonucleotides, whether consecutive or not. However, by “DNA molecule” or “RNA molecule” it may also refer to a DNA molecule containing one or more modified or unmodified ribonucleotides, whether consecutive or not, or an RNA molecule containing one or more modified or unmodified deoxyribonucleotides, whether consecutive or not.

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a Cas12f polypeptide). As used herein, the term “complex” can refer to a grouping of a guide nucleic acid, a polypeptide, and a target nucleic acid.

As used herein, the term “activity” refers to a biological activity. In some embodiments, the activity includes enzymatic activity, e.g., catalytic ability of an effector. For example, the activity can include nuclease activity, e.g., DNA nuclease activity, dsDNA endonuclease activity, guide sequence-specific (on-target) dsDNA endonuclease activity, guide sequence-independent (off-target) dsDNA endonuclease activity.

As used herein, the term “guide sequence-specific (on-target) dsDNA cleavage” may be termed as “dsDNA cleavage” for short unless otherwise indicated.

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or cohesive ends.

As used herein, the meanings of “cleaving a nucleic acid” or “modifying a nucleic acid” may overlap. Modifying a nucleic acid includes not only modification of a mononucleotide but also insertion or deletion of a nucleic acid fragment.

As used herein, the term “on-target” refers to binding, cleavage, and/or editing of an intended or expected region of DNA, for example, by Cas12f of the disclosure.

As used herein, the term “off-target” refers to binding, cleavage, and/or editing of an unintended or unexpected region of DNA, for example, by Cas12f of the disclosure. In some embodiments, a region of DNA is an off-target region when it differs from the region of DNA intended or expected to be bound, cleaved and/or edited by 1, 2, 3, 4, 5, 6, 7, 8, 9, 10 or more nucleotides.

As used herein, if a DNA sequence, for example, 5′-ATGC-3′ is transcribed to an RNA sequence, with each dT (deoxythymidine, or “T” for short) in the primary sequence of the DNA sequence replaced with a U (uridine) and each dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “protospacer adjacent motif’ or “PAM” refers to a short sequence (or a motif) adjacent to a protospacer sequence on the nontarget strand of a dsDNA recognized by CRISPR complexes.

As used herein, the term “adjacent” includes instances wherein there is no nucleotide between the protospacer sequence and the PAM and also instances wherein there are a small number (e.g., 1, 2, 3, 4, or 5) of nucleotides between the protospacer sequence and the PAM. As used herein, A “immediately adjacent (to)” B, A “immediately 5′ to” B, and A “immediately 3′ to” B mean that there is no nucleotide between A and B.

As described herein, the guide sequence is so designed to be capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the one or more polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit a Cas12f polypeptide that is complexed with a guide nucleic acid comprising the guide sequence or a function domain (e.g., a deaminase domain) associated (e.g., fused) with the Cas12f polypeptide to act (e.g., cleave, deaminize) at or near the target sequence or its complement (e.g., a sequence of a target DNA or its complement).

For the purpose of hybridization, in some embodiments, the guide sequence is reversely complementary to a target sequence. As used herein, the term “complementary” refers to the ability of nucleobases of a first polynucleotide sequence, such as a guide sequence, to base pair with nucleobases of a second polynucleotide sequence, such as a target sequence, by traditional Watson-Crick base-pairing. Two complementary polynucleotide sequences are able to non-covalently bind under appropriate temperature and solution ionic strength conditions. In some embodiments, a first polynucleotide sequence (e.g., a guide sequence) comprises 100% (fully) complementarity to a second nucleic acid (e.g., a target sequence). In some embodiments, a first polynucleotide sequence (e.g., a guide sequence) is complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide sequence (e.g., a guide sequence) that has a certain level of complementarity to a second polynucleotide sequence (e.g., a target sequence) such that the first polynucleotide sequence (e.g., a guide sequence) can hybridize to the second polynucleotide sequence (e.g., a target sequence) with sufficient affinity to permit a Cas12f polypeptide that is complexed with the first polynucleotide sequence or a nucleic acid comprising the first polynucleotide sequence or a function domain associated (e.g., fused) with the Cas12f polypeptide to act (e.g., cleave, deaminize) on the target sequence or its complement (e.g., a sequence of a target DNA or its complement). In some embodiments, a guide sequence that is substantially complementary to a target sequence has 100% or less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence.

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. The comparison of sequences and determination of percent identity between two sequences can be accomplished using a mathematical algorithm. As is well known in the art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, the sequence identity is calculated by global alignment, for example, using the Needleman-Wunsch algorithm and an online tool at ebi.ac.uk/Tools/psa/emboss_needle/. In some embodiments, the sequence identity is calculated by local alignment, for example, using the Smith-Waterman algorithm and an online tool at ebi.ac.uk/Tools/psa/emboss_water/.

As used herein, the term “variant” refers to an entity that shows significant structural identity with a reference entity (e.g., a wild-type sequence) but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprising a plurality of amino acids having designated positions relative to one another in linear or three-dimensional space and/or contributing to a particular biological function; a nucleic acid may have a characteristic sequence element comprising a plurality of nucleotide residues having designated positions relative to one another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide (e.g., a nuclease described herein) that is at least 60%, 65%, 70%, 75%, 80%, 85%, 86%, 87%, 88%, 89%, 90%, 91%, 92%, 93%, 94%, 95%, 96%, 97%, 98% or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide, e.g., nuclease activity. In some embodiments, a variant polypeptide lacks one or more of the biological activities of the reference polypeptide. In some embodiments, a variant polypeptide shows a reduced level of one or more biological activities (e.g., nuclease activity, e.g., off-target nuclease activity) as compared with the reference polypeptide. In some embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2%, or 1% of the residues in the variant are substituted as compared with the parent or reference polypeptide. In some embodiments, a variant has 10, 9, 8, 7, 6, 5, 4, 3, 2, or 1 substituted residue as compared with a parent or reference polypeptide. Often, a variant has a very small number (e.g., fewer than 5, 4, 3, 2, or 1) of substituted functional residues (i.e., residues that participate in a particular biological activity). In some embodiments, a variant has not more than 5, 4, 3, 2, or 1 additions or deletions, and often has no additions or deletions, as compared with the parent or reference polypeptide. Moreover, any additions or deletions are typically fewer than about 25, about 20, about 19, about 18, about 17, about 16, about 15, about 14, about 13, about 12, about 11, about 10, about 9, about 8, about 7, about 6, and commonly are fewer than about 5, about 4, about 3, or about 2 residues. In some embodiments, the parent or reference polypeptide is a wild type. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

Conservative substitutions of non-critical amino acids of a protein may be made without affecting the normal functions of the protein. Conservative substitutions refer to the substitution of amino acids with chemically or functionally similar amino acids. In some embodiments, a conservative amino acid substitution refers to an amino acid substitution that does not alter the relative charge or size characteristics of the protein in which the amino acid substitution was made. In some embodiments, a “conservative substitution” refers to a substitution of an amino acid made among amino acids within the following groups: i) methionine, isoleucine, leucine, valine, ii) phenylalanine, tyrosine, tryptophan, iii) lysine, arginine, histidine, iv) alanine, glycine, v) serine, threonine, vi) glutamine, asparagine and vii) glutamic acid, aspartic acid.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the description of “a variant (e.g., a Cas12f polypeptide) comprising an amino acid mutation (e.g., substitution) at a given position (e.g., position 52) of a given polypeptide (e.g., SEQ ID NO: 1)” or similar description means that the polypeptide as set forth in the amino acid sequence of the given polypeptide serves as a parent or reference polypeptide, and the variant is a variant of the parent or reference polypeptide and comprises an amino acid mutation at a position of the amino acid sequence of the variant corresponding to the given position of the amino acid sequence of the given polypeptide. The position of the amino acid mutation in the amino acid sequence of the variant may be the same as the given position of the given polypeptide, for example, when the variant comprises just an amino acid substitution as compared with the given polypeptide and has the same length as the given polypeptide. The position of the amino acid mutation in the amino acid sequence of the variant may also be different from the given position of the given polypeptide, for example, when the variant comprises a N-terminal truncation as compared with the given polypeptide and the first N-terminal amino acid of the variant is not corresponding to the first N-terminal amino acid of the given polypeptide but to an amino acid within the given polypeptide, but the position of the amino acid mutation can be determined by alignment of the variant and the given polypeptide to identify the corresponding amino acids in their sequences as understood by a skilled in the art. For example, if the variant has a N-terminal truncation of 20 amino acids as compared with the given polypeptide, then the variant comprising an amino acid mutation at position 52 of a given polypeptide means that the variant comprises an amino acid mutation at position 32 of the variant since position 32 in the variant is corresponding to position 52 in the given polypeptide as determined by alignment of the variant and the given polypeptide.

As used herein, the description of “a variant (e.g., a Cas12f polypeptide) comprising a given amino acid substitution (e.g., D52R) relative to a given polypeptide (e.g., SEQ ID NO: 1)” means that the polypeptide as set forth in the amino acid sequence of the given polypeptide serves as a parent or reference polypeptide that does not comprise the given amino acid substitution, and the variant is a variant of the parent or reference polypeptide and comprises an amino acid substitution having the same type of substitution as the given amino acid substitution and at a position in the amino acid sequence of the variant corresponding to the position of the given amino acid substitution. For example, a Cas12f polypeptide comprising an amino acid substitution D52R relative to SEQ ID NO: 1 refers to the fact that the amino acid sequence of SEQ ID NO: 1 comprises amino acid D at position 52, and the Cas12f polypeptide comprises amino acid R at a position corresponding to position 52 of the amino acid sequence of SEQ ID NO: 1. The corresponding relationship of positions in two amino acid sequences as determined by alignment is explained in the previous paragraph.

As used herein, the terms “upstream” and “downstream” refer to relative positions within a single nucleic acid (e.g., DNA) sequence in a nucleic acid. “Upstream” and “downstream” relate to the 5′ to 3′ direction, respectively, in which transcription occurs. For a first sequence and a second sequence present on the same strand of a single nucleic acid written in 5′ to 3′ direction, the first sequence is upstream of the second sequence when the 3′ end of the first sequence is on the left side of the 5′ end of the second sequence, and the first sequence is downstream of the second sequence when the 5′ end of the first sequence is on the right side of the 3′ end of the second sequence. For example, a promoter is usually at the upstream of a sequence under the regulation of the promoter; and on the other hand, a sequence under the regulation of a promoter is usually at the downstream of the promoter.

As used herein, the term “regulatory element” refers to a DNA sequence that controls or impacts one or more aspects of transcription and/or expression is intended to include promoters, enhancers, silencers, termination signals, internal ribosome entry sites (IRES), and other expression control elements (e.g., transcription termination signals such as polyadenylation signals and poly-U sequences). Regulatory elements include those that direct constitutive expression of a nucleotide sequence in many types of host cells and those that direct expression of a nucleotide sequence only in certain host cells (e.g., tissue-specific regulatory sequences). Regulatory elements may also direct expression in a time-dependent manner, e.g., in a cell cycle-dependent or developmental stage-dependent manner, which may or may not be tissue or cell type specific.

As used herein, the term “operably linked” refers to a juxtaposition wherein the components described are in a relationship permitting them to function in their intended manner. A regulatory element “operably linked” to a functional element is associated in such a way that transcription, expression, and/or activity of the functional element is achieved under conditions compatible with the regulatory element. In some embodiments, “operably linked” regulatory elements are contiguous (e.g., covalently linked) with the functional elements of interest; in some embodiments, regulatory elements act in trans to or otherwise at a distance from the functional elements of interest.

As used herein, the term “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in vivo” means inside the body of an organism, and the terms “ex vivo” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., preventing or delaying the worsening of a disease), preventing or delaying the spread (e.g., metastasis) of a disease, preventing or delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and prolonging survival. Also encompassed by the term is a reduction of pathological consequence of a disease (such as cancer). The methods of the disclosure contemplate any one or more of these aspects of treatment.

As used herein, the term “disease” includes the terms “disorder” and “condition” and is not limited to those specific diseases that have been medically or clinically defined.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise. That is, articles “a/an” and “the” are used herein to refer to one or more than one (i.e., at least one) grammatical object of the article. For example, “an element” means one element or more than one element, e.g., two elements.

As used herein, the term “and/or” in a phrase such as “A and/or B” is intended to mean either or both of the alternatives, including both A and B, A or B, A (alone), and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” used herein has the same meaning as “about X to about Y.”

As used herein, the terms “about” and “approximately,” in reference to a number, is used herein to include numbers that fall within a range of 20%, 15%, 10%, 9%, 8%, 7%, 6%, 5%, 4%, 3%, 2%, or 1% in either direction (greater than or less than) of the number unless otherwise stated or otherwise evident from the context (except where such number would exceed 100% of a possible value).

As used herein, a numerical range includes the end values of the range, and each specific value within the range, for example, “16 to 100 nucleotides” includes 16 nucleotides and 100 nucleotides, and each specific value between 16 and 100, e.g., 17, 23, 34, 52, 78.

As used herein, the terms “comprise”, “include”, “contain”, and “have” are to be understood as implying that a stated element or a group of elements is included, but not excluding any other element or a group of elements, unless the context requires otherwise. In certain embodiments, the terms “comprise”, “include”, “contain”, and “have” are used synonymously.

As used herein, the phrase “consist essentially of” is intended to include any element listed after the phrase “consist essentially of” and is limited to other elements that do not interfere with or contribute to the activities or actions specified in the disclosure of the listed elements. Thus, the phrase “consist essentially of” is intended to indicate that the listed elements are required, but no other elements are optional, and may or may not be present depending on whether they affect the activities or actions of the listed elements.

As used herein, the phrase “consist of” means including but limited to any element after the phrase “consist of”. Thus, the phrase “consist of” indicates that the listed elements are required, and that no other elements can be present.

As used herein, the term “comprises” also encompasses the terms “consists essentially of” and “consists of”. It is understood that the “comprising” embodiments of the disclosure described herein also include “consisting essentially of” and “consisting” embodiments.

Throughout the specification, reference to “one embodiment”, “embodiment”, “a specific embodiment”, “a related embodiment”, “an embodiment”, “another embodiment”, or “a further embodiment” or a combination thereof means that specific elements, features, structures, or characteristics described in connection with the embodiment are included in at least one embodiment of the disclosure. Accordingly, the appearances of the foregoing phrases in various places throughout the specification are not necessarily all referring to the same embodiments. Furthermore, specific elements, features, structures, or characteristics may be combined in any suitable manner in one or more embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIGS. 1A-1F: Identification and characterization of CRISPR loci and Cas proteins of Class 2, Type V-F CRISPR systems. FIG. 1A, Maximum-likelihood tree of identified Cas12f1 and previously reported Cas12f1. The evolutionary distance scale of 0.08 is shown. FIG. 1B, Scheme of Cas12f1-induced EGFP activation in HEK293T cells. Transfection of plasmids expressing Cas12f1 and sgRNA activated EGFP. FIG. 1C, Various Cas12f1 mediated EGFP activation efficiency determined by flow cytometry. Values and error bars represent mean and s.d. (n=3). The two most efficient Cas12f1s selected for further study were highlighted in red. FACS gating strategy shown in Supplementary FIG. 7e. FIG. 1D, Protein organization of SpCas9, LbCas12a, Un1Cas12f1_ge4.1, OsCas12f1, and RhCas12f1. Nuclease domains including RuvC and HNH, as well as protein length are indicated. FIG. 1E, Comparison of DNA sequence size of OsCas12f1, RhCas12f1, and other commonly used CRISPR systems. FIG. 1F, WebLogos of the PAM sequences for OsCas12f1 and RhCas12f1.

FIGS. 2A-2K: Rational protein engineering and sgRNA optimization for high-efficiency Cas12f1. FIG. 2A, Scheme of protein engineering strategy. Mutants showing higher EGFP activation were selected for further optimization. FIG. 2B, The first round high-efficiency mutant screen of OsCas12f1. The wild-type OsCas12f1 (WTOsCas12f1), Un1Cas12f1_ge4.1, SpCas9, and the mutant selected for next round screen are indicated. FIG. 2C, Second round enhanced OsCas12f1 variants screen by combining D52R with other arginine substitution mutants. Values and error bars represent mean and s.d. (n=3). FIG. 2D, Engineering strategy for OsCas12f1 sgRNA. FIG. 2E, results of sgRNA engineering for OsCas12f1. The optimal sgRNA (Os-sg1.1) chosen for further engineering is marked in red. Values and error bars represent mean and s.d. (n=3). FIG. 2F, Second round sgRNA engineering by including C-G base pair substitution on Os-sg1.1. Values and error bars represent mean and s.d. (n=3). FIG. 2G, Increased EGFP activation efficiency by combining OsCas12f1 mutant (T132R+D52R) and sgRNA variant (Os-sg2.6). enOsCas12f1 is indicated with red triangle. Values and error bars represent mean and s.d. (n=3). FIG. 2H, Enhanced mutant screen of RhCas12f1. Each dot indicates one mutant. FIGS. 2I and 2J, Optimization of RhCas12f1 sgRNA to increase activity. The marked sgRNA (Rh-sg1.1) was selected for further optimization. Values and error bars represent mean and s.d. (n=3). FIG. 2K, Combination of Rh-sg1.1 variant with protein variant further increased the EGFP activation efficiency of Rh-sg1.1 variant. The best combination is indicated as enRhCas12f1 marked with red triangle. Values and error bars represent mean and s.d. (n=3).

FIGS. 3A-3F: PAM preferences of enOsCas12f1 and enRhCas12f1. FIG. 3A, PAM preferences of OsCas12f1, enOsCas12f1 and Un1Cas12f1_ge4.1 analyzed by GFP activation system of Example 1. Values and error bars represent mean and s.d. (n=3). FIG. 3B, Comparison of RhCas12f1- and enRhCas12f1-preferred PAM. Values and error bars represent mean and s.d. (n=3). FIGS. 3C and 3D, Validation of the PAM preferences of enOsCas12f, enRhCas12f1, and Un1Cas12f1_ge4.1 at endogenous loci. Values and error bars represent mean and s.d. (n=3). FIGS. 3E and 3F, Summary of indel efficiencies of enOsCas12f1, Un1Cas12f1_ge4.1, and enRhCas12f1. Values and error bars represent mean and s.d. from biologically independent experiments.

FIGS. 4A-4F: Comprehensive validation of genomic editing efficiency of enOsCas12f1 and enRhCas12f1 in human cells. FIG. 4A, Distribution of all exon-located target sites that are accessible for enOsCas12f1 (5′-NTTC PAM), enRhCas12f1 (5′-CCCA PAM), and Un1Cas12f1_ge4.1 (5′-TTTR PAM), and the indel frequencies are indicated by mean values of three replicates, as determined by NGS. The exon (gray solid squares) is connected by intron (lines), and UTRs are shown as hollow boxes. FIG. 4B, Indel frequencies of enOsCas12f1, enRhCas12f1, and Un1Cas12f1_ge4.1 at endogenous genomic loci. Each dot represents a single target site, and each value means an average of three replicates. Bars represent means. FIG. 4C, Comparison of editing efficiencies of enOsCas12f1 and Un1Cas12f1_ge4.1 targeted by same sgRNAs at PCSK9 and TTR loci. Values and error bars represent mean and s.d. (n=3). FIG. 4D, Average indel frequency of enOsCas12f1 and Un1Cas12f1_ge4.1 at 5′-TTC PAM and 5′-TTTR PAM target sites. Each dot represents a single target site, and each value means an average of 3 replicates. Error bars represent mean and s.d. FIG. 4E, Comparison of editing efficiencies of enOsCas12f1 and SpG targeted by same sgRNAs at endogenous target loci. Values and error bars represent mean and s.d. (n=3). FIG. 4F, The distribution of mutant alleles by enOsCas12f1-mediated disruption at TTR locus, and the top 10 mutant alleles are represented.

FIGS. 5A-5E: Specificities of enOsCas12f1- and enRhCas12f1-mediated genome editing in human cells. FIG. 5A, Effects of 1 bp or 2 bp mismatches in sgRNA on activities of enOsCas12f1 at PCSK9 locus. Values and error bars represent mean and s.d. (n=3). FIG. 5B, Mismatch tolerance of enRhCas12f1 at PCSK9-sg32. Values and error bars represent mean and s.d. (n=3). FIG. 5C, Off-target efficiency of LbCas12a, enOsCas12f1, and Un1Cas12f1_ge4.1 at in silico predicted off-target sites, determined by targeted deep sequencing. FIGS. 5D and 5E, PEM-seq genome-widely quantified the translocation efficiencies induced by off-target indels by enOsCas12f1 and enRhCas12f1. Circos plot shows the off-target sites that were linked to the bait DSB (red triangle, FIG. 5D). Percentages of translocation, germline, and editing efficiency calculated by PEM-seq analysis of enOsCas12f1, Un1Cas12f1_ge4.1, LbCas12a, SpCas9, and enRhCas12f1 (FIG. 5E).

FIGS. 6A-6M: Tunable enOsCas12f1-mediated in vitro and in vivo deletion of human DMD exon 51 and engineering enOsCas12f1 for epigenome editing and gene activation. FIG. 6A, Strategy for generating humanized DMD mutation mouse with human exon 51 replacement and exon 52 deletion. Deletion of exon 51 can restore dystrophin expression. Two sgRNAs located before (5′ sgRNA) and after (3′ sgRNA) exon 51 are designed to delete exon 51. FIG. 6B, enOsCas12f1- and SpCas9-mediated deletion of DMD exon 51 by paired sgRNAs in HEK293T cells. The exon 51 deletion bands were marked by red asterisk. This experiment was repeated two times, showing similar results. FIG. 6C, Scheme representing the strategy for destabilized enOsCas12f1 (DD-enOsCas12f1). FIG. 6D, Overview of intramuscular injection of single AAV9 system in humanized mouse. FIG. 6E, The in vivo editing efficiencies of enOsCas12f1 and DD-enOsCas12f1 were tested by genomic PCR. This experiment was repeated two times, showing similar results. FIG. 6F, Western blotting for detecting recovery of dystrophin (DMD) by enOsCas12f1 and DD-enOsCas12f1 in DMD model mouses. Vinculin (VCL) protein level was used as internal control. FIG. 6G, Percentage of recovered dystrophin by western blotting analysis. Values and error bars represent mean and s.d. (n=1 for KO, n=6 for enOsCas12f1). FIG. 6H, DMD immunofluorescence staining. FIG. 6I, Percentage of dystrophin positive fibers in enOsCas12f1 and DD-enOsCas12f1 treated muscles. Values and error bars represent mean and s.d. (n=3). FIG. 6J, GFP silencing activity of miniCRISPRoff-v1˜v4 and CRISPRoff-v2. The stably GFP expressing HEK293T cells generated by piggyBac system were used. Values and error bars represent mean and s.d. (n=3). FIG. 6K, DNA methylation level on the Snrp promoter region. FIG. 6L, Design strategy for denOsCas12f1-VPR adopted from Xu et al. The TRE3G-GFP reporter cell line was created by piggyBac system in HEK293T cells. FIG. 6M, GFP activation efficiencies of denOsCas12f1-VRP. sgRNA containing random non-targeting spacer sequence served as non-target (NT) control. Values and error bars represent mean and s.d. (n=3).

FIGS. 7A-7F. Strategy for flow cytometry gating and Cas12f1 candidate prediction. FIG. 7A, Scheme representing native CRISPR-Cas loci encoding OsCas12f1 and RhCas12f1. FIG. 7B, Predicted tracrRNA structure by RNAfold. FIGS. 7C and 7D, In silico prediction of base paring between tracrRNA and crRNA of OsCas12f1 (7c) and RhCas12f1 (7d). FIG. 7E, Gating strategy used for evaluating EGFP activation efficiency. Gate set on the non-targeting control was used to analyze the EGFP activation efficiency of targeting group. FIG. 7F, Screen for functional Cas12f1 in HEK293T cells. Values and error bars represent mean and s.d. (n=3).

FIGS. 8A-8E. Efficiency validation of genome editing by Cas12f1 in human cells. FIGS. 8A-8E, Indel efficiencies at endogenous genes in HEK293T cells as determined by TIDER. All values and error bars represent mean and s.d. from n=2 biologically independent experiments.

FIGS. 9A-9E. Optimal parameter sets of OsCas12f1 and RhCas12f1. FIGS. 9A and 9B, Optimal spacer length for OsCas12f1 (FIG. 9A) and RhCas12f1 (FIG. 9B). Values and error bars represent mean and s.d. (n=3). FIG. 9C, Alignment of OsCas12f1 and RhCas12f1 with Un1Cas12f1 to identify the conserved residues of RuvC active site, which is marked by red box. FIGS. 9D and 9E, Validation of the enzymatic activity sites of OsCas12f1 (FIG. 9D) and RhCas12f1 (FIG. 9E). TTTC-CCATTACAGTAGGAGCATAC (SEQ ID NO: 214) and CCCA-CCATTACAGTAGGAGCATAC (SEQ ID NO: 214) targeting spacer sequences with respective PAMs were used for assessing the GFP activation efficiencies of OsCas12f1 and RhCas12f1, respectively. Values and error bars represent mean and s.d. (n=3).

FIGS. 10A-10E. Characterization of OsCas12f1- and RhCas12f1-mediated cleavage. FIG. 10A, SDS-PAGE analysis of purified OsCas12f1, RhCas12f1, enOsCas12f1, and enRhCas12f1 proteins. FIG. 10B, Linear plasmids cleavage at different temperature by OsCas12f1 and RhCas12f1. FIG. 10C, OsCas12f1 and RhCas12f1 cut both supercoiled and linear plasmids in vitro. FIGS. 10C and 10E, Run-off sequencing of OsCas12f1- (FIG. 10D) and RhCas12f1- (FIG. 10E) cleaved products. Red triangles indicate the cleavage sites. These experiments were repeated at least two times, showing similar results.

FIGS. 11A-11B. OsCas12f1-sgRNA and RhCas12f1-sgRNA complex formation. FIGS. 11A and 11B, Size-exclusion chromatography profiles of OsCas12f1 (FIG. 11A) and RhCas12f1 (FIG. 11B) with or without its sgRNA. UV absorbance at 280 nm and 260 nm were shown in solid and dashed lines, respectively. The molecular weights of standard marker proteins are indicated. Both OsCas12f1 and RhCas12f1 could form dimer with its sgRNA, which was indicated by pink and blue arrows, respectively, at least under the test conditions. The peak fractions were analyzed by SDS-PAGE. These experiments were repeated three times, showing similar results.

FIGS. 12A-12B. Protein alignment of OsCas12f1 and RhCas12f1 with Un1Cas12f1. FIG. 12A, Predicted domain architecture of OsCas12f1 and RhCas12f1 by alignment with Un1Cas12f1. ZF, zinc finger domain; REC, recognition domain; WED, wedge domain; RuvC, RuvC nuclease domain; TNB, target nucleic acid-binding domain. The maximum-likelihood regions of OsCas12f1 and RhCas12f1 for RNA and/or DNA recognition (region1˜3) are indicated. FIG. 12B, protein alignment of OsCas12f1, RhCas12f1, and Un1Cas12f1.

FIG. 13. Mutagenesis strategy for screening of enOsCas12f1 and enRhCas12f1. enOsCas12f1 was shown as an example. Region1˜3 of OsCas12f1 were divided into 11 segments containing 17 amino acid residues in length. Eleven backbone mutants of OsCas12f1 were generated by replacing the above mentioned segments with BpiI recognition sequence by PCR and Gibson assembly method using NEBuilder HiFi DNA Assembly Master Mix (New England Biolabs). The specific mutation was then introduced by incorporation of annealed oligos containing the mutation by BpiI digestion and T4 DNA ligase ligation.

FIGS. 14A-14I. Engineering and optimization of enCas12f1. FIG. 14A, Saturation mutagenesis analysis at D52 site of OsCas12f1. Data shown here represents values of results from n=1 experiment. FIG. 14B, Evaluating the efficiency of OsCas12f1-D52R+Os-sg1.1 combination variant. Target: TTTC-CCATTACAGTAGGAGCATAC (SEQ ID NO: 214). Values and error bars represent mean and s.d. (n=3). FIG. 14C, Increased EGFP activation efficiency by Os-sg2.6. Target: TTTC-CCATTACAGTAGGAGCATAC (SEQ ID NO: 214). Values and error bars represent mean and s.d. (n=3). FIGS. 14D-14F, Validation of OsCas12f1-D52R+Os-sg2.6 by reporter containing different endogenous protospacer sequences. Target-d: CTTC-TTGTGCTGGACGGTGACGTA (SEQ ID NO: 511); target-e: TTTC-ATTGGCTTTGATTTCCCTAG (SEQ ID NO: 486); target-f: TTTC-CCTAGGGTCCAGCTTCAAAT (SEQ ID NO: 512). Values and error bars represent mean and s.d. (n=3). FIG. 14G, Increased EGFP activation efficiency of enOsCas12f variant by combining OsCas12f1 mutant and sgRNA variant. The best combination is represented as enOsCas12f1. Reporter containing DMD_2 protospacer sequence (SEQ ID NO: 487) was used. Values represent mean of results from n=2 biologically independent experiments. FIG. 14H, Efficiency of enOsCas12f1 at endogenous DMD locus in HEK293T cells. Values and error bars represent mean and s.d. (n=3). FIG. 14I, Efficiency of enRhCas12f1 at endogenous PCSK9 locus in HEK293T cells. Values and error bars represent mean and s.d. (n=3).

FIGS. 15A-15B. In vitro PAM preferences of enOsCas12f1 and enRhCas12f1. WebLogos of the in vitro PAM sequences for enOsCas12f1 (FIG. 15A) and enRhCas12f1 (FIG. 15B).

FIGS. 16A-16D. enRhCas12f1-mediated gene disruption in human cells. FIG. 16A, Size and position distribution of indels induced by enOsCas12f1. FIG. 16B, The top 10 mutant alleles by enRhCas12f1 mediated disruption at PCSK9 locus. FIGS. 16C-16D, Size and position distribution of indels induced by enRhCas12f1.

FIG. 17. Mismatch tolerance of enOsCas12f1 and enRhCas12f1. Impact of 1 bp mismatched sgRNA on the GFP activation efficiencies of enOsCas12f1 and enRhCas12f1. 5′-TTTC PAM- and 5′-CCCA PAM-adjacent protospacer sequences were used for OsCas12f1 and RhCas12f1, respectively. Values and error bars represent mean and s.d. (n=3).

FIGS. 18A-18D. Deletion of DMD exon 51 by DD-enOsCas12f1. FIG. 18A, Indel frequencies induced by enOsCas12f1, Un1Cas12f1_ge4.1, and enRhCas12f1 at the 5′ and 3′ region flanking DMD exon 51 in HEK293T cells. Target sites for SpCas9 from Ousterout et al. Values and error bars represent mean and s.d. (n=3). FIG. 18B, RT-PCR across DMD exon 51 showed a smaller band with exon 51 deletion in treated muscle. FIG. 18C, Percentage of exon 51 deletion calculated by TA cloning of RT-PCR product. Values and error bars represent mean and s.d. (n=3). FIG. 18D, Representative chromatogram of the expected deletion PCR product.

FIG. 19. Cloning strategy for enOsCas12f1-mediated epigenome editing (miniCRISPRoff). Scheme of denOsCas12f1 fused with epigenetic editors (miniCRISPRoff) for gene silencing. CRISPRoff-v2 design from Nunez, J. K. et al., 2021.

FIGS. 20A-20B. Gating strategy used for assessing the efficiency of miniCRISPRoff and denOsCas12f1-VPR. FIG. 20A, GFP repression efficiency of miniCRISPRoffs and CRISPRoff-v2 at 5 days post transfection in Snrp-GFP HEK293T cells. FIG. 20B, GFP activation efficiency induced by denOsCas12f1-VPR at 3 days post transfection in TRE3G-GFP HEK293T cells.

FIG. 21. Uncropped images. The red rectangles indicate the cropping location.

FIG. 22 shows a schematic of the two plasmids in the fluorescent reporter assay.

FIG. 23 shows the cleavage activities of various CRISPR-Cas12f systems of the disclosure.

FIG. 24 is a schematic illustrating an exemplary target dsDNA, an exemplary guide nucleic acid, and an exemplary Cas12f.

The figures herein are for illustrative purposes only and are not necessarily drawn to scale.

### DETAILED DESCRIPTION OF THE EXAMPLE EMBODIMENTS

### Overview

The disclosure provides Cas12f polypeptides, and Cas12f polypeptides with high spacer sequence-specific (on-target) dsDNA cleavage activity and/or low spacer sequence-independent (off-target) dsDNA cleavage activity based on parent or reference Cas12f polypeptides, and fusions and uses thereof.

In some embodiments, the parent or reference Cas12f polypeptide may be: (i) any one of SEQ ID NOs: 1-34 of the disclosure or a known Cas12f polypeptide, (ii) a naturally-occurring ortholog, paralog, or homolog of any one of (i); (iii) a Cas12f polypeptide having a sequence identity of at least about 40%, 50%, 60%, 70%, 80%, 85%, 90%, 91%, 92%, 93%, 94%, 95%, 96%, 97%, 98%, or 99% to any one of (i) and (ii); or (iv) any mutant or variant of (i) to (iii). The parent or reference Cas12f polypeptide may be a wild type or not.

### Representative Cas12f Polypeptides

As representatives of the disclosure, in an aspect, the disclosure provides a Cas12f polypeptide comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 1-34 (optionally any one of SEQ ID NOs: 1, 2, 4, 5, 15, 28, 29, 31, and 32).

In some embodiments, the Cas12f polypeptide is not any one of SEQ ID NOs: 1-34 (optionally any one of SEQ ID NOs: 1, 2, 4, 5, 15, 28, 29, 31, and 32).

### Characterization of Cas12f Polypeptide

In some aspects of the disclosure, the Cas12f polypeptide of the disclosure has or retains or has improved endonuclease activity against a target DNA for on-target DNA cleavage. Still for the purpose of on-target DNA cleavage, the Cas12f polypeptide of the disclosure may not only have on-target endonuclease activity but also substantially lack off-target endonuclease activity such that it can have specificity for a target DNA. On the other hand, the Cas12f polypeptide of the disclosure can be engineered to substantially lack endonuclease activity (either on-target or off-target) but retain its ability of complexing with a guide nucleic acid and thus being guided to a target DNA, so as to indirectly guide a functional domain associated with the Cas12f polypeptide to the target DNA. Therefore, the characterization of the Cas12f polypeptide of the disclosure is not limited to its ability of on-target DNA cleavage.

In some embodiments, the Cas12f polypeptide has a function (e.g., a modified function that is either increased or decreased compared to that) of any one of SEQ ID NOs: 1-34 (optionally any one of SEQ ID NOs: 1, 2, 4, 5, 15, 28, 29, 31, and 32) (e.g., an ability to form a complex with a guide nucleic acid capable of forming a complex with any one of SEQ ID NOs: 1-34 (optionally any one of SEQ ID NOs: 1, 2, 4, 5, 15, 28, 29, 31, and 32); and/or, a guide sequence-specific dsDNA cleavage activity).

In some embodiments, the Cas12f polypeptide has guide sequence-specific (on-target) dsDNA cleavage activity.

In some embodiments, the Cas12f polypeptide substantially retains the guide sequence-specific (on-target) dsDNA cleavage activity of any one of SEQ ID NOs: 1-34 (optionally any one of SEQ ID NOs: 1, 2, 4, 5, 15, 28, 29, 31, and 32).

### Increased On-Target Cleavage

In some embodiments, the Cas12f polypeptide has an increased guide sequence-specific (on-target) dsDNA cleavage activity compared to that of any one of SEQ ID NOs: 1-34 (optionally any one of SEQ ID NOs: 1, 2, 4, 5, 15, 28, 29, 31, and 32) when both are used in combination with a same guide nucleic acid, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some embodiments, the Cas12f polypeptide comprises an amino acid substitution at position 46, 49, 50, 52, 53, 54, 56, 57, 62, 63, 66, 70, 71, 72, 119, 120, 127, 132, 136, 141, 144, 146, 147, 148, 150, 264, 292, 293, 311, 313, 314, and/or 315 of SEQ ID NO: 1 (OsCas12f1 (ME-B.3)).

In some embodiments, the Cas12f polypeptide comprises an amino acid substitution at position 10, 11, 13, 14, 15, 17, 18, 19, 20, 27, 28, 31, 32, 40, 44, 47, 49, 51, 52, 55, 56, 59, 61, 63, 65, 68, 71, 84, 91, 94, 96, 99, 111, 112, 124, 125, 126, 127, 128, 129, 130, 131, 139, 140, 141, 146, 147, 150, 151, 156, 160, 163, 167, 170, 173, 178, 179, 180, 183, 192, 193, 194, 195, 196, 197, 198, 199, 200, 201, 202, 206, 215, 224, 225, 226, 227, 230, 235, 249, 254, 256, 257, 264, 265, 266, 269, 270, 272, 273, 276, 280, 283, 292, 295, 303, 309, 311, 313, 314, 316, 318, 319, 320, 321, 334, 337, 341, 344, 346, 349, 358, 363, 365, 366, 367, 368, 371, 372, 374, 375, 377, 380, 382, 393, 399, 403, 404, 406, 408, 409, 410, 411, 413, and/or 414 of SEQ ID NO: 2 (RhCas12f1 (ME-A.1)).

Typically, amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)).

In some embodiments, the amino acid substitution is a substitution with a positively charged amino acid residue (such as, Lysine (Lys/K), Arginine (Arg/R), Histidine (His/H)), and optionally a substitution with Arginine (Arg/R).

In some embodiments, the Cas12f polypeptide comprises an amino acid substitution D52R and/or T132R relative to SEQ ID NO: 1.

In some embodiments, the Cas12f polypeptide comprises substitutions D52R and T132R relative to SEQ ID NO: 1.

In some embodiments, the Cas12f polypeptide comprises the amino acid sequence of SEQ ID NO: 226, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 226.

In some embodiments, the Cas12f polypeptide comprises an amino acid substitution A56R, Y125R, S130R, T131R, I264R, L270R, and/or A273R relative to SEQ ID NO: 2.

In some embodiments, the Cas12f polypeptide comprises an amino acid substitution L270R relative to SEQ ID NO: 2.

In some embodiments, the Cas12f polypeptide comprises the amino acid sequence of SEQ ID NO: 227, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 227.

### Decreased Off-Target Cleavage

In some embodiments, the Cas12f polypeptide substantially lacks guide sequence-independent (off-target) dsDNA cleavage activity.

In some embodiments, the Cas12f polypeptide substantially lacks the guide sequence-independent (off-target) dsDNA cleavage activity of any one of SEQ ID NOs: 1-34 (optionally any one of SEQ ID NOs: 1, 2, 4, 5, 15, 28, 29, 31, and 32).

In some embodiments, the Cas12f polypeptide has a decreased guide sequence-independent (off-target) dsDNA cleavage activity compared to that of any one of SEQ ID NOs: 1-34 (optionally any one of SEQ ID NOs: 1, 2, 4, 5, 15, 28, 29, 31, and 32) when both are used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

### Endonuclease Deficient (Dead) Cas12f Polypeptide

In some aspects, the disclosure provides a Cas12f polypeptide that is endonuclease deficient, which means the Cas12f polypeptide is substantially incapable of functioning as an endonuclease to cleave (either double strands or a single strand of) a dsDNA or a ssDNA, either against a target DNA or against a non-target DNA (For convenience of experiment design, performance, and evaluation, the defect of endonuclease activity is usually indicated by substantial loss of spacer sequence-specific dsDNA cleavage activity against a target DNA). Such a Cas12f polypeptide is named as “dead Cas12f (dCas12f)” and may be generated based on the parent or reference Cas12f polypeptide, for example, by mutating one or more functional domains of the parent or reference Cas12f polypeptide that is/are responsible for endonuclease activity.

In some embodiments, the Cas12f polypeptide is further engineered to substantially lack guide sequence-specific (on-target) dsDNA cleavage activity.

In some embodiments, the Cas12f polypeptide substantially lacks the guide sequence-specific (on-target) dsDNA cleavage activity of any one of SEQ ID NOs: 1-34 (optionally any one of SEQ ID NOs: 1, 2, 4, 5, 15, 28, 29, 31, and 32).

In some embodiments, the Cas12f polypeptide has a decreased guide sequence-specific (on-target) dsDNA cleavage activity compared to that of any one of SEQ ID NOs: 1-34 (optionally any one of SEQ ID NOs: 1, 2, 4, 5, 15, 28, 29, 31, and 32) when both used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the Cas12f polypeptide comprises an amino acid substitution at position 44, 79, 81, 82, 125, 131, 133, 138, 149, 151, 153, 228, 268, 270, 271, 274, 275, 277, 279, 282, 287, 291, 305, 308, 312, and/or 406 of SEQ ID NO: 1.

In some embodiments, the Cas12f polypeptide comprises an amino acid substitution at position 4, 7, 9, 23, 30, 33, 34, 35, 37, 38, 39, 41, 42, 46, 60, 62, 67, 69, 72, 75, 76, 77, 78, 80, 81, 82, 86, 90, 93, 97, 98, 101, 105, 107, 108, 114, 116, 121, 123, 135, 137, 143, 145, 148, 162, 165, 177, 185, 187, 189, 190, 207, 208, 209, 210, 212, 216, 217, 218, 219, 220, 231, 243, 278, 289, 290, 293, 296, 297, 302, 305, 307, 308, 310, 326, 327, 328, 329, 332, 336, 340, 347, 350, 356, 359, 362, 376, 378, 381, 388, 390, 391, 392, 395, and/or 396 of SEQ ID NO: 2.

Typically, amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)).

In some embodiments, the amino acid substitution is a substitution with (1) a positively charged amino acid residue (such as, Lysine (Lys/K), Arginine (Arg/R), Histidine (His/H)), and optionally a substitution with Arginine (Arg/R); or (2) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F)), and optionally a substitution with Alanine (Ala/A).

In some embodiments, the Cas12f polypeptide comprises an amino acid substitution D228A and/or D406A relative to SEQ ID NO: 1.

In some embodiments, the Cas12f polypeptide comprises the amino acid sequence of SEQ ID NO: 221 or 222, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 221 or 222.

In some embodiments, the Cas12f polypeptide comprises amino acid substitutions D52R and T132R relative to SEQ ID NO: 1.

In some embodiments, the Cas12f polypeptide comprises amino acid substitutions D52R, T132R, D228A, and D406A relative to SEQ ID NO: 1.

In some embodiments, the Cas12f polypeptide comprises the amino acid sequence of SEQ ID NO: 513 (denOsCas12f1 (OsCas12f1-D52R+T132R+D228A+D406A)), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 513.

In some embodiments, the Cas12f polypeptide comprises an amino acid substitution D210A and/or D388A relative to SEQ ID NO: 2.

In some embodiments, the Cas12f polypeptide comprises the amino acid sequence of SEQ ID NO: 223 or 224, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 223 or 224.

In some embodiments, the Cas12f polypeptide comprises an amino acid substitution L270R relative to SEQ ID NO: 2.

In some embodiments, the Cas12f polypeptide comprises amino acid substitutions D210A, L270R, and D388A relative to SEQ ID NO: 2.

In some embodiments, the Cas12f polypeptide comprises the amino acid sequence of SEQ ID NO: 515 (denRhCas12f1 (RhCas12f1-D210A+L270R+D388A)), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 515.

### Cas12f Nickase

In some aspects, the disclosure provides a Cas12f polypeptide that is not completely endonuclease deficient but the endonuclease activity is not against the double strand of a dsDNA but against one strand (the sense or nonsense strand; or the target or nontarget strand) of a dsDNA or a ssDNA, which means the Cas12f polypeptide is substantially incapable of functioning as a dsDNA endonuclease to cleave double strands of a dsDNA, either against a target DNA or against a non-target DNA, but is substantially capable of functioning as a ssDNA endonuclease to cleave a ssDNA or “nick” one strand of a dsDNA. Such a Cas12f polypeptide is named as “nickase” and may be generated based on the parent or reference Cas12f polypeptide, for example, by mutating one or more functional domains of the parent or reference Cas12f polypeptide that is/are responsible for endonuclease activity.

In some embodiments, the Cas12f polypeptide is further engineered to be a nickase.

### Fusion Protein

In some aspects, the disclosure provides a fusion protein comprising the Cas12f polypeptide and a functional domain. In some embodiments, the functional domain is a heterologous functional domain. Such a function protein may also be regarded as a Cas12f polypeptide further comprising a functional domain fused to the Cas12f polypeptide.

In some embodiments, the Cas12f polypeptide further comprises a functional domain fused to the Cas12f polypeptide.

In some embodiments, the functional domain is selected from the group consisting of a nuclear localization signal (NLS), a nuclear export signal (NES), a base editing domain, for example, a deaminase or a catalytic domain thereof, a base excising domain, an uracil glycosylase inhibitor (UGI) or a catalytic domain thereof, an uracil glycosylase (UNG) or a catalytic domain thereof, a methylpurine glycosylase (MPG) or a catalytic domain thereof, a methylase or a catalytic domain thereof, a demethylase or a catalytic domain thereof, an transcription activating domain (e.g., VP64 or VPR), an transcription inhibiting domain (e.g., KRAB moiety or SID moiety), a reverse transcriptase or a catalytic domain thereof, an exonuclease (e.g., T5E (SEQ ID NO: 449)) or a catalytic domain thereof, a destabilized domain (e.g., destabilized domains (DD) of E. coli dihydrofolate reductase (ecDHFR)), a histone residue modification domain, a nuclease catalytic domain (e.g., FokI), a transcription modification factor, a light gating factor, a chemical inducible factor, a chromatin visualization factor, a targeting polypeptide for providing binding to a cell surface portion on a target cell or a target cell type, a reporter (e.g., fluorescent) polypeptide or a detection label (e.g., GST, HRP, CAT, GFP, HcRed, DsRed, CFP, YFP, BFP), a localization signal, a polypeptide targeting moiety, a DNA binding domain (e.g., MBP, Lex A DBD, Gal4 DBD), an epitope tag (e.g., His, myc, V5, FLAG, HA, VSV-G, Trx, etc), a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase, a functional domain exhibiting activity to modify a target DNA, selected from the group consisting of: methyltransferase activity, DNA repair activity, DNA damage activity, dismutase activity, alkylation activity, dealkylation activity, depurination activity, oxidation activity, deoxidation activity, pyrimidine dimer forming activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, and a catalytic domain thereof, and a functional fragment (e.g., a functional truncation) thereof, and any combination thereof.

In some embodiments, the NLS comprises or is SV40 NLS (such as, SEQ ID NO: 216; coded by, such as, SEQ ID NO: 217), bpSV40 NLS (BP NLS, bpNLS), or NP NLS (Xenopus laevis Nucleoplasmin NLS, nucleoplasmin NLS) (such as, SEQ ID NO: 218; coded by, such as, SEQ ID NO: 219).

### Base Editing

In some embodiments, the base editing domain is capable of substituting a base of a nucleotide with a different base.

In some embodiments, the base editing domain is capable of deaminating a base of a nucleotide.

In some embodiments, the base editing domain comprises a deaminase domain capable of deaminating a base (e.g., an adenine, a guanine, a cytosine, a thymine, an uracil) of a nucleotide. In some embodiments, the deaminase domain is capable of deaminating an adenine (A) to a hypoxanthine (I). In some embodiments, the deamination of the adenine to the hypoxanthine converts the adenosine (A) or deoxyadenosine (dA) containing the adenine to a guanosine (G) or deoxyguanosine (dG). In some embodiments, the deaminase domain is capable of deaminating a cytosine (C) to an uracil (U). In some embodiments, the deamination of the cytosine to the uracil converts the cytidine (C) or deoxycytidine (dC) containing the cytosine to a uridine (U) or a deoxythymidine (dT).

In some embodiments, the base editing domain is capable of excising a base (e.g., an adenine, a guanine, a cytosine, a thymine, an uracil) of a nucleotide.

In some embodiments, the base editing domain comprises a base excising domain capable of excising a base of a nucleotide.

In some embodiments, the base editing domain comprises a deaminase domain and a base excising domain.

In some embodiments, the deaminase domain is tRNA adenosine deaminase (TadA), or the deaminase domain thereof, or a functional variant or fragment thereof, e.g., TadA8e, TadA8.17, TadA8.20, TadA9, TadA8EV106W, TadA8EV106W+D108Q TadA-CDa, TadA-CDb, TadA-CDc, TadA-CDd, TadA-CDe, TadA-dual, T_{AD}AC-1.2, T_{AD}AC-1.14, T_{AD}AC-1.17, T_{AD}AC-1.19, T_{AD}AC-2.5, T_{AD}AC-2.6, T_{AD}AC-2.9, T_{AD}AC-2.19, T_{AD}AC-2.23, TadA8e-N46L, TadA8e-N46P.

In some embodiments, the deaminase domain is an apolipoprotein B mRNA-editing complex (APOBEC) family deaminase, an activation induced deaminase (AID), a cytidine deaminase 1 from Petromyzon marinus (pmCDA1), or the deaminase domain thereof, or a functional variant or fragment thereof, e.g., APOBEC1, APOBEC2, APOBEC3A, APOBEC3B, APOBEC3C, APOBEC3D, APOBEC3F, APOBEC3G, APOBEC3H.

In some embodiments, the deaminase or catalytic domain thereof is an adenine deaminase (e.g., TadA, such as, TadA8e, TadA8.17, TadA8.20, TadA9) or a catalytic domain thereof, for example, TadA8e-V106W, TadA8e-W106V.

In some embodiments, the deaminase or catalytic domain thereof is a cytidine deaminase (e.g., APOBEC, such as, APOBEC3, for example, APOBEC3A, APOBEC3B, APOBEC3C; DddA) or a catalytic domain thereof, for example, hAPOBEC3-W104A.

In some embodiments, the UGI is human UGI domain.

In some embodiments, the Cas12f polypeptide comprises amino acid substitutions D52R, T132R, D228A, and D406A relative to SEQ ID NO: 1, and a base editing domain, for example, a deaminase or a catalytic domain thereof.

In some embodiments, the Cas12f polypeptide comprises the amino acid sequence of any one of SEQ ID NOs: 260-265, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 260-265.

In some embodiments, the functional domain comprises a reverse transcriptase (RT) or a catalytic domain thereof. In some embodiments, the guide nucleic acid further comprises or is used in combination with a reverse transcription donor RNA (RT donor RNA) comprising a primer binding site (PBS) and a template sequence. For details of prime editing with Class 2, Type V Cas proteins, references is made to WO2022256440A3, which is incorporated herein by reference in its entirety.

### System

The Cas12f polypeptide of the disclosure may be used in combination with and guided by a guide nucleic acid to a target DNA to function on the target DNA.

In another aspect, the disclosure provides a system comprising:
(1) a Cas12f polypeptide of the disclosure, or a polynucleotide encoding the Cas12f polypeptide; and
(2) a guide nucleic acid or a polynucleotide encoding the guide nucleic acid, the guide nucleic acid comprising:
(i) a scaffold sequence capable of forming a complex with the Cas12f polypeptide; and
(ii) a guide sequence capable of hybridizing to a target sequence of a target DNA, thereby guiding the complex to the target DNA.

In some embodiments, the system is a non-naturally occurring or engineered system.

In some embodiments, the system is a complex comprising the Cas12f polypeptide complexed with the guide nucleic acid. In some embodiments, the complex further comprises the target DNA hybridized with the target sequence.

In some embodiments, the Cas12f polypeptide comprises an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 1-34 (optionally any one of SEQ ID NOs: 1, 2, 4, 5, 15, 28, 29, 31, and 32).

In some embodiments, the Cas12f polypeptide is a mutant of any one of SEQ ID NOs: 1-34 (optionally any one of SEQ ID NOs: 1, 2, 4, 5, 15, 28, 29, 31, and 32) as described herein.

In another aspect, the disclosure provides a guide nucleic acid comprising:
(1) a scaffold sequence capable of forming a complex with the Cas12f polypeptide of the disclosure, and
(2) a guide sequence capable of hybridizing to a target sequence of a target DNA, thereby guiding the complex to the target DNA.

In some embodiments, the guide nucleic acid is a guide RNA (gRNA), e.g., a single guide RNA (sgRNA). In some embodiments, the guide nucleic acid comprises a crRNA. In some embodiments, the guide nucleic acid comprises a tracrRNA.

In some embodiments, the scaffold sequence is 5′ to the spacer sequence.

In some embodiments, the guide nucleic acid further comprises a polyU sequence having at least four consecutive U (uridine) 3′ to the guide sequence.

In some embodiments, the polyU sequence further comprises one A (adenosine) downstream of the at least four consecutive U.

In some embodiments, the sequence encoding the polyU sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO:220; or a sequence having at most 1, 2, 3, 4, 5, 6, 7, 8, 9, or 10 nucleotide differences, whether consecutive or not, compared to the sequence of SEQ ID NO: 220.

### Design of Protospacer Sequence/Target Sequence; Target Site

For the purpose of the disclosure, in some embodiments, the protospacer sequence or target sequence is located such that the target DNA is specifically modified by the Cas12f polypeptide.

To facilitate the evaluation of selected protospacer sequences or target sequence and designed guide sequences in mouse models, in some embodiments, the protospacer sequence or target sequence is located such that a mouse target DNA is specifically modified by the Cas12f polypeptide. In some embodiments, the protospacer sequence or target sequence is located such that both a human target DNA and a mouse target DNA are specifically modified by the Cas12f polypeptide. That is, the protospacer sequence or target sequence is selected to be cross-reactive to both human and mouse species.

In some embodiments, the protospacer sequence is a stretch of contiguous nucleotides identified from the nontarget strand of the target DNA by identifying the stretch of contiguous nucleotides immediately 3′ to the PAM on the nontarget strand. In some embodiments, the PAM is 5′-TTN or 5′-CCN, wherein N is A, T, G, or C. The protospacer sequence is the reversely complementary sequence of the target sequence.

In some embodiments, the protospacer sequence is a stretch of about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or a stretch of contiguous nucleotides of the target DNA in a numerical range between any two of the preceding values, e.g., a stretch of from about 16 to about 50, or from about 17 to about 22 contiguous nucleotides. In some embodiments, the protospacer sequence is a stretch of about 20 contiguous nucleotides of the target DNA.

In some embodiments, the protospacer sequence comprises about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or contiguous nucleotides in a numerical range between any two of the preceding values, e.g., from about 16 to about 50, or from about 17 to about 22 contiguous nucleotides of the target DNA. In some embodiments, the protospacer sequence comprises about 20 contiguous nucleotides of the target DNA.

In some embodiments, the target sequence is a stretch of contiguous nucleotides identified from the target strand of the target DNA. The target sequence is the reversely complementary sequence of the protospacer sequence.

In some embodiments, the target sequence is a stretch of about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the target strand of the target DNA, or a stretch of contiguous nucleotides on the target strand of the target DNA in a numerical range between any two of the preceding values, e.g., a stretch of from about 16 to about 50, or from about 17 to about 22 contiguous nucleotides. In some embodiments, the target sequence is a stretch of about 20 contiguous nucleotides on the target strand of the target DNA.

In some embodiments, the target sequence comprises about or at least about 16 contiguous nucleotides of the target DNA, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or in a numerical range between any two of the preceding values, e.g., from about 16 to about 50, or from about 17 to about 22 contiguous nucleotides of the target DNA. In some embodiments, the target sequence comprises about 20 contiguous nucleotides of the target DNA.

In some embodiments, the target sequence comprises about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the target strand of the target DNA, or contiguous nucleotides in a numerical range between any two of the preceding values, e.g., from about 16 to about 50, or from about 17 to about 22 contiguous nucleotides on the target strand of the target DNA. In some embodiments, the target sequence comprises about 20 contiguous nucleotides on the target strand of the target DNA.
In some embodiments, the reversely complementary sequence of the target sequence is immediately 3′ to a protospacer adjacent motif (PAM); optionally, wherein the PAM is 5′-TTN or 5′-CCN, wherein N is A, T, G, or C.

In some embodiments, the nontarget strand is the sense strand of the target DNA.

In some embodiments, the nontarget strand is the antisense strand of the target DNA.

In some embodiments, the target strand is the sense strand of the target DNA.

In some embodiments, the target strand is the antisense strand of the target DNA.

In some embodiments, the protospacer sequence or target sequence is located within Exon 1 of the target DNA.

In some embodiments, the protospacer sequence or target sequence is located within about 50, 100, 150, 200, 250, 300, or more 5′ end nucleotides of Exon 1 of the target DNA.

In some embodiments, the target DNA comprises a pathogenic mutation.

In some embodiments, the target DNA comprises a premature stop codon (e.g., TAG).

In some embodiments, the target DNA is a dsDNA, such as, a eukaryotic dsDNA, e.g., a gene in a eukaryotic cell.

In some embodiments, the target DNA is human target DNA, non-human primate target DNA, or mouse target DNA.

In some embodiments, the target DNA is in a eukaryotic cell, for example, a human cell, a non-human primate cell, or a mouse cell.

### Design of Guide Sequence According to Protospacer/Target Sequence

In some embodiments, the guide sequence is about or at least about 16 nucleotides in length, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any two of the preceding values, e.g., in a length of from about 16 to about 50 nucleotides, or from about 17 to about 22 nucleotides. In some embodiments, the guide sequence is about 20 nucleotides in length.

In some embodiments, (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence. In some embodiments, the guide sequence is about 100% (fully), reversely complementary to the target sequence.

### Selection of Protospacer/Target/Guide Sequence; Effect of System

In some embodiments, the protospacer sequence, the target sequence, or the guide sequence is selected such that the target DNA is modified by the system of the disclosure. In some embodiments, the modification decreases or eliminates the transcription of the target DNA and/or translation of a transcript (e.g., mRNA) of the target DNA.

In some embodiments, the level of the transcript (e.g., mRNA) of the target DNA is decreased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell model or the animal model, compared to the level of the transcript (e.g., mRNA) of the target DNA in the same cell model or animal model that does not receive the administration.

In some embodiments, the level of the transcript (e.g., mRNA) of the target DNA is increased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell model or the animal model, compared to the level of the transcript (e.g., mRNA) of the target DNA in the same cell model or animal model that does not receive the administration.

In some embodiments, the level of the expression product (e.g., protein) of the target DNA is decreased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell or the animal model, compared to the level of the expression product (e.g., protein) of the target DNA in the same cell model or animal model that does not receive the administration.

In some embodiments, the level of the expression product (e.g., protein) of the target DNA is increased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell or the animal model, compared to the level of the expression product (e.g., protein) of the target DNA in the same cell model or animal model that does not receive the administration. In some embodiments, the expression product is a functional mutant of the expression product of the target DNA.

### Overall Structure of Guide Nucleic Acid

In some embodiments, the guide nucleic acid is a single molecule.

In some embodiments, the guide nucleic acid comprises one guide sequence capable of hybridizing to one target sequence.

In some embodiments, the guide nucleic acid comprises a plurality (e.g., 2, 3, 4, 5 or more) of the guide sequences capable of hybridizing to a plurality of the target sequences, respectively.

In some embodiments, the guide nucleic acid comprises, from 5′ to 3′, the direct repeat sequence, the guide sequence, the direct repeat sequence, the guide sequence, and the direct repeat sequence.

In some embodiments, the guide nucleic acid comprises one scaffold sequence and one guide sequence.

In some embodiments, the guide nucleic acid comprises one scaffold sequence 5′ to one guide sequence. In some embodiments, the guide nucleic acid comprises one scaffold sequence 3′ to one guide sequence.

In some embodiments, the guide nucleic acid comprises one or more scaffold sequence and/or one or more guide sequence, provided that the guide nucleic acid does not comprise one scaffold sequence and one guide sequence.

In some embodiments, the guide nucleic acid comprises, from 5′ to 3′, one scaffold sequence, one guide sequence, and one scaffold sequence, wherein scaffold sequences are the same or different.

In some embodiments, the guide nucleic acid comprises, from 5′ to 3′, one guide sequence, one scaffold sequence, and one guide sequence, wherein guide sequences are the same or different.

In some embodiments, the guide nucleic acid comprises, from 5′ to 3′, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one guide sequence, one scaffold sequence, one guide sequence, and one scaffold sequence, wherein scaffold sequences are the same or different, and wherein guide sequences are the same or different.

In some embodiments, the guide nucleic acid comprises, from 5′ to 3′, one scaffold sequence, one guide sequence, one scaffold sequence, one guide sequence, and one scaffold sequence, wherein scaffold sequences are the same or different, and wherein guide sequences are the same or different.

In some embodiments, the guide nucleic acid comprises, from 5′ to 3′, one guide sequence, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one scaffold sequence, one guide sequence, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one guide sequence, one scaffold sequence, one guide sequence, one scaffold sequence, one guide sequence, and one scaffold sequence, wherein scaffold sequences are the same or different, and wherein guide sequences are the same or different.

In some embodiments, the guide nucleic acid comprises a linker or no linker between any adjacent scaffold sequence and guide sequence. In some embodiments, the guide nucleic acid comprises no linker between any adjacent scaffold sequence and guide sequence.

### Multiple Guide Nucleic Acid

The system of the disclosure may comprise or encode one guide nucleic acid or comprise or encode multiple (e.g., 2, 3, 4, or more) guide nucleic acids, e.g., for the purpose of improving the editing efficiency of the system on target DNA.

In some embodiments, the system further comprises one or more additional guide nucleic acids, or the first polynucleotide sequence further comprises one or more additional sequences encoding one or more additional guide nucleic acids, each of the additional guide nucleic acids comprising:
(1) an additional scaffold sequence capable of forming a complex with the Cas12f polypeptide, and
(2) an additional guide sequence capable of hybridizing to an additional target sequence on a target strand of the target DNA or an additional target sequence on the transcript thereof, thereby guiding the complex to the target DNA or the transcript.

In some embodiments, the additional protospacer sequence is on the same strand as the protospacer sequence.

In some embodiments, the additional protospacer sequence is on the different strand from the protospacer sequence.

In some embodiments, the additional protospacer sequence is the same or different from the protospacer sequence.

In some embodiments, the additional target sequence is the same or different from the target sequence.

In some embodiments, the additional guide sequence is the same or different from the guide sequence.

In some embodiments, the additional scaffold sequence is the same or different from the scaffold sequence. In some embodiments wherein the system comprises the same Cas12f polypeptide and multiple guide nucleic acids, the scaffold sequences of the multiple guide nucleic acids may be the same or different (e.g., different by no more than 5, 4, 3, 2, or 1 nucleotide) to be compatible to the same Cas12f polypeptide. In some embodiments wherein that the system comprises different Cas12f polypeptides and multiple guide nucleic acids, the scaffold sequences of the multiple guide nucleic acids may be different to be compatible to the different Cas12f polypeptides.

In some embodiments, the additional guide nucleic acid and the guide nucleic acid are operably linked to or under the regulation of the same regulatory element (e.g., promoter) or separate regulatory elements (e.g., promoters).

In some embodiments, the system comprises two or more guide nuclei acids comprising two or more guide sequences capable of hybridizing to two or more target sequences of the same target DNA or different target DNAs, wherein the two or more guide sequences are the same or different, and wherein the two or more target sequences are the same or different.

### Nature and Modification of Guide Nucleic Acid

In some embodiments, the guide nucleic acid (e.g., the guide nucleic acid, the additional guide nucleic acid) is an RNA. In some embodiments, the guide nucleic acid is an unmodified guide RNA. In some embodiments, the guide nucleic acid is a modified guide RNA. In some embodiments, the guide nucleic acid comprises a modification. In some embodiments, the guide nucleic acid is a modified RNA containing a modified ribonucleotide. In some embodiments, the guide nucleic acid is a modified RNA containing a deoxyribonucleotide. In some embodiments, the guide nucleic acid is a modified RNA containing a modified deoxyribonucleotide. In some embodiments, the guide nucleic acid comprises a modified or unmodified deoxyribonucleotide and a modified or unmodified ribonucleotide.

### Scaffold Sequence

For the purpose of the disclosure, the scaffold sequence is compatible with the Cas12f polypeptide of the disclosure and is capable of complexing with the Cas12f polypeptide. The scaffold sequence may be a naturally occurring scaffold sequence identified along with the Cas12f polypeptide, or a variant thereof maintaining the ability to complex with the Cas12f polypeptide. Generally, the ability to complex with the Cas12f polypeptide is maintained as long as the secondary structure of the variant is substantially identical to the secondary structure of the naturally occurring scaffold sequence. A nucleotide deletion, insertion, or substitution in the primary sequence of the scaffold sequence may not necessarily change the secondary structure of the scaffold sequence (e.g., the relative locations and/or sizes of the stems, bulges, and loops of the scaffold sequence do not significantly deviate from that of the original stems, bulges, and loops). For example, the nucleotide deletion, insertion, or substitution may be in a bulge or loop region of the scaffold sequence so that the overall symmetry of the bulge and hence the secondary structure remains largely the same. The nucleotide deletion, insertion, or substitution may also be in the stems of the scaffold sequence so that the lengths of the stems do not significantly deviate from that of the original stems (e.g., adding or deleting one base pair in each of two stems correspond to 4 total base changes).

Referring to FIG. 24, in some embodiments, the scaffold sequence is a fusion of tracrRNA sequence with repeat sequence of crRNA with or without a loop.

In some embodiments, the scaffold sequence comprises a tracrRNA sequence of any one of SEQ ID NOs: 111-144.

In some embodiments, the scaffold sequence comprises a repeat sequence of any one of SEQ ID NOs: 145-178.

In some embodiments, the crRNA sequence comprises a repeat sequence of any one of SEQ ID NOs: 145-178 and a guide sequence.

In some embodiments, the tracrRNA sequence comprises an anti-repeat sequence at its 3′ end that can form a duplex with the repeat sequence.

The repeat sequence is derived from the direct repeat (DR) sequence identified along with the cognate Cas12f polypeptide. In some embodiments, the repeat sequence is derived from the direct repeat sequence of any one of SEQ ID NOs: 179-212.

In some embodiments, the scaffold sequence or the additional scaffold sequence has substantially the same secondary structure as the secondary structure of any one of SEQ ID NOs: 73-106 (optionally any one of SEQ ID NOs: 73, 74, 76, 77, 87, 100, 101, 103, and 104).

In some embodiments, the scaffold sequence or the additional scaffold sequence:
(i) comprises the polynucleotide sequence of any one of SEQ ID NOs: 73-106 (optionally any one of SEQ ID NOs: 73, 74, 76, 77, 87, 100, 101, 103, and 104); or
(ii) comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 73-106 (optionally any one of SEQ ID NOs: 73, 74, 76, 77, 87, 100, 101, 103, and 104).

### Increased On-Target Cleavage

Engineering or optimization strategy may be applied to the scaffold sequence of the guide nucleic acid of the disclosure to assist in the on-target cleavage by the Cas12f polypeptide of the disclosure.

In some embodiments, the scaffold sequence leads to an increased guide sequence-specific (on-target) dsDNA cleavage activity compared to that led by any one of SEQ ID NOs: 73-106 (optionally any one of SEQ ID NOs: 73, 74, 76, 77, 87, 100, 101, 103, and 104) when both are used in otherwise identical guide nucleic acid in combination with a same Cas12f polypeptide (e.g., the Cas12f polypeptide of any preceding claim), e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some embodiments, the scaffold sequence comprises a base pair substitution of a thermodynamically unstable base pair (e.g., a A-U base pair or a mismatched base pair) with a G-C base pair.

In some embodiments, the scaffold sequence comprises a base pair substitution of a thermodynamically unstable base pair (e.g., a A-U base pair or a mismatched base pair) with a G-C base pair relative to SEQ ID NO: 73 and comprises the polynucleotide sequence of any one of SEQ ID NOs: 234-236, 239-242, 244-247, and 250-251, or a polynucleotide sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of any one of SEQ ID NOs: 234-236, 239-242, 244-247, and 250-251; optionally, wherein the scaffold sequence comprises the polynucleotide sequence of SEQ ID NO: 244.

In some embodiments, the scaffold sequence comprises a base pair substitution of a thermodynamically unstable base pair (e.g., a A-U base pair or a mismatched base pair) with a G-C base pair relative to SEQ ID NO: 74 and comprises the polynucleotide sequence of SEQ ID NO: 257, or a polynucleotide sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of SEQ ID NO: 257.

### Specific Combination of Cas12f or Mutant Thereof and Scaffold Sequence or Mutant Thereof

The scaffold sequence of the guide nucleic acid of the disclosure is required to be compatible with the Cas12f polypeptide of the disclosure so as to allow the complexing of the Cas12f polypeptide of the disclosure and the guide nucleic acid of the disclosure. One scaffold sequence may be compatible with several Cas12f polypeptides, and vice versa. Non-limiting combinations are provided in below.

In some embodiments, the Cas12f polypeptide comprises SEQ ID NO: 1 or a mutant thereof as defined in any preceding claim (e.g., SEQ ID NO: 226), and wherein the scaffold sequence comprises SEQ ID NO: 73 or a mutant thereof as defined in any preceding claim (e.g., SEQ ID NO: 244).

In some embodiments, the Cas12f polypeptide comprises SEQ ID NO: 2 or a mutant thereof as defined in any preceding claim (e.g., SEQ ID NO: 227), and wherein the scaffold sequence comprises SEQ ID NO: 74 or a mutant thereof as defined in any preceding claim (e.g., SEQ ID NO: 257).

In some embodiments, the Cas12f polypeptide comprises SEQ ID NO: 3 or a mutant thereof, and wherein the scaffold sequence comprises SEQ ID NO: 75 or a mutant thereof as defined in any preceding claim.

In some embodiments, the Cas12f polypeptide comprises SEQ ID NO: 4 or a mutant thereof, and wherein the scaffold sequence comprises SEQ ID NO: 76 or a mutant thereof as defined in any preceding claim.

In some embodiments, the Cas12f polypeptide comprises SEQ ID NO: 5 or a mutant thereof, and wherein the scaffold sequence comprises SEQ ID NO: 77 or a mutant thereof as defined in any preceding claim.

In some embodiments, the Cas12f polypeptide comprises SEQ ID NO: 6 or a mutant thereof, and wherein the scaffold sequence comprises SEQ ID NO: 78 or a mutant thereof as defined in any preceding claim.

In some embodiments, the Cas12f polypeptide comprises SEQ ID NO: 7 or a mutant thereof, and wherein the scaffold sequence comprises SEQ ID NO: 79 or a mutant thereof as defined in any preceding claim.

In some embodiments, the Cas12f polypeptide comprises SEQ ID NO: 8 or a mutant thereof, and wherein the scaffold sequence comprises SEQ ID NO: 80 or a mutant thereof as defined in any preceding claim.

In some embodiments, the Cas12f polypeptide comprises SEQ ID NO: 9 or a mutant thereof, and wherein the scaffold sequence comprises SEQ ID NO: 81 or a mutant thereof as defined in any preceding claim.

In some embodiments, the Cas12f polypeptide comprises SEQ ID NO: 10 or a mutant thereof, and wherein the scaffold sequence comprises SEQ ID NO: 82 or a mutant thereof as defined in any preceding claim.

In some embodiments, the Cas12f polypeptide comprises SEQ ID NO: 11 or a mutant thereof, and wherein the scaffold sequence comprises SEQ ID NO: 83 or a mutant thereof as defined in any preceding claim.

In some embodiments, the Cas12f polypeptide comprises SEQ ID NO: 12 or a mutant thereof, and wherein the scaffold sequence comprises SEQ ID NO: 84 or a mutant thereof as defined in any preceding claim.

In some embodiments, the Cas12f polypeptide comprises SEQ ID NO: 13 or a mutant thereof, and wherein the scaffold sequence comprises SEQ ID NO: 85 or a mutant thereof as defined in any preceding claim.

In some embodiments, the Cas12f polypeptide comprises SEQ ID NO: 14 or a mutant thereof, and wherein the scaffold sequence comprises SEQ ID NO: 86 or a mutant thereof as defined in any preceding claim.

In some embodiments, the Cas12f polypeptide comprises SEQ ID NO: 15 or a mutant thereof, and wherein the scaffold sequence comprises SEQ ID NO: 87 or a mutant thereof as defined in any preceding claim.

In some embodiments, the Cas12f polypeptide comprises SEQ ID NO: 16 or a mutant thereof, and wherein the scaffold sequence comprises SEQ ID NO: 88 or a mutant thereof as defined in any preceding claim.

In some embodiments, the Cas12f polypeptide comprises SEQ ID NO: 17 or a mutant thereof, and wherein the scaffold sequence comprises SEQ ID NO: 89 or a mutant thereof as defined in any preceding claim.

In some embodiments, the Cas12f polypeptide comprises SEQ ID NO: 18 or a mutant thereof, and wherein the scaffold sequence comprises SEQ ID NO: 90 or a mutant thereof as defined in any preceding claim.

In some embodiments, the Cas12f polypeptide comprises SEQ ID NO: 19 or a mutant thereof, and wherein the scaffold sequence comprises SEQ ID NO: 91 or a mutant thereof as defined in any preceding claim.

In some embodiments, the Cas12f polypeptide comprises SEQ ID NO: 20 or a mutant thereof, and wherein the scaffold sequence comprises SEQ ID NO: 92 or a mutant thereof as defined in any preceding claim.

In some embodiments, the Cas12f polypeptide comprises SEQ ID NO: 21 or a mutant thereof, and wherein the scaffold sequence comprises SEQ ID NO: 93 or a mutant thereof as defined in any preceding claim.

In some embodiments, the Cas12f polypeptide comprises SEQ ID NO: 22 or a mutant thereof, and wherein the scaffold sequence comprises SEQ ID NO: 94 or a mutant thereof as defined in any preceding claim.

In some embodiments, the Cas12f polypeptide comprises SEQ ID NO: 23 or a mutant thereof, and wherein the scaffold sequence comprises SEQ ID NO: 95 or a mutant thereof as defined in any preceding claim.

In some embodiments, the Cas12f polypeptide comprises SEQ ID NO: 24 or a mutant thereof, and wherein the scaffold sequence comprises SEQ ID NO:96 or a mutant thereof as defined in any preceding claim.

In some embodiments, the Cas12f polypeptide comprises SEQ ID NO: 25 or a mutant thereof, and wherein the scaffold sequence comprises SEQ ID NO: 97 or a mutant thereof as defined in any preceding claim.

In some embodiments, the Cas12f polypeptide comprises SEQ ID NO: 26 or a mutant thereof, and wherein the scaffold sequence comprises SEQ ID NO: 98 or a mutant thereof as defined in any preceding claim.

In some embodiments, the Cas12f polypeptide comprises SEQ ID NO: 27 or a mutant thereof, and wherein the scaffold sequence comprises SEQ ID NO: 99 or a mutant thereof as defined in any preceding claim.

In some embodiments, the Cas12f polypeptide comprises SEQ ID NO: 28 or a mutant thereof, and wherein the scaffold sequence comprises SEQ ID NO: 100 or a mutant thereof as defined in any preceding claim.

In some embodiments, the Cas12f polypeptide comprises SEQ ID NO: 29 or a mutant thereof, and wherein the scaffold sequence comprises SEQ ID NO: 101 or a mutant thereof as defined in any preceding claim.

In some embodiments, the Cas12f polypeptide comprises SEQ ID NO: 30 or a mutant thereof, and wherein the scaffold sequence comprises SEQ ID NO: 102 or a mutant thereof as defined in any preceding claim.

In some embodiments, the Cas12f polypeptide comprises SEQ ID NO: 31 or a mutant thereof, and wherein the scaffold sequence comprises SEQ ID NO: 103 or a mutant thereof as defined in any preceding claim.

In some embodiments, the Cas12f polypeptide comprises SEQ ID NO: 32 or a mutant thereof, and wherein the scaffold sequence comprises SEQ ID NO: 104 or a mutant thereof as defined in any preceding claim.

In some embodiments, the Cas12f polypeptide comprises SEQ ID NO: 33 or a mutant thereof, and wherein the scaffold sequence comprises SEQ ID NO: 105 or a mutant thereof as defined in any preceding claim.

In some embodiments, the Cas12f polypeptide comprises SEQ ID NO: 34 or a mutant thereof, and wherein the scaffold sequence comprises SEQ ID NO: 106 or a mutant thereof as defined in any preceding claim.

### Regulation of Guide Nucleic Acid

In some embodiments, the polynucleotide encoding the guide nucleic acid is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the guide nucleic acid is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

### Regulation of Cas12f Polypeptide

In some embodiments, the polynucleotide encoding the Cas12f polypeptide is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the polynucleotide encoding the Cas12f polypeptide is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a human synapsin (hSyn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a OTOF promoter, a GRK1 promoter, a CRX promoter, a NRL promoter, a MECP2 promoter, a mMECP2 promoter, a hMECP2 promoter, an APP promoter, and a RCVRN promoter.

### Delivery

Various ways of delivery can be applied to the Cas12f polypeptide of the disclosure or the system of the disclosure as needed in practices.

In yet another aspect, the disclosure provides a polynucleotide encoding the Cas12f polypeptide of the disclosure, e.g., any one of SEQ ID NO: 39-72.

In yet another aspect, the disclosure provides a delivery system comprising (1) the Cas12f polypeptide of the disclosure, the polynucleotide of the disclosure, or the system of the disclosure; and (2) a delivery vehicle.

In yet another aspect, the disclosure provides a vector comprising the polynucleotide of the disclosure. In some embodiments, the vector encodes a guide nucleic acid of the disclosure. In some embodiments, the vector is a plasmid vector, a recombinant AAV (rAAV) vector (vector genome), or a recombinant lentivirus vector.

In yet another aspect, the disclosure provides a recombinant AAV (rAAV) particle comprising the rAAV vector genome of the disclosure. A simple introduction of AAV for delivery may refer to “Adeno-associated Virus (AAV) Guide” (addgene.org/guides/aav/).

Adeno-associated virus (AAV), when engineered to delivery, e.g., a protein-encoding sequence of interest, may be termed as a (r)AAV vector, a (r)AAV vector particle, or a (r)AAV particle, where “r” stands for “recombinant”. And the genome packaged in AAV vectors for delivery may be termed as a (r)AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into ear cells (e.g., inner hair cells). In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV.PHP.eB, a member of the Clade to which any of the AAV1-AAV13 belong, or a functional variant (e.g., a functional truncation) thereof, encapsidating the rAAV vector genome. In some embodiments, the serotype of the capsid is AAV9 or a functional variant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10¹², above 5×10¹², or above 1×10¹³ vg/ml.

Instead of packaging a single strand (ss)DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a Cas13 coding sequence encoding a Cas13 polypeptide covers either a Cas13 DNA coding sequence from which a Cas13 polypeptide is expressed (indirectly via transcription and translation) or a Cas13 RNA coding sequence from which a Cas13 polypeptide is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

In yet another aspect, the disclosure provides a ribonucleoprotein (RNP) comprising the Cas12f polypeptide of the disclosure and a guide nucleic acid of the disclosure.

In yet another aspect, the disclosure provides a lipid nanoparticle (LNP) comprising an RNA (e.g., mRNA) encoding the Cas12f polypeptide of the disclosure and a guide nucleic acid of the disclosure.

### Method of Modification

The CRISPR-Cas12f system of the disclosure comprising the Cas12f polypeptide of the disclosure has a wide variety of utilities, including modifying (e.g., cleaving, deleting, inserting, translocating, inactivating, or activating) a target DNA in a multiplicity of cell types. The CRISPR-Cas12f systems have a broad spectrum of applications requiring high cleavage activity and small sizes, e.g., drug screening, disease diagnosis and prognosis, and treating various genetic disorders.

The methods and/or the systems of the disclosure can be used to modify a target DNA, for example, to modify the translation and/or transcription of one or more genes of the cells. For example, the modification may lead to increased transcription/translation/expression of a gene. In other embodiments, the modification may lead to decreased transcription/translation/expression of a gene.

In yet another aspect, the disclosure provides a method for modifying a target DNA, comprising contacting the target DNA with the system of the disclosure, the vector of the disclosure, the ribonucleoprotein of the disclosure, or the lipid nanoparticle of the disclosure, wherein the guide sequence is capable of hybridizing to a target sequence of the target DNA, wherein the target DNA is modified by the complex.

In some embodiments, the target DNA is in a cell.

In some embodiments, the modification comprises one or more of cleavage, base editing, repairing, and exogenous sequence insertion or integration of the target DNA.

### Cells

The methods of the disclosure can be used to introduce the systems of the disclosure into a cell and cause the cell to alter the production of one or more cellular produces, such as antibody, starch, ethanol, or any other desired products. Such cells and progenies thereof are within the scope of the disclosure.

In yet another aspect, the disclosure provides a cell comprising the system of the disclosure. In some embodiments, the cell is a eukaryote. In some embodiments, the cell is a human cell.

In yet another aspect, the disclosure provides a cell modified by the system of the disclosure or the method of the disclosure. In some embodiments, the cell is a eukaryote. In some embodiments, the cell is a human cell. In some embodiments, the cell is modified in vitro, in vivo, or ex vivo.

In some embodiments, the cell is a stem cell. In some embodiments, the cell is not a human embryonic stem cell. In some embodiments, the cell is not a human germ cell.

In some embodiments, the cell is a prokaryotic cell.

In some embodiments, the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell).

In some embodiments, the cell is from a plant or an animal.

In some embodiments, the plant is a dicotyledon. In some embodiments, the dicotyledon is selected from the group consisting of soybean, cabbage (e.g., Chinese cabbage), rapeseed, Brassica, watermelon, melon, potato, tomato, tobacco, eggplant, pepper, cucumber, cotton, alfalfa, eggplant, grape.

In some embodiments, the plant is a monocotyledon. In some embodiments, the monocotyledon is selected from the group consisting of rice, corn, wheat, barley, oat, sorghum, millet, grasses, Poaceae, Zizania, Avena, Coix, Hordeum, Oryza, Panicum (e.g., Panicum miliaceum), Secale, Setaria (e.g., Setaria italica), Sorghum, Triticum, Zea, Cymbopogon, Saccharum (e.g., Saccharum officinarum), Phyllostachys, Dendrocalamus, Bambusa, Yushania.

In some embodiments, the animal is selected from the group consisting of pig, ox, sheep, goat, mouse, rat, alpaca, monkey, rabbit, chicken, duck, goose, fish (e.g., zebra fish).

In some embodiments, the cell is a eukaryotic cell, such as a mammalian cell, including a human cell (a primary human cell or an established human cell line). In some embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In some embodiments, the cell is from fish (such as salmon), bird (such as poultry bird, including chick, duck, goose), reptile, shellfish (e.g., oyster, claim, lobster, shrimp), insect, worm, yeast, etc. In some embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

### Pharmaceutical Composition

In yet another aspect, the disclosure provides a pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL.

In some embodiments, the pharmaceutical composition is an injection.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Method of Treatment

In yet another aspect, the disclosure provides a method for diagnosing, preventing, or treating a disease in a subject in need thereof, comprising administering to the subject (e.g., a therapeutically effective dose of) the system of the disclosure, the vector of the disclosure, the ribonucleoprotein of the disclosure, the lipid nanoparticle of the disclosure, the cell of the disclosure, or the pharmaceutical composition of the disclosure, wherein the disease is associated with a target DNA, wherein the guide sequence is capable of hybridizing to a target sequence of the target DNA, wherein the target DNA is modified by the complex, and wherein the modification of the target DNA diagnose, prevents, or treats the disease.

In some embodiments, the disease is selected from the group consisting of Angelman syndrome (AS), Alzheimer's disease (AD), transthyretin amyloidosis (ATTR), transthyretin amyloid cardiomyopathy (ATTR-CM), cystic fibrosis (CF), hereditary angioedema, diabetes, progressive pseudohypertrophic muscular dystrophy, Duchenne muscular dystrophy (DMD), Becker muscular dystrophy (BMD), spinal muscular atrophy (SMA), alpha-1-antitrypsin deficiency, Pompe disease, myotonic dystrophy, Huntington's disease (HTT), fragile X syndrome, Friedreich ataxia, amyotrophic lateral sclerosis (ALS), frontotemporal dementia, hereditary chronic kidney disease, hyperlipidemia, Leber congenital amaurosis (LCA), sickle cell disease, thalassemia (e.g., β-thalassemia), Parkinson's disease (PD), myelodysplastic syndrome (MDS), retinitis pigmentosa (RP), age-related macular degeneration (AMD), Hepatitis B, nonalcoholic fatty liver disease (NAFLD), Acquired Immune Deficiency Syndrome, corneal dystrophy (CD), hypercholesterolemia, familial hypercholesterolemia (FH), heart disease (e.g., hypertrophic cardiomyopathy (HCM)), and cancer.

In some embodiments, the target DNA encodes a mRNA, a tRNA, a ribosomal RNA (rRNA), a microRNA (miRNA), a non-coding RNA, a long non-coding (lnc) RNA, a nuclear RNA, an interfering RNA (iRNA), a small interfering RNA (siRNA), a ribozyme, a riboswitch, a satellite RNA, a microswitch, a microzyme, or a viral RNA.

In some embodiments, the target DNA is a eukaryotic DNA.

In some embodiments, the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

In some embodiments, the target DNA is in a eukaryotic cell, for example, a human cell, a non-human primate cell, or a mouse cell.

In some embodiments, the administrating comprises local administration or systemic administration.

In some embodiments, the administrating comprises intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administration is injection or infusion.

In some embodiments, the subject is a human, a non-human primate, or a mouse.

In some embodiments, the level of the transcript (e.g., mRNA) of the target DNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target DNA in the subject prior to the administration.

In some embodiments, the level of the transcript (e.g., mRNA) of the target DNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target DNA in the subject prior to the administration.

In some embodiments, the level of the expression product (e.g., protein) of the target DNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target DNA in the subject prior to the administration.

In some embodiments, the level of the expression product (e.g., protein) of the target DNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target DNA in the subject prior to the administration. In some embodiments, the expression product is a functional mutant of the expression product of the target DNA.

In some embodiments, the median survival of the subject suffering from the disease but receiving the administration is 5 days, 10 days, 20 days, 30 days, 2 months, 3 months, 4 months, 5 months, 6 months, 7 months, 8 months, 9 months, 10 months, 11 months, 12 months, 1.5 year, 2 years, 2.5 years, 3 years, 4 years, 5 years, 6 years, 7 years, 8 years, 9 years, 10 years or more longer than that of a subject or a population of subjects suffering from the disease and not receiving the administration.

The therapeutically effective dose may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

For example, the therapeutically effective dose of the rAAV particle may be about 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration.

### Method of Detection

In yet another aspect, the disclosure provides a method of detecting a target DNA, comprising contacting the target DNA with the system of the disclosure, wherein the target DNA is modified by the complex, and wherein the modification detects the target DNA. In some embodiments, the modification generates a detectable signal, e.g., a fluorescent signal.

### Kits

In yet another aspect, the disclosure provides a kit comprising the Cas12f polypeptide of the disclosure, the system of the disclosure, the polynucleotide of the disclosure, the vector of the disclosure, the RNP of the disclosure, the LNP of the disclosure, the delivery system of the disclosure, the cell of the disclosure, or the pharmaceutical composition of the disclosure, or any one, two, or all components of the same.

In some embodiments, the kit further comprises an instruction to use the component(s) contained therein, and/or instructions for combining with additional component(s) that may be available or necessary elsewhere.

In some embodiments, the kit further comprises one or more buffers that may be used to dissolve any of the component(s) contained therein, and/or to provide suitable reaction conditions for one or more of the component(s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 Celsius degree.

Further embodiments are illustrated in the following Examples which are given for illustrative purposes only and are not intended to limit the scope of the disclosure.

### EXAMPLES

Among others, two hypercompact Cas12f1 from Oscillibacter sp. (OsCas12f1) and Ruminiclostridium herbifermentans (RhCas12f1) were presented herein. Through protein engineering and sgRNA optimization, enhanced OsCas12f1 (enOsCas12f1) and enhanced RhCas12f1 (enRhCas12f1) systems were generated, showing both high on-target cleavage activity and low off-target cleavage activity, and also a wide range of target recognition in human cells. Furthermore, enOsCas12f1 and its inducible version achieved efficient restoration of dystrophin in humanized mdx mice by single AAV delivery. Additionally, enOsCas12f1 was engineered for both epigenome editing and gene activation.

### Material and Methods

Unless otherwise specified, the experimental methods used in the Examples are conventional.

Unless otherwise specified, the materials, reagents, etc., used in the Examples are commercially available.

Unless otherwise specified, the following materials and experimental methods were used in the Examples.

### Ethical Statement

All the research in the disclosure complies with all relevant ethical regulations, and animal experiments have been approved by the Animal Care and Use Committee of HuidaGene Therapeutics Co., Ltd, Shanghai, China.

### Computational Analysis of CRISPR-Cas12f Systems and PAM Prediction

More than 200,000 bacteria genomes were downloaded from NCBI database. Firstly, the applicant used TBLASTN and UnCas12f protein to identify Cas12f-containing sequences of bacteria genomes downloaded from NCBI with E value<1e-10. Then, “0.Cas-Finder.pl” script was used to annotate the CRISPR array and Cas proteins of Cas12f-containing sequences. The applicant further used “1.Cas12f-Finder.pl” to annotate the Cas12f proteins with conserve RuvC and Zn finger domain.

Then, the definition of the 5′ boundary of crRNA depends on the prediction of anti-repeat in tracrRNA. The direct repeats of mature Cas12s′ crRNAs are generally in the 3′ end sequence of about 22 nt. Therefore, the applicant used the 22 nt sequence at the 3′ end of DR to search the non-coding sequence between the Cas12f gene and CRISPR array.

The applicant defined the non-coding sequence containing at least 9 A-U/C-G pairs, and at least 65% of A-U/C-G/G-U pairs with 22 nt sequence at the 3′ end of DR as the anti-repeat sequence. The applicant further extended 150 nt upstream of anti-repeat to obtain potential tracrRNA sequences. Then, using RNAfold to predict the secondary structure of the potential tracrRNA sequences, the applicant retained the sequences with conservative secondary structure in Cas12f family. Based on the above principles, the applicant wrote “2.Cas12f.tracrRNA.Finder.pl” script to predict the tracrRNA sequences of Cas12f variants.

The applicant initially predicted the PAMs for 34 CRISPR-Cas12f systems by CRISPRTarget, ten of these CRISPR-Cas12f systems were successfully predicted (Table 1). The PAMs of the other CRISPR-Cas12f systems were then predicted based on the protein homology with those Cas12f1 whose PAMs were successfully obtained by CRISPRTarget.

### Plasmids Construction and Purification of Cas12f1 Proteins

Human codon-optimized Cas12f1 proteins and sgRNA were synthesized and cloned to generate pCAG_NLS-Cas12f-NLS_pA_pU6_gRNA scaffold-2× BpiI_pCMV_mCherry_pA by NEBuilder (New England Biolabs). The spacer sequences were annealed and ligated to BpiI sites.

For the generation of Cas12f1 mutants, region 1˜3 of OsCas12f1 and RhCas12f1 were divided into 11 segments containing 17 amino acid residues in length. Eleven backbone mutants for OsCas12f1 and RhCas12f1, respectively, were generated by replacing the above mentioned 11 segments with BpiI recognition sequence by PCR and Gibson assembly method using NEBuilder HiFi DNA Assembly Master Mix (New England Biolabs). The specific mutation is then introduced by incorporation of annealed oligos containing mutation by BpiI digestion and T4 DNA ligase ligation.

Full length OsCas12f1, enOsCas12f1, RhCas12f1, or enRhCas12f1 was cloned into pET-32a to express Cas12f1 proteins with C-terminal 6×His. Plasmids were transformed into Escherichia coli BL21(DE3) cells and grown at 37° C. to OD₆₀₀ of 0.6 and then induced for protein expression by 0.5 mM IPTG incubated at 18° C. overnight. Cells were harvested and lysed by sonication in Buffer A (50 mM Tris-HCl (PH=8.0), 50 mM imidazole, 1.5 M NaCl). After centrifugation, the supernatant was gathered and loaded onto the HisTrap HP column (Cytiva) and eluted with Buffer B (50 mM Tris-HCl (PH=8.0), 600 mM imidazole, 1.5 M NaCl). The eluted protein was exchanged into Buffer C containing 20 mM Tris-HCl (PH=8.0), 0.3 M NaCl, 1 mM DTT, and 2% (v/v) glycerol. The protein was then loaded on a HiTrap Heparin HP column (Cytiva), equilibrated with Buffer C, and eluted using a linear gradient of increasing NaCl concentration from 0.3 M to 2.0 M. The obtained protein was stored in Buffer D (25 mM Tris-HCl (PH=8.0), 150 mM NaCl, 2 mM DTT and 1 mM MgCl₂). For long-term storage, the protein was supplemented with 10% (v/v) glycerol, then flash-frozen in liquid nitrogen and stored at −80° C.

sgRNA Synthesis

The sgRNAs were prepared by in vitro transcription using a MEGA shortscript T7 kit (Life Technologies) and purified by a MEGA clear kit (Life Technologies). DNA templates for T7 transcription were generated by PCR using primers containing a T7 promoter. Sequences of these sgRNAs are provided in Table 5.

### In Vitro Cleavage Assay and PAM Characterization

Cas12f1 ribonucleoprotein (RNP, 1 μM) complexes were assembled by mixing Cas12f1 protein with sgRNA at 1:1 molar ratio followed by incubation assembly buffer (10 mM Tris-HCl, pH 7.5, 100 mM NaCl, 1 mM EDTA, 1 mM DTT) at 37° C. for 30 min. Five nM of supercoiled or linear plasmids containing target sequences were incubated with 250 nM Cas12f1 RNP in reaction buffer (2.5 mM Tris-HCl, pH 7.5, 25 mM NaCl, 0.25 mM DTT, and 10 mM MgCl2) at 46° C. or indicated temperature for testing optimal temperature for one hour. The reaction was stopped with quenching buffer (20 mM EDTA, 0.1 mg/ml proteinase K). The digested product was analyzed with 1% of agarose gel. For run-off sequencing the digested product was purified and subjected to Sanger sequencing.

In vitro PAM characterization was performed as previously described. Briefly, the dsDNA library with 7-bp random sequences followed by protospacer sequence was created by PCR with primer with 7N. The in vitro cleavage was performed as above mentioned. The cleaved product with 7 N sequence was gel purified, adapter ligated, and PCR for NGS. The top 1000 enriched PAM sequences were used to draw PAM motifs by WebLogo.

### Size-Exclusion Chromatography

To validate the Cas12f1-sgRNA complex formation, Cas12f1 RNP was assembled in vitro with 4:3 molar ratio of protein:gRNA in buffer D at 37° C. for 30 min and analyzed on Superdex 200 Increase 10/300 column (Cytiva), equilibrated with Buffer D. Buffer E (20 mM Tris-HCl (PH=8.0), 500 mM NaCl, 1 mM DTT and 5 mM MgCl₂) was used for analysis of Cas12f1 protein without sgRNA in view of the fact that OsCas12f1 protein could not be eluted from the column equilibrated with Buffer D, which may be due to non-specific interaction with the resin. The Gel Filtration Standard (Bio-Rad, #1511901) was used for calibration.

### Cell Culture, Transfection, and Flow Cytometry Analysis

HEK293T cells (Stem Cell Bank, Chinese Academy of Sciences) cultured in DMEM supplemented with 10% FBS and penicillin/streptomycin were seeded on 24-well poly-D-lysine coated plates (Corning). For EGFP activation assay, transfection was conducted following the manufacturer's manual with 3.2 μl of PEI (Polyscience) and 1.6 μg of plasmids (0.8 μg of reporter plasmids+0.8 μg of Cas12f expressing plasmids). Forty-eight hours after transfection, flow cytometry analysis was performed to evaluate the EGFP activation efficiency. For analyzing the indel efficiency of endogenous gene, HEK293T cells were transfected with 2 μl of PEI and 1 μg of plasmids expressing Cas12f and sgRNA cassette. The mCherry-positive cells were collected by FACS sorting at 72 h after transfection.

### Indel Efficiency Analysis at Human Endogenous Genomic Loci

Eight thousand sorted cells were harvested for genomic DNA extraction by addition of 20 μl of lysis buffer (Vazyme) following the manufacturer's manual. For TIDER test, the genomic region in the vicinity of Cas nuclease target site was amplified by Phanta Max Super-Fidelity DNA Polymerase (Vazyme) using nested PCR. Purified PCR products were Sanger sequenced and analyzed as previously described. For deep sequencing analysis, the targeted genomic region was amplified by Phanta Max Super-Fidelity DNA Polymerase (Vazyme) using nested PCR, primers with barcode were used. PCR products were purified by Gel extraction kit (Vazyme) and sequenced on an Illumina HiSeq X System (150-bp paired-end reads). Forward reads were aligned to the reference sequences using BWA (v0.7.17-r1188) with parameter of “bwa mem -A2 -O3 -E1”. At each target, editing was calculated as the percentage of total reads containing desired edits without indels within a 10-bp window of the cut site. The target site information is provided in Table 4.

### PEM-seq Analysis

PEM-seq in HEK293 cells was performed as previously described. Briefly, expression plasmids for enOsCas12f1, LbCas12a, SpCas9, and Un1Cas12f_ge4.1 targeted at target 36, as well as enRhCas12f1 and SpCas9 targeted at PCSK9 were transfected into HEK293 cells by PEI, respectively, and after 72 h, positive cells were harvested for DNA extraction. The 20 μg genomic DNA was fragmented with a peak length of 300-700 bp by Covaris sonication. DNA fragments were tagged with biotin by a one-round biotinylated primer extension at 5′-end, and then primer removal by AMPure XP beads and purified by streptavidin beads. The single-stranded DNA on streptavidin beads is ligased with a bridge adapter containing 14-bp RMB, and PCR product was performed nested PCR for enriching DNA fragment containing the bait DSB and tagged with illumine adapter sequences. The prepared sequencing library was sequenced on a Hi-seq 2500, with a 2×150 bp.

### Animals

All animal experiments were performed and approved by the Animal Care and Use Committee of HuidaGene Therapeutics Co., Ltd, Shanghai, China. Mice were housed in a barrier facility with a 12-hour light/dark cycle and 18-23° C. with 40-60% humidity. Diet and water were accessible at all times. DMD mice were generated in the C57BL/6 J background using the CRISPR-Cas9 system. Duchenne muscular dystrophy (DMD) is the most common sex-linked lethal disease in man, thus male mice were selected for this study.

### Intramuscular Injection

rAAV9 particles were produced by PackGene Biotech (Guangzhou, China), and applied iodixanol density gradient centrifugation for purification. For intramuscular injection, DMD mice were anesthetized, and TA (tibialis anterior) muscle was injected with 50 μL of AAV9 (5×10¹¹ vg) preparations or with same volume saline solution. 3 weeks after rAAV9 intramuscular injection, mice were anesthetized and euthanized, and TA (tibialis anterior) muscle was collection.

### RT-PCR and TA Cloning

Muscle total mRNA was extracted, and cDNA was synthesized using a HiScript II One Step RT-PCR Kit (Vazyme, P611-01) following the manufacturer's protocol. Then, each 20 μl PCR reaction contained approximately 2 μl cDNA, 0.25 μM of each of forward and reverse primers, and 10 μl of Ex taq (Takara, RR001A) was performed on a C1000 Touch Thermal Cycler (Bio-Rad). Amplification conditions consisted of an initial hold for 5 min followed by 35 cycles of 95° C. for 30 s, 60° C. for 30 s, and 72° C. for 30 s. PCR products were analyzed by gel electrophoresis.

For detected RNA splicing, TA cloning was performed according to the protocol of the pEASY-T5 Zero Cloning Kit (TransGen Biotech, CT501-01). Brief, PCR products were used agarose gel electrophoresis to verify the quality and quantity. 4 μl PCR products and pEASY-T5 Zero Cloning vector were gently mixed well, incubate at room temperature for 10 minutes, and then add the ligated products to 50 μl of Trams 1-T1 phage resistant chemically competent cell and plated on LB/Amp+, followed by sequencing with M13F.

### Western Blot

Muscle samples were homogenized with RIPA buffer supplemented with protease inhibitor cocktail. Lysate supernatants were quantified with Pierce BCA protein assay kit (Thermo Fisher Scientific, 23225) and adjusted to an identical concentration using H₂O. Samples were mixed with in NuPAGE LDS sample buffer (Invitrogen, NP0007) and 10% β-mercaptoethanol followed by boiled at 70° C. for 10 min. 20 μg total protein per lane was loaded into 3 to 8% tris-acetate gel (Invitrogen, EA03752BOX) and electrophoresed for 1 hours at 200 V. Protein was transferred on a PVDF membrane under the wet condition at 350 mA for 3.5 hours. The membrane was blocked in 5% non-fat milk in TBST buffer and then incubated with primary antibody labeling specific protein. After washing three times with TBST, the membrane was further incubated with HRP conjugated secondary antibody (1:1000 dilution, Beyotime, A0216) specific to the IgG of the species of primary antibody against dystrophin (1:1000 dilution, Sigma, D8168) and vinculin (1:1000 dilution, CST, 13901 S). The target proteins were visualized with Chemiluminescent substrates (Invitrogen, WP20005).

### Immunofluorescence

Tissues were collected and mounted in optimal cutting temperature (OCT) compound and snap-frozen in liquid nitrogen. Serial frozen cryosections (10 μm) were fixed for 2 hours in 37° C. followed by permeabilized with PBS+0.4% Triton-X for 30 min. After washing with PBS, samples were blocked with 10% goat serum for 1 hours at room temperature. Then, the slides were incubated overnight at 4° C. with primary antibodies against dystrophin (1:100 dilution, Abcam, ab15277) and spectrin (1:500 dilution, Millipore, MAB1622). After that, samples were washed extensively PBS and incubated with compatible secondary antibodies (Alexa Fluor 488 AffiniPure donkey anti-rabbit IgG (1:1000 dilution, Jackson ImmunoResearch labs, 711-545-152) or Alexa Fluor 647 AffiniPure donkey anti-mouse IgG (1:1000 dilution, Jackson ImmunoResearch labs, 715-605-151)) and DAPI for 2 h at room temperature. Samples were washed for 10 min with PBS and repeated three times. And then, slides were sealed with fluoromount-G mounting medium. All images were visualized under Nikon C2. The amount of dystrophin-positive muscle fibers is represented as a percentage of total spectrin-positive muscle fibers.

Efficiency Detection on miniCRISPRoff

One microgram of mCherry containing plasmids expressing miniCRISPRoff and CRIPSRoff were transfected into Snrp-GFP stablely expressed HEK293T cells. Two days after transfection, mCherry-positive cells were sorted and cultured for FACS analysis at the indicated time.

For bisulfite sequencing analysis, genomic DNA was treated by BisulFlash DNA Modification Kit (EPIGENTEK) as the manufacturer's protocols. PCR amplicon of GAPDH-Snrp promoter was purified and cloned into TA cloning vector (VAYZYME). Colonies were randomly picked for Sanger sequencing.

### Statistics and Reproducibility

Frequency, mean, and standard deviations were calculated using GraphPad Prism 8. Whole-genome sequencing analysis was conducted using BWA (v0.7.17-r1188) with parameter of “bwa mem -A2 -O3 -E1”. PEM-seq data analysis was performed using PEM-Q pipeline with default parameters. Two or three biologically independent replicates were performed, which was demonstrated in the figure legend. In this study, no statistical method was used to predetermine sample size, and no data were excluded from the analyses. The experiments were not randomized, and the Investigators were not blinded to allocation during experiments and outcome assessment.

### Example 1. Identification and Characterization of Class 2, Type V-F CRISPR-Cas (Cas12f) Systems

34 previously undocumented and uncharacterized CRISPR-Cas12f systems (Table 1) were identified using self-developed computational pipeline to annotate Cas12f orthologs, CRISPR array, tracrRNAs, and PAM preferences. The amino acid sequences of the Cas12f1 proteins of the 34 identified systems and the 4 reported Cas12f systems (controls; Table 1) are set forth in SEQ ID NOs: 1-38, respectively. The codon-optimized coding sequences for the 34 identified Cas12f1 proteins are set forth in SEQ ID NOs: 39-72, respectively. The direct repeat (DR) sequences accompanying the Cas12f1 proteins are set forth in SEQ ID NOs: 179-212, respectively. The reported CRISPR-Cas12f systems were used as control for comparison.

TABLE 1
List of identified and reported CRISPR-Cas12f systems

| | | | PAM used for |
|---|---|---|---|
| | | | GFP activation |
| | | | efficiency |
| | name | Species/source | evaluation |
| Undocumented | OsCas12f1 | Oscillibacter sp. | 5′-TTTC |
| and | (ME-B.3) | | |
| uncharacterized | | | |
| CRISPR-Cas12f | | | |
| systems | | | |
| | RhCas12f1 | Ruminiclostridium | 5′-CCCA/ |
| | (ME-A.1) | herbifermentans | TCCA |
| | Ob2Cas12f1 | Oscillospiraceae bacterium | 5′-TTTC |
| | (ME-B.4) | | |
| | Ob3Cas12f1 | Oscillospiraceae bacterium | 5′-TTTC |
| | (ME-B.5) | | |
| | Cb1Cas12f1 | Clostridia bacterium | 5′-TTTC |
| | (ME-B.14) | | |
| | Cb2Cas12f1 | Clostridia bacterium | 5′-TTTC |
| | (ME-B.1) | | |
| | Cb5Cas12f1 | Clostridiales bacterium | 5′-GTTC |
| | (ME-B.18) | | |
| | Ob1Cas12f1 | Oscillospiraceae bacterium | 5′-GTTC |
| | (ME-B.15) | | |
| | EsCas12f1 | Eubacterium siraeum | 5′-TTTC |
| | (ME-B.16) | | |
| | Pt1Cas12f1 | Parageobacillus | 5′-TTTA |
| | (ME-B.19) | thermoglucosidasius | |
| | RhgCas12f1 | Ruminiclostridium hungatei | 5′-TTTA |
| | (ME-B.2) | | |
| | Bc1Cas12f1 | Bacillus cereus | 5′-TTCA |
| | (ME-B.10) | | |
| | BfCas12f1 | Bacillus fungorum | 5′-TTCA |
| | (ME-B.8) | | |
| | BtCas12f1 | Bacillus thuringiensis | 5′-TTCA |
| | (ME-B.6) | | |
| | HsCas12f1 | Hydrothermal sediment microbial | 5′-TTTC |
| | (ME-B.12) | communities from Guaymas | |
| | | Basin, California, USA | |
| | MsCas12f1 | Marine sediment microbial | 5′-TTTC |
| | (ME-B.13) | communities from Aarhus Bay | |
| | | station M5, Denmark | |
| | ScCas12f1 | Sediment core sample microbial | 5′-TTTG |
| | (ME-B.11) | community from Chocolate Pots | |
| | | hot springs, Yellowstone National | |
| | | Park, Wyoming, USA | |
| | Un2Cas12f1 | Uncultured bacterium | 5′-TTTG |
| | (ME-B.20) | | |
| | CiCas12f1 | Clostridium ihumii | 5′-GGAG |
| | (ME-B.7) | | |
| | CpCas12f1 | Clostridium paraputrificum | 5′-GGAG |
| | (ME-B.9) | | |
| | SvCas12f1 | Sarcina ventriculi | 5′-GGAG |
| | (ME-B.17) | | |
| | AoCas12f1 | Anaerobic oil degrading | 5′-TCCA |
| | (ME-A.7) | microbial communities from River | |
| | | Tyne estuarine sediment | |
| | Bc2Cas12f1 | Bacillus cereus | 5′-TCCA |
| | (ME-A.5) | | |
| | CdCas12f1 | Clostridioides difficile | 5′-ATCA |
| | (ME-A.3) | | |
| | Cs1Cas12f1 | Clostridium sporogenes | 5′-ACCA |
| | (ME-A.4) | | |
| | Cb3Cas12f1 | Clostridium botulinum | 5′-TCCA |
| | (ME-A.10) | | |
| | Cb4Cas12f1 | Clostridium baratii | 5′-TCCA |
| | (ME-A.11) | | |
| | BsCas12f1 | Blautia sp. M16 M6_ctg015 | 5′-TCCA |
| | (ME-A.12) | | |
| | Pt2Cas12f1 | Parageobacillus | 5′-TCCA |
| | (ME-A.9) | thermoglucosidasius | |
| | CrCas12f1 | Cellulosilyticum ruminicola | 5′-TCCA |
| | (ME-A.8) | | |
| | ChCas12f1 | Clostridium hiranonis | 5′-TCCA |
| | (ME-A.2) | strain DSM 13275 | |
| | Cs2Cas12f1 | Clostridium sp | 5′-TCCA |
| | (ME-A.6) | | |
| | PhCas12f1 | Peptacetobacter hiranonis | 5′-TCCA |
| | (ME-A.13) | strain | |
| | OpbCas12f1 | Opitutae bacterium | 5′-CCCA |
| Reported | CnCas12f1 | Clostridium novyi | 5′-ACCT |
| CRISPR-Cas12f | | | |
| systems | | | |
| | Un1Cas12f1 | Uncultured bacterium | 5′-TTTG |
| | (Un1Cas12f1_ | | |
| | ge4.1) | | |
| | SpCas12f1 | Syntrophomonas palmitatica | 5′-GTTC |
| | AsCas12f1 | Acidibacillus sulfuroxidans | 5′-TTTA |

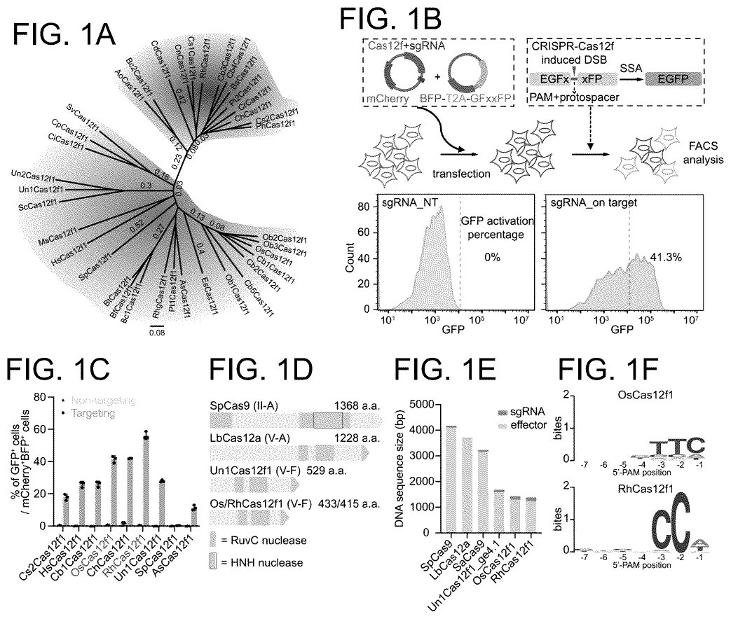

To evaluate the efficiency of the spacer sequence-specific (on-target) dsDNA cleavage (“dsDNA cleavage” for short unless otherwise indicated) in eukaryotic cells by these CRISPR-Cas12f systems, an enhanced green fluorescent protein (EGFP) reporter system activatable by single-strand annealing (SSA)-mediated repair pathway in HEK293T cells was designed (FIG. 1B and FIG. 22). See also CN 202111290670.8, CN 202111289092.6, CN 202210081981.1, PCT/CN2022/129376, and PCT/CN2023/073420 for the disclosure of similar assay, each of which is incorporated herein by reference in its entirety. This reporter system relied on co-transfection with a reporter plasmid and an expression plasmid.

The reporter plasmid (FIG. 1B and FIG. 22) carried a BFP-T2A-GFxxFP expression cassette with a deactivated EGFP coding sequence (GFxxFP) harboring an insertion sequence (SEQ ID NO: 213; containing 5′ PAM [symbol] which is replaceable to adapt to the PAM preference of various Cas12 proteins, premature stop codon [symbol] to prevent expression of EGFP, and 3′ PAM [symbol] to adapt to Cas9 protein) between EGFx (EGFP CDS 1-561 bp) and xFP (EGFP CDS 112-720 bp) (referring to Table 1 for PAM for each Cas12f1 protein). The BFP indicated successful transfection and expression of the reporter plasmid in host cells.

PAM & stop codon containing
insertion sequence
SEQ ID NO: 213 [symbol]
CCATTACAG [symbol] GAGCATAC [symbol] ,
protospacer sequence/
targeting spacer sequence
SEQ ID NO: 214
CCATTACAGTAGGAGCATAC,
SEQ ID NO: 215
non-targeting spacer sequence
GGGTCTTCGAGAAGACCT,

The expression plasmid (FIG. 1B and FIG. 22) carried an expression cassette for each of the Cas12f1 proteins and its sgRNA targeting the insertion sequence in the reporter plasmid and mCherry indicating successful transfection and expression of the expression plasmid in host cells.

Each of the Cas12f1 proteins was tagged with a SV40 nuclear localization sequence (SV40 NLS) (SEQ ID NO: 216; coded by SEQ ID NO: 217) at its N-terminal and a nucleoplasmin NLS (NP NLS, npNLS) (SEQ ID NO: 218; coded by SEQ ID NO: 219) at its C-terminal.

SV40 NLS amino acid sequence
SEQ ID NO: 216
PKKKRKV,
SV40 NLS coding sequence
SEQ ID NO: 217
CCTAAGAAGAAGAGAAAGGTG,
NP NLS amino acid sequence
SEQ ID NO: 218
KRPAATKKAGQAKKKK,
NP NLS coding sequence,
SEQ ID NO: 219
AAAAGGCCGGCGGCCACGAAGAAGGCC
GGCCAGGCAAAGAAGAAGAAG

The polynucleotide sequences of the scaffold sequences of the sgRNAs corresponding to the 34 identified systems and the 4 reported systems are set forth in SEQ ID NOs: 73-110, respectively.

The sgRNA encoded on the expression plasmid was composed of, from 5′ to 3′ direction, one scaffold sequence (one of SEQ ID NOs: 73-110), one targeting spacer sequence (SEQ ID NO: 214) capable of hybridizing to the insertion sequence (SEQ ID NO: 213) in the reporter plasmid, and one stabilizing sequence (SEQ ID NO: 220) for increased sgRNA stability, with no linker between any two of the preceding components. Each of the scaffold sequences of SEQ ID NO: 73-106 was composed of, from 5′ to 3′ direction, one tracrRNA (one of SEQ ID NOs: 111-144), one GAAA tetraloop as a linker, and one repeat sequence (one of SEQ ID NOs: 145-178). In other words, the sgRNA was composed of, from 5′ to 3′ direction, one tracrRNA (one of SEQ ID NOs: 111-144), one GAAA tetraloop as a linker, one crRNA, and one stabilizing sequence (SEQ ID NO: 220), with no linker between any two of the preceding components, wherein the crRNA was composed of, from 5′ to 3′ direction, one repeat sequence (one of SEQ ID NOs: 145-178) and one targeting spacer sequence (SEQ ID NO: 214) with no linker therebetween. A non-targeting (NT) spacer sequence (SEQ ID NO: 215) incapable of hybridizing to the insertion sequence (SEQ ID NO: 213) was used in place of the spacer sequence (SEQ ID NO: 214) as a negative control. It is noted that in the scaffold sequence (SEQ ID NO: 88) of MsCas12f1 (ME-B.13), the tracrRNA (SEQ ID NO: 126) is direct fused to the repeat sequence (SEQ ID NO: 160) without the GAAA tetraloop. Each of the repeat sequences (SEQ ID NOs: 145-178) is derived from the corresponding DR sequence (SEQ ID NOs: 179-212).

SEQ ID NO: 220
stabilizing sequence
TTTTATTTTTTT,

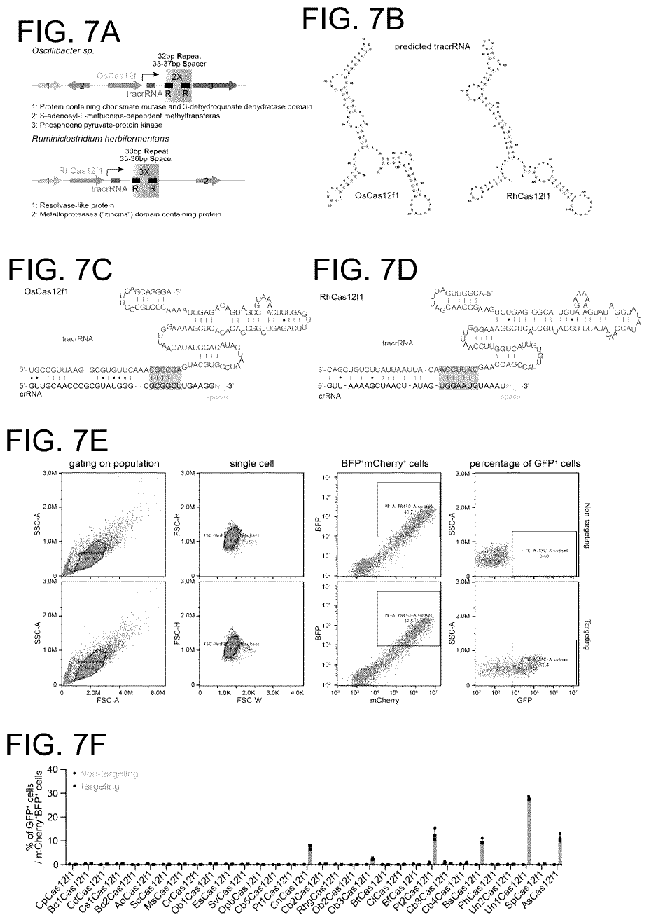

The DSBs generated in the reporter plasmid by the dsDNA cleavage by the Cas12f1 protein as guided by the sgRNA targeting the insertion sequence would induce SSA-mediated repair of the GFxxFP coding sequence, consequently activating EGFP expression (FIG. 1B and FIG. 7E) indicating dsDNA cleavage, which was represented by the percentage proportion of GFP positive cells in mCherry & BFP dual-positive cells (% of GFP⁺ cells/mCherry⁺BPF⁺ cells).

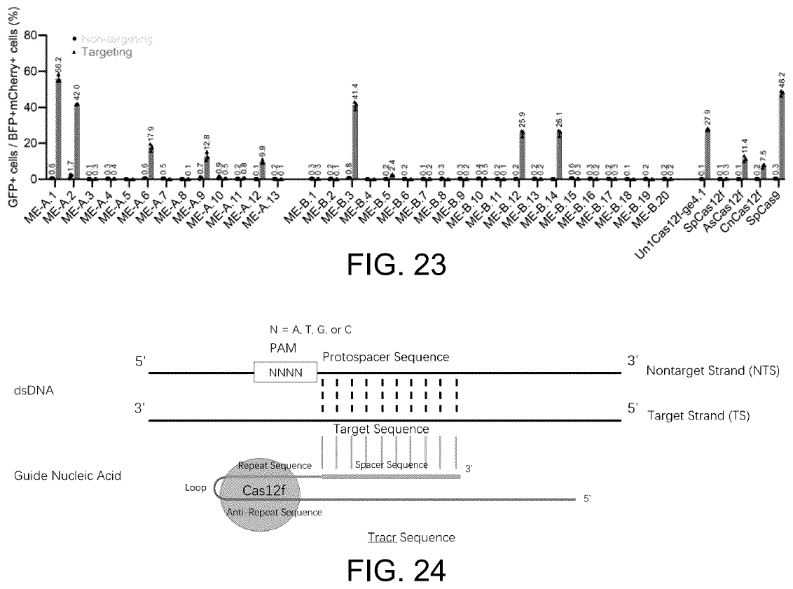

Using this fluorescent screen method, nine identified CRISPR-Cas12f systems (FIG. 1C, FIG. 7F, FIG. 23) were functionally characterized to show dsDNA cleavage activity: OsCas12f1 (ME-B.3, SEQ ID NO: 1), RhCas12f1 (ME-A.1, SEQ ID NO: 2), Ob3Cas12f1 (ME-B.5, SEQ ID NO: 4), Cb1Cas12f1 (ME-B.14, SEQ ID NO: 5), HsCas12f1 (ME-B.12, SEQ ID NO: 15), BsCas12f1 (ME-A.12, SEQ ID NO: 28), Pt2Cas12f1 (ME-A.9, SEQ ID NO: 29), ChCas12f1 (ME-A.2, SEQ ID NO: 31), and Cs2Cas12f1 (ME-A.6, SEQ ID NO: 32).

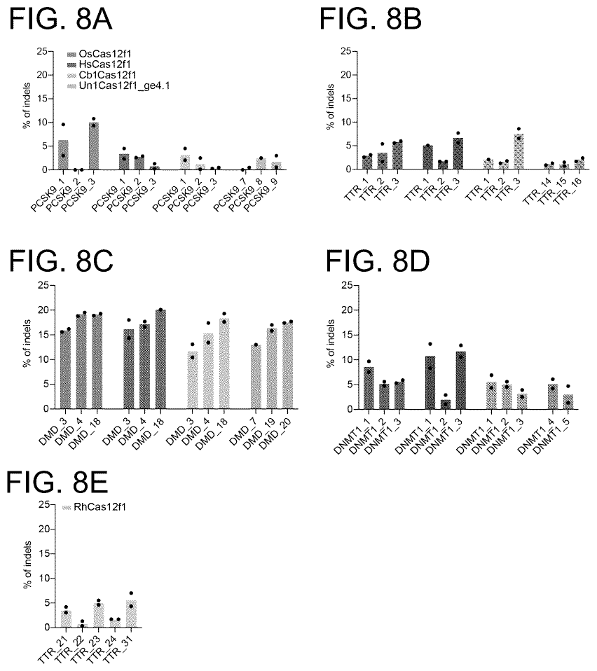

Based on the observations of robust EGFP activation by OsCas12f1, HsCas12f1, Cb1Cas12f1, and RhCas12f1 in HEK293T cells, the frequency of indel generated by these Cas12f1 at endogenous genomic loci (PCKS9, TTR, DMD, and DNMT1 genes) was also validated. The dsDNA cleavage activity (genomic editing efficiency) was represented by % indel. The results showed that the genomic editing efficiencies of OsCas12f1, HsCas12f1, Cb1Cas12f1, and RhCas12f1 were modest, with indel frequencies ranging from about 1% to about 20% at various target loci (FIGS. 8A-8E). For FIGS. 8A-8D, the 4 groups of columns are OsCas12f1, HsCas12f1, Cb1Cas12f1, and Un1Cas12f1_ge4.1 from the left side to the right side.

The two CRISPR-Cas12f systems OsCas12f1 (433 aa) and RhCas12f1 (415 aa) with the highest dsDNA cleavage activity (as represented by GPF activation efficiency) were selected for further study, which recognized 5′ T-rich PAM (e.g., 5′-TTTC) and 5′ C-rich PAM (e.g., 5′-CCCA/TCCA), respectively. Both OsCas12f1 and RhCas12f1 are hypercompact, with a gene size that is less than half of SpCas9, LbCas12a, and SaCas9 (FIGS. 1D and 1E).

Further, the in vitro cleavage of a DNA fragment library containing 7-bp random sequence indicated that OsCas12f1 and RhCas12f1 recognized 5′ PAMs of 5′-{C/T;T/C/A;T/C/A;C/A/T} (i.e., in the four-letter 5′ PAM, the first nucleotide can be C or T; the second nucleotide can be T or C or A; the third nucleotide can be T or C or A; and the fourth nucleotide can be C or A or T) and 5′-{N;C/A/G;C;A/T/G} (i.e., in the four-letter 5′ PAM, the first nucleotide can be A or T or G or C; the second nucleotide can be C or A or G; the third nucleotide can be Cl; and the fourth nucleotide can be A or T or G), respectively (FIG. 1F).

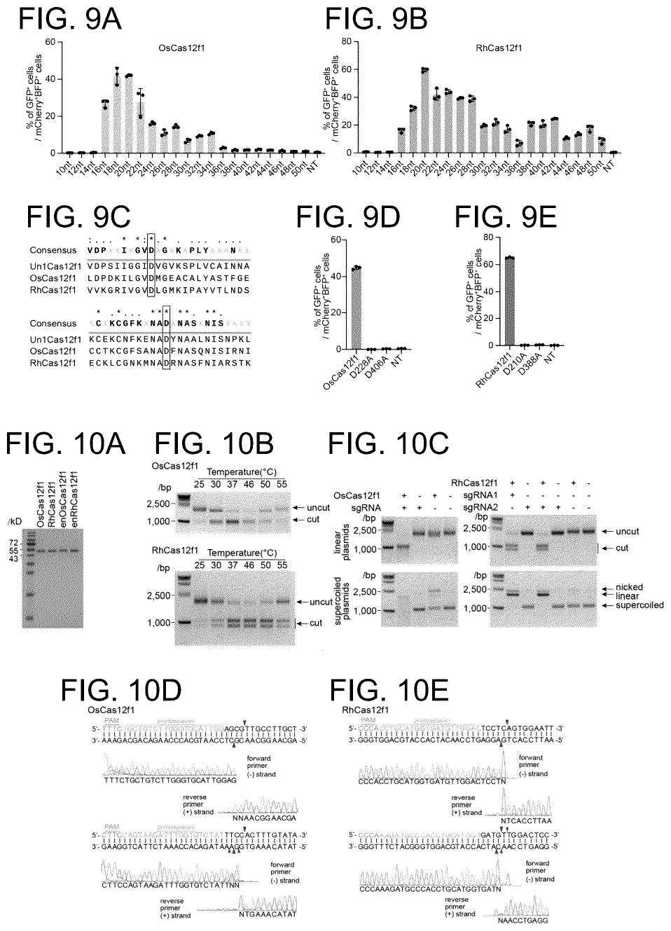

The effects of spacer length on cleavage efficiency of OsCas12f1 and RhCas12f1 were explored by designing insertion sequences and corresponding sgRNAs with various lengths of target sequences and corresponding spacer sequences, which showed that a length of at least 16 nt and an optimal length of 20 nt worked for both OsCas12f1 and RhCas12f1 (FIGS. 9A and 9B).

By introducing point mutations that resulted in D228A (SEQ ID NO: 221) or D406A (SEQ ID NO: 222) residue conversions in the conserved active sites of the RuvC domain, the cleavage activity of OsCas12f1 was abolished, generating an endonuclease deficient (dead) OsCas12f1 variant (FIGS. 9C and 9D). Similarly, RhCas12f1 could be inactivated through nonsynonymous point mutations leading to D210A (SEQ ID NO: 223) or D388A (SEQ ID NO: 224) conversion mutations, generating an endonuclease deficient (dead) RhCas12f1 variant (FIG. 9E). Each of the single mutations was in the conserved active sites of the RuvC domain of the Cas12f1 proteins.

The biochemical properties of OsCas12f1 and RhCas12f1 proteins (FIG. 10A) were tested. Linear plasmids cleavage assay suggested that both OsCas12f1 and RhCas12f1 were dsDNA cleavage active at a wide range of temperature, preferring 37° C.-50° C. (FIG. 10B). The cleavage activities of OsCas12f1 and RhCas12f1 were validated on both supercoiled and linear plasmids (FIG. 10C). To characterize the dsDNA cleavage pattern of OsCas12f1 and RhCas12f1, run-off sequencing of in vitro cleavage products was performed, indicating that OsCas12f1 and RhCas12f1 cut dsDNA at sites of 21-25 bp downstream of the 5′-PAM with sticky ends (FIGS. 10D and 10E).

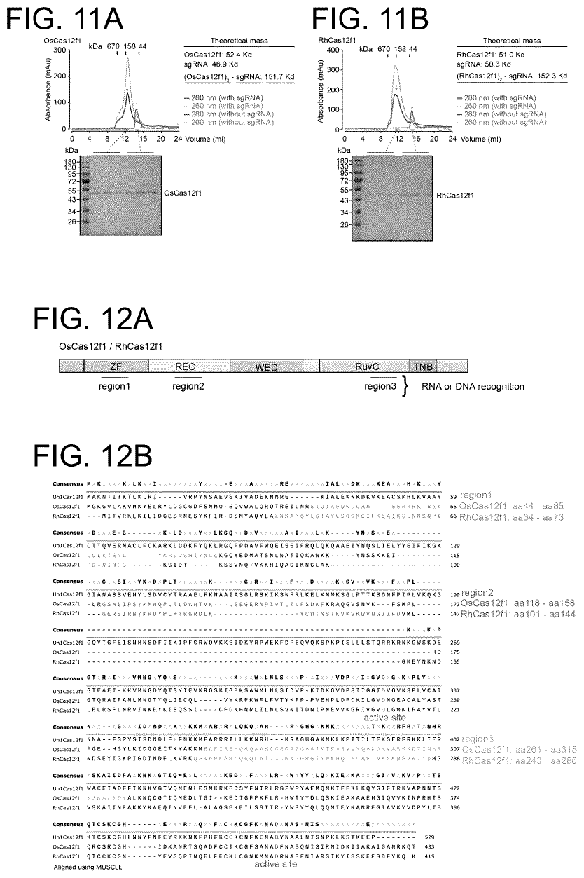

Size-exclusion chromatography was performed to determine the complex formation of the Cas12f1 protein with its sgRNA, suggesting that both OsCas12f1 and RhCas12f1 could form dimer in presence of sgRNA at least in the tested condition, which was similar to that of Un1Cas12f1 (FIGS. 11A-11B).

Taken together, these results indicated that OsCas12f1 and RhCas12f1 offer hypercompact DNA editing tools with modest genomic editing efficiency and relatively wide target range.

### Example 2. Arginine Substitution in the REC/RuvC Domains and C-G Base Pair Replacement in the sgRNA Enhanced Cleavage Efficiency of OsCas12f1 and RhCas12f1

In order to increase the cleavage efficiency of OsCas12f1 and RhCas12f1, these Cas12f1 proteins were engineered through mutagenesis and screening for higher efficiency variants using the same GFP activation reporter system, as described above (FIG. 1B and FIG. 22).

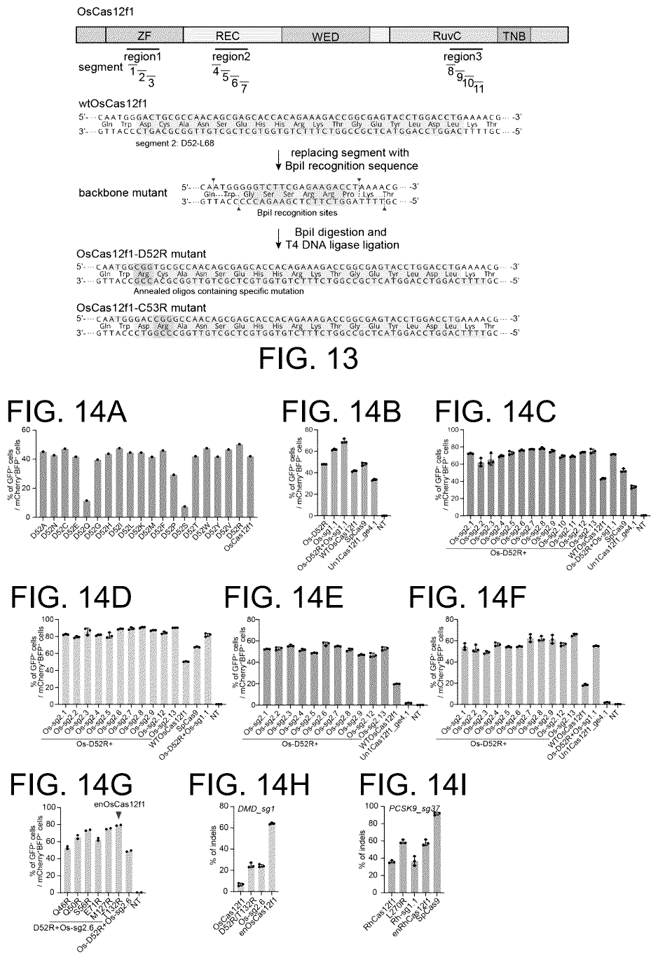

Based on the protein alignment of OsCas12f1 and RhCas12f1 with Un1Cas12f1, three regions that potentially responding for binding nuclei acids were defined (FIGS. 12A-12B). Amino acids (except for positively charged residues including lysine, arginine, and histidine) in the region1˜3 of OsCas12f1 and RhCas12f1 were individually mutated into arginine by mutagenesis method as previously reported (FIG. 13, Table 2).

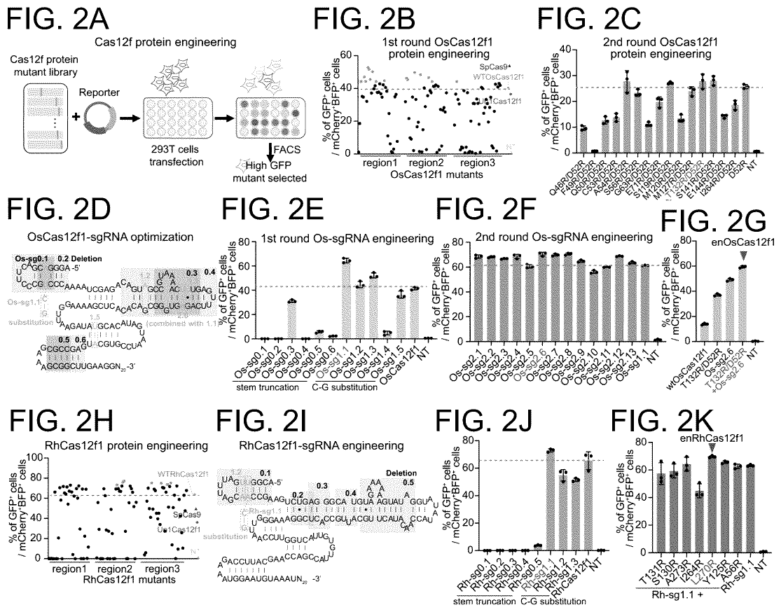

Two mutant libraries were generated in the first round within these three regions, each containing over 100 mutants of OsCas12f1 or RhCas12f1. These mutants were then individually co-transfected with the reporter plasmid into HEK293T cells, and EGFP activation efficiency evaluation by the reporter system in Example 1 was quantified by flow cytometry (FIG. 2A). Although most mutants showed similar or lower efficiency to that of wild-type OsCas12f1 (WTOsCas12f1), a subset of mutants exhibited increased activity (FIG. 2B and Table 2). The most efficient OsCas12f1 mutant, D52R (OsCas-D52R; SEQ TD NO: 225), showed 1.31-fold improvement over WTOsCas2f1 (FIG. 2B). To determine whether substitution with other amino acids could further enhance cleavage efficiency over that of the D52R variant, D52 was mutagenized to saturation and found that the R substitution indeed conferred a better or slightly better OsCas12f1 nuclease activity (FIG. 14A). “NT” refers to a negative control using a non-targeting spacer (SEQ ID NO: 215).

TABLE 2
Cleavage activity of OsCas12f1 and RhCas12f1 mutants

| | Cleav- | | Cleav- | | Cleav- |
|---|---|---|---|---|---|
| | age | | age | | age |
| | activ- | | activ- | | activ- |
| Sample | ity (%) | Sample | ity (%) | Sample | ity (%) |
| Os-aa44 | 1.13 | Os-aa122 | 9.8 | Os-aa270 | 4.27 |
| Os-aa45 | 18.6 | Os-aa123 | 9.08 | Os-aa271 | 3.74 |
| Os-aa46 | 44.2 | Os-aa124 | 34.7 | Os-aa272 | 38.6 |
| Os-aa47 | 35.1 | Os-aa125 | 2.12 | Os-aa274 | 0.66 |
| Os-aa48 | 34.7 | Os-aa127 | 49.7 | Os-aa275 | 4.16 |
| Os-aa49 | 47 | Os-aa128 | 33.3 | Os-aa276 | 37.4 |
| Os-aa50 | 44.2 | Os-aa129 | 16.6 | Os-aa277 | 0.75 |
| Os-aa51 | 32.1 | Os-aa130 | 19.4 | Os-aa279 | 1.52 |
| Os-aa52 | 52.8 | Os-aa131 | 1.47 | Os-aa280 | 20 |
| Os-aa53 | 51.7 | Os-aa132 | 48.4 | Os-aa282 | 1.13 |
| Os-aa54 | 45.5 | Os-aa133 | 1.26 | Os-aa283 | 30.8 |
| Os-aa55 | 37.1 | Os-aa134 | 14.8 | Os-aa285 | 30.7 |
| Os-aa56 | 50.2 | Os-aa136 | 40.3 | Os-aa287 | 0.32 |
| Os-aa57 | 39.8 | Os-aa137 | 28.9 | Os-aa288 | 33.5 |
| Os-aa62 | 41.6 | Os-aa138 | 2.69 | Os-aa289 | 30.9 |
| Os-aa63 | 44.3 | Os-aa140 | 35.5 | Os-aa290 | 18.1 |
| Os-aa64 | 37.5 | Os-aa141 | 46.6 | Os-aa291 | 1.02 |
| Os-aa65 | 37.7 | Os-aa142 | 38.5 | Os-aa292 | 40.6 |
| Os-aa66 | 42.8 | Os-aa143 | 37.4 | Os-aa293 | 39.9 |
| Os-aa67 | 36.7 | Os-aa144 | 44.2 | Os-aa295 | 24.8 |
| Os-aa68 | 34.6 | Os-aa146 | 41.3 | Os-aa297 | 28.4 |
| Os-aa70 | 42.2 | Os-aa147 | 41.4 | Os-aa298 | 33.3 |
| Os-aa71 | 44.7 | Os-aa148 | 42.1 | Os-aa300 | 30.7 |
| Os-aa72 | 40.6 | Os-aa149 | 0.95 | Os-aa302 | 19.5 |
| Os-aa73 | 38.3 | Os-aa150 | 41.3 | Os-aa303 | 28.4 |
| Os-aa74 | 11 | Os-aa151 | 2.48 | Os-aa304 | 34.6 |
| Os-aa77 | 24.7 | Os-aa152 | 27.1 | Os-aa305 | 4.9 |
| Os-aa78 | 6.18 | Os-aa153 | 4.88 | Os-aa308 | 3.65 |
| Os-aa79 | 1.4 | Os-aa154 | 26.7 | Os-aa309 | 26 |
| Os-aa81 | 0.82 | Os-aa155 | 28.3 | Os-aa311 | 42.5 |
| Os-aa82 | 2.97 | Os-aa156 | 25.6 | Os-aa312 | 1.79 |
| Os-aa83 | 34.9 | Os-aa158 | 37.3 | Os-aa313 | 40.9 |
| Os-aa84 | 36.4 | Os-aa261 | 17.7 | Os-aa314 | 43 |
| Os-aa85 | 7.67 | Os-aa262 | 36.5 | Os-aa315 | 41.9 |
| Os-aa118 | 31.9 | Os-aa264 | 44.2 | SpCas9 | 53.9 |
| Os-aa119 | 46.3 | Os-aa266 | 33.3 | Un1Cas12f1_ge4.1 | 36.5 |
| Os-aa120 | 45.4 | Os-aa267 | 30.5 | WTOsCas12f1 | 38.7 |
| Os-aa121 | 21.6 | Os-aa268 | 2.55 | WTOsCas12f1_NT | 0.55 |
| | Cleav- | | Cleav- | | Cleav- |
| | age | | age | | age |
| | activ- | | activ- | | activ- |
| Sample | ity (%) | Sample | ity (%) | Sample | ity (%) |
| Rh-aa2 | 32.4 | Rh-aa145 | 0 | Rh-aa286 | 10.5 |
| Rh-aa3 | 57.9 | Rh-aa146 | 68.5 | Rh-aa288 | 12.6 |
| Rh-aa4 | 0.094 | Rh-aa147 | 65 | Rh-aa289 | 0.16 |
| Rh-aa7 | 0.022 | Rh-aa148 | 4.04 | Rh-aa290 | 0 |
| Rh-aa9 | 0.069 | Rh-aa150 | 64.2 | Rh-aa292 | 70.2 |
| Rh-aa10 | 69.2 | Rh-aa151 | 63.6 | Rh-aa293 | 0.04 |
| Rh-aa11 | 69.2 | Rh-aa152 | 38.4 | Rh-aa294 | 45.5 |
| Rh-aa12 | 58.5 | Rh-aa154 | 23.4 | Rh-aa295 | 66.7 |
| Rh-aa13 | 67 | Rh-aa155 | 55.5 | Rh-aa296 | 0.039 |
| Rh-aa14 | 68.5 | Rh-aa156 | 74 | Rh-aa297 | 0.28 |
| Rh-aa15 | 71.5 | Rh-aa157 | 58.6 | Rh-aa300 | 61.2 |
| Rh-aa17 | 68.3 | Rh-aa158 | 12 | Rh-aa302 | 0 |
| Rh-aa18 | 69.1 | Rh-aa160 | 73.4 | Rh-aa303 | 66.1 |
| Rh-aa19 | 75.8 | Rh-aa161 | 22.5 | Rh-aa304 | 59.1 |
| Rh-aa20 | 70.4 | Rh-aa162 | 0.024 | Rh-aa305 | 0.063 |
| Rh-aa22 | 61 | Rh-aa163 | 72.9 | Rh-aa306 | 8.71 |
| Rh-aa23 | 0 | Rh-aa165 | 0.079 | Rh-aa307 | 0.11 |
| Rh-aa25 | 60.3 | Rh-aa166 | 54.3 | Rh-aa308 | 0 |
| Rh-aa26 | 13.5 | Rh-aa167 | 66.3 | Rh-aa309 | 73.6 |
| Rh-aa27 | 70.1 | Rh-aa169 | 42.2 | Rh-aa310 | 0.039 |
| Rh-aa28 | 68 | Rh-aa170 | 63.3 | Rh-aa311 | 77.8 |
| Rh-aa29 | 43.4 | Rh-aa172 | 49.9 | Rh-aa312 | 58.5 |
| Rh-aa30 | 0 | Rh-aa173 | 70.1 | Rh-aa313 | 77.3 |
| Rh-aa31 | 64 | Rh-aa174 | 23.1 | Rh-aa314 | 67.4 |
| Rh-aa32 | 65.2 | Rh-aa175 | 6.73 | Rh-aa315 | 50.9 |
| Rh-aa33 | 0.25 | Rh-aa176 | 50.2 | Rh-aa316 | 78.7 |
| Rh-aa34 | 0.15 | Rh-aa177 | 0.018 | Rh-aa318 | 68.1 |
| Rh-aa35 | 0.12 | Rh-aa178 | 65 | Rh-aa319 | 77.4 |
| Rh-aa37 | 0.28 | Rh-aa179 | 62.1 | Rh-aa320 | 66.7 |
| Rh-aa38 | 0.083 | Rh-aa180 | 65.5 | Rh-aa321 | 70 |
| Rh-aa39 | 0.042 | Rh-aa183 | 69.9 | Rh-aa322 | 55.5 |
| Rh-aa40 | 66.2 | Rh-aa185 | 1.31 | Rh-aa323 | 58.6 |
| Rh-aa41 | 0.044 | Rh-aa186 | 58.3 | Rh-aa324 | 52.9 |
| Rh-aa42 | 0.047 | Rh-aa187 | 4.52 | Rh-aa326 | 1.18 |
| Rh-aa43 | 20.7 | Rh-aa188 | 58.5 | Rh-aa327 | 0.36 |
| Rh-aa44 | 70.5 | Rh-aa189 | 0.16 | Rh-aa328 | 1.35 |
| Rh-aa45 | 58.2 | Rh-aa190 | 2.63 | Rh-aa329 | 1.4 |
| Rh-aa46 | 0.02 | Rh-aa191 | 50.2 | Rh-aa330 | 46.7 |
| Rh-aa47 | 71.8 | Rh-aa192 | 68.5 | Rh-aa331 | 58.3 |
| Rh-aa49 | 65.3 | Rh-aa193 | 72.2 | Rh-aa332 | 1.39 |
| Rh-aa51 | 64.3 | Rh-aa194 | 72.2 | Rh-aa333 | 51.3 |
| Rh-aa52 | 71.3 | Rh-aa195 | 73 | Rh-aa334 | 62 |
| Rh-aa53 | 54.2 | Rh-aa196 | 71.6 | Rh-aa335 | 41.4 |
| Rh-aa55 | 67.1 | Rh-aa197 | 76.4 | Rh-aa336 | 0.06 |
| Rh-aa56 | 73 | Rh-aa198 | 74.1 | Rh-aa337 | 67.8 |
| Rh-aa59 | 66.3 | Rh-aa199 | 73.1 | Rh-aa338 | 27.2 |
| Rh-aa60 | 4.22 | Rh-aa200 | 72.8 | Rh-aa340 | 0.072 |
| Rh-aa61 | 62.2 | Rh-aa201 | 75 | Rh-aa341 | 67.2 |
| Rh-aa62 | 0.023 | Rh-aa202 | 67 | Rh-aa343 | 61.2 |
| Rh-aa63 | 69.6 | Rh-aa204 | 58.3 | Rh-aa344 | 64.5 |
| Rh-aa64 | 60.2 | Rh-aa206 | 70.3 | Rh-aa345 | 35.1 |
| Rh-aa65 | 72.4 | Rh-aa207 | 0.068 | Rh-aa346 | 64.1 |
| Rh-aa66 | 29.5 | Rh-aa208 | 0.039 | Rh-aa347 | 1.04 |
| Rh-aa67 | 0.091 | Rh-aa209 | 0 | Rh-aa349 | 67.3 |
| Rh-aa68 | 69.3 | Rh-aa210 | 0.041 | Rh-aa350 | 0.057 |
| Rh-aa69 | 0.12 | Rh-aa211 | 34.1 | Rh-aa351 | 33.3 |
| Rh-aa70 | 22.4 | Rh-aa212 | 0.022 | Rh-aa352 | 48.1 |
| Rh-aa71 | 69 | Rh-aa213 | 9.2 | Rh-aa353 | 39.3 |
| Rh-aa72 | 0.35 | Rh-aa215 | 62.6 | Rh-aa354 | 58.7 |
| Rh-aa73 | 11.6 | Rh-aa216 | 1.21 | Rh-aa355 | 17.5 |
| Rh-aa75 | 4.49 | Rh-aa217 | 0.06 | Rh-aa356 | 0 |
| Rh-aa76 | 0 | Rh-aa218 | 0.14 | Rh-aa357 | 33.2 |
| Rh-aa77 | 0.74 | Rh-aa219 | 0.042 | Rh-aa358 | 62.4 |
| Rh-aa78 | 0.024 | Rh-aa220 | 1.33 | Rh-aa359 | 0.11 |
| Rh-aa80 | 0.069 | Rh-aa221 | 54 | Rh-aa360 | 26.1 |
| Rh-aa81 | 1.34 | Rh-aa222 | 58.6 | Rh-aa362 | 0.072 |
| Rh-aa82 | 0.11 | Rh-aa223 | 49.8 | Rh-aa363 | 68.6 |
| Rh-aa83 | 24.2 | Rh-aa224 | 67.6 | Rh-aa364 | 56.7 |
| Rh-aa84 | 67.2 | Rh-aa225 | 63.8 | Rh-aa365 | 68.6 |
| Rh-aa85 | 38.2 | Rh-aa226 | 69.2 | Rh-aa366 | 67 |
| Rh-aa86 | 0.091 | Rh-aa227 | 67.9 | Rh-aa367 | 63 |
| Rh-aa90 | 0.021 | Rh-aa228 | 55.1 | Rh-aa368 | 66.2 |
| Rh-aa91 | 71.8 | Rh-aa230 | 69.7 | Rh-aa369 | 22.5 |
| Rh-aa92 | 45.9 | Rh-aa231 | 0.023 | Rh-aa371 | 68.3 |
| Rh-aa93 | 0.18 | Rh-aa232 | 8.12 | Rh-aa372 | 66.1 |
| Rh-aa94 | 63.5 | Rh-aa233 | 58.3 | Rh-aa373 | 47.6 |
| Rh-aa96 | 64.5 | Rh-aa234 | 59.2 | Rh-aa374 | 66.5 |
| Rh-aa97 | 0.11 | Rh-aa235 | 67.5 | Rh-aa375 | 67.8 |
| Rh-aa98 | 0.024 | Rh-aa236 | 56.1 | Rh-aa376 | 0.22 |
| Rh-aa99 | 62.4 | Rh-aa237 | 6.1 | Rh-aa377 | 66.5 |
| Rh-aa101 | 0.78 | Rh-aa238 | 38.2 | Rh-aa378 | 0.12 |
| Rh-aa102 | 55.9 | Rh-aa240 | 43.8 | Rh-aa380 | 67.6 |
| Rh-aa104 | 29.2 | Rh-aa243 | 3.84 | Rh-aa381 | 0.053 |
| Rh-aa105 | 0.024 | Rh-aa244 | 6 | Rh-aa382 | 65.5 |
| Rh-aa107 | 0.16 | Rh-aa246 | 57.2 | Rh-aa383 | 59.8 |
| Rh-aa108 | 0.055 | Rh-aa249 | 70.1 | Rh-aa385 | 30.3 |
| Rh-aa111 | 69.5 | Rh-aa251 | 49.8 | Rh-aa386 | 41.5 |
| Rh-aa112 | 62.6 | Rh-aa252 | 41.1 | Rh-aa387 | 9.82 |
| Rh-aa113 | 28.5 | Rh-aa254 | 75.3 | Rh-aa388 | 0.079 |
| Rh-aa114 | 0.15 | Rh-aa255 | 49.3 | Rh-aa390 | 0.12 |
| Rh-aa115 | 27.4 | Rh-aa256 | 69.1 | Rh-aa391 | 0.068 |
| Rh-aa116 | 0.025 | Rh-aa257 | 67.6 | Rh-aa392 | 0.087 |
| Rh-aa118 | 49 | Rh-aa258 | 45.1 | Rh-aa393 | 69.3 |
| Rh-aa120 | 25 | Rh-aa260 | 39.7 | Rh-aa394 | 56.6 |
| Rh-aa121 | 0.046 | Rh-aa261 | 45.2 | Rh-aa395 | 0.039 |
| Rh-aa123 | 0.1 | Rh-aa263 | 34 | Rh-aa396 | 0.042 |
| Rh-aa124 | 69.3 | Rh-aa264 | 75 | Rh-aa398 | 60.5 |
| Rh-aa125 | 73.3 | Rh-aa265 | 70.8 | Rh-aa399 | 71.2 |
| Rh-aa126 | 69.5 | Rh-aa266 | 70.2 | Rh-aa401 | 59.2 |
| Rh-aa127 | 67 | Rh-aa268 | 27.4 | Rh-aa402 | 58.9 |
| Rh-aa128 | 66.9 | Rh-aa269 | 66.1 | Rh-aa403 | 65.5 |
| Rh-aa129 | 72.2 | Rh-aa270 | 73.8 | Rh-aa404 | 70.6 |
| Rh-aa130 | 76.1 | Rh-aa271 | 51.3 | Rh-aa406 | 68 |
| Rh-aa131 | 77.2 | Rh-aa272 | 71.7 | Rh-aa407 | 60.2 |
| Rh-aa133 | 27.2 | Rh-aa273 | 75.3 | Rh-aa408 | 66.1 |
| Rh-aa135 | 0 | Rh-aa274 | 13.8 | Rh-aa409 | 65.2 |
| Rh-aa137 | 0.087 | Rh-aa275 | 50.8 | Rh-aa410 | 65.4 |
| Rh-aa138 | 55.6 | Rh-aa276 | 63.2 | Rh-aa411 | 66.1 |
| Rh-aa139 | 72.1 | Rh-aa278 | 0.21 | Rh-aa413 | 69.9 |
| Rh-aa140 | 69.6 | Rh-aa280 | 72.3 | Rh-aa414 | 67.7 |
| Rh-aa141 | 66 | Rh-aa281 | 9.5 | SpCas9 | 49.7 |
| Rh-aa142 | 51.8 | Rh-aa282 | 58.9 | Un1Cas12f1_ge4.1 | 36.1 |
| Rh-aa143 | 0 | Rh-aa283 | 79.7 | WTRhCas12f1 | 61.5 |
| Rh-aa144 | 56 | Rh-aa284 | 42.4 | WTRhCas12f1_NT | 0.62 |
| | | Rh-aa285 | 25.8 | | |

Second round iteration screen was performed by mutating OsCas12f1-D52R with one additional mutation that was identified as an enhanced OsCas12f1 mutant in the first round screen. Using a library containing 15 double mutants of OsCas12f1, it was found that R substitution at A54, S119, T132, and S141 further increased the activity of OsCas12f1-D52R (FIG. 2C). Thus, the most efficient OsCas12f1 mutant containing T132R+D52R double mutation (OsCas12f1-D52R+T132R; SEQ ID NO: 226) was selected for further engineering.

A stabilizing sequence 5′-TTTTATTTTTTT-3′ was fused to the 3′ of sgRNAs for increased stability and hence improved editing efficiency, and an sgRNA optimization strategy was adopted to the scaffold sequence of sgRNA, including truncation or deletion of base pairs in the RNA stem region (FIG. 2D and Table 3).

The A-U or mismatched base pairs was replaced in the scaffold sequences of sgRNAs with thermodynamically stable C-G base pair, which increased sgRNA stability (FIG. 2D and Table 3). These sgRNA variants resulted in substantially higher OsCas12f1-mediated cleavage activity as measured by the reporter system in Example 1, especially for Os-sg1.1 (SEQ ID NO: 234), which contained A-U substituted to C-G at the stem 1 region of the tracrRNA and showed 1.56-fold increasement in GFP activation efficiency over WTOsCas12f1 (SEQ ID NO: 73) (FIG. 2E). Thus, the Os-sg1.1 variant (SEQ ID NO: 234) was selected for further optimization of OsCas12f1. Based on the first round optimization of OsCas12f1 sgRNA, it was speculated that substitution with C-G base pair in sgRNA could be of benefit to increasing OsCas12f1 activity. To confirm this hypothesis, more base pairs on Os-sg1.1 were substituted with C-G base pairs, creating a sgRNA library with 13 variants. Through the second round sgRNA screen, several sgRNA variants were identified showing higher activity than that of Os-sg1.1. Among these sgRNA variants, Os-sg2.6 (SEQ ID NO: 244) outperformed over other variants (FIG. 2F).

TABLE 3
Scaffold sequence variants

| sgRNA variant | | Scaffold sequences |
|---|---|---|
| deletion | OsCas12f1 | AGGGACTTCCCCCAAAATCGAGACAGTAGCCGTAAAACTTTGAGTTTCAGA |
| version | _sg0.1 | GTGGGCGACACACTCGAAAAGGTTAAGATATGCACATAGTAATCCGTGCAT |
| | (SEQ ID | GAGCCGCGAAAGCGGCTTGAAGG |
| | NO: 228) | |
| | OsCas12f1 | ATCGAGACAGTAGCCGTAAAACTTTGAGTTTCAGAGTGGGCGACACACTCG |
| | _sg0.2 | AAAAGGTTAAGATATGCACATAGTAATCCGTGCATGAGCCGCGAAAGCGGC |
| | (SEQ ID | TTGAAGG |
| | NO: 229) | |
| | OsCas12f1 | AGGGACGACTTCCCGTCCCAAAATCGAGACAGTAGCCAGTTTGGCGACACA |
| | _sg0.3 | CTCGAAAAGGTTAAGATATGCACATAGTAATCCGTGCATGAGCCGCGAAAG |
| | (SEQ ID | CGGCTTGAAGG |
| | NO: 230) | |
| | OsCas12f1 | AGGGACGACTTCCCGTCCCAAAATCGAGAGTTTCTCGAAAAGGTTAAGATA |
| | _sg0.4 | TGCACATAGTAATCCGTGCATGAGCCGCGAAAGCGGCTTGAAGG |
| | (SEQ ID | |
| | NO: 231) | |
| | OsCas12f1 | AGGGACGACTTCCCGTCCCAAAATCGAGACAGTAGCCGTAAAACTTTGAGT |
| | _sg0.5 | TTCAGAGTGGGCGACACACTCGAAAAGGTTAAGATATGCACATAGTAATCC |
| | (SEQ ID | GTGCATGAGGAAACTTGAAGG |
| | NO: 232) | |
| | OsCas12f1 | AGGGACGACTTCCCGTCCCAAAATCGAGACAGTAGCCGTAAAACTTTGAGT |
| | _sg0.6 | TTCAGAGTGGGCGACACACTCGAAAAGGTTAAGATATGCACATAGTAATCC |
| | (SEQ ID | GTGCATGAAAGAAGG |
| | NO: 233) | |
| G: | OsCas12f1 | AGGGcCGACTTCCCGgCCCAAAATCGAGACAGTAGCCGTAAAACTTTGAGT |
| C | _sg1.1 | TTCAGAGTGGGCGACACACTCGAAAAGGTTAAGATATGCACATAGTAATCC |
| substitution | (SEQ ID | GTGCATGAGCCGCGAAAGCGGCTTGAAGG |
| | NO: 234) | |
| | OsCas12f1 | AGGGACGACTTCCCGTCCCAAAATCGAGACAGTcGCCGTAAAACTTTGAGT |
| | _sg1.2 | TTCAGAGTGGGCGACACACTCGAAAAGGTTAAGATATGCACATAGTAATCC |
| | (SEQ ID | GTGCATGAGCCGCGAAAGCGGCTTGAAGG |
| | NO: 235) | |
| | OsCas12f1 | AGGGACGACTTCCCGTCCCAAAATCGAGACAGTAGCCGTAAAACTCTGAGT |
| | sg1.3 | TTCAGAGTGGGCGACACACTCGAAAAGGTTAAGATATGCACATAGTAATCC |
| | (SEQ ID | GTGCATGAGCCGCGAAAGCGGCTTGAAGG |
| | NO: 236) | |
| | OsCas12f1 | AGGGACGACTTCCCGTCCCAAAATCGAGACAGTCGCCAGTTTGGCGACACA |
| | _sg1.4 | CTCGAAAAGGTTAAGATATGCACATAGTAATCCGTGCATGAGCCGCGAAAG |
| | (SEQ ID | CGGCTTGAAGG |
| | NO: 237) | |
| | OsCas12f1 | AGGGACGACTTCCCGTCCCAAAATCGAGACAGTAGCCGTAAAACTTTGAGT |
| | _sg1.5 | TTCAGAGTGGGCGACACACTCGAAAAGGTTAAGATACGCACATAGTAATCC |
| | (SEQ ID | GTGCgTGAGCCGCGAAAGCGGCTTGAAGG |
| | NO: 238) | |
| | OsCas12f1 | AGGGcCGACTTCCCGgCCCAAAATCGAGACAGTcGCCGTAAAACTCTGAGT |
| | _sg2.1 | TTCAGAGTGGGCGACACACTCGAAAAGGTTAAGATATGCACATAGTAATCC |
| | (SEQ ID | GTGCATGAGCCGCGAAAGCGGCTTGAAGG |
| | NO: 239) | |
| | OsCas12f1 | AGGGcCGACTTCCCGgCCCAAAATCGAGACAGTAGCCGTAAAACTTTGAGT |
| | _sg2.2 | TTCAGAGTGGGCLACACACTCGAAAAGGTTAAGATATGCACATAGTAATCC |
| | (SEQ ID | GTGCATGAGCCGCGAAAGCGGCTTGAAGG |
| | NO: 240) | |
| | OsCas12f1 | AGGGCCGACTTCCCGgCCCAAAAgCGAGACAGTAGCCGTAAAACTTTGAGT |
| | _sg2.3 | TTCAGAGTGGGCGACACACTCGCAAAGGTTAAGATATGCACATAGTAATCC |
| | (SEQ ID | GTGCATGAGCCGCGAAAGCGGCTTGAAGG |
| | NO: 241) | |
| | OsCas12f1 | AGGGcCGACTTCCCGgCCCAAAATCGcGACAGTAGCCGTAAAACTTTGAGT |
| | _sg2.4 | TTCAGAGTGGGCGACACACgCGAAAAGGTTAAGATATGCACATAGTAATCC |
| | (SEQ ID | GTGCATGAGCCGCGAAAGCGGCTTGAAGG |
| | NO: 242) | |
| | OsCas12f1 | AGGGCCGACTTCCCGgCCCAAAATCGAGACAGTAGCCGTAAAgCTTTGAGT |
| | _sg2.5 | TTCAGAGcGGGCGACACACTCGAAAAGGTTAAGATATGCACATAGTAATCC |
| | (SEQ ID | GTGCATGAGCCGCGAAAGCGGCTTGAAGG |
| | NO: 243) | |
| | OsCas12f1 | AGGGcCGACTTCCCGgCCCAAAATCGAGACAGTAGCCGTAAAACgTTGAGT |
| | sg2.6 | TTCAGCGTGGGCGACACACTCGAAAAGGTTAAGATATGCACATAGTAATCC |
| | (SEQ ID | GTGCATGAGCCGCGAAAGCGGCTTGAAGG |
| | NO: 244) | |
| | OsCas12f1 | AGGGcCGACTTCCCGgCCCAAAATCGAGACAGTAGCCGTAAAACTTCGAGT |
| | _sg2.7 | TTCgGAGTGGGCGACACACTCGAAAAGGTTAAGATATGCACATAGTAATCC |
| | (SEQ ID | GTGCATGAGCCGCGAAAGCGGCTTGAAGG |
| | NO: 245) | |
| | OsCas12f1 | AGGGcCGACTTCCCGgCCCAAAATCGAGACAGTAGCCGTAAAACTTTGcGT |
| | _sg2.8 | TgCAGAGTGGGCGACACACTCGAAAAGGTTAAGATATGCACATAGTAATCC |
| | (SEQ ID | GTGCATGAGCCGCGAAAGCGGCTTGAAGG |
| | NO: 246) | |
| | OsCas12f1 | AGGGcCGACTTCCCGgCCCAAAATCGAGACAGTAGCCGTAAAACTTTGAGT |
| | _sg2.9 | TTCAGAGTGGGCGACACACTCGAAAAGGTTAAGATgTGCACATAGTAATCC |
| | (SEQ ID | GTGCACGAGCCGCGAAAGCGGCTTGAAGG |
| | NO: 247) | |
| | OsCas12f1 | AGGGcCGACTTCCCGgCCCAAAATCGAGACAGTAGCCGTAAAACTTTGAGT |
| | _sg2.10 | TTCAGAGTGGGCGACACACTCGAAAAGGTTAAGATATGCACATAGTAATCC |
| | (SEQ ID | GTGCATGAGCCGCGAAAGCGGCTCGAAGG |
| | NO: 248) | |
| | OsCas12f1 | AGGGCCGACTTCCCGgCCCAAAATCGAGACAGTAGCCGTAAAACTTTGAGT |
| | _sg2.11 | TTCAGAGTGGGCGACACACTCGAAAAGGTTAAGATATGCACATAGTAATCC |
| | (SEQ ID | GTGCATGcGCCGCGAAAGCGGCgTGAAGG |
| | NO: 249) | |
| | OsCas12f1 | AGGGcCGACTTCCCGgCCCAAAATCGAGACAGTcGCCGTAAAACTTTGAGT |
| | _sg2.12 | TTCAGAGTGGGCGACACACTCGAAAAGGTTAAGATATGCACATAGTAATCC |
| | (SEQ ID | GTGCATGAGCCGCGAAAGCGGCTTGAAGG |
| | NO: 250) | |
| | OsCas12f1 | AGGGcCGACTTCCCGgCCCAAAATCGAGACAGTAGCCGTAAAACTCTGAGT |
| | _sg2.13 | TTCAGAGTGGGCGACACACTCGAAAAGGTTAAGATATGCACATAGTAATCC |
| | (SEQ ID | GTGCATGAGCCGCGAAAGCGGCTTGAAGG |
| | NO: 251) | |
| deletion | RhCas12f1 | AAGTCTGAGGGCATGTAGAAAAAAGTATAGGTATATACCAACATACTTGCA |
| version | _sg0.1 | TTGCCACTCGGAAAGGGTTAACCTTGGTCATTGTGTTACCGACCAAGCATT |
| | (SEQ ID | CCAGAAATGGAATGTAAAT |
| | NO: 252) | |
| | RhCas12f1 | ACGGTTGATTTAGCAACCGAAGTGGGCATGTAGAAAAAAGTATAGGTATAT |
| | _sg0.2 | ACCAACATACTTGCATTGCCACAAAGGGTTAACCTTGGTCATTGTGTTACC |
| | (SEQ ID | GACCAAGCATTCCAGAAATGGAATGTAAAT |
| | NO: 253) | |
| | RhCas12f1 | ACGGTTGATTTAGCAACCGAAGTCTGGCATGTAGAAAAAAGTATAGGTATA |
| | _sg0.3 | TACCAACATACTTGCATTGCCGGAAAGGGTTAACCTTGGTCATTGTGTTAC |
| | (SEQ ID | CGACCAAGCATTCCAGAAATGGAATGTAAAT |
| | NO: 254) | |
| | RhCas12f1 | ACGGTTGATTTAGCAACCGAAGTCTGAGGGGTAGAAAAAAGTATAGGTATA |
| | _sg0.4 | TACCAACATACTTGCCCACTCGGAAAGGGTTAACCTTGGTCATTGTGTTAC |
| | (SEQ ID | CGACCAAGCATTCCAGAAATGGAATGTAAAT |
| | NO: 255) | |
| | RhCas12f1 | ACGGTTGATTTAGCAACCGAAGTCTGAGGGCATGGTATAGGTATATACCAA |
| | _sg0.5 | CATACCATTGCCACTCGGAAAGGGTTAACCTTGGTCATTGTGTTACCGACC |
| | (SEQ ID | AAGCATTCCAGAAATGGAATGTAAAT |
| | NO: 256) | |
| G: | RhCas12f1 | ACGGCTGATTTAGCAgCCGAAGTCTGAGGGCATGTAGAAAAAAGTATAGGT |
| C | _sg1.1 | ATATACCAACATACTTGCATTGCCACTCGGAAAGGGTTAACCTTGGTCATT |
| substitution | (SEQ ID | GTGTTACCGACCAAGCATTCCAGAAATGGAATGTAAAT |
| | NO: 257) | |
| | RhCas12f1 | ACGGTCGATTTAGCgACCGAAGTCTGAGGGCATGTAGAAAAAAGTATAGGT |
| | _sg1.2 | ATATACCAACATACTTGCATTGCCACTCGGAAAGGGTTAACCTTGGTCATT |
| | (SEQ ID | GTGTTACCGACCAAGCATTCCAGAAATGGAATGTAAAT |
| | NO: 258) | |
| | RhCas12f1 | ACGGCCGATTTAGCggCCGAAGTCTGAGGGCATGTAGAAAAAAGTATAGGT |
| | _sg1.3 | ATATACCAACATACTTGCATTGCCACTCGGAAAGGGTTAACCTTGGTCATT |
| | (SEQ ID | GTGTTACCGACCAAGCATTCCAGAAATGGAATGTAAAT |
| | NO: 259) | |

It was further determined whether the respective increases in cleavage activity of OsCas12f1 through Cas protein engineering and sgRNA engineering were additive effects. The Os-sg1.1 sgRNA variant was first used to guide the OsCas12f1-D52R protein variant. This combined variant system showed higher cleavage activity than either variant system alone (Cas12f1 variant plus WT sgRNA, or WT Cas12f1 plus sgRNA variant) (FIG. 14B). Os-sg2.6 was then used to guide OsCas12f1-D52R, which outperformed over D52R+Os-sg1.1 combination variant system (FIGS. 14C-14F). Lastly, by comminating T132R with D52R+Os-sg2.6, generating the most efficient combination variant system, named as “enOsCas12f1” system, composed of OsCas12f1-D52R+T132R (SEQ ID NO: 226) and Os-sg2.6 scaffold sequence (SEQ ID NO: 244) (FIG. 2G and FIG. 14G). The enOsCas12f1 exhibited 9.4-fold increasement than that of WT OsCas12f1 at DMD locus (FIG. 14H).

For generating enRhCas12f1, seven high-performance protein variants (T131R, S130R, A273R, I264R, L270R, Y125R, A56R) were chosen for combination with the most efficient sgRNA variants, Rh-sg1.1 (SEQ ID NO: 257) (FIGS. 2H-2J). Among these combination variants, RhCas12f1-L270R (SEQ ID NO: 227)+Rh-sg1.1 scaffold sequence (SEQ ID NO: 257) combination variant system (named as “enRhCas12f1” system) outperformed over others, showing 1.61-fold improvement over WTRhCas12f1 system at endogenous PCSK9 locus (FIG. 2K and FIG. 14I).

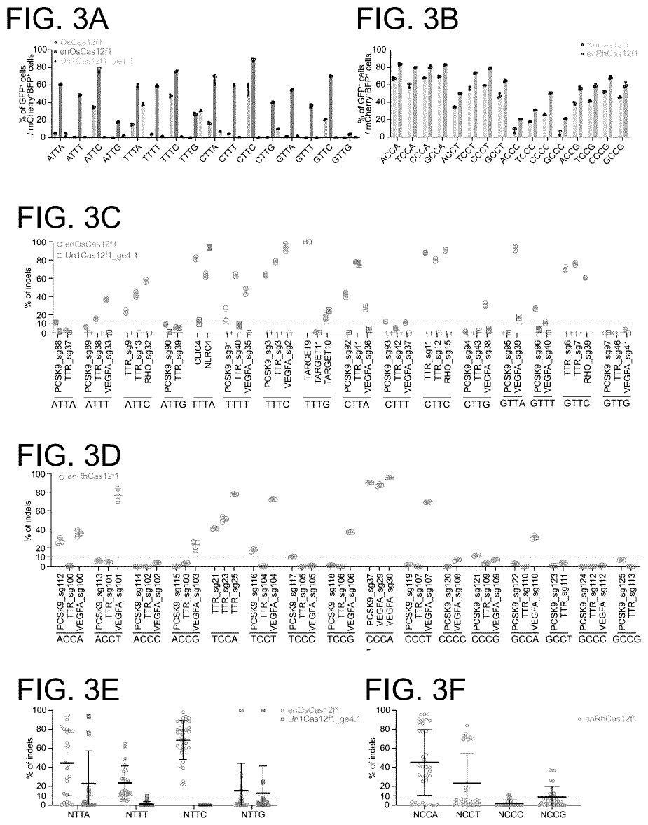

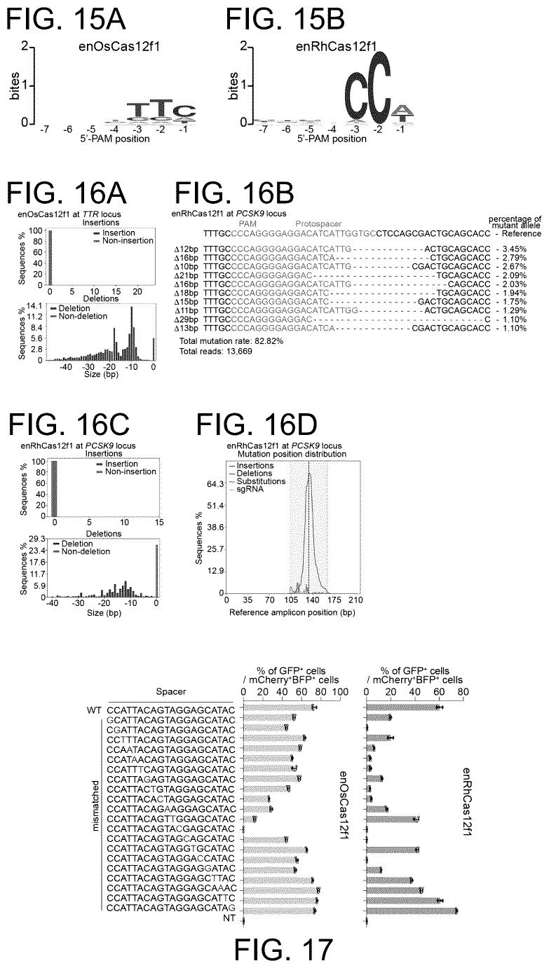

In addition, the in vitro PAM characterization assay was performed to determine the PAM preference of the engineered Cas12f1 proteins, indicating the enOsCas12f1 preferred PAM 5′-TTH (H=not G) than 5′-TTG, while enRhCas12f1 preferred PAM 5′-CCD (D=not C) (FIGS. 15A-15B). Based on the in vitro PAM characterization results, the PAM preferences of different Cas12f1 proteins were further compared, including OsCas12f1, enOsCas12f1, and Un1Cas12f1_ge4.1, in HEK293T cells using the GFP activation reporter with fixed T at position −2 and −3 of 5′-PAM (5′-NTTN). The GFP activation results suggested that enOsCas12f1 recognized PAM 5′-TTN, showing a broader target range than WT OsCas12f1 and Un1Cas12f1_ge4.1 that preferred 5′-YTTH (Y=C or T, H=not G) and 5′-TTTR (R=A or G), respectively (FIG. 3A). The reporter with fixed C at position −2 and −3 of 5′-PAM (5′-NCCN) was used for RhCas12f1 and enRhCas12f1. The efficiency of enRhCas12f1 at all 5′-CCN PAM sites was improved compared to that of WT RhCas12f1 (FIG. 3B). Additionally, the indel frequency analysis at 44 endogenous loci further confirmed that enOsCas12f1 was active at 5′-NTTN target sites with >10% indel at 5′-TTC (12 out of 12 sites), 5′-TTA (7 out of 9 sites), 5′-TTT (9 out of 11 sites) and 5′-TTG (4 out of 11 sites), indicating the PAM preference of enOsCas12f1 as 5′-TTC>5′-TTA>5′-TTT>5′-TTG (FIGS. 3C and 3E). As expected, Un1Cas12f1_ge4.1 induced indels predominately at the 5′-TTTR sites, showing >10% indel at 5′-TTA (4 out of 9 sites) and 5′-TTG (2 out of 11 sites) (FIGS. 3C and 3E). The PAM preference of enRhCas12f1 was also analyzed by evaluating the indel frequency at 45 endogenous loci, revealing that enRhCas12f1 achieved >10% indel at 5′-CCA (9 out of 12 sites), 5′-CCT (4 out of 11 sites) and 5′-CCG (3 out of 11 sites), suggesting enRhCas12f1 could recognize 5′-CCD PAM (D=not C) (FIGS. 3D and 3F).

Therefore, the protein engineering, which may increase the binding ability of the Cas12f1 proteins to nucleic acids, combined with C-G base pair substitution in the scaffold sequence of sgRNA, can improve the cleavage activity of OsCas12f1 and RhCas12f1 and broaden the target range of OsCas12f1.

### Example 3. enOsCas12f1 and enRhCas12f1 Enable Robust Genomic Editing in Human Cells

It was further evaluated whether enOsCas12f1 and enRhCas12f1 could efficiently edit endogenous genomic loci in human cells. To comprehensively compare the editing efficiencies of enOsCas12f1, enRhCas12f1, and the published high-performance Cas12f1, Un1Cas12f1_ge4.1, their targeting at all access sites in the exons of PCSK9, TTR, and VEGFA was quantified, based strictly on PAM sequence without consideration for potential sgRNA and target feature contributing toward Cas nuclease activity, such as GC content. In total, the indel frequency was quantified at 30 sites targeted by enOsCas12f1 (5′-NTTC PAM), 61 sites targeted by enRhCas12f1 (5′-TCCA and 5′-CCCA PAM), and 27 sites targeted by Un1Cas12f1_ge4.1 (5′-TTTR PAM).

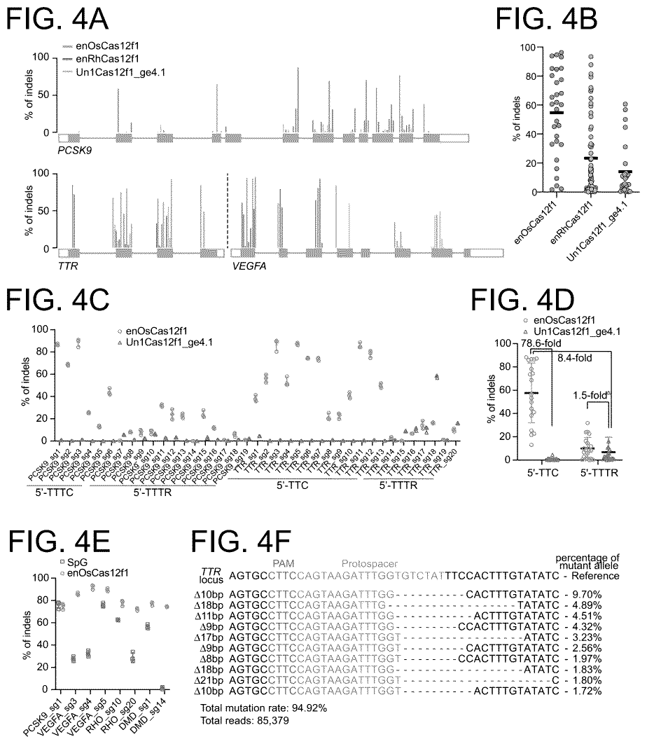

The results showed that enOsCas12f1 induced indels (>1%) in all of 30 tested sites with a maximal efficiency of 96.2%, while enRhCas12f1 induced indels (>1%) in 53 of the 61 tested loci with a maximal efficiency of 93.3%. By contrast, Un1Cas12f1_ge4.1 generated relatively lower indel frequencies (>1%) in 22 sites across 27 tested loci, with a maximal efficiency of 60.6% (FIG. 4A). On average, enOsCas12f1 (54.7±29.8%, mean±s.d.) thus showed 3.9-fold higher efficiency and enRhCas12f1 (23.3±26.8%) showed 1.7-fold higher efficiency than Un1Cas12f1_ge4.1 (14.0±18.1%), respectively (FIG. 4B). When assessing the indel frequency induced by enOsCas12f1 and Un1Cas12f1_ge4.1 targeted by exactly the same sgRNAs at PCSK9 and TTR loci, it was found that enOsCas12f1 showed 78.6-fold higher indel frequency on average at 5′-TTC PAM sites than that of Un1Cas12f1_ge4.1, and 8.4-fold higher efficiency at their own preferred 5′-PAM (5′-TTC for enOsCas12f1 and 5′-TTTR for Un1Cas12f1_ge4.1) (FIGS. 4C and 4D). According to these advantages of enOsCas12f1 over Un1Cas12f1_ge4.1, up to 54.4±29.9% and 59.1±23.1% editing efficiency was achieved at therapeutic target loci PCSK9 and TTR, respectively, while Un1Cas12f1_ge4.1 showed relatively lower editing efficiency, with an average efficiency of 2.3±1.9% and 15.2±18.7% (FIGS. 4C and 4D). Additionally, the activity of enOsCas12f1 and SpG was compared by indel analysis at endogenous sites of 5′-TTC-N₂₀-3′-NGN at PCKS9, VEGFA, RHO, and DMD loci, indicating that enOsCas12f1 outperformed SpG at these target sites (FIG. 4E).

High throughput sequencing of target loci revealed that both enOsCas12f1 and enRhCas12f1 predominantly generated deletions that altered the protospacer sequences rather than insertions (FIG. 4F, FIGS. 16A-16D). The center of the deletion position was located at the PAM-distal region outside of the protospacer sequences (FIG. 4F, FIGS. 16A-16D), which was similar to that of Un1Cas12f1 and AsCas12f1.

### Example 4. The Specificity of enOsCas12f1- and enRhCas12f1-Mediated Genome Editing

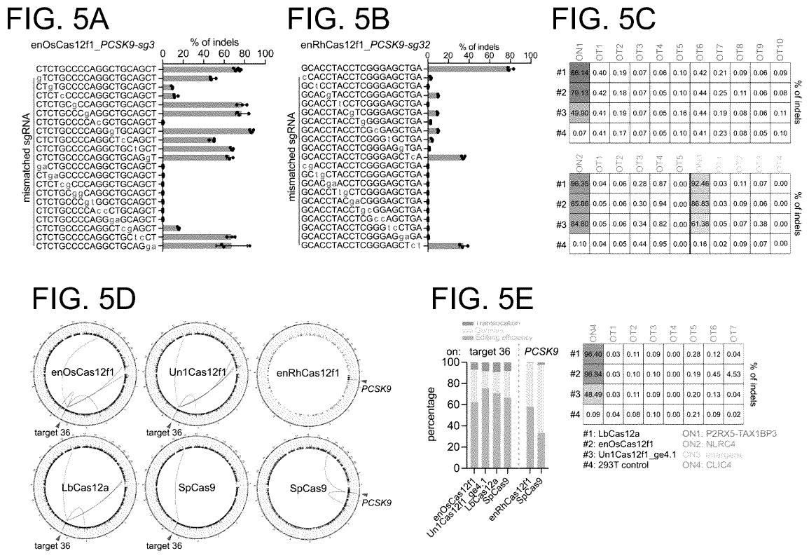

The mismatch tolerance of enOsCas12f1 and enRhCas12f1 was first evaluated by tilling single or adjacent two mismatches in spacer sequences. For the PCSK9 locus, enOsCas12f1 did not tolerate single mismatch at positions 3/5/11, while the mismatches at other positions slightly reduced enOsCas12f1-mediated editing efficiency (FIG. 5A), which was also validated by GFP activation system (FIG. 17). Two adjacent mismatches at position 1-16 substantially reduced enOsCas12f1 activity (FIG. 5A).

The mismatch tolerance of enRhCas12f1 was assessed at endogenous PCSK9 locus or by GFP activation reporter system, indicating that enRhCas12f1 partially tolerates base pair mismatches at PAM-distal region, especially at positions 19 and 20, while the mismatches close to PAM could substantially reduce the activity of enRhCas12f1 (FIG. 5B and FIG. 17).

Targeted deep sequencing was performed at in-silico predicted off-target sites (P2RX5-TAX1BP3, an intergenic region, NLRC4 and CLIC4). The targeted deep sequencing indicated that the on-target editing efficiency of enOsCas12f1 was comparable to that of LbCas12a, and slightly higher than that of Un1Cas12f1_ge4.1. Similar to LbCas12a and Un1Cas12f1_ge4.1, enOsCas12f1 showed strikingly low off-target effects at the potential off-target sites, while a low off-target effect was found at CLIC4 OT7 site for enOsCas12f1 (FIG. 5C).

Finally, PEM-seq was performed to quantify the genome-wide editing specificities of enOsCas12f1 and enRhCas12f1. When targeting target 36 site, five off-target sites were found to be induced by enOsCas12f1 and Un1Cas12f1_ge4.1, four and one of the off-target sites were found for LbCas12a and SpCas9, respectively (FIG. 5D). enOsCas12f1 exhibited 7.03% of translocation rate, which was comparable to that of Un1Cas12f1_ge4.1 (8.44%), LbCas12a (9.22%), and SpCas9 (8.19%) when targeting target 36 site (FIG. 5E). enRhCas12f1 showed no detectable off-target site with low translocation efficiency when targeting PCSK9 locus, while 2 off-target sites were found for SpCas9 (FIGS. 5D and 5E). Together, these results suggested that enOsCas12f1 and enRhCas12f1 exhibited high genomic editing efficiency with a wide target range and low off-target effects.

### Example 5. enOsCas12f1-Mediated In Vivo Genome Editing by Single AAV Delivery and enOsCas12f1-Based Epigenome Editing and Gene Activation

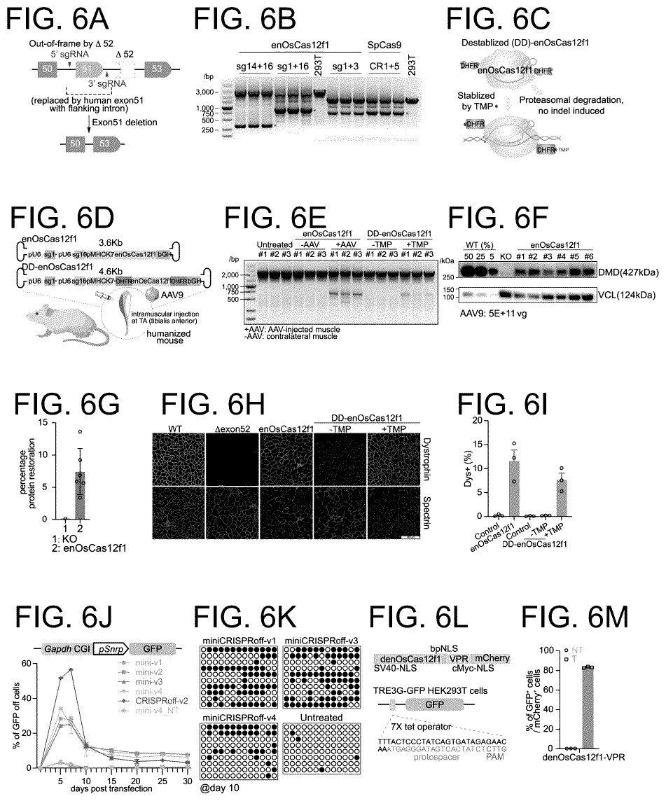

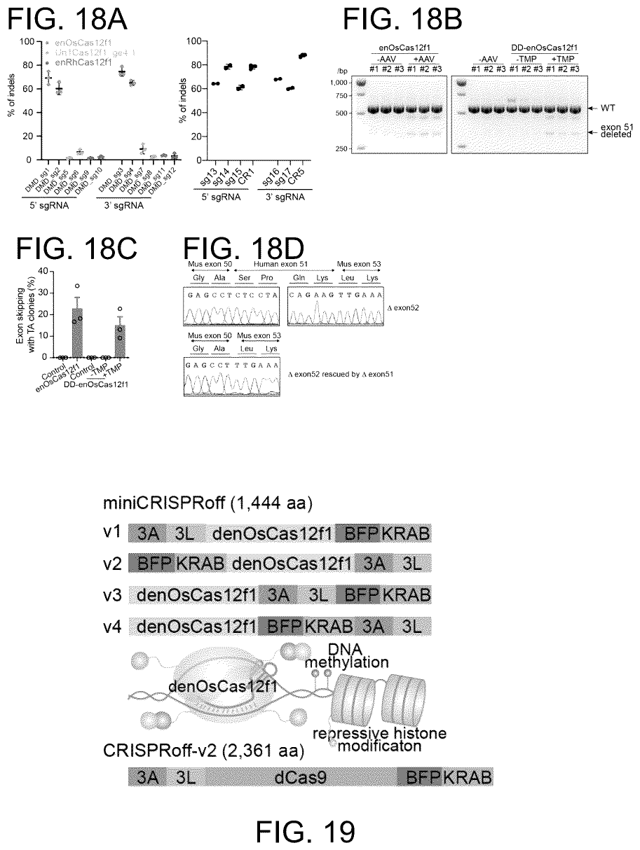

The considerably small size of enOsCas12f1 suggested that its expression cassette could be packaged with multiple sgRNAs in a single rAAV vector, which could enable its therapeutic application to treat genetic disorders that require large fragment deletions, such as Duchenne muscular dystrophy (DMD). To test whether enOsCas12f1 could be harnessed for DMD exon 51 deletion, efficient sgRNAs flanking exon 51 (5′gRNA and 3′gRNA) were firstly screened, indicated that enOsCas12f1 efficiently induced indels, while enRhCas12f1 and Un1Cas12f1_ge4.1 exhibited relatively low editing efficiency at four of the target sites (FIG. 18A; the Cas proteins from the left side to the right side are enOsCas12f1, Un1Cas12f1_ge4.1, and enRhCas12f1). Efficient 5′gRNA was then combined with 3′ sgRNA to target enOsCas12f1 to DMD exon 51 in HEK293T cells (FIG. 6A). PCR-based assays revealed robust genomic deletion of exon 51 (˜1700 bp deletion) by enOsCas12f1 targeted by sg1 (SEQ ID NO: 486)+sg16 (SEQ ID NO: 501), which was more efficient than that of SpCas9 (˜850 bp deletion), although the indel frequency of individual sgRNA of enOsCas12f1 was lower than that of SpCas9 (FIG. 6B and FIG. 18A).

Precisely controlling of enOsCas12f1 activity across multiple dimensions such as dose and timing could undoubtedly reduce the potential toxicity and off-target effects induced by enOsCas12f1, especially for in vivo scenario where enOsCas12f1 is constitutively expressed via AAV delivery. To achieve precisely controlled enOsCas12f1, enOsCas12f1 was fused with the destabilized domains (DD) of E. coli dihydrofolate reductase (ecDHFR). The newly synthesized DD-enOsCas12f1 protein (SEQ ID NO: 260) is rapidly targeted for proteasomal degradation, which can be blocked by the small molecule trimethoprim (TMP) (FIG. 6C).

To assess the in vivo deletion efficiencies of DMD exon 51 induced by enOsCas12f1 and DD-enOsCas12f1, a mouse model of DMD with exon 52 deletion and exon 51 replaced by human exon 51 with flanking intron sequences was created (FIG. 6A). Deletion of exon 52 prematurely terminates protein production of dystrophin, which can be restored by removal of exon 51. AAV serotype 9 (AAV9) was used for local delivery of enOsCas12f1 or DD-enOsCas12f1 as well as its sgRNA expression cassette to skeletal muscle (FIG. 6D). Because of the single AAV-packageable size of CRISPR-OsCas12f1 system, AAV9s was injected into the tibialis anterior muscle with lower titer than that of SpCas9 that needs dual AAV due to its large size. PCR-based detection across the genomic locus indicated the expected ˜1700 bp deletion (FIG. 6E). RT-PCR of mRNA extracted from whole muscle showed the transcripts with exon 51 deletion at efficiency of 22.7±9.2% (mean±s.d.) for enOsCas12f1, while 15.0±7.0% for DD-OsCas12f1 (FIGS. 18B-18D). Western blotting of whole muscle and immunostaining results further confirmed that the protein production of dystrophin was rescued by enOsCas12f1 and DD-enOsCas12f1 (FIGS. 6F-6H). Restoration of dystrophin protein occurred in 11.6±4.0% and 7.6±2.4% of myofibers treated by enOsCas12f1 and DD-enOsCas12f1, respectively (FIG. 6I).

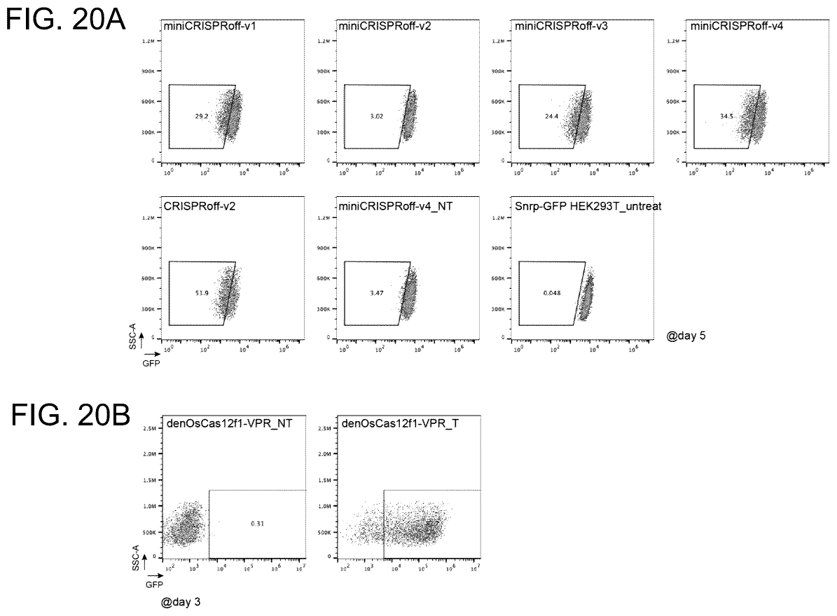

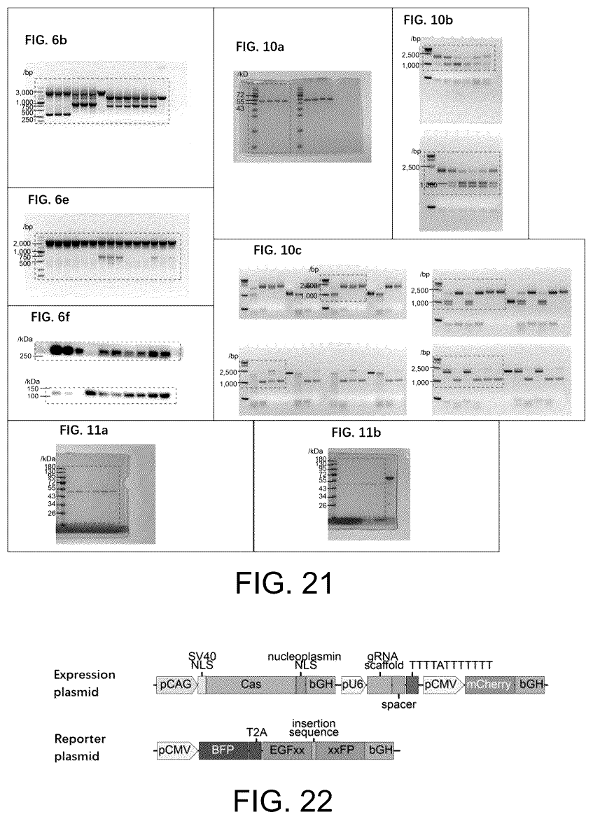

Next, the efficiency of enOsCas12f1-mediated epigenome editing was tested, which was named miniCRISPRoff (1444 aa), by adopting the strategy of CRISPRoff, with protein size at 2,361 aa. Four version of miniCRISPRoff (v1-v4; SEQ ID NOs: 261-264, respectively) were generated with dead enOsCas12f1 (denOsCas12f1 (OsCas12f1-D52R+T132R+D228A+D406A), SEQ ID NO: 513) (FIG. 19), among which miniCRISPRoff-v1, v3, and v4 silenced GFP in the GAPDH-Snrp-GFP stably expressed HEK293T cells (FIG. 6J and FIG. 20A). Bisulfite sequencing indicated that the Snrp promoter was highly methylated by the treatment with miniCRISPRoffs (FIG. 6K). Finally, the ability of enOsCas12f1-mediated gene activation was assessed by fusing denOsCas12f1 (SEQ ID NO: 513) with VPR (enOsCas12f1-VPR; SEQ ID NO: 265), which showed a robust gene activation of GFP in TRE3G-GFP HEK293T cells (FIGS. 6L and 6M, and FIG. 20B). Totally, these results indicated that enOsCas12f1 can be engineered as versatile genome and epigenome editors.

All the protospacer sequences and spacer sequences used in the above Examples are listed in Table 4.

### Discussion

Although compact Cas12f orthologs have been tested in the genome editing delivered by a single AAV vector in human cells, their relatively low editing efficiency and restricted PAM requirement constrained their further application. Here, a set of Class 2, Type V-F CRISPR-Cas (Cas12f) subfamily members from bacteria was characterized, and nine that are functional in human cells were identified (FIGS. 1A-1F). By protein engineering combined with sgRNA optimization, enOsCas12f1 system and enRhCas12f1 system were obtained (FIGS. 2A-2K), showing significantly higher genomic editing efficiency and broader target range than that of Un1Cas12f1_ge4.1, which is the most efficient Cas12f reported to date and is comparable with SpCas9 (FIGS. 3A-4F). The discovery of enOsCas12f1 and enRhCas12f1 systems greatly expanded the target range of Cas12f systems. Un1Cas12f1_ge4.1 required 5′-TTTR (R=A or G) PAM, while enOsCas12f1 was active at 5′-NTTN containing loci. Thus, enOsCas12f1 broadened the target range as much as 8-fold over that of Un1Cas12f1_ge4.1. The 5′-NCCN PAM of enRhCas12f1 is also a promising compensation for the 5′-T-rich PAM constrain of enOsCas12f1 and Un1Cas12f1_ge4.1 (FIGS. 3A-3F).

Rational protein engineering combined with sgRNA optimization, which enable enhanced interaction of Cas protein with nucleic acid or sgRNA, and increased sgRNA stability, has been validated in the current study. It is worth to note that the efficiencies of both OsCas12f1 and RhCas12f1 were substantially improved by substituting the A-U base pair in the first stem of sgRNA with G-C base pair (FIGS. 2A-2K).

enOsCas12f1 enables robust and specific genomic editing in vitro and in vivo and can be applied for efficient deletion of large fragment in human genome, such as ˜1700 bp deletion of exon 51 of dystrophin (FIGS. 5A-6M). It has been shown that increased off-target mutations and DNA damage response could be triggered by constitutive nuclease activity of Cas proteins. Acute manipulation of the activity of enOsCas12f1 within indicated time window and specific type of cells is a promising way to reduce these potential unexpected side effects. By conjugating the destabilized domains of ecDHFR to enOsCas12f1 (DD-enOsCas12f1), highly specific regulation of enOsCas12f1-mediated gene editing was achieved in vivo. It is worth mentioning that DD-enOsCas12f1 together with two sgRNAs could be packaged into a single AAV vector, which circumvents obstacles related to the larger size of Cas9/12 that cannot be packaged into a single AAV. Additionally, cell type specific promoters that usually contain longer sequences can be used for driving expression of enOsCas12f1 and DD-enOsCas12f1 to achieve more precise control of OsCas12f1 activity using systematic delivery by AAVs, which is undoubtedly safer for therapeutic application.

The hypercompact size of enOsCas12f1 (433 aa) and enRhCas12f1 (415 aa) could potentially enable their use in derivative genome engineering applications, including base editing, prime editing, retron editing, epigenome editing, and gene expression regulation. Here, enOsCas12f1 was engineered for sufficient epigenome editing (miniCRISPRoff) and gene activation (enOsCas12f1-VPR). It is interesting to engineer miniCRISPRoff for more efficient and smaller size that can be packaged by single AAV in the future.

In summary, enOsCas12f1 and enRhCas12f1 represent high-performance gene editing tools with versatile applications, and the temporally and spatially controlled DD-enOsCas12f1 is a promising platform for gene therapy.

TABLE 4
Sequence of target loci for indel frequency.

| | | | | | SEQ ID NO |
|---|---|---|---|---|---|
| | | | | | of |
| | | | | | proto- |
| | | | | | spacer/ |
| Genomic | | | protospacer/ | | spacer |
| loci | sgRNA | PAM | spacer sequences | Figure | sequences |
| PCSK9 | PC_sK9_s | TTTC | CCGGTGGTCACTCTGTATGC | FIGS. 3A, 3B, | 266 |
| | g1 | | | 3C, 3D and FIG. | |
| | | | | 8A | |
| | PC_sK9_s | TTTC | CGTCTTTGACTCTAAGGCCC | FIGS. 3A, 3B, | 267 |
| | g2 | | | 3C, 3D and FIG. | |
| | | | | 8A | |
| | PC_sK9_s | TTTC | CTCTGCCCCAGGCTGCAGCT | FIGS. 3A, 3B, | 268 |
| | g3 | | | 3cC 3D and | |
| | | | | FIG. 8A | |
| | PC_sK9_s | TTTC | CAGGTCATCACAGTTGGGGC | FIGS. 3A and 3B | 269 |
| | g4 | | | | |
| | PC_sK9_s | TTTC | TCCAGGAGTGGGAAGCGGCG | FIGS. 3A, 3B, | 270 |
| | g5 | | | 3C and 3D | |
| | PC_sK9_s | TTTC | CTCGGGCTCTGGCAGGTGAC | FIGS. 3A, 3B, | 271 |
| | g6 | | | 3C and 3D | |
| | PC_sK9_s | TTTG | ACTCTAAGGCCCAAGGGGGC | FIGS. 3A, 3B, | 272 |
| | g7 | | | 3C, 3D and FIG. | |
| | | | | 8A | |
| | PC_sK9_s | TTTG | GGGGTGAGGGTGTCTACGCC | FIGS. 3A, 3B, | 273 |
| | g8 | | | 3C, 3D and FIG. | |
| | | | | 8A | |
| | PC_sK9_s | TTTG | CATTCCAGACCTGGGGCATG | FIGS. 3A, 3B, | 274 |
| | g9 | | | 3C, 3D and FIG. | |
| | | | | 8A | |
| | PC_sK9_s | TTTA | TTCGGAAAAGCCAGCTGGTC | FIGS. 3A, 3B, | 275 |
| | g10 | | | 3C and 3D | |
| | PC_sK9_s | TTTG | CCCAGAGCATCCCGTGGAAC | FIGS. 3A, 3B, | 276 |
| | g11 | | | 3C and 3D | |
| | PC_sK9_s | TTTG | TTCCTCCCAGGCCTGGAGTT | FIGS. 3A, 3B, | 277 |
| | g12 | | | 3C and 3D | |
| | PC_sK9_s | TTTG | GGGACCAACTTTGGCCGCTG | FIGS. 3A, 3B, | 278 |
| | g13 | | | 3C and 3D | |
| | PC_sK9_s | TTTG | GCCGCTGTGTGGACCTCTTT | FIGS. 3A, 3B, | 279 |
| | g14 | | | 3C and 3D | |
| | PC_sK9_s | TTTG | CCCCAGGGGAGGACATCATT | FIGS. 3A, 3B, | 280 |
| | g15 | | | 3C and 3D | |
| | PC_sK9_s | TTTG | TGTCACAGAGTGGGACATCA | FIGS. 3A, 3B, | 281 |
| | g16 | | | 3C and 3D | |
| | PC_sK9_s | TTTG | GCAGAGAAGTGGATCAGTCT | FIGS. 3A, 3B, | 282 |
| | g17 | | | 3C and 3D | |
| | PC_sK9_s | TTTG | CAGGTTGGCAGCTGTTTTGC | FIGS. 3A, 3B, | 283 |
| | g18 | | | 3C and 3D | |
| | PC_sK9_s | TTTG | CAGGACTGTATGGTCAGCAC | FIGS. 3A, 3B, | 284 |
| | g19 | | | 3C and 3D | |
| | PC_sK9_s | CCCA | TCCCTACACCCGCACCTTGG | FIGS. 3A and 3B | 285 |
| | g20 | | | | |
| | PC_sK9_s | CCCA | CCTCTCGCAGTCAGAGCGCA | FIGS. 3A and 3B | 286 |
| | g21 | | | | |
| | PC_sK9_s | CCCA | GGCTGCCCGCCGGGGATACC | FIGS. 3A and 3B | 287 |
| | g22 | | | | |
| | PC_sK9_s | CCCA | AAAAGGGTGGCTCACCAGCT | FIGS. 3A and 3B | 288 |
| | g23 | | | | |
| | PC_sK9_s | CCCA | TGTCGACTACATCGAGGAGG | FIGS. 3A and 3B | 289 |
| | g24 | | | | |
| | PC_sK9_s | CCCA | GAGCATCCCGTGGAACCTGG | FIGS. 3A and 3B | 290 |
| | g25 | | | | |
| | PC_sK9_s | CCCA | CAAATGTCGCCTTGGAAAGA | FIGS. 3A and 3B | 291 |
| | g26 | | | | |
| | PC_sK9_s | CCCA | TCAGACGGCCGTGCTTACCT | FIGS. 3A and 3B | 292 |
| | g27 | | | | |
| | PC_sK9_s | CCCA | CCTGGCAGGGGTGGTCAGCG | FIGS. 3A and 3B | 293 |
| | g28 | | | | |
| | PC_sK9_s | CCCA | GGCCTGGAGTTTATTCGGAA | FIGS. 3A and 3B | 294 |
| | g29 | | | | |
| | PC_sK9_s | CCCA | CCCGCCAGGGGCAGCAGCAC | FIGS. 3A and 3B | 295 |
| | g30 | | | | |
| | PC_sK9_s | CCCA | GCCCTCGCCAGGCGCTGGCA | FIGS. 3A and 3B | 296 |
| | g31 | | | | |
| | PC_sK9_s | CCCA | GCACCTACCTCGGGAGCTGA | FIGS. 3A and 3B | 297 |
| | 32 | | | | |
| | PC_sK9_ | CCCA | CCTCCTCACCTTTCCAGGTC | FIGS. 3A and 3B | 298 |
| | g33 | | | | |
| | PC_sK9_s | CCCA | AGACCAGCCGGTGACCCTGG | FIGS. 3A and 3B | 299 |
| | g34 | | | | |
| | PC_sK9_s | CCCA | GGGTCACCGGCTGGTCTTGG | FIGS. 3A and 3B | 300 |
| | g35 | | | | |
| | PC_sK9_s | CCCA | AAGTCCCCAGGGTCACCGGC | FIGS. 3A and 3B | 301 |
| | g36 | | | | |
| | PC_sK9_s | CCCA | GGGGAGGACATCATTGGTGC | FIGS. 3A and 3B | 302 |
| | g37 | | | | |
| | PC_sK9_s | CCCA | CTCTGTGACACAAAGCAGGT | FIGS. 3A and 3B | 303 |
| | g38 | | | | |
| | PC_sK9_s | CCCA | ACCTGGTGGCCGCCCTGCCC | FIGS. 3A and 3B | 304 |
| | g39 | | | | |
| | PC_sK9_s | CCCA | TGGGTGCTGGGGGGCAGGGC | FIGS. 3A and 3B | 305 |
| | g40 | | | | |
| | PC_sK9_s | CCCA | CCCTGCCATCCTGCTTACCT | FIGS. 3A and 3B | 306 |
| | g41 | | | | |
| | PC_sK9_s | CCCA | GGCCCTTTTTGCAGGTTGGC | FIGS. 3A and 3B | 307 |
| | g42 | | | | |
| | PC_sK9_s | CCCA | GATGAGGAGCTGCTGAGCTG | FIGS. 3A and 3B | 308 |
| | g43 | | | | |
| | PC_sK9_s | CCCA | CTCCTGGAGAAACTGGAGCA | FIGS. 3A and 3B | 309 |
| | g44 | | | | |
| | PC_sK9_s | CCCA | TTTCCGTCTTTGACTCTAAG | FIGS. 3A and 3B | 310 |
| | g45 | | | | |
| | PC_sK9_s | CCCA | AGGGGGCAAGCTGGTCTGCC | FIGS. 3A and 3B | 311 |
| | g46 | | | | |
| | PC_sK9_s | CCCA | CAACGCTTTTGGGGGTGAGG | FIGS. 3A and 3B | 312 |
| | g47 | | | | |
| | PC_sK9_s | CCCA | AAAGCGTTGTGGGCCCGGCA | FIGS. 3A and 3B | 313 |
| | g48 | | | | |
| | PC_sK9_s | CCCA | GGCCAACTGCAGCGTCCACA | FIGS. 3A and 3B | 314 |
| | g49 | | | | |
| | PC_sK9_s | CCCA | TGCTGGCCTCAGCTGGTGGA | FIGS. 3A and 3B | 315 |
| | g50 | | | | |
| | PC_sK9_s | CCCA | GCCTCCTACCTGTGAGGACG | FIGS. 3A and 3B | 316 |
| | g51 | | | | |
| | PC_sK9_s | CCCA | GGGCAAGCCCAGCCTCCTAC | FIGS. 3A and 3B | 317 |
| | g52 | | | | |
| | PC_sK9_s | CCCA | GGCTGCAGCTCCCACTGGGA | FIGS. 3A and 3B | 318 |
| | g53 | | | | |
| | PC_sK9_s | CCCA | CTGGGAGGTGGAGGACCTTG | FIGS. 3A and 3B | 319 |
| | g54 | | | | |
| | PC_sK9_s | CCCA | CAAGCCGCCTGTGCTGAGGC | FIGS. 3A and 3B | 320 |
| | g55 | | | | |
| | PC_sK9_s | CCCA | CGCACTGGTTGGGCTGACCT | FIGS. 3A and 3B | 321 |
| | g56 | | | | |
| | PC_sK9_s | CCCA | GGTCTGGAATGCAAAGTCAA | FIGS. 3A and 3B | 322 |
| | g57 | | | | |
| | PC_sK9_s | CCCA | GGACGTGGGAGGTCCCAGGG | FIGS. 3A and 3B | 323 |
| | g58 | | | | |
| TTR | TTR_sg1 | TTTC | TGAACACATGCACGGCCACA | FIGS. 3A, 3B, 3C, | 324 |
| | | | | 3D and FIG. 8A | |
| | TTR_sg2 | TTTC | GCTCCAGATTTCTAATACCA | FIGS. 3A, 3B, 3C, | 325 |
| | | | | 3D and FIG. 8A | |
| | TTR_sg3 | TTTC | TGCCTCCAGACACACTGCTA | FIGS. 3A, 3B, 3C, | 326 |
| | | | | 3D and FIG. 8A | |
| | TTR_sg4 | TTTC | ACACCTTATAGGAAAACCAG | FIGS. 3A, 3B, 3C | 327 |
| | | | | and 3D | |
| | TTR_sg5 | CTTC | TCATCGTCTGCTCCTCCTCT | FIGS. 3A, 3B, 3C | 328 |
| | | | | and 3D | |
| | TTR_sg6 | GTTC | TAGATGCTGTCCGAGGCAGT | FIGS. 3A, 3B, 3C | 329 |
| | | | | and 3D | |
| | TTR_sg7 | GTTC | AGAAAGGCTGCTGATGACAC | FIGS. 3A, 3B, 3C | 330 |
| | | | | and 3D | |
| | TTR_sg8 | GTTC | TTTGGCAACTTACCCAGAGG | FIGS. 3A, 3B, 3C | 331 |
| | | | | and 3D | |
| | TTR_sg9 | ATTC | CTCCTCAGTIGTGAGCCCAT | FIGS. 3A, 3B, 3C | 332 |
| | | | | and 3D | |
| | TTR_sg10 | CTTC | TACAAATTCCTCCTCAGTTG | FIGS. 3A, 3B, 3C | 333 |
| | | | | and 3D | |
| | TTR_sg11 | CTTC | CAGTAAGATTTGGTGTCTAT | FIGS. 3A, 3B, 3C | 334 |
| | | | | and 3D | |
| | TTR_sg12 | CTTC | TCTCATAGGTGGTATTCACA | FIGS. 3A, 3B, 3C | 335 |
| | | | | and 3D | |
| | TTR_sg13 | ATTC | ACAGCCAACGACTCCGGCCC | FIGS. 3A, 3B, 3C | 336 |
| | | | | and 3D | |
| | TTR_sg14 | TTTG | TGGTATTAGAAATCTGGAGC | FIGS. 3A, 3B, 3C, | 337 |
| | | | | 3D and FIG. 8A | |
| | TTR_sg15 | TTTG | TTAACTTCTCACGTGTCTTC | FIG. 3A, 3B and | 338 |
| | | | | FIG. 8B | |
| | TTR_sg16 | TTTG | ACCATCAGAGGACACTTGGA | FIG. 3A, 3B, 3C, | 339 |
| | | | | 3D and FIG. 8A | |
| | TTR_sg17 | TTTG | GCAACTTACCCAGAGGCAAA | FIGS. 3A, 3B, 3C | 340 |
| | | | | and 3D | |
| | TTR_sg18 | TTTG | TAGAAGGGATATACAAAGTG | FIGS. 3A, 3B, 3C | 341 |
| | | | | and 3D | |
| | TTR_sg19 | TTTG | TATATCCCTTCTACAAATTC | FIGS. 3A, 3B, 3C | 342 |
| | | | | and 3D | |
| | TTR_sg20 | TTTG | GTGTCTATTTCCACTTTGTA | FIGS. 3A, 3B, 3C | 343 |
| | | | | and 3D | |
| | TTR_sg21 | TCCA | GACTTTCACACCTTATAGGA | FIG. 3A, 3B and | 344 |
| | | | | FIG. 8E | |
| | TTR_sg22 | TCCA | GACTCACTGGTTTTCCTATA | FIG. 3A, 3B and | 345 |
| | | | | FIG. 8E | |
| | TTR_sg23 | TCCA | CTTTGTATATCCCTTCTACA | FIG. 3A, 3B and | 346 |
| | | | | FIG. 8E | |
| | TTR_sg24 | TCCA | GTAAGATTTGGTGTCTATTT | FIG. 3A, 3B and | 347 |
| | | | | FIG. 8E | |
| | TTR_sg25 | TCCA | GCAAGGCAGAGGAGGAGCAG | FIG. 3A and 3B | 348 |
| | TTR_sg26 | TCCA | AGTGTCCTCTGATGGTCAAA | FIG. 3A and 3B | 349 |
| | TTR_sg27 | CCCA | GGGCACCGGTGAATCCAAGT | FIG. 3A and 3B | 350 |
| | TTR_sg28 | CCCA | GGTGTCATCAGCAGCCTTTC | FIG. 3A and 3B | 351 |
| | TTR_sg29 | CCCA | GAGGCAAATGGCTCCCAGGT | FIG. 3A and 3B | 352 |
| | TTR_sg30 | CCCA | TGCAGCTCTCCAGACTCACT | FIG. 3A and 3B | 353 |
| | TTR_sg31 | TCCA | TGAGCATGCAGAGGTGAGTA | FIG. 8E | 354 |
| VEGFA | VEGFA_sg2 | TTTC | GTCCAACTTCTGGGCTGTTC | FIGS. 3A and 3B | 355 |
| | VEGFA_sg3 | TTTC | GGAGGCCCGACCGGGGCCGG | FIGS. 3A and 3B | 356 |
| | VEGFA_sg4 | TTTC | TGCTGTCTTGGGTGCATTGG | FIGS. 3A and 3B | 357 |
| | VEGFA_sg5 | TTTC | TGTCCTCAGTGGTCCCAGGC | FIGS. 3A and 3B | 358 |
| | VEGFA_sg6 | TTTC | CAGATTATGCGGATCAAACC | FIGS. 3A and 3B | 359 |
| | VEGFA_sg7 | TTTC | CAGAAAATCAGTTCGAGGAA | FIGS. 3A and 3B | 360 |
| | VEGFA_sg8 | TTTC | CCTTTCCTCGAACTGATTTT | FIGS. 3A and 3B | 361 |
| | VEGFA_sg9 | TTTC | GTTTTTGCCCCTTTCCCTTT | FIGS. 3A and 3B | 362 |
| | VEGFA_sg10 | TTTC | TTGCGCTTTCGTTTTTGCCC | FIGS. 3A and 3B | 363 |
| | VEGFA_sg11 | TTTC | CTTTTGCCTTTTTGCAGTCC | FIGS. 3A and 3B | 364 |
| | VEGFA_sg12 | TTTC | TCCGCTCTGAGCAAGGCCCA | FIGS. 3A and 3B | 365 |
| | VEGFA_sg13 | TTTG | TTGTGCTGTAGGAAGCTCAT | FIGS. 3A and 3B | 366 |
| | VEGFA_sg14 | TTTG | CCCCTTTCCCTTTCCTCGAA | FIGS. 3A and 3B | 367 |
| | VEGFA_sg15 | TTTG | CCTTTTTGCAGTCCCTGTGG | FIGS. 3A and 3B | 368 |
| | VEGFA_sg16 | TTTG | CAGTCCCTGTGGGCCTTGCT | FIGS. 3A and 3B | 369 |
| | VEGFA_sg17 | TTTG | TTTGTACAAGATCCGCAGAC | FIGS. 3A and 3B | 370 |
| | VEGFA_sg18 | TTTG | TACAAGATCCGCAGACGTGT | FIGS. 3A and 3B | 371 |
| | VEGFA_sg19 | TTTG | CAGGAACATTTACACGTCTG | FIGS. 3A and 3B | 372 |
| | VEGFA_sg20 | CCCA | GCCCCAGCTACCACCTCCTC | FIGS. 3A and 3B | 373 |
| | VEGFA_sg21 | CCCA | GCTACCACCTCCTCCCCGGC | FIGS. 3A and 3B | 374 |
| | VEGFA_sg22 | CCCA | GAAGTTGGACGAAAAGTTTC | FIGS. 3A and 3B | 375 |
| | VEGFA_sg23 | CCCA | GGCCCTGGCCCGGGCCTCGG | FIGS. 3A and 3B | 376 |
| | VEGFA_sg24 | CCCA | CAGCCCGAGCCGGAGAGGGA | FIGS. 3A and 3B | 377 |
| | VEGFA_sg25 | CCCA | AGACAGCAGAAAGTTCATGG | FIGS. 3A and 3B | 378 |
| | VEGFA_sg26 | CCCA | GGCTGCACCCATGGCAGAAG | FIGS. 3A and 3B | 379 |
| | VEGFA_sg27 | CCCA | TGGCAGAAGGAGGAGGGCAG | FIGS. 3A and 3B | 380 |
| | VEGFA_sg28 | CCCA | CTGAGGAGTCCAACATCACC | FIGS. 3A and 3B | 381 |
| | VEGFA_sg29 | CCCA | CCTGCATGGTGATGTTGGAC | FIGS. 3A and 3B | 382 |
| | VEGFA_sg30 | CCCA | AAGATGCCCACCTGCATGGT | FIGS. 3A and 3B | 383 |
| | VEGFA_sg31 | CCCA | CTTCCCAAAGATGCCCACCT | FIGS. 3A and 3B | 384 |
| | P2RX5-TAX1BP3 | TTTA | CACATAGGCCATTCAGAAAC | FIG. 4B | 385 |
| | NLRC4 | TTTA | GAGGGAGACACAAGTTGATA | FIG. 4B | 386 |
| | intergene | TTTA | AGAACACATACCCCTGGGCC | FIG. 4B | 387 |
| | CLIC4 | TTTA | CCCTGGCTACCTCCCCTACC | FIG. 4B | 388 |
| | Target 36 for PEM-seq | TTTA | AGAACACATACCCCTGGGCC | FIG. 4C | 389 |
| | PCSK9 for PEM-seq | CCCA | GGGGAGGACATCATTGGTGC | FIG. 4C | 390 |
| | miniCRISPRoff at Snrp | CTTC | TTGTGCAGTGCCAGGTGAAA | FIG. 5J | 391 |
| | CRISPRoff at Snrp | 3′-TGG | CTCCTCAGAACCAAGCGTC | FIG. 5J | 392 |
| DNMT1 | DNMT1_sg1 | TTTC | CCTCACTCCTGCTCGGTGAA | FIG. 8D | 393 |
| | DNMT1_sg2 | TTTC | TCAAGGGGCTGCTGTGAGGA | FIG. 8D | 394 |
| | DNMT1_sg3 | TTTC | CCTTCAGCTAAAATAAAGGA | FIG. 8D | 395 |
| | DNMT1_sg4 | TTTG | GCTCAGCAGGCACCTGCCTC | FIG. 8D | 396 |
| | DNMT1_sg5 | TTTG | GTCAGGTTGGCTGCTGGGCT | FIG. 8D | 397 |
| | For enOsCas12f1 & | ATTA | TAGGCATGAGCCGCTGCACC | FIG. 14A | 398 |
| | Un1Cas12f1_ge4.1 | | | | |
| | | ATTA | TGCGGATCAAACCTCACCAA | FIG. 14A | 399 |
| | | ATTT | CACATCTGAGCTGGCTTTCC | FIG. 14A | 400 |
| | | ATTT | TAAGGGAGAAAATAGGTCCC | FIG. 14A | 401 |
| | | ATTT | GTTGTGCTGTAGGAAGCTCA | FIG. 14A | 402 |
| | | ATTC | CTCCTCAGTIGTGAGCCCAT | FIG. 14A | 403 |
| | | ATTC | ACAGCCAACGACTCCGGCCC | FIG. 14A | 404 |
| | | ATTC | TACATCTTCACCCACCAGGG | FIG. 14A | 405 |
| | | ATTG | TGTGGACAGCATGTATATGT | FIG. 14A | 406 |
| | | ATTG | CAGCAGCCCCCGCATCGCAT | FIG. 14A | 407 |
| | | TTTA | CCCTGGCTACCTCCCCTACC | FIG. 14A | 408 |
| | | TTTA | GAGGGAGACACAAGTTGATA | FIG. 14A | 409 |
| | | TTTT | CTGTGTCAGTTTGTGCCACC | FIG. 14A | 410 |
| | | TTTT | AAGGGAGAAAATAGGTCCCC | FIG. 14A | 411 |
| | | TTTT | CGTCCAACTTCTGGGCTGTT | FIG. 14A | 412 |
| | | TTTC | CTCTGCCCCAGGCTGCAGCT | FIG. 14A | 413 |
| | | TTTC | TGCCTCCAGACACACTGCTA | FIG. 14A | 414 |
| | | TTTC | GTCCAACTTCTGGGCTGTTC | FIG. 14A | 415 |
| | | TTTG | TACTTTGTCCTCCGGTTCTG | FIG. 14A | 416 |
| | | TTTG | ACTTTAGTGACTAGCCGCCA | FIG. 14A | 417 |
| | | TTTG | GGTTCTCTCTATAGCCATTG | FIG. 14A | 418 |
| | | CTTA | CTGATCTGGACAAAAGCAAA | FIG. 14A | 419 |
| | | CTTA | CTGGAAGGCACTTGGCATCT | FIG. 14A | 420 |
| | | CTTA | CCTTGGCATGGTGGAGGTAG | FIG. 14A | 421 |
| | | CTTT | CCTCTGCCCCAGGCTGCAGC | FIG. 14A | 422 |
| | | CTTT | CTGAACACATGCACGGCCAC | FIG. 14A | 423 |
| | | CTTT | CTGCTGTCTTGGGTGCATTG | FIG. 14A | 424 |
| | | CTTC | CAGTAAGATTTGGTGTCTAT | FIG. 14A | 425 |
| | | CTTC | TCTCATAGGTGGTATTCACA | FIG. 14A | 426 |
| | | CTTC | ATGGTCCTAGGTGGCTTCAC | FIG. 14A | 427 |
| | | CTTG | TGGGTGCCAAGGTCCTCCAC | FIG. 14A | 428 |
| | | CTTG | GATTCACCGGTGCCCTGGGT | FIG. 14A | 429 |
| | | CTTG | GGTGCATTGGAGCCTTGCCT | FIG. 14A | 430 |
| | | GTTA | AATAGATCAGAGAGGCCAGG | FIG. 14A | 431 |
| | | GTTA | GTGACCCAGCCAGCCATACC | FIG. 14A | 432 |
| | | GTTT | GTGCCACCACCATACCGCCA | FIG. 14A | 433 |
| | | GTTT | GATCCGCATAATCTGGAAAG | FIG. 14A | 434 |
| | | GTTC | TAGATGCTGTCCGAGGCAGT | FIG. 14A | 435 |
| | | GTTC | AGAAAGGCTGCTGATGACAC | FIG. 14A | 436 |
| | | GTTC | CGGAACTGCATGCTCACCAC | FIG. 14A | 437 |
| | | GTTG | GGCTGACCTCGTGGCCTCAG | FIG. 14A | 438 |
| | | GTTG | CCAAAGAACCCTCCCACAGG | FIG. 14A | 439 |
| | | GTTG | TGCTGTAGGAAGCTCATCTC | FIG. 14A | 440 |
| | For enRhCas12f1 | ACCA | ATGATGTCCTCCCCTGGGGC | FIG. 14B | 441 |
| | | ACCA | AATCTTACTGGAAGGCACTT | FIG. 14B | 442 |
| | | ACCA | CGGCTCCTCCGAAGCGAGAA | FIG. 14B | 443 |
| | | ACCT | CTTTGCCCCAGGGGAGGACA | FIG. 14B | 444 |
| | | ACCT | CTGCATGCTCATGGAATGGG | FIG. 14B | 445 |
| | | ACCT | TGGCATGGTGGAGGTAGAGC | FIG. 14B | 446 |
| | | ACCC | TGGGGACTTTGGGGACCAAC | FIG. 14B | 447 |
| | | ACCC | TCGAAGGTCTGTATACTCAC | FIG. 14B | 448 |
| | | ACCC | TGGTGGACATCTTCCAGGAG | FIG. 14B | 449 |
| | | ACCG | GCTGGTCTTGGGCATTGGTG | FIG. 14B | 450 |
| | | ACCG | GTGAATCCAAGTGTCCTCTG | FIG. 14B | 451 |
| | | ACCG | CTTACCTTGGCATGGTGGAG | FIG. 14B | 452 |
| | | TCCA | GACTTTCACACCTTATAGGA | FIG. 14B | 453 |
| | | TCCA | CTTTGTATATCCCTTCTACA | FIG. 14B | 454 |
| | | TCCA | GCAAGGCAGAGGAGGAGCAG | FIG. 14B | 455 |
| | | TCCT | CCCCTGGGGCAAAGAGGTCC | FIG. 14B | 456 |
| | | TCCT | CCTCAGTTGTGAGCCCATGC | FIG. 14B | 457 |
| | | TCCT | CCGAAGCGAGAACAGCCCAG | FIG. 14B | 458 |
| | | TCCC | CTGGGGCAAAGAGGTCCACA | FIG. 14B | 459 |
| | | TCCC | TTCTACAAATTCCTCCTCAG | FIG. 14B | 460 |
| | | TCCC | GGCCCGAGCTAGCACTTCTC | FIG. 14B | 461 |
| | | TCCG | TGGAGGTTGCCTGGCACCTA | FIG. 14B | 462 |
| | | TCCG | AGGCAGTCCTGCCATCAATG | FIG. 14B | 463 |
| | | TCCG | AAGCGAGAACAGCCCAGAAG | FIG. 14B | 464 |
| | | CCCA | GGGGAGGACATCATTGGTGC | FIG. 14B | 465 |
| | | CCCA | CCTGCATGGTGATGTTGGAC | FIG. 14B | 466 |
| | | CCCA | AAGATGCCCACCTGCATGGT | FIG. 14B | 467 |
| | | CCCT | GGGGACTTTGGGGACCAACT | FIG. 14B | 468 |
| | | CCCT | TCTACAAATTCCTCCTCAGT | FIG. 14B | 469 |
| | | CCCT | GGTGGACATCTTCCAGGAGT | FIG. 14B | 470 |
| | | CCCC | TGGGGCAAAGAGGTCCACAC | FIG. 14B | 471 |
| | | CCCC | GCATCGCATCAGGGGCACAC | FIG. 14B | 472 |
| | | CCCG | CTGGTCCTCAGGGAACCAGG | FIG. 14B | 473 |
| | | CCCG | TTTGCCCCTCACTTGGTAGA | FIG. 14B | 474 |
| | | CCCG | CATCGCATCAGGGGCACACA | FIG. 14B | 475 |
| | | GCCA | CCAGGTTGGGGGTCAGTACC | FIG. 14B | 476 |
| | | GCCA | AGTGCCTTCCAGTAAGATTT | FIG. 14B | 477 |
| | | GCCA | TCCAATCGAGACCCTGGTGG | FIG. 14B | 478 |
| | | GCCT | CAACTCGGCCAGGGTGAGCT | FIG. 14B | 479 |
| | | GCCT | TCCAGTAAGATTTGGTGTCT | FIG. 14B | 480 |
| | | GCCC | CAGGCTGCAGCTCCCACTGG | FIG. 14B | 481 |
| | | GCCC | ATGCAGCTCTCCAGACTCAC | FIG. 14B | 482 |
| | | GCCC | TCCTCCTTCTGCCATGGGTG | FIG. 14B | 483 |
| | | GCCG | CCTGTGCTGAGGCCACGAGG | FIG. 14B | 484 |
| | | GCCG | TGCATGTGTTCAGAAAGGCT | FIG. 14B | 485 |
| DMD | DMD_sg1 | TTTC | ATTGGCTTTGATTTCCCTAG | FIG. 17A, | 486 |
| | | | | FIGS. | |
| | | | | 14D-14F | |
| | DMD_sg2 | TTTC | CCTAGGGTCCAGCTTCAAAT | FIG. 17A | 487 |
| | DMD_sg3 | TTTC | CCACCAGTTCTTAGGCAACT | FIG. 17A | 488 |
| | DMD_sg4 | TTTC | TCTCTCAGCAAACACATTAC | FIG. 17A | 489 |
| | DMD_sg5 | TTTG | ATTTCCCTAGGGTCCAGCTT | FIG. 17A | 490 |
| | DMD_sg6 | TTTG | AAGCTGGACCCTAGGGAAAT | FIG. 17A | 491 |
| | DMD_sg7 | TTTG | CTGAGAGAGAAACAGTTGCC | FIG. 17A | 492 |
| | DMD_sg8 | TTTA | CTCTCCTAGACCATTTCCCA | FIG. 17A | 493 |
| | DMD_sg9 | GCCA | ATGAAACGTTCTTGTCTTAG | FIG. 17A | 494 |
| | DMD_sg10 | CCCA | GTATAAAATACAGAGCTAAG | FIG. 17A | 495 |
| | DMD_sg11 | CCCA | CCAGTTCTTAGGCAACTGTT | FIG. 17A | 496 |
| | DMD_sg12 | TCCA | CCAATCACTTTACTCTCCTA | FIG. 17A | 497 |
| | DMD_sg13 | GTTC | CTAGGGCAGAGAACAGGATT | FIG. 17A | 498 |
| | DMD_sg14 | TTTC | TGGCATTGTCATACGTGTAT | FIG. 17A | 499 |
| | DMD_sg15 | CTTC | AATCAATATAGGGCCACACA | FIG. 17A | 500 |
| | DMD_sg16 | CTTC | TGTATTCAAGCTCAAGGCCT | FIG. 17A | 501 |
| | DMD_sg17 | GTTC | TGCTACTTACTGGGAATTTG | FIG. 17A | 502 |
| | | | TTGTGCTGGACGGTGACGTA | FIGS. 14D-14F | 511 |
| | | | CCTAGGGTCCAGCTTCAAAT | FIG. 14D-14F | 512 |

TABLE 5
sgRNA IVT

| | | SEQ ID |
|---|---|---|
| sgRNA for IVT | sequences | NO: |
| OsCas12f1 sg1 | AGGGACGACTTCCCGTCCCAAAATCGAGACAGTAGCCGTAAAACT | 503 |
| | TTGAGTTTCAGAGTGGGCGACACACTCGAAAAGGTTAAGATATGC | |
| | ACATAGTAATCCGTGCATGAGCCGCGAAAGCGGCTTGAAGGTGCT | |
| | GTCTTGGGTGCATTGG | |
| OsCas12f1 sg2 | AGGGACGACTTCCCGTCCCAAAATCGAGACAGTAGCCGTAAAACT | 504 |
| | TTGAGTTTCAGAGTGGGCGACACACTCGAAAAGGTTAAGATATGC | |
| | ACATAGTAATCCGTGCATGAGCCGCGAAAGCGGCTTGAAGGCAGT | |
| | AAGATTTGGTGTCTAT | |
| enOsCas12f1 sg1 | AGGGCCGACTTCCCGGCCCAAAATCGAGACAGTAGCCGTAAAACG | 505 |
| | TTGAGTTTCAGCGTGGGCGACACACTCGAAAAGGTTAAGATATGC | |
| | ACATAGTAATCCGTGCATGAGCCGCGAAAGCGGCTTGAAGGTGCT | |
| | GTCTTGGGTGCATTGG | |
| enOsCas12f1 sg2 | AGGGCCGACTTCCCGGCCCAAAATCGAGACAGTAGCCGTAAAACG | 506 |
| | TTGAGTTTCAGCGTGGGCGACACACTCGAAAAGGTTAAGATATGC | |
| | ACATAGTAATCCGTGCATGAGCCGCGAAAGCGGCTTGAAGGCAGT | |
| | AAGATTTGGTGTCTAT | |
| RhCas12f1 sg1 | ACGGTTGATTTAGCAACCGAAGTCTGAGGGCATGTAGAAAAAAGT | 507 |
| | ATAGGTATATACCAACATACTTGCATTGCCACTCGGAAAGGGTTA | |
| | ACCTTGGTCATTGTGTTACCGACCAAGCATTCCAGAAATGGAATG | |
| | TAAATCCTGCATGGTGATGTTGGAC | |
| RhCas12f1 sg2 | ACGGTTGATTTAGCAACCGAAGTCTGAGGGCATGTAGAAAAAAGT | 508 |
| | ATAGGTATATACCAACATACTTGCATTGCCACTCGGAAAGGGTTA | |
| | ACCTTGGTCATTGTGTTACCGACCAAGCATTCCAGAAATGGAATG | |
| | TAAATAAGATGCCCACCTGCATGGT | |
| enRhCas12f1 sg1 | ACGGCTGATTTAGCAGCCGAAGTCTGAGGGCATGTAGAAAAAAGT | 509 |
| | ATAGGTATATACCAACATACTTGCATTGCCACTCGGAAAGGGTTA | |
| | ACCTTGGTCATTGTGTTACCGACCAAGCATTCCAGAAATGGAATG | |
| | TAAATCCTGCATGGTGATGTTGGAC | |
| enRhCas12f1 sg2 | ACGGCTGATTTAGCAGCCGAAGTCTGAGGGCATGTAGAAAAAAGT | 510 |
| | ATAGGTATATACCAACATACTTGCATTGCCACTCGGAAAGGGTTA | |
| | ACCTTGGTCATTGTGTTACCGACCAAGCATTCCAGAAATGGAATG | |
| | TAAATAAGATGCCCACCTGCATGGT | |

Various modifications and variations of the described products, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

### Exemplary Sequences

| | | SEQ |
|---|---|---|
| | | ID |
| Cas | Amino acid sequence | NO |
| OsCas12f1 | MGKGVLAKVMKYELRYLDGCGDFSNMQEQVWALQRQTREILNRSIQIAFQWDCANSEHHRKTGEYLDLKTETGYKRLD | 1 |
| (ME-B.3) | GHIYNCLKGQYEDMATSNLNATIQKAWKKYNSSKKEILRGSMSIPSYKMNQPLTLDKNTVKLSEGERNPIVTLTLFSD | |
| | KFKRAQGVSNVKFSMPLHDGTQRAIFANLMNGTYQLGECQLVYKRPKWFLFVTYKFPPVEHPLDPDKILGVDMGEACA | |
| | LYASTFGEHGYLKIDGGEITKYAKKMEARIRSMQKQAAHCGEGRIGHGTKTRVSVVYQAKDKVARFRDTINHRYSKAL | |
| | IDYALKNQCGTIQMEDLTGIKEDTGFPKFLRHWTYYDLQSKIEAKAAEHGIQVVKINPRHTSQRCSRCGHIDKANRTS | |
| | QADFCCTKCGFSANADFNASQNISIRNIDKIIAKAIGANRKQT* | |
| RhCas12f1 | MITVRKLKILIDGESRNESYKFIRDSMYAQYLALNKAMSYLGTAYLSRDKEIFKEAIKSLNNSNPIFDNINFGKGIDT | 2 |
| (ME-A.1) | KSSVNQTVKKHIQADIKNGLAKGERSIRNYKRDYPLMTRGRDLKFFYCDTNSTKVKVKWVNGIIFDVMLGKEYNKNDL | |
| | ELRSFLNRVINKEYKISQSSICFDKHNRLILNLSVNITDNIPNEVVKGRIVGVDLGMKIPAYVTLNDSEYIGKPIGDI | |
| | NDFLKVRKQFKERKERLQKQLAINKGGRGITNKMQLMDAFTNKEKNFANTYNHGVSKAIINFAKKYKAEQINVEFLAL | |
| | AGSEKEILSSTIRYWSYYQLQQMIEYKANREGIAVKYVDPYLTSQTCCKCGNYEVGQRINQELFECKLCGNKMNADRN | |
| | ASFNIARSTKYISSKEESDFYKQLK* | |
| Ob2Cas12f1 | MSKGSLAKVMKYELRYLDGAGSFEQMQERLWVLQRQTREILNRSTQISFHWDYTSREHFEQTGQYLDVFSETGYKRLD | 3 |
| (ME-B.4) | GYIYSRVKDSCGDMASGNINATLQKAWNKYGTSKLDVLRGQMSLPSYKKDQPLVIEKHNIRLSMDGQQALAEITLFSN | |
| | KFKKENSLSSNVRFAFQLHDGTQRRILNSVLSGEYGLGQCQLVYDRPKWFLLLTYTFTPQNRQLDPDRILGVDLGECY | |
| | ALCASVFGEYGSLRIEGGEVTAYAKKLEARKRSLQKQAAVCGEGRKGHGTKTRVADAYQMQDRIANFRDTVNHRYSKA | |
| | LIDYALKNQCGTIQMEDLSGIRQDTGFPKFLQHWTYYDLQSKIENKAKEHDIRIVKINPRYTSQRCSKCGAIDSGSRT | |
| | SQARFCCTKCGFTANADYNASQNISIKGIDLLIEKELGAKAE* | |
| Ob3Cas12f1 | MGKGEISKVMKYELRYLDGSGSFEEMQQRVWALQRKTREIQNRTVQIAFHWDYINREHFIQTGNNLNVLQETGYKRLD | 4 |
| (ME-B.5) | GYIYDRLKGQSAEMSGANLNATIQTAWKKYNSAKPKVLSGTMSVPSFKRDQPLIINSNCVKFSRSESECLAELTLFSR | |
| | EYKKEHDLSSNVRFAIRLHDSTQRSILERVLSGAYRKGQCQLVYQRPKWFLFLTYSFFPMQHDLDPEKYLGVDLGECC | |
| | ALYASSVGEYGSLKLEGGEITAFAKQLEARKRSMQKQAAYCGEGRIGHGTKTRVADVYKMENRIANFRDTVNHRYSKA | |
| | LIDYAVKHQYGTIQMEDLSGIKNDTGFPKFLRHWTYFDLQEKIDAKAREHGIHVVKVNPQYTSQRCSKCGSIDSRNRK | |
| | SQKEFCCLNCGYKVNADENASQNLSIKGIDVIIQKYIGAKSKQTENNG* | |
| Cb1Cas12f1 | MAKGTVTKVMKYELRYLSGFSDFHAMQQAVWGLQRQSREILNKTIQMAFHWDYISRENFNANGVYLDVKAETGYKTYD | 5 |
| (ME-B.14) | GYIYNSLKSAYADMAAANLNAAIQKAWKKYKDAKMEVLRGTMSTPSYRSDQPVLINKNCVKLFDGGVRLTLFSDRFKR | |
| | ENNLNGNLEFAVQLHDGTQRSIFANLLNGTYALGQCQLVYDKRKWFLLVTYIFTPEKHELDPEKILGVDLGQTYALYA | |
| | SSVCARGTFRIEGGEAAECAHRLEQRKRSLQQQARFCGEGRVGHGTKTRVAAVYSAGDKIASYRDSINHRYSKALVEY | |
| | AVKNGYGTIQMEDLIGIQNDLDHPKRLQHWTYYDLQTKIENKAKEHGVGVVKVNPRYTSQRCSRCGHIERENRPTQKV | |
| | FCCKACGFEGNADYNASQNLSMRNIDKIIEKELSAKGE* | |
| Cb2Cas12f1 | MCALTKIMKYELRYLDGFPDFSAMQNAVWPLQRQTREILNRTIQEAYRWDYFSATKKKETGEYPDLQKETGYKRLDGY | 6 |
| (ME-B.1) | IYHVLSPDYPDFSSSGVNATIQKAWKKYKSSKADVWKGEMSLPSYKSDQPIVLHAKQIKLSGDTRAAAATLSLFSNKF | |
| | KKEHEISGNVQFAITLHDNTQRTIYQKLRNGEYKLSESQLVYDKKKWFLYLAYSFNPAEHALDPEKILGVDMGEKFAL | |
| | YASSFGEYGHFKIEGSEVTEYAKALERRRRSLQQQARYCGEGRIGHGTKTRVGVVYREEDRIANFRSTINHRYSKALI | |
| | EYAVKNGYGTIQMENLTGIKENLQFPRRLQHWTYYDLQSKIEAKAKEHGIAVVKVNPKHTSQRCSRCGHIAAENRPKQ | |
| | EVFQCVKCGYACNADFNASQNISIKDIEKLIQETIGANPK* | |
| Cb5Cas12f1 | MAKKGNSQKKQIVKVMKYELKYEKGCADFNEMQNELWKLQRQTREVMNRTIQLCYHWSYVQAEYCKQHGCARRDVKPC | 7 |
| (ME-B.18) | DVYETNATSLDGYIYQLLKVEYPDFFMKNLNATLRKAHQKYDALLFDIQEGNSSIPSFKKDQPLIFEKKAICISKCLP | |
| | DKRQITLSCFSDSYIDAHPTLDKITFTVRARSASEKSIFDHIISGKYALGTSQLVYEKKKWFFLLSYKFTPESVDVNP | |
| | EKVLGVDLGVVNALCAGSVENPHDSLFIKGTEAIEQIRRLEARKRDLQKQARYPGDGRIGHGTKTRVSPVYQTRDAIA | |
| | RMQDTLNHRWSRALIDFACKKGYGTIQMEDLSGIKAMESEKPYLKHWTYFDLQSKIIYKAEEKGIRVVKVNPKCTSRR | |
| | CSACGYISKENRKNQAEFLCVNCGYHHNADYNAAQNLSIPQIDRLIEKQLKEQESEESEAGTNPK* | |
| Ob1Cas12f1 | MAEKTIVKVMKFELRYIDGAGEFSEMQKHLWELQKQTREVLNKTIQMGYALECKRFAHHDKTGQWLDDKELTGSKYKA | 8 |
| (ME-B.15) | VADYINAELKEDYNIFYSDCRNSTVRKAYKKFKDAKNKIFSGEMSLPSYRSNQPIIIHNRNVIIRGNAESALVGLKVF | |
| | SDGFKALHGFPAAVNFKLCVKDGTQRAIIENVISEIYKISESQLIYDNKKWFLILAYRFTQKKNDLNPDKILGVDLGV | |
| | KFAVYASSIGEYGSFRIKGGEVTEFIKRLEKRKKSLQNQATVCGDGRIGHGTKTRVADVYKARDKISNFQDTINHRYS | |
| | RAIVDYARKNGYGTIQLEKLDNSIEKKGDYSPVLVHWTYYDLRTKMEYKAAEYGIKVIAVEPKYTSQRCSKCGYISSE | |
| | NRKTQESFECIKCGYKCNADFNASQNLSVRDIDRIIDEYLGANPELT* | |
| EsCas12f1 | MVCNKVVKIALICDQIDKDGKDVNYNDIYKLLWDLQKQTREAKNKVIRLCWEWSGYSSEYFKTHEEYPKDKEILGISL | 9 |
| (ME-B.16) | RSYLYNRIKGDYNLYSGNLSQSAKIAYIEYKNSLTDVLRGDKSIINYRENQPLDIKNKAIQLLYENDNFFVRVALINK | |
| | DKRKELNFKDCSVRFKLLVKDDSTRTILERCFDEVYTITASKIMYNKKKKQWYINLGYKFTKEIDKTLDKDRILGVDL | |
| | GVINPLVASVYGSYDRLIIGGGEIDKFRKRVEANKVQMLKQGKYCGDGRIGHGVNTRNKPAYNIEDKISRFRDTVNHK | |
| | YSKAVVDYAVKNNCGTIQMEDLKGITQNKNERYLKNWTYFDLQTKIEYKAKALGIEVKYKNPKYTSQRCSKCGHIAEE | |
| | NRPEQKTFKCVKCGFKVNADYNASQNLAIKDIDKIIEQYYNKG* | |
| Pt1Cas12f1 | MKYTKVMRYQIIKPLNAEWDELGMVLRDIQKETRAALNKTIQLCWEYQGFSADYKQIHGQYPKPKDVLGYTSMHGYAY | 10 |
| (ME-B.19) | DRLKNEFSKIASSNLSQTIKRAVDKWNSDLKEILRGDRSIPNFRKDCPIDIVKQSTKIQKCNDGYVLSLGLINREYKN | |
| | ELGRKNGVFDVLIKANDKTQQTILERIINGDYTYTASQIINHKNKWFINLTYQFETKETALDPNNVMGVDLGIVYPVY | |
| | IAFNNSLHRYHIKGGEIERFRRQVEKRKRELLNQGKYCGDGRKGHGYATRTKSIESISDKIARFRDTCNHKYSRFIVD | |
| | MALKHNCGIIQMEDLTGISKESTFLKNWTYYDLQQKIEYKAREAGIQVIKIEPQYTSQRCSKCGYIDKENRQEQATFK | |
| | CIECGFKTNADYNAARNIAIPNIDKIIRKTLKMQ* | |
| RhgCas12f1 | MATKVMRYQIIKPIDCNWDLFGKVLRDIQYDTRQIMNRTIQYCWEWQGYSSDYKIAKGEYPKTRETFGYSDMRGYAYD | 11 |
| (ME-B.2) | KLKSIYQRLNTANLTTSITRAVQRWKTDTKDVIRGDKSIACFRADVPIDLHNKSMNIEKSDDGYIVALSLASNIYKKE | |
| | LDRNSGQFSVLINEGNKSNRDVLDRCIAGQYKISASQILREKNKWFLNLSYSFEISKPDKSRDNILGIDVGIVHPVYM | |
| | AVYNSPARRSISGGEIDNFRKQVQKRIKELQLQGKQCGEGRIGHGIKTRVKPIEFAKDKVANFRNTINHKYSKAIVEF | |
| | AIKNGCGIIQMEDLKGINTDNVFLKNWTYYDLQQKVKYKAELEGIEVKLIDPQYTSQRCCKCGYIHRDNRPEQAKFKC | |
| | IDCGFEVNADYNASLNIATPDIDKIILEFLKCET* | |
| Bc1Cas12f1 | MGVTIKIMKYQILCPMNVDWTIFEKHLRNLTYQVRTISNRTIQQLWEFDALSFDYFKERGTYPTVQDLYGCTQKKIDG | 12 |
| (ME-B.10) | YIYHTLQSKYPDIHKGNMSTTLQKIIKTWKSRRNEIRKGEMSIPSFRNRIPIDLHNNSVDIIKEKNGDYIAGISLFSR | |
| | DFHKENGDVPKGKIFVKLGTQKQKSMKVILDRLINQTYSKGACMIHKYKNKWYLSITYKFNAIKENKFDKELIMGIDM | |
| | GGINTVYFAFNEGFIRSNIKSDEIKMENERIRQRRINLLKQSKYCSNSRTGKGRTKRLQPIDVLSNKIAKFRNSTNHK | |
| | YANYIVKQCLKHNCGRIQMELLKGISKNDKVLKDWTYFDLQEKIKNQAEIYGIEVIKVVPAYTSQRCSQCGYICKENR | |
| | CTQAMFECKQCGYKTHADYNAAKNISTYDIENIINKQLAVQSKLHSKKCMEEYIEELGYLD* | |
| BfCas12f1 | MSTVVKVMKYQIICPVNIEWKAFETYLRTLSYQVRTIGNRTIQKLWDFDNQSLNHFRENGVYPSAQQLYGCTQKTISG | 13 |
| (ME-B.8) | YIYDQLKEEYQDMNKANMSTTLQKTIKTWNSRKKEIRSGEMSIPSFRNNLPIDIHGNSIQITKEKSGDYIASLSLFSS | |
| | NFIIENNLPNGKIQVKLSTRKQNSMKVILDRIIENTYAKGACMLHKHKNKWYLSIIYKPTVKEEHKFEEDLVMGIDMG | |
| | KINVLYFAFNKGWIRGAISGEEIEAFRKKIEHRRISLLRQGKYCSGNRVGKGREKRIKPIDVLNNKIAKFRNATNHKY | |
| | ANYIVQQCLKYNCGTIQLENLQGISKEQTFLKNWTYFDLQEKIKQQAHQYGMKVVTIDPSYTSKRCSECGYIHKNNRK | |
| | SQSTFECQQCNLKVHADYNAAKNISIYNIEKVIQKQLKLQEKLNSKKFTEQYIEQVENIN* | |
| BtCas12f1 | MSIAVKVMKYQIVCPVNIEWKTFEIYLRTLSYHFRTIGNRTIQKLWEYDNQSLKHFKDTGQYPSAQQLYGCTQKTISG | 14 |
| (ME-B.6) | YIYDQLKEEYQDINKANMSTTLQKTIKTWNSRKKEIWSGEMSIPSFRNNLPIDIHGNSIQIIKEKSGDYIASVSLFSS | |
| | KFIKENDLPNGKILVKLSTRKQNSMKVILDRIIDSTYAKGACMLHKHKKKWYLSITYKSNIKEELKFDEDLIMGIDMG | |
| | KINVLYFAFNKGLVRGAISGEEIEAFRKKIEHRRISLLRQGKYCSGNRIGKGRKKRIKPIEVLNDKIAKFRTATNHKY | |
| | ANYIVQQCLKYNCGTIQLEDLQGISKEQTFLKNWTYFDLQEKIKNQANQYGIKVVKIDPSYTSQRCSECGYIHKNNRQ | |
| | NQSTFECQQCSFKVHADYNAAKNISVYNIEKVIQRQLKLQEKLNLTKYKEQYIEQMENIN* | |
| HsCas12f1 | MRALENQKPLKSIKKPVCKISRTLSVPIQRPCGYVWNDFGHLLCIIRNDVAQAYNMAMSESYLYFSERENYKREHGKY | 15 |
| (ME-B.12) | PKVEQLAKRNVYKKLTENFPHIGTGILATIANKVESKLKKEYVEVMLKGTKSVSNYKKGTPIPIRAQGWKERTFKRKR | |
| | KDKMTFHLLSKKAEQSKSLDFLKDEKGKIPCSFTVRIALKKLNNSQRAVYNRIWAGEYKAGAIDILQRKGKWFINISY | |
| | HMLETKRLEKQLDKNVIVGVDLGIVNGVVCAVSNDAYDRLVLRKDIEGFRKQIWKRKHLAWKSTRRGGKGRKYYLRMS | |
| | DSLKKKEHNFRNTLYHDWTRKVIDYALKHGAKVIQIEDLSGLVEAKKKMKKGVLKNWVISDFVEKLTYKAEEYGIEIV | |
| | KVNPRYTSQRCHKCGHIEKDNRKEQSKFVCLKCGHSCNADFNAAKNIATKNIADIISASLPQT* | |
| MsCas12f1 | MTDEQARLQKVATFQIVKPVNMDWREFRKLLRDVRYRLWRLGNMAVSEAYLTFHKKYRMGQAQSDGAHKLSVLDKRLR | 16 |
| (ME-B.13) | QALIDEGVRVEELSRYSRKGAVSGYICGAFEKTKLSAIKSKSKWRDIINGRASLPVFRRDLAIPINCSDCQPRMIERT | |
| | EAGEYQVDLRICLQDKELAPNGYPRVLLSTAKISDGQRAVLERLVSNKTNSLPGYRHRFFEIKEKRGKWFLSVSYDFP | |
| | RAEAGKLHQDIIVGVDLGWSVPLYAALNKGYARIGWKKLEPLAKRIRHLQKQVKGRRLSMQRGGQADLAGPTARMGHG | |
| | RRRNLQAIEKLEGKINDAYTTLNHQLSHCIIEFAKNNGAGVIQIEDLRGLADELRATFIGQNWRYHQLQEFIKYKAEE | |
| | AGIKVVPPVNPFYTSRRCSVCGYLHKDFTFEYRQVNRKNGMSVMFECPECSKKAKEEGKEYKALNADYNAARNLATAN | |
| | IEEKIRLQCKEQGIEYTELPKS* | |
| ScCas12f1 | MKDYIRKTLSLRILRPYYGEEIEKEIAAAKKKSQAEGGDGALDNKFWDRLKAEHPEIISSREFYDLLDAIQRETTLYY | 17 |
| (ME-B.11) | NRAISKLYHSLIVEREQVSTAKALSAGPYHEFREKFNAYISLGLREKIQSNFRRKELARYQVALPTAKSDTFPIPIYK | |
| | GFDKNGKGGFKVREIENGDFVIDLPLMAYHRVGGKAGREYIELDRPPAVLNVPVILSTSRRRANKTWFRDEGTDAEIR | |
| | RVMAGEYKVSWVEILQRKRFGKPYGGWYVNFTIKYQPRDYGLDPKVKGGIDIGLSSPLVCAVINSLARLTIRDNDLVA | |
| | FNRKAMARRRTLLRQNRYKRSGHGSANKLKPIEALTEKNELYRKAIMRRWAREAADFFRQHRAATVNMEDLTGIKDRE | |
| | DYFSQMLRCYWNYSQLQTMLENKLKEYGIAVKYIEPKDTSKTCHSCGHVNEYFDFNYRSAHKFPMFKCEKCGVECGAD | |
| | YNAARNIAQA* | |
| Un2Cas12f1 | MEVQKTVMKTLSLRILRPLYSQEIEKEIKEEKERRKQAGGTGELDGGFYKKLEKKHSEMFSFDRLNLLLNQLQREIAK | 18 |
| (ME-B.20) | VYNHAISELYIATIAQGNKSNKHYISSIVYNRAYGYFYNAYIALGICSKVEANFRSNELLTQQSALPTAKSDNFPIVL | |
| | HKQKGAEGEDGGFRISTEGSDLIFEIPIPFYEYNGENRKEPYKWVKKGGQKPVLKLILSTFRRQRNKGWAKDEGTDAE | |
| | IRKVTEGKYQVSQIEINRGKKLGEHQKWFANFSIEQPIYERKPNRSIVGGLDVGIRSPLVCAINNSFSRYSVDSNDVF | |
| | KFSKQVFAFRRRLLSKNSLKRKGHGAAHKLEPITEMTEKNDKFRKKIIERWAKEVTNFFVKNQVGIVQIEDLSTMKDR | |
| | EDHFFNQYLRGFWPYYQMQTLIENKLKEYGIEVKRVQAKYTSQLCSNPNCRYWNNYFNFEYRKVNKFPKFKCEKCNLE | |
| | ISADYNAARNLSTPDIEKFVAKATKGINLPEK* | |
| CiCas12f1 | MKTTEKNVLMTKCIKVTLNRCVNYNMKEIMNIIREMQYLSSKAYNLATNYLYIWDTNSMNFKNLYEEKIVDKDLLGKS | 19 |
| (ME-B.7) | KSAWIENRMNEIMKGFLINNVAQARQDVINKYNKSKKDGLFIGKVTLPSYKMNGKVVIHNKAYRFSKNEGYFVEIGLF | |
| | NKEKKEELNCDWIKFKLDKIDSNKKATIYKILNGDYKQGSAQLHINKKGKIEFIISYSFERENSIKLDKNRTLGIDIG | |
| | IVNIAAMAIWDNNKQEWELTRYSHNLISGNEAIALRQKYYKLGLRNKELEKNINRELHELEEKEYRGLSTNIISGHNL | |
| | TYKRIMLNSKRIRLSQSCKWCGNSKVGHGRRVRCKQVDKIGNKIERFKDTFNHKYSRYIVDFAVKNNCGIIQMENLKN | |
| | FNPSEKFLKDWPYFDLQTKIEYKAKEYGIEVIKVNPKYTSKRCSRCGCINELNRDCKKNQSKFKCVNDECNNYENADI | |
| | NAAKNIALPYIDKIIEQCLETNKVV* | |
| CpCas12f1 | MKLNKCIKVTLVKCLNYDYKEIKQIIRDFNYTACKASNKAMRMWFFHTQDMIDKKNKYKEFNQIQYEKDTYGKSYRNV | 20 |
| (ME-B.9) | IEGEMKKIMPLANTSNVGTLHQQLVQNDWSRLKKDILSCKANLPTYKLSTPYFIKNDNFKLRNHNGYFVDIAFFNKEG | |
| | LKQYGYKAGHKFEFQIDKLDGNKKSTINKIINGEYKQGSAQLSISNKGKIELIISYSFEKEEVPVLDKNKILGIDLGI | |
| | TNVATMSVYDSMREQYDYFSWKTNVISGKELIAFRQKYYNLRRDMSIASKTAGQGRCGHGYKTKMKSVNKVRNKIANF | |
| | ADTYNHKISKYIIEFAIKNNCGVIQVEDLSGATADTHNKMLKDWSYYDLQQKIEYKAKEQGIEVIKVNPKYTSKRCSK | |
| | CGCIHEDNRDCRNNQAKFECKVCGYNENADINASKNIAIPDIDNIIKGTEILHSKENKAS* | |
| SvCas12f1 | MTTKCVQVAIEYSSNNILKEVDFYKELRDLQYNSYLACNRAISYMYENDMQNFIIKETDLPRSDDKKLYGKSFAAWIE | 21 |
| (ME-B.17) | NRMNEYMPGALSNNVAQTRQFVVNRYKNDKKAGLLKGNVSLTTFKRINPIIIHNNAYNIIETPKGLGAEIGFFNLPKQ | |
| | KELGIKRVNFLFPKLGSSEKSIIRRLLDKSYKQGAMQISYNQKKKKWMATISFSFNLEEIKTNENLVMGIDLGVSKVA | |
| | TLSIYDASKYEYIKMSFKDTCIDGTELMHYRQKLESRRKALSIASKWASDNNRGHGYKTKMEKANYMGRKYNNFRDTY | |
| | NHKVSRYIVDVAIKYRVGLIQMEDLSGFSEQQQESLLKNWSYYDLQQKIKYKAEENGIRVYFINPKYTSQRCSKCGNI | |
| | DKENRKTQESFSCTVCNYKDNADVNASKNIAIPDIEKIIEEQVKKQY* | |
| AoCas12f1 | MITTRKLKLAIVSDNKNEAYSFIRDETRNQNRALNVAYSHLYFEYIAQEKLKHSDAEYQEHLAKYQELASKKYQEFLK | 22 |
| (ME-A.7) | VKEKAKSDETLQAKVDKAREAYNKAQEKVYKIEKDYSKKAREIYQQSVGLAKQTRIDKLLKNQFNLHYDTVDRVGGTA | |
| | ISHFTNDMKSGVLQGKRSLRNYKSSNPLMIRARSMKVYEENSDYYIKWIKDITFKIIISAGSKQRQNIGELKSVLVNI | |
| | IEGNYKACDSSIGVDKDLILNLSIDIPITKENIFIPNRVVGVDLGLKIPAYVSVNDTPYIKRAIGNINDFLKVRTQLQ | |
| | SQRRRLQKALQSTNGGKGRNKKMQGLERLQAKEKNFVNTYNHFLSKNIVDFAVKNNAGMIHMEELKFDKVKHKSLLRN | |
| | WSYYQLQTMIEYKAKREGIEVYYVDASYTSQTCSKCGNLEEGQRETQDTFVCKKCGYSVNADYNASQNIAKAKTIKEE | |
| | NQQ* | |
| Bc2Cas12f1 | MILTRKIKLVIVSENREEGYNLIRTEIREQHKALNLAYNHLYFEHNAIQKLKQNDEDYKQKRNKLQELINKKYEEHQK | 23 |
| (ME-A.5) | AKNLEKKEALREAYNNKKQELYNFEKEYNEKARQTYQQVVGFTQQTRVRNLINRECNLMSDTKDGITSKVTQDYKNDC | |
| | KAGLLIGKRSLRNYKKDNPLLVRGRSLKFYKEDGDYFIKWNKGTIFKCILHIRKKNVVELQSVLENVLLGAYKVCDSS | |
| | IGFNNKDMILNLSLNIPDKETQGYIPGRVVGVDLGLKIPAYLSLSDKVYVRKGIGSIDDFLRVRTQMQKRRRRLQKSL | |
| | AAVKGGKGREKKLKALDHLKGKEANFAKTYNHELSTQIVTFAVKNQAGQINMEFLEFDKMKNKSLLRNWSYYQLQIMV | |
| | EYKAKREGIIIKYVDAYLTSQTCSKCDHYEDGQREKQENFMCKNCGLEVNADYNASQNIAKSTSYISDSTESEYHKKK | |
| | QQVLKEILGENDIMNEQLSLFNNCDDIA* | |
| CdCas12f1 | MISTRKIKVRCDDSTFYTFFRQEQREQNKALNIGIGIIHANAVLHNVDSGAEKKLKKSIEGLQGKIDKLNKDLEKEKI | 24 |
| (ME-A.3) | TDKKKEEVLKAIETNKKILDGEKKVFKESEEYRKGIDELFKNTYLKSNTLDHVLDSMVNIQYKRTLSLVTQRIKKDYS | |
| | NDFVGIITGQQSLRNYRNDNPLMISNQQLNFKYIDDTFYLDIMCGYRLEVVLGKRDNENVNELKSTLEKVISKEYKVC | |
| | DSSMQFSKNNKDVILNLVIDIPQNSNVYKPVEGRILGVDLGVAVPIYMCLNDDTYKRKGLGDINNFLRVRQQMQTRRR | |
| | KLQKDLTLTNGGKGRKKKTQLLDKLQENERNFVKTYSHALSKRVVEFAKSNKCEYINIEKLTKDGFDNIILRNWSYFE | |
| | LQKMIEYKAEREGITVRYVNPAYTSQKCSRCGEIDKENRQTQANFKCTKCGFELNADHNAAINIARSIEFV* | |
| Cs1Cas12f1 | MNTVRKIKLTILGDTETRNKQYKWIKDEQYNQYRALNLSMTYMVTNLMLKNNESGLENRKEKDILKIENKIKKDEGSL | 25 |
| (ME-A.4) | KKELAKKKINEEKIENIKSNIEELKSEKEKLENELKNIKEYRSNIDEEFKKMYVDDLYNVLNKISFQHEDMKSLVTQR | |
| | VKKDFNNDVKEIMRGDRSVRNYKRNFPILTRGRDLKFQYIEKSEDIEIKWIEGIKFKCILGKPSKSLELKHALHKVIN | |
| | KEYKVCDSSLQFDKNNNLILNLTLDIPQDNKYEKITNRVVGVDLGLKIPAYVALNDTKYIRKAIGSIDDFLKVRTQMQ | |
| | SRVRKLQKSLQVVRGGKGRNKKMKALERFREKERNFARNYNHFLSYNIVKFALDNKAEQINLELLEMKKTQNKSILRN | |
| | WSYYQLQNFIEYKAERVGIKVKYIDPYHTSQTCSECGNYEEGQRVEQDTFVCKRCWHKMNADYNAARNIAMSYNYISK | |
| | KEESEYYKNNKNMV* | |
| Cb3Cas12f1 | MNTVRKIKIIINNENNELRKEQYKFIRDSQYAQYQGLNRCMGYLMSGFYVNNMDIKSEEFKTWQKGVINSANFFQEIS | 26 |
| (ME-A.10) | FGKGIDSKSSITQKVKKDFSIALKNGLAKGERNINNYKRIAPLMTRGRNLKFKYDDNELDILINWVNKIQFKCVLGEH | |
| | KNSLELQHTLHKVINNEYKIGQSSLYFNKKNELILILTIDIPTAKSSYEPIKDRILGVDLGMAVPVYMSINDNSYIKK | |
| | SLGSYSEFAKVRKQFKERRNRLYKQLEACKGGRGRKDKLKAMNQFKEKEKNFAKTYNHFLSKNIVEFALKNKCEFIHL | |
| | EKIESKGLENSVLANWTYYDLQEKIIYKAKREGIGIKFVNSSYTSQTCSKCNYVDKENRKTQAKFICKNCGFKANADY | |
| | NASQNISKSKEFIK* | |
| Cb4Cas12f1 | MNIVKKIKLRIIDNDKELCKKQYLGFTEEQKKELIDKQYKFIRDSQYQQYLGFNRAMGFLMSGYYANNMDIKSDNFKE | 27 |
| (ME-A.11) | HQKKLTNSLYIFDDIKFGVGIDSKSLIVQRVKKDFSTALKNGLAKGERSVTNYKRTYPLLTRHRSIKFLYAENELDIY | |
| | LDWVNKIRFRCELGNHKNSLELQHTLRKVITGEYKISDSSLEFNKKNELILNLNLNIPETKATFIKDRTLGVDLGMAI | |
| | PAYVSLSDTPYIRKGFGSYEEFAKVRNQFKDRRKRLLKQLSLVAGGKGRAKKLHSMEFLKNKEKQFAKTYNHSLSKKI | |
| | IDFALKNNCEYINLEDIKSTSLEDRVLGQWGYYQLQEQIEYKAKLVGIKVRKVKAAYTSQTCSECGNIDKENRKNQST | |
| | FKCTNEDCKLNKKGINADWNASINIARSKEFIK* | |
| BsCas12f1 | MITVRKVKLIVNSEEAEEINRTYKFIRDSMYAQYQGLNRCMGYLLSGYYANGMDIKSDGFKNHMKTIKNSLNIFDDIN | 28 |
| (ME-A.12) | FGIGIDSKSAITQKVKKDFSTSLKNGLAKGERGATNYKRNFPLMTRGRDVKISYLEDTNTFVIKWVNKIEFKVILGQK | |
| | DNIELSHTLHKIINKEYTLGQCTFEFDKNNKLLLALNINIPDNLISKNKEIIPGRVLGVDLGVKVPAMICLNDNTFIK | |
| | KSIGSYNEFFKVRSQFKARRERLYKQLESSNGGKGRKHKLKATMQFRDKEKNFARTYNHFLSKNIIEFAQKYTCETIN | |
| | LEELNKKGFDNNLLGKWGYYQLQSMIEYKAERVGIKVKYVDPAFTSQTCSKCGYVDEENRITQDKFECQKCGFTLNAD | |
| | HNAAINIARK* | |
| Pt2Cas12f1 | MIAVKKLKLTIVEEEEKRKEQYKFIRDSQYAQYQGLNLAMGILTSAYLVSGRDIKSDLFKDSQKSLINSNEIFNGINF | 29 |
| (ME-A.9) | GKGIDTKSSITQKVKKDFSTSLKNGLAKGERGFTNYKRDFPLMTRGRDLKFYEEDKEFYIKWVNKIVEKILIGRKDKN | |
| | KVELIHTLNKVLNKEYKVSQSSLQFDKNNKLILNLTIDIPYKKVDEIVKDRVCGVDMGIAIPIYVALNDVSYVREGMG | |
| | TIDEFMKQRLQFQSRRRRLQQQLKNVNGGKGRKDKLKGLESLREKEKSWVKTYNHALSKRVVEFAKKNKCEYIHLEKL | |
| | TKDGFGDRLLRNWSYYELQEMIKYKADRVGIKVKHVNPAYTSQTCSECGHADKENRETQAKFKCLECGFEANADYNAA | |
| | RNIAKSDKFVK* | |
| CrCas12f1 | MIAVRKLKIMVLCDDESKKNEQYKFLRDSQYAQYLGLNRAMSFLAKEYLSGDKERFKEAKKKLINTCECYQNINFGTG | 30 |
| (ME-A.8) | IDSKSQITQKVKKDLQADIKNGLARGERSIRNYRRTFPLITRGRDLKFSYNGDEIIIKWVNKIYFKVLIGRKDKNYLE | |
| | LMHTLEKIINGEYKVCTSSIQIDKKLILNLTLEIPDKVKKEFQENRVLGVDLGIKFPAYACVSDNTYVRRSFGSIDEF | |
| | LKVRIQFDKRRKRIQQQLQNVKGGKGRKDKLQALDRMRDCERKWVRNYNHALSKRIIDFAFRNKCGIIHLEKLEKDGF | |
| | KNKLLRNWSYYELQDMIGYKAEREGIVVKYVEPAYTSQTCSKCGYVDRENRPSQEHFLCKECGFEINADHNAAINIAR | |
| | SNKVIVDK* | |
| ChCas12f1 | MITVRKLKLTIINDDETKRNEQYKFIRDSQYAQYQGLNLAMSVLTNAYLSSNRDIKSDLFKETQKNLKNSSHIFDDIT | 31 |
| (ME-A.2) | FGKGTDNKSLINQKVKKDFNSAIKNGLARGERNITNYKRTFPLMTRGTALKFSYKDDCSDEIIIKWVNKIVFKVVIGR | |
| | KDKNYLELMHTLNKVINGEYKVGQSSIYFDKSNKLILNLTLYIPEKKDDDAINGRTLGVDLGIKYPAYVCLNDDTFIR | |
| | QHIGESLELSKQREQFRNRRKRLQQQLKNVKGGKGREKKLAALDKVAVCERNFVKTYNHTISKRIIDFAKKNKCEFIN | |
| | LEQLTKDGFDNIILSNWSYYELQNMIKYKADREGIKVRYVNPAYTSQKCSKCGYIDKENRPTQEKFKCIKCGFELNAD | |
| | HNAAINISRLEE* | |
| Cs2Cas12f1 | MITVRKLKLTIVGDEQTRKEQYKIIRDEQYQQYKALNLCMTLLNTHNILNSYNTGSENKLNSQIEKLDNKIEKNKIEL | 32 |
| (ME-A.6) | KKGNLKESKIEKLNKSILELTKEKEKLQQEYLSASKYRSDIDEKLKDMYIKDMYTVVQSQVNFKSKDMMSLVTQRAKK | |
| | DFSNALKNGMARGERSLINYKRDFPLMTRGERWLKFKYNEESDDIYIDWLHDIKFKVILGYKKNENSIELRHTLHKVI | |
| | NKEYKICDSSMQFDRNNNLILNLTLDIPNKESKGYVEGRTLGVDLGIKYPAYVCLSDDTYKRKSIGCAEDFIRVREQI | |
| | RGRRYRLQKQLSMVKGGKGRDKKLRALDRVREAERNFVKTYNHMISKNIIKFAKEHNCEYIHLEKLTKDGFPDIILSK | |
| | WSYYELQNMIEYKSDREGIKVRYIDPAYTSQTCSKCGHIDKENRINQEKFKCVKCGFELNADHNASINISRSNKYLK* | |
| PhCas12f1 | MKTTRKLKLTIIGDEETRKEQYKIIREEQYQQYKALNLCMTLLNTHNILNSYNTGAENKLNAQIDSIDKKIEQAKKEL | 33 |
| (ME-A.13) | EKKGLKESKVSKLKETIEFLENDREKLKDEYLNSSKFRSDIDEKMKEMYIKDMYTVVQNQVNFRARDMMSLVTQRARK | |
| | DFKNSLKNGMAKGERSLTNYKRDFPLMTRGERWLKFEYDKDSDDILINWIHGIKFKVLLGYKKNENSIELRHTLHKVI | |
| | NKEYKICDSSMQFDRNNNLILNLTLDIPDKQNNNYIEKRTLGVDLGIKYPAYVCLNDDTYIRSHIGESLELLKQREQF | |
| | KDRRKRLQQQLKNVKGGKGRNKKLSALNKLSDNERNFARTYNHMISKRIVEFAKKHRCEFINLEKLTKDGFDNNILSN | |
| | WSYYELQNMIEYKAKREGIEVRYIDPAYTSQKCSRCGYIDKENRQTQEKFKCLKCEFEINADHNAAINIARALD* | |
| OpbCas12f1 | MSEQEAAQEGTKLLAKTLTFGLGNPMGFKSKGSVLVELTEDQRKAIYNGLRDASTVVARIINLLNSREYIRQIMKVPE | 34 |
| | ELVAQFKPNYSLVKGPLKRLGIEEAEQVAGSVLSQTFALGVKPDFQGEHGKGLLLKGERQIPLHRTDGTHPIPQRATE | |
| | TRLFQVEKNFYVAMQVFAETWAKKQELPSGWLAFPIKVKPRDKTMAGQLLKTIGGEWKLKNSRLMRNPRTGGNRWLGQ | |
| | IVVAFAPEPFKKMTRSVVMGIDLGVNVPACLHISENGKPLPWAMMVGRGRDMLNTRNLIRSEIVHIIKALKSKDSPLD | |
| | GKARAIYRDKLRDLRKRERRVMKMASQTVAARIADTAKRHGAGTWQMEDLSPDIKTDQPWLARNWAPGMLLDAVRWQA | |
| | RQCGAELVMVNPAYTSQRCARCGHIDPQNRPKQTDFKCMACGHEDNADKNAARNLSVVGIEKLIADFKAPNGAVQ* | |
| CnCas12f1 | MITVRKIKLTIMGDKDTRNSQYKWIRDEQYNQYRALNMGMTYLAVNDILYMNESGLEIRTIKDLKDCEKDIDKNKKEI | 35 |
| | EKLTARLEKEQNKKNSSSEKLDEIKYKISLVENKIEDYKLKIVELNKILEETQKERMDIQKEFKEKYVDDLYQVLDKI | |
| | PFKHLDNKSLVTQRIKADIKSDKSNGLLKGERSIRNYKRNFPLMTRGRDLKFKYDDNDDIEIKWMEGIKFKVILGNRI | |
| | KNSLELRHTLHKVIEGKYKICDSSLQFDKNNNLILNLILDIPIDIVNKKVSGRVVGVDLGLKIPAYCALNDVEYIKKS | |
| | IGRIDDFLKVRTQMQSRRRRLQIAIQSAKGGKGRVNKLQALERFAEKEKNFAKTYNHFLSSNIVKFAVSNQAEQINME | |
| | LLSLKETQNKSILRNWSYYQLQTMIEYKAQREGIKVKYIDPYHTSQTCSKCGNYEEGQRESQADFICKKCGYKVNADY | |
| | NAARNIAMSNKYITKKEESKYYKIKESMV* | |
| Un1Cas12f1 | MAKNTITKTLKLRIVRPYNSAEVEKIVADEKNNREKIALEKNKDKVKEACSKHLKVAAYCTTQVERNACLFCKARKLD | 36 |
| | DKFYQKLRGQFPDAVFWQEISEIFRQLQKQAAEIYNQSLIELYYEIFIKGKGIANASSVEHYLSDVCYTRAAELFKNA | |
| | AIASGLRSKIKSNFRLKELKNMKSGLPTTKSDNFPIPLVKQKGGQYTGFEISNHNSDFIIKIPFGRWQVKKEIDKYRP | |
| | WEKFDFEQVQKSPKPISLLLSTQRRKRNKGWSKDEGTEAEIKKVMNGDYQTSYIEVKRGSKIGEKSAWMLNLSIDVPK | |
| | IDKGVDPSIIGGIDVGVKSPLVCAINNAFSRYSISDNDLFHFNKKMFARRRILLKKNRHKRAGHGAKNKLKPITILTE | |
| | KSERFRKKLIERWACEIADFFIKNKVGTVQMENLESMKRKEDSYFNIRLRGFWPYAEMQNKIEFKLKQYGIEIRKVAP | |
| | NNTSKTCSKCGHLNNYFNFEYRKKNKFPHFKCEKCNFKENADYNAALNISNPKLKSTKEEP* | |
| SpCas12f1 | MGESVKAIKLKILDMFLDPECTKQDDNWRKDLSTMSRFCAEAGNMCLRDLYNYFSMPKEDRISSKDLYNAMYHKTKLL | 37 |
| | HPELPGKVANQIVNHAKDVWKRNAKLIYRNQISMPTYKITTAPIRLQNNIYKLIKNKNKYIIDVQLYSKEYSKDSGKG | |
| | THRYFLVAVRDSSTRMIFDRIMSKDHIDSSKSYTQGQLQIKKDHQGKWYCIIPYTFPTHETVLDPDKVMGVDLGVAKA | |
| | VYWAFNSSYKRGCIDGGEIEHFRKMIRARRVSIQNQIKHSGDARKGHGRKRALKPIETLSEKEKNFRDTINHRYANRI | |
| | VEAAIKQGCGTIQIENLEGIADTTGSKFLKNWPYYDLQTKIVNKAKEHGITVVAINPQYTSQRCSMCGYIEKTNRSSQ | |
| | AVFECKQCGYGSRTICINCRHVQVSGDVCEECGGIVKKENVNADYNAAKNISTPYIDQIIMEKCLELGIPYRSITCKE | |
| | CGHIQASGNTCEVCGSTNILKPKKIRKAK* | |
| AsCas12f1 | MIKVYRYEIVKPLDLDWKEFGTILRQLQQETRFALNKATQLAWEWMGFSSDYKDNHGEYPKSKDILGYTNVHGYAYHT | 38 |
| | IKTKAYRLNSGNLSQTIKRATDRFKAYQKEILRGDMSIPSYKRDIPLDLIKENISVNRMNHGDYIASLSLLSNPAKQE | |
| | MNVKRKISVIIIVRGAGKTIMDRILSGEYQVSASQIIHDDRKNKWYLNISYDFEPQTRVLDLNKIMGIDLGVAVAVYM | |
| | AFQHTPARYKLEGGEIENFRRQVESRRISMLRQGKYAGGARGGHGRDKRIKPIEQLRDKIANFRDTTNHRYSRYIVDM | |
| | AIKEGCGTIQMEDLINIRDIGSRFLQNWTYYDLQQKIIYKAEEAGIKVIKIDPQYTSQRCSECGNIDSGNRIGQAIFK | |
| | CRACGYEANADYNAARNIAIPNIDKIIAESIK* | |

Cas codon-optimized coding sequence
SEQ ID NO: 39, OsCas12f1 (ME-B.3) codon-optimized coding sequence
ATGGGAAAGGGCGTGCTGGCCAAGGTGATGAAATACGAGCTGAGATACCTGGATGGTTGTGGCGACTTCAGCAATATGCAGGAGCAGGTGTGGG
CCCTGCAGCGGCAGACACGGGAAATCCTGAACAGATCCATCCAAATCGCCTTCCAATGGGACTGCGCCAACAGCGAGCACCACAGAAAGACCGG
CGAGTACCTGGACCTGAAAACGGAAACCGGCTACAAGAGACTGGATGGCCACATCTACAACTGCCTGAAGGGCCAGTACGAGGACATGGCCACA
TCTAACCTGAACGCCACCATCCAGAAGGCTTGGAAGAAGTATAACTCCAGCAAGAAGGAAATCCTGAGGGGCAGCATGAGCATCCCCAGCTACA
AGATGAACCAGCCTCTGACACTGGACAAGAATACCGTGAAACTGTCTGAGGGCGAGCGGAACCCAATCGTGACCCTGACACTGTTTAGCGACAA
GTTCAAGCGGGCCCAGGGCGTGTCCAACGTGAAGTTTAGCATGCCTCTGCACGACGGCACCCAGAGAGCCATCTTCGCCAACCTGATGAACGGC
ACCTACCAGCTGGGAGAGTGCCAGCTGGTGTACAAACGGCCTAAGTGGTTCCTGTTCGTGACATACAAGTTCCCCCCCGTGGAACATCCTCTCG
ATCCTGACAAGATTCTGGGCGTCGACATGGGCGAGGCCTGCGCGCTTTATGCCTCTACATTCGGCGAGCACGGCTACCTGAAGATCGATGGAGG
CGAGATTACAAAGTACGCCAAGAAGATGGAAGCTAGAATCCGGAGCATGCAGAAGCAGGCTGCTCACTGTGGCGAAGGCAGAATCGGGCACGGC
ACCAAAACAAGAGTGTCTGTGGTGTACCAGGCCAAGGACAAGGTGGCCAGATTCAGAGATACCATCAACCACAGATACTCTAAGGCCCTGATCG
ACTACGCCCTGAAGAACCAGTGTGGCACCATCCAGATGGAAGATCTGACCGGCATCAAGGAAGATACAGGATTTCCAAAGTTCCTGAGACATTG
GACCTACTACGACCTGCAGAGCAAGATCGAGGCTAAGGCCGCCGAGCACGGCATCCAAGTTGTCAAGATCAACCCTAGACACACCAGCCAGCGC
TGCAGCAGATGTGGACACATCGACAAAGCCAATAGAACCAGCCAAGCTGATTTCTGCTGCACCAAGTGCGGCTTCAGCGCCAATGCCGACTTTA
ATGCCAGCCAGAACATCAGCATCAGAAACATCGACAAGATTATCGCCAAGGCTATCGGCGCCAACCGGAAGCAGACC
SEQ ID NO: 40, RhCas12f1 (ME-A.1) codon-optimized coding sequence
ATGATTACAGTGCGGAAGCTGAAGATTCTGATCGACGGCGAGAGCAGGAACGAGAGCTACAAGTTCATCCGGGACAGCATGTACGCCCAGTACC
TGGCCCTGAACAAGGCCATGAGCTACCTGGGTACCGCCTACCTGAGCAGAGATAAGGAGATCTTCAAGGAGGCCATCAAGTCTCTCAACAACTC
TAATCCTATCTTCGACAACATTAACTTCGGCAAGGGAATCGATACCAAGAGCAGCGTGAACCAGACAGTGAAGAAACACATCCAGGCCGACATT
AAGAATGGCCTGGCCAAGGGCGAAAGATCCATCCGGAACTACAAGCGTGACTACCCCCTGATGACCAGAGGCAGAGATCTGAAGTTTTTCTACT
GCGACACCAATAGCACCAAGGTGAAGGTGAAATGGGTGAACGGCATCATCTTTGACGTGATGCTGGGAAAGGAATACAACAAGAATGATCTAGA
GCTGAGATCTTTTCTGAACAGAGTCATCAACAAGGAATATAAGATCTCCCAAAGCAGCATCTGCTTCGACAAGCACAACAGACTGATCCTGAAT
CTGAGCGTGAACATCACCGACAACATCCCCAACGAGGTGGTGAAGGGCAGAATTGTGGGCGTCGACCTGGGCATGAAAATCCCAGCTTATGTGA
CACTGAACGACAGCGAGTACATCGGAAAACCTATCGGCGACATCAACGACTTCCTGAAGGTGCGGAAGCAGTTCAAAGAAAGAAAGGAGCGGCT
GCAGAAGCAGCTGGCCATCAACAAGGGCGGCAGAGGCATCACCAACAAGATGCAGCTGATGGACGCCTTCACCAACAAGGAAAAGAACTTCGCT
AATACCTACAACCACGGCGTTTCTAAGGCAATCATCAACTTTGCTAAGAAGTACAAGGCCGAGCAGATCAACGTGGAGTTCCTGGCTCTGGCCG
GCAGCGAGAAGGAAATCCTGAGCTCCACAATCCGCTACTGGTCCTACTATCAACTGCAACAGATGATCGAGTACAAGGCCAACCGGGAAGGCAT
CGCCGTGAAGTACGTGGACCCTTACCTGACCTCACAGACCTGCTGCAAGTGCGGCAACTACGAGGTGGGACAGAGAATCAACCAGGAGCTGTTC
GAGTGTAAACTGTGTGGCAATAAAATGAATGCCGATAGAAACGCCAGCTTCAACATCGCCAGAAGCACAAAGTACATCAGCTCTAAAGAGGAAA
GCGACTTCTACAAACAGCTCAAA
SEQ ID NO: 41, Ob2Cas12f1 (ME-B.4) codon-optimized coding sequence
ATGAGCAAGGGCAGCCTTGCTAAGGTGATGAAATACGAGCTGAGATACCTGGATGGCGCCGGCTCCTTCGAGCAGATGCAGGAGAGACTGTGGG
TGCTGCAGAGGCAGACGCGGGAAATCCTGAACAGAAGCACCCAAATCAGCTTTCACTGGGACTACACCTCTAGAGAGCATTTCGAACAGACCGG
CCAGTACCTGGACGTGTTTAGCGAGACAGGCTACAAGAGACTGGATGGATATATCTACTCCCGGGTCAAGGACAGCTGCGGCGACATGGCCAGC
GGCAACATCAACGCCACACTGCAGAAGGCTTGGAACAAGTACGGCACATCTAAACTGGACGTGCTGCGGGGCCAGATGTCCCTGCCATCTTATA
AGAAGGACCAGCCTCTGGTGATCGAAAAGCACAACATCAGACTGAGCATGGACGGCCAGCAGGCCCTGGCTGAGATCACCCTGTTCAGCAACAA
ATTCAAGAAGGAAAACAGCCTGTCCAGCAACGTGCGGTTCGCCTTTCAGCTGCACGACGGCACCCAGCGCAGAATCCTGAACAGCGTGCTGTCT
GGCGAGTACGGCCTGGGCCAATGTCAGCTGGTTTACGACAGACCTAAGTGGTTCCTGCTGCTGACCTACACCTTCACCCCCCAGAATAGACAGC
TGGACCCTGATAGAATCCTGGGCGTGGACCTCGGAGAGTGCTACGCCCTCTGTGCCTCTGTGTTCGGCGAATACGGCTCTCTGAGAATTGAGGG
CGGAGAGGTGACCGCCTACGCCAAGAAGCTGGAAGCCCGGAAGCGGAGCCTGCAGAAACAGGCCGCTGTGTGCGGCGAGGGAAGAAAGGGACAC
GGCACCAAGACAAGAGTGGCCGACGCCTACCAGATGCAGGACCGGATCGCCAATTTTCGGGACACCGTCAACCACAGATACTCCAAAGCCCTGA
TCGACTACGCCCTGAAGAACCAGTGCGGCACAATCCAGATGGAAGATCTGAGCGGCATCCGGCAAGATACCGGATTCCCCAAGTTCCTGCAACA
CTGGACCTACTACGACCTGCAAAGCAAGATCGAGAACAAGGCCAAGGAACACGACATCAGAATCGTGAAGATCAATCCTAGATATACCAGCCAG
CGGTGCAGCAAATGTGGCGCCATCGATAGCGGCAGCAGAACATCGCAGGCTAGATTCTGCTGCACCAAGTGCGGCTTCACAGCCAATGCCGATT
ACAACGCTAGCCAGAACATCAGCATTAAGGGCATCGACCTGCTGATCGAGAAGGAACTGGGAGCCAAGGCCGAG
SEQ ID NO: 42, Ob3Cas12f1 (ME-B.5) codon-optimized coding sequence
ATGGGCAAGGGCGAGATCTCTAAAGTGATGAAGTACGAGCTGAGATACCTGGACGGCTCTGGCAGCTTCGAGGAAATGCAGCAGAGAGTGTGGG
CCCTCCAGCGCAAGACCAGAGAGATCCAAAACAGAACAGTGCAGATCGCCTTCCACTGGGACTACATCAACAGAGAACATTTCATCCAGACCGG
AAACAACCTGAATGTGCTGCAAGAAACCGGCTACAAAAGGCTGGACGGCTATATCTATGATAGACTGAAAGGACAGAGCGCCGAGATGAGCGGA
GCTAATCTGAATGCTACAATCCAGACAGCTTGGAAGAAGTACAACAGCGCTAAACCTAAGGTGCTGAGCGGCACCATGAGCGTTCCTTCCTTTA
AGAGAGATCAGCCTCTGATCATCAACAGCAACTGCGTGAAATTCTCTAGATCTGAGAGCGAGTGCCTGGCCGAGCTGACCCTGTTCAGCCGGGA
ATACAAGAAAGAGCACGACCTGTCCAGCAACGTGCGGTTCGCCATCAGGCTGCACGATAGCACCCAGCGGAGCATTCTGGAAAGAGTCCTGAGT
GGCGCCTACAGAAAGGGCCAGTGCCAGCTGGTGTACCAGCGCCCCAAGTGGTTCCTGTTTCTGACCTACTCTTTTTTCCCAATGCAGCACGACC
TGGATCCTGAGAAATATCTGGGCGTGGACCTCGGCGAATGTTGCGCCCTCTACGCCAGCTCTGTGGGCGAGTACGGAAGCCTGAAGCTGGAAGG
CGGGGAGATCACCGCCTTTGCCAAGCAGCTGGAGGCCAGAAAGCGGTCCATGCAGAAACAGGCCGCTTACTGCGGCGAGGGCAGAATCGGACAC
GGCACAAAGACAAGAGTGGCCGATGTGTACAAGATGGAAAACAGAATCGCCAACTTCCGGGACACTGTGAACCACAGATACAGCAAGGCCCTGA
TCGACTACGCCGTGAAGCACCAGTACGGCACAATCCAGATGGAAGATCTGAGCGGTATTAAGAACGACACCGGCTTCCCCAAGTTCCTGCGGCA
CTGGACCTACTTCGACCTGCAGGAGAAGATCGACGCCAAGGCCAGAGAGCACGGCATCCACGTGGTGAAGGTCAATCCTCAGTACACCAGCCAG
CGGTGCAGCAAGTGCGGCAGCATCGACAGCAGAAACCGGAAGTCCCAGAAGGAATTCTGCTGTCTGAACTGTGGCTACAAGGTGAACGCCGACT
TCAACGCTTCCCAAAATCTGAGCATCAAGGGCATCGACGTGATCATCCAAAAGTACATTGGAGCCAAGAGCAAGCAGACCGAGAACAACGGC
SEQ ID NO: 43, Cb1Cas12f1 (ME-B.14) codon-optimized coding sequence
ATGGCCAAAGGCACCGTGACCAAGGTGATGAAATACGAGCTGAGATACCTGTCCGGCTTCAGCGACTTCCACGCCATGCAGCAGGCCGTGTGGG
GCCTGCAGAGACAGTCTAGAGAGATTCTGAACAAGACAATCCAAATGGCCTTTCACTGGGATTACATCAGCAGAGAAAATTTCAACGCCAACGG
CGTGTACCTGGATGTGAAGGCCGAAACCGGCTACAAGACCTACGACGGCTACATCTACAACAGCCTGAAGAGCGCCTACGCCGATATGGCCGCT
GCAAACCTCAACGCCGCCATCCAGAAGGCCTGGAAGAAGTACAAGGACGCCAAGATGGAAGTCCTGAGAGGCACCATGTCAACACCTAGCTATA
GATCCGATCAGCCTGTGCTGATCAACAAGAACTGCGTGAAGCTGTTCGACGGCGGAGTGCGGCTGACCCTGTTCTCTGATAGATTCAAGAGAGA
GAACAACCTGAATGGCAATTTGGAGTTCGCCGTGCAGCTGCACGACGGCACCCAGCGGAGCATCTTCGCCAATCTTCTGAACGGCACCTACGCT
CTGGGCCAGTGCCAGCTGGTGTATGACAAGCGGAAGTGGTTCCTGCTGGTGACCTACATCTTCACCCCAGAAAAGCACGAGCTGGACCCTGAGA
AGATCCTGGGCGTGGACCTGGGCCAGACATATGCTCTGTACGCCAGCAGCGTGTGCGCCAGAGGCACATTCAGAATCGAGGGAGGTGAGGCCGC
TGAGTGCGCCCACAGACTGGAACAGCGCAAGAGAAGCCTGCAACAACAAGCTAGATTTTGCGGAGAAGGCAGAGTGGGCCACGGCACAAAGACC
AGAGTGGCCGCTGTGTACTCTGCTGGCGACAAAATCGCCTCTTACCGGGACAGCATCAACCACAGATACAGCAAGGCCCTGGTCGAGTACGCCG
TGAAGAATGGATACGGCACCATCCAGATGGAAGATCTGACAGGCATCCAGAACGACCTGGATCACCCTAAGAGGCTGCAGCACTGGACATACTA
CGACCTGCAGACCAAGATTGAGAACAAGGCCAAAGAGCACGGAGTGGGCGTTGTGAAGGTGAACCCCAGATACACCAGCCAGCGGTGTAGCAGA
TGCGGCCATATCGAGAGAGAAAACAGGCCCACACAGAAAGTCTTTTGTTGTAAGGCCTGCGGCTTCGAGGGGAATGCCGACTACAACGCCAGCC
AGAACCTGTCCATGCGGAACATCGACAAGATCATCGAGAAGGAACTGAGCGCCAAAGGAGAA
SEQ ID NO: 44, Cb2Cas12f1 (ME-B.1) codon-optimized coding sequence
ATGTGCGCCCTGACAAAGATCATGAAGTACGAGCTGAGATACCTGGATGGCTTCCCTGATTTTTCTGCCATGCAGAACGCTGTGTGGCCCCTGC
AGCGGCAGACCCGGGAAATCCTGAACAGGACAATCCAGGAGGCCTATCGGTGGGACTACTTCAGCGCCACCAAGAAGAAAGAAACCGGCGAGTA
CCCCGACCTGCAGAAGGAAACCGGCTACAAAAGACTGGACGGCTACATCTACCACGTGCTGAGCCCCGACTACCCTGATTTCTCTAGCAGTGGA
GTCAATGCTACAATCCAGAAGGCCTGGAAGAAATACAAGTCCTCTAAGGCCGACGTGTGGAAGGGCGAAATGAGCCTGCCTAGCTACAAGAGCG
ACCAGCCTATCGTGCTGCACGCCAAGCAGATCAAGCTGAGCGGAGATACCAGAGCCGCCGCTGCTACCCTGTCTCTGTTCAGCAACAAGTTCAA
GAAGGAGCACGAGATCAGCGGCAACGTGCAGTTCGCCATCACACTGCACGACAACACCCAGAGAACCATCTACCAGAAGCTGAGAAACGGCGAG
TACAAGCTGTCCGAGAGCCAGCTGGTGTACGACAAGAAGAAGTGGTTCCTGTACCTGGCCTACAGCTTCAACCCTGCCGAACATGCCCTGGACC
CTGAGAAGATCCTGGGCGTGGACATGGGCGAGAAATTCGCCCTGTATGCCAGCAGCTTCGGCGAGTACGGCCATTTCAAGATCGAGGGAAGCGA
GGTGACCGAGTATGCGAAGGCTCTGGAAAGAAGAAGGAGATCTCTGCAACAGCAGGCCCGGTACTGCGGCGAGGGCAGAATCGGCCACGGCACA
AAGACCAGAGTGGGCGTGGTGTACAGAGAGGAAGATAGAATCGCCAACTTCAGATCCACCATTAACCACAGATACAGCAAGGCCCTGATCGAGT
ACGCCGTGAAGAACGGATATGGCACCATTCAGATGGAAAACCTCACAGGTATCAAGGAGAACCTGCAATTTCCAAGACGGCTGCAGCACTGGAC
CTACTACGACCTGCAGAGCAAGATCGAAGCCAAAGCCAAAGAACACGGCATCGCCGTCGTGAAGGTGAACCCCAAGCACACCAGCCAGCGCTGC
AGCAGATGCGGACACATCGCTGCCGAGAACCGGCCTAAACAGGAGGTGTTCCAATGTGTTAAGTGCGGCTACGCTTGTAATGCCGATTTTAACG
CCAGCCAAAATATCTCCATCAAGGACATCGAAAAGCTGATCCAGGAGACAATTGGCGCCAATCCTAAG
SEQ ID NO: 45, Cb5Cas12f1 (ME-B.18) codon-optimized coding sequence
ATGGCCAAGAAGGGTAATTCTCAGAAGAAGCAGATCGTGAAAGTTATGAAATACGAGCTGAAGTATGAAAAGGGATGTGCCGACTTCAACGAGA
TGCAAAACGAGCTGTGGAAGCTGCAAAGACAGACCAGAGAAGTGATGAATAGAACCATCCAGCTGTGCTATCACTGGAGCTACGTGCAGGCAGA
GTACTGCAAACAACACGGCTGTGCCAGACGGGACGTCAAGCCTTGCGACGTGTACGAAACCAACGCCACCTCTCTGGACGGCTACATCTACCAG
CTGCTGAAGGTGGAATACCCAGATTTCTTCATGAAGAACCTGAATGCCACACTGAGAAAGGCTCACCAGAAGTACGACGCCCTGCTGTTTGACA
TTCAGGAGGGCAACTCTAGCATCCCCAGTTTCAAGAAGGACCAGCCTCTGATCTTTGAGAAGAAAGCCATCTGCATCAGCAAGTGTCTGCCTGA
CAAGCGGCAGATTACCCTGTCCTGCTTCAGCGACAGCTACATCGACGCCCACCCAACCCTGGATAAGATCACCTTCACCGTGCGGGCCAGAAGC
GCCAGCGAGAAAAGCATCTTCGACCACATCATCTCCGGCAAGTACGCCCTGGGCACAAGCCAGCTCGTGTACGAGAAGAAGAAGTGGTTCTTCC
TGCTGAGCTACAAGTTCACACCTGAGAGCGTGGACGTGAACCCCGAGAAGGTGCTGGGCGTGGACCTGGGAGTGGTGAACGCCCTCTGCGCCGG
CAGCGTGGAAAACCCTCATGATTCTCTGTTCATCAAGGGCACAGAGGCTATCGAGCAGATCCGGCGGCTGGAAGCTAGAAAGCGGGACCTGCAG
AAGCAAGCCAGATACCCCGGCGACGGCAGAATCGGCCACGGCACCAAGACCAGGGTGAGCCCTGTGTATCAGACACGGGATGCCATCGCCAGAA
TGCAGGACACCCTGAACCACAGATGGTCCAGAGCCCTGATTGACTTCGCCTGCAAGAAAGGCTACGGCACAATCCAGATGGAAGATCTGAGCGG
CATCAAGGCCATGGAATCTGAGAAGCCTTACCTGAAGCACTGGACCTACTTCGATCTGCAGAGCAAGATCATCTACAAGGCCGAAGAAAAGGGC
ATCAGAGTGGTCAAGGTGAATCCCAAGTGTACCAGCAGACGGTGCAGCGCTTGTGGATATATCTCCAAAGAGAACCGCAAGAACCAGGCTGAGT
TCCTGTGCGTCAACTGCGGCTACCACCACAACGCCGATTACAACGCCGCTCAGAACCTGTCTATCCCTCAGATCGATAGACTGATCGAGAAGCA
GCTTAAGGAACAGGAGAGCGAGGAATCCGAGGCCGGAACAAACCCTAAG
SEQ ID NO: 46, Ob1Cas12f1 (ME-B.15) codon-optimized coding sequence
ATGGCCGAAAAGACCATCGTGAAAGTGATGAAATTTGAGCTGAGATACATCGACGGTGCTGGCGAGTTCTCCGAGATGCAGAAACATCTGTGGG
AGCTTCAGAAGCAGACCAGAGAAGTGCTGAACAAAACCATTCAGATGGGCTACGCCCTCGAATGCAAGCGGTTTGCCCACCACGACAAGACAGG
ACAGTGGCTGGATGACAAAGAGCTGACCGGATCCAAGTACAAGGCTGTCGCTGACTATATCAACGCTGAACTGAAGGAAGATTACAACATCTTC
TACAGCGACTGTAGAAACAGCACAGTGCGGAAGGCCTACAAGAAGTTCAAGGACGCCAAGAACAAGATCTTCAGCGGCGAGATGAGCCTGCCTT
CTTATAGAAGCAACCAGCCAATCATCATCCACAACAGAAATGTTATCATCAGAGGCAACGCCGAGAGCGCCCTGGTGGGCCTGAAGGTGTTCAG
CGACGGATTTAAGGCCCTGCACGGCTTCCCTGCCGCCGTCAACTTTAAGCTGTGCGTGAAGGACGGCACCCAGCGGGCCATCATCGAGAACGTG
ATCAGCGAGATCTACAAGATCAGCGAGTCTCAGCTGATCTATGATAACAAGAAATGGTTCCTGATCCTGGCCTACAGATTCACCCAGAAGAAGA
ACGACCTGAATCCCGACAAGATCCTGGGAGTTGATCTGGGCGTGAAGTTCGCCGTGTACGCCAGCAGCATCGGCGAATACGGCAGCTTCAGAAT
TAAGGGAGGCGAAGTGACCGAGTTCATCAAGAGACTGGAGAAAAGAAAGAAGTCCCTGCAGAATCAGGCCACAGTGTGTGGAGACGGCCGCATC
GGCCACGGCACTAAAACACGGGTGGCCGATGTGTACAAGGCCAGAGACAAGATCAGCAATTTCCAGGACACCATCAACCACAGATACTCTAGAG
CTATCGTGGACTACGCCAGAAAGAACGGCTACGGCACCATCCAACTGGAAAAGCTCGATAATAGCATTGAGAAGAAAGGCGATTACAGCCCTGT
GCTGGTGCACTGGACCTACTACGACCTGAGGACAAAGATGGAATACAAGGCAGCCGAGTACGGCATCAAAGTGATCGCCGTGGAACCCAAGTAC
ACCTCTCAAAGATGCAGCAAGTGTGGCTACATCTCTTCTGAGAACAGAAAGACCCAGGAGAGCTTCGAGTGCATCAAATGCGGCTACAAGTGCA
ACGCCGACTTCAACGCCTCCCAAAACCTGAGCGTGCGGGACATCGACAGAATCATCGATGAGTACCTGGGCGCCAACCCTGAACTGACA
SEQ ID NO: 47, EsCas12f1 (ME-B.16) codon-optimized coding sequence
ATGGTTTGTAACAAGGTGGTGAAGATCGCTCTGATCTGCGACCAGATCGATAAGGATGGAAAGGACGTGAACTACAATGACATCTACAAGCTGC
TGTGGGACCTTCAGAAACAGACAAGAGAAGCCAAGAACAAGGTCATCAGACTGTGCTGGGAGTGGTCCGGCTACTCTAGCGAGTATTTCAAAAC
CCACGAGGAATATCCTAAGGATAAAGAGATCCTGGGCATCAGCCTGAGATCCTACCTGTACAATAGAATCAAGGGCGACTACAACCTGTACAGC
GGCAACTTGTCTCAATCCGCCAAAATCGCCTACATCGAGTACAAGAACAGCCTCACCGACGTGCTGCGGGGCGATAAAAGCATCATCAACTACA
GGGAGAACCAGCCACTGGACATCAAGAACAAGGCCATACAGCTGCTGTACGAGAACGACAACTTTTTCGTGCGGGTGGCCCTGATCAACAAGGA
CAAGCGGAAGGAGCTGAACTTCAAGGACTGCAGCGTGCGGTTTAAGCTGCTGGTGAAGGATGATAGCACACGGACCATCCTGGAAAGATGCTTC
GACGAGGTGTACACAATCACCGCCAGCAAGATCATGTACAACAAGAAGAAGAAGCAGTGGTACATCAACCTGGGATACAAATTCACCAAGGAAA
TCGACAAGACACTGGATAAGGATAGAATCCTCGGCGTGGACCTGGGCGTGATCAACCCCCTGGTGGCTAGCGTGTACGGCAGCTACGACAGACT
GATCATTGGAGGCGGAGAAATCGACAAGTTCAGAAAGCGGGTCGAGGCCAACAAAGTGCAGATGCTGAAACAGGGCAAGTACTGCGGCGACGGC
AGAATCGGCCACGGCGTGAACACCAGAAACAAACCTGCTTATAACATCGAGGACAAAATTAGCAGATTCCGGGACACCGTGAATCATAAGTATT
CTAAGGCTGTGGTCGACTACGCCGTGAAGAACAACTGCGGTACAATCCAGATGGAAGATCTGAAAGGCATTACACAGAACAAGAATGAGAGATA
CCTGAAGAATTGGACCTACTTCGATCTGCAGACCAAGATCGAGTACAAGGCCAAGGCACTGGGCATCGAAGTGAAGTACAAGAATCCTAAGTAC
ACCAGCCAAAGATGTTCTAAATGCGGCCACATCGCCGAGGAAAACCGCCCCGAGCAGAAAACCTTCAAGTGCGTGAAGTGTGGATTTAAGGTGA
ACGCCGACTACAACGCCAGCCAGAATCTGGCCATCAAGGACATCGACAAGATCATCGAACAGTACTACAACAAGGGC
SEQ ID NO: 48, Pt1Cas12f1 (ME-B.19) codon-optimized coding sequence
ATGAAGTACACCAAGGTGATGAGATACCAGATCATCAAGCCACTGAATGCCGAGTGGGACGAGCTGGGAATGGTGCTGCGGGACATCCAAAAGG
AAACCAGAGCCGCCCTGAACAAGACCATCCAGCTGTGCTGGGAGTACCAGGGCTTTTCCGCCGATTACAAGCAGATCCACGGCCAGTACCCCAA
GCCTAAAGATGTGCTGGGCTACACCTCTATGCACGGCTATGCCTACGACAGGCTGAAGAATGAGTTCAGCAAGATCGCTTCTAGCAACCTGAGC
CAGACGATCAAAAGAGCCGTGGACAAGTGGAACAGCGACCTGAAAGAGATCCTGAGAGGCGATAGAAGCATCCCTAACTTCCGGAAGGACTGCC
CTATCGATATTGTGAAGCAGAGTACCAAGATCCAGAAATGTAATGACGGCTACGTGCTCAGCCTGGGCCTGATCAACCGGGAATATAAGAACGA
GCTGGGAAGAAAGAACGGAGTGTTCGACGTGCTGATCAAAGCTAATGATAAAACCCAGCAAACAATCCTGGAAAGAATCATCAACGGCGACTAC
ACCTACACCGCCTCTCAGATCATTAATCACAAGAACAAGTGGTTCATCAACCTGACATACCAGTTCGAAACCAAGGAGACAGCCCTGGACCCTA
ACAACGTGATGGGCGTGGACCTGGGAATCGTCTACCCTGTGTACATCGCCTTCAACAACAGCCTGCACAGATACCACATCAAGGGCGGCGAGAT
TGAGAGATTCCGCCGGCAGGTGGAAAAGCGGAAGAGAGAACTGCTGAACCAGGGCAAGTACTGCGGCGACGGCAGAAAGGGCCACGGCTACGCC
ACAAGAACAAAGTCCATCGAGAGCATCTCCGACAAGATCGCCAGATTTAGAGATACCTGCAACCATAAGTACAGCCGGTTCATCGTGGATATGG
CCCTGAAGCACAACTGCGGCATTATCCAGATGGAAGATCTCACCGGCATCAGCAAGGAATCTACATTCCTGAAGAACTGGACCTACTACGACCT
GCAGCAGAAGATCGAGTACAAGGCCAGAGAGGCCGGAATCCAGGTTATCAAGATCGAACCCCAGTACACAAGCCAACGGTGCAGCAAATGTGGA
TATATCGACAAGGAAAACAGACAAGAGCAGGCCACCTTCAAGTGTATCGAGTGCGGATTTAAGACCAACGCCGACTACAACGCTGCTAGGAACA
TCGCCATCCCCAATATCGATAAAATCATCAGAAAGACACTGAAGATGCAG
SEQ ID NO: 49, RhgCas12f1 (ME-B.2) codon-optimized coding sequence
ATGGCTACAAAGGTGATGAGATACCAGATTATCAAGCCTATCGATTGCAATTGGGACCTGTTCGGCAAGGTGCTGAGAGACATCCAGTATGATA
CAAGACAGATCATGAACCGGACCATCCAGTACTGCTGGGAGTGGCAGGGCTACAGCAGCGACTACAAGATCGCCAAGGGCGAGTACCCTAAGAC
CCGGGAAACCTTCGGCTACAGCGATATGCGGGGCTACGCCTACGACAAGCTGAAATCAATCTACCAGAGGCTGAACACAGCCAATCTGACCACC
AGCATAACAAGAGCCGTGCAGCGGTGGAAAACCGATACCAAGGACGTGATCAGAGGCGACAAATCCATCGCCTGCTTCAGAGCCGACGTGCCAA
TCGACCTGCACAACAAGAGCATGAACATTGAGAAGAGCGACGACGGCTACATCGTGGCCCTGAGCCTGGCCAGCAACATCTACAAGAAGGAACT
GGACAGAAATTCTGGCCAGTTCAGCGTGCTGATCAATGAGGGCAACAAGTCTAATCGGGACGTCCTGGATAGATGTATCGCTGGACAGTATAAG
ATTAGCGCTTCTCAGATCCTGCGGGAAAAGAACAAGTGGTTCCTGAACCTGTCCTACTCTTTTGAGATCAGCAAGCCCGATAAGTCTAGAGACA
ACATCCTGGGAATCGACGTTGGAATCGTGCACCCCGTGTACATGGCCGTGTACAACAGCCCTGCTAGAAGGAGCATCAGCGGCGGCGAAATCGA
CAACTTCCGCAAGCAGGTGCAGAAAAGAATCAAAGAGCTGCAGCTGCAAGGCAAACAATGTGGCGAGGGCAGAATCGGCCACGGCATCAAGACA
AGAGTGAAACCTATCGAGTTTGCCAAAGACAAGGTCGCCAACTTCCGGAACACCATCAACCACAAGTACTCCAAGGCTATCGTGGAATTCGCCA
TTAAGAACGGCTGTGGAATCATCCAAATGGAAGATCTGAAAGGCATCAACACCGACAACGTGTTCCTCAAGAACTGGACCTACTACGACCTGCA
GCAGAAGGTGAAGTACAAGGCCGAGCTGGAAGGAATCGAGGTGAAGCTGATCGACCCCCAGTACACCAGCCAGCGGTGCTGCAAGTGTGGCTAT
ATCCATAGAGATAACAGACCTGAGCAGGCCAAGTTCAAGTGCATCGACTGCGGCTTCGAGGTGAACGCCGATTACAATGCCAGCCTCAACATCG
CCACCCCTGACATCGACAAGATCATCCTGGAATTTCTGAAGTGCGAGACA
SEQ ID NO: 50, Bc1Cas12f1 (ME-B.10) codon-optimized coding sequence
ATGGGCGTGACAATCAAGATCATGAAATACCAGATCCTGTGCCCTATGAACGTCGACTGGACAATCTTCGAGAAACATCTGAGAAATCTGACCT
ACCAGGTGCGGACCATCAGCAACAGAACCATCCAGCAGCTGTGGGAGTTCGACGCCCTGAGCTTTGATTACTTTAAGGAACGAGGAACATACCC
TACAGTCCAGGACCTGTACGGCTGCACCCAGAAGAAGATCGACGGCTACATCTACCACACACTGCAGAGCAAGTATCCCGACATCCACAAAGGC
AACATGAGCACCACCCTGCAGAAGATCATCAAAACCTGGAAGTCTAGAAGAAATGAGATCAGAAAGGGCGAGATGAGCATCCCTAGCTTCAGAA
ACAGAATTCCCATCGACCTGCACAACAACAGCGTGGACATCATCAAGGAAAAGAACGGCGATTATATCGCCGGCATCTCTCTGTTCAGCAGAGA
TTTCCACAAGGAGAACGGCGACGTGCCAAAGGGCAAGATCTTCGTGAAACTGGGAACCCAGAAACAGAAATCCATGAAAGTGATCCTGGATAGA
CTGATCAATCAGACCTACAGCAAAGGCGCTTGTATGATCCACAAGTACAAGAACAAGTGGTATCTGAGCATCACCTACAAGTTTAACGCTATCA
AGGAAAACAAGTTCGACAAGGAACTGATCATGGGAATCGATATGGGCGGAATCAACACCGTGTACTTCGCCTTTAACGAGGGCTTCATCCGGAG
CAACATCAAGTCCGACGAGATCAAGATGTTCAACGAACGGATCAGACAGAGAAGGATTAATCTGCTTAAGCAGTCTAAATACTGCAGCAACAGC
AGAACAGGCAAGGGCCGGACCAAGCGCCTGCAACCTATCGATGTGCTGTCCAATAAGATCGCCAAGTTCCGGAACTCTACAAACCACAAATACG
CCAATTACATTGTGAAGCAGTGTCTGAAGCACAATTGCGGCAGAATCCAGATGGAACTGCTGAAGGGAATTTCTAAGAACGACAAGGTTCTGAA
GGACTGGACCTACTTCGACCTGCAGGAGAAGATCAAGAACCAGGCCGAGATCTACGGCATCGAAGTGATCAAGGTGGTGCCTGCCTACACCAGC
CAGCGGTGTAGCCAATGTGGCTACATCTGCAAGGAGAACAGATGCACACAGGCCATGTTCGAGTGCAAGCAGTGCGGCTACAAGACCCACGCCG
ATTATAACGCCGCTAAGAACATCTCCACCTACGACATCGAGAACATCATCAACAAGCAACTGGCCGTGCAGAGCAAGCTGCACAGCAAGAAGTG
CATGGAAGAGTACATCGAGGAACTGGGCTACCTGGAC
SEQ ID NO: 51, BfCas12f1 (ME-B.8) codon-optimized coding sequence
ATGTCTACCGTGGTCAAAGTGATGAAGTACCAGATCATTTGTCCTGTGAACATCGAGTGGAAGGCTTTTGAGACATATCTGCGGACCCTTTCTT
ATCAGGTTAGAACAATCGGCAACCGCACTATTCAGAAGCTGTGGGACTTCGATAACCAGAGCCTGAATCACTTTCGCGAGAATGGCGTGTACCC
CAGCGCTCAGCAGCTCTACGGTTGCACCCAGAAAACCATCAGTGGATATATCTACGACCAGCTGAAAGAGGAGTATCAGGACATGAACAAGGCG
AACATGTCGACCACCCTGCAGAAAACCATCAAGACCTGGAACTCGCGTAAGAAGGAAATCCGCAGCGGCGAGATGTCTATTCCTAGCTTCCGGA
ACAACCTCCCCATCGACATCCATGGCAATTCCATCCAGATCACCAAGGAGAAGTCCGGGGACTACATTGCCTCCCTGTCTCTCTTTTCAAGCAA
CTTCATCATCGAAAACAACCTGCCCAACGGCAAGATTCAAGTCAAGCTGTCCACTCGCAAGCAGAACTCCATGAAGGTGATCCTGGACAGAATC
ATCGAGAACACCTACGCGAAGGGTGCCTGCATGCTGCACAAGCACAAAAACAAATGGTACTTGAGCATCATCTACAAGCCGACAGTAAAGGAGG
AACATAAGTTCGAGGAAGATCTGGTGATGGGCATCGACATGGGAAAGATCAACGTGCTGTACTTCGCCTTCAACAAGGGCTGGATCAGAGGCGC
CATCTCCGGGGAGGAGATTGAGGCCTTCAGAAAGAAAATTGAGCACAGGCGGATCTCTCTGCTGAGACAGGGCAAATACTGCAGCGGAAACCGG
GTGGGCAAGGGCAGAGAGAAGCGGATCAAGCCTATCGACGTGCTCAACAACAAGATTGCCAAGTTTCGCAATGCAACCAACCACAAGTACGCCA
ACTACATCGTGCAGCAGTGCCTGAAATACAACTGCGGCACCATCCAGCTGGAAAACCTGCAAGGCATCTCCAAAGAACAGACGTTCCTGAAGAA
CTGGACCTATTTCGACCTGCAGGAGAAAATCAAGCAACAGGCCCACCAGTACGGCATGAAGGTGGTGACAATTGATCCAAGCTACACCAGTAAG
AGGTGTTCTGAATGTGGCTACATCCACAAGAATAACCGCAAGAGCCAGTCCACATTCGAGTGCCAGCAGTGTAATTTGAAAGTGCACGCCGATT
ACAACGCCGCTAAGAACATCAGCATCTACAACATCGAGAAGGTCATCCAGAAGCAACTGAAGCTCCAGGAAAAACTGAACAGCAAGAAGTTCAC
CGAGCAGTACATCGAGCAGGTGGAGAACATCAAT
SEQ ID NO: 52, BtCas12f1 (ME-B.6) codon-optimized coding sequence
ATGTCTATCGCCGTGAAAGTGATGAAATATCAGATCGTTTGTCCTGTGAACATCGAGTGGAAAACCTTCGAGATCTACCTGAGAACCCTGTCTT
ACCACTTCCGGACAATCGGCAATAGAACCATCCAGAAACTGTGGGAGTACGACAACCAAAGCCTGAAGCACTTCAAGGACACCGGCCAGTACCC
CAGCGCCCAGCAGCTGTACGGATGTACCCAGAAAACCATCTCTGGCTACATCTACGACCAGCTGAAAGAGGAATACCAGGACATCAACAAGGCC
AATATGAGCACAACACTGCAGAAAACCATTAAGACCTGGAACAGCAGAAAGAAAGAAATCTGGTCCGGAGAGATGAGCATCCCTAGCTTCAGAA
ACAACCTGCCTATCGACATCCACGGCAACAGCATCCAGATCATCAAGGAAAAGAGCGGCGACTACATCGCTTCTGTGTCTCTGTTCAGCAGCAA
GTTCATCAAGGAAAACGACCTGCCCAACGGCAAGATACTTGTGAAGCTGAGCACAAGAAAGCAAAATAGCATGAAGGTCATCCTGGACAGAATT
ATCGACAGCACCTACGCCAAAGGAGCTTGTATGCTGCACAAACACAAGAAGAAATGGTATCTGTCCATAACATACAAGTCCAACATCAAGGAAG
AGCTGAAGTTCGACGAGGATCTGATCATGGGCATCGATATGGGCAAGATCAACGTCCTGTACTTCGCCTTCAACAAGGGCCTGGTGAGAGGCGC
CATCTCTGGCGAAGAAATCGAAGCCTTTAGGAAGAAGATCGAGCACAGAAGAATCTCTCTGCTGCGGCAGGGCAAGTACTGCAGCGGAAACCGG
ATCGGCAAGGGCAGAAAGAAGCGGATTAAGCCTATCGAGGTGCTGAATGATAAGATCGCTAAATTCAGAACAGCCACCAACCACAAGTACGCCA
ACTACATCGTGCAGCAGTGCCTGAAGTACAACTGCGGCACCATCCAGCTCGAGGACCTGCAAGGCATCAGCAAAGAACAGACCTTCCTGAAGAA
CTGGACCTACTTCGACCTGCAGGAGAAGATCAAGAATCAGGCCAATCAATACGGCATCAAGGTGGTGAAGATCGATCCTAGCTACACCAGCCAG
CGGTGCAGCGAGTGCGGATATATCCACAAGAACAACAGACAGAACCAGAGTACCTTTGAGTGCCAGCAATGCAGCTTTAAGGTGCACGCCGACT
ACAACGCCGCCAAGAACATCTCCGTGTACAACATCGAGAAGGTGATCCAGCGGCAGCTGAAGCTGCAGGAGAAGCTCAACCTGACAAAGTACAA
GGAGCAGTATATCGAGCAGATGGAAAATATTAAC
SEQ ID NO: 53, HsCas12f1 (ME-B.12) codon-optimized coding sequence
ATGAGAGCCCTGGAAAACCAGAAGCCTCTGAAGTCTATCAAGAAACCTGTGTGTAAAATCAGCAGAACCCTGTCTGTGCCCATTCAGCGGCCTT
GTGGCTACGTGTGGAACGATTTCGGCCACCTGCTGTGCATCATCAGAAACGACGTGGCCCAGGCCTATAATATGGCCATGTCTGAGAGCTACCT
GTACTTCTCCGAGAGAGAGAATTACAAGCGGGAACACGGCAAATATCCTAAGGTGGAACAACTGGCTAAGCGCAACGTGTACAAGAAGTTGACA
GAGAACTTCCCCCACATCGGCACCGGCATCCTGGCCACCATCGCCAACAAGGTGGAATCTAAACTGAAGAAAGAGTACGTGGAAGTGATGCTGA
AGGGCACAAAGTCCGTGTCCAACTACAAGAAGGGTACACCTATACCAATCCGGGCTCAGGGCTGGAAGGAACGGACCTTTAAGAGAAAGAGAAA
GGACAAGATGACCTTCCACCTGCTGAGCAAGAAGGCCGAGCAGAGCAAGAGCCTGGATTTCCTGAAGGACGAGAAGGGCAAGATCCCCTGCAGC
TTCACCGTGCGGATCGCTCTGAAGAAGCTGAACAACTCCCAGAGAGCCGTGTACAACAGAATCTGGGCCGGCGAGTACAAGGCTGGCGCTATCG
ACATCCTGCAGCGGAAGGGCAAGTGGTTCATCAACATCTCTTATCACATGCTGGAGACAAAGCGGCTGGAAAAGCAGCTGGACAAGAATGTGAT
CGTGGGAGTGGACCTGGGAATCGTCAACGGCGTGGTGTGCGCCGTCAGCAACGACGCCTACGACAGACTCGTGCTGCGGAAGGACATCGAGGGC
TTTAGAAAGCAGATCTGGAAAAGAAAGCACCTGGCCTGGAAGAGCACCAGGCGGGGCGGAAAGGGAAGAAAGTACTACCTGAGGATGAGCGACA
GCCTCAAGAAGAAAGAGCACAACTTCAGAAATACCCTGTACCACGACTGGACCAGAAAAGTGATCGACTACGCCCTGAAGCACGGCGCCAAAGT
TATCCAGATCGAAGATCTGAGCGGCCTGGTGGAAGCCAAGAAGAAAATGAAGAAAGGCGTGCTGAAGAACTGGGTGATCAGCGACTTCGTCGAG
AAGCTGACATACAAAGCTGAGGAATACGGCATTGAGATCGTGAAGGTGAACCCCAGATACACCAGCCAGCGTTGCCATAAGTGTGGACATATCG
AGAAGGACAACCGGAAAGAGCAAAGCAAGTTCGTGTGCCTGAAGTGCGGCCACAGCTGCAACGCCGATTTTAACGCCGCCAAGAATATCGCCAC
AAAGAATATCGCCGACATCATCTCCGCCAGCCTGCCTCAGACC
SEQ ID NO: 54, MsCas12f1 (ME-B.13) codon-optimized coding sequence
ATGACCGACGAGCAGGCCAGACTGCAGAAAGTGGCCACATTTCAGATCGTCAAGCCTGTGAACATGGACTGGCGGGAGTTCAGAAAGCTGCTGA
GAGATGTGCGGTACAGACTCTGGAGATTAGGCAATATGGCCGTGTCTGAGGCTTATCTGACCTTTCACAAGAAGTACAGAATGGGCCAGGCCCA
GAGCGATGGCGCCCACAAGCTGAGCGTCCTGGACAAGCGCCTGCGGCAGGCTCTGATCGACGAGGGCGTTAGAGTGGAAGAACTGAGCAGATAC
AGCCGGAAGGGCGCCGTGTCCGGCTACATCTGTGGCGCCTTCGAGAAAACCAAGCTGAGCGCCATCAAGTCCAAGAGCAAGTGGCGGGACATCA
TCAATGGCAGAGCATCTCTGCCTGTGTTCAGACGGGACCTGGCCATCCCCATCAACTGCAGCGACTGCCAGCCTAGAATGATCGAGAGAACAGA
GGCCGGCGAGTACCAGGTGGACCTGAGGATTTGTCTGCAAGATAAGGAGCTGGCCCCTAACGGCTACCCCAGGGTGCTGCTGTCTACAGCTAAG
ATCAGCGACGGCCAGAGAGCCGTGCTGGAGAGACTCGTCAGCAACAAGACCAACAGCCTGCCTGGATACAGGCACAGATTCTTCGAGATCAAGG
AGAAGCGGGGCAAGTGGTTCCTGTCTGTGTCCTACGACTTCCCTAGAGCTGAAGCCGGCAAACTGCACCAGGACATCATCGTGGGCGTGGACCT
GGGCTGGTCCGTGCCACTGTACGCCGCTCTGAACAAGGGCTACGCCAGAATCGGCTGGAAGAAGCTGGAACCACTGGCCAAGCGGATTCGGCAC
CTGCAAAAGCAGGTGAAGGGACGGAGACTGAGCATGCAGCGGGGAGGCCAGGCCGACCTGGCTGGTCCTACCGCCCGGATGGGACACGGCAGAA
GAAGAAACCTGCAGGCCATTGAGAAGCTGGAAGGAAAGATCAACGACGCCTACACCACCCTGAATCACCAACTGAGCCACTGCATCATCGAGTT
CGCCAAGAACAACGGCGCTGGCGTGATCCAGATCGAAGATCTGAGAGGCCTGGCTGATGAGCTGCGGGCGACCTTCATTGGACAGAACTGGCGA
TACCACCAGCTGCAGGAGTTTATCAAATATAAGGCCGAGGAAGCCGGCATCAAGGTGGTGCCCCCCGTGAATCCTTTCTACACCAGCAGACGTT
GCAGCGTGTGCGGCTACCTGCATAAGGACTTCACCTTCGAGTACAGACAGGTTAACCGGAAGAACGGCATGAGCGTGATGTTCGAGTGCCCCGA
GTGCAGCAAGAAAGCCAAGGAAGAGGGAAAAGAGTACAAGGCCCTTAATGCCGATTACAACGCCGCCAGAAACCTGGCCACAGCCAACATCGAG
GAAAAGATCAGACTGCAATGTAAAGAACAGGGCATCGAATATACAGAACTGCCTAAGTCT
SEQ ID NO: 55, ScCas12f1 (ME-B.11) codon-optimized coding sequence
ATGAAGGACTACATCAGAAAGACCCTGTCCCTGAGAATCCTGCGGCCCTACTACGGCGAGGAAATCGAGAAAGAGATCGCCGCCGCCAAGAAGA
AGTCCCAGGCCGAGGGGGGAGACGGCGCTCTGGACAACAAGTTCTGGGACCGGCTGAAGGCCGAGCACCCTGAGATCATCTCTAGCAGAGAGTT
CTACGACCTCCTGGATGCCATTCAGCGGGAGACAACCCTGTATTACAATAGAGCCATCAGCAAGCTCTACCACAGCCTGATCGTGGAAAGAGAG
CAGGTGTCTACCGCCAAGGCCCTGAGCGCTGGCCCTTACCACGAGTTCAGAGAAAAGTTCAACGCTTATATCAGCCTGGGCCTTAGAGAGAAGA
TCCAGAGCAACTTCAGAAGAAAGGAACTGGCCAGATACCAGGTGGCCCTGCCTACAGCCAAGAGCGACACCTTCCCTATCCCCATCTACAAGGG
CTTCGACAAGAACGGAAAAGGCGGCTTTAAGGTTAGAGAAATCGAGAACGGCGACTTTGTGATCGACCTGCCCCTGATGGCCTACCACAGAGTG
GGCGGAAAGGCTGGCAGAGAGTACATCGAACTGGACCGGCCTCCAGCCGTGCTGAACGTGCCTGTGATCCTGTCTACAAGCCGGAGAAGGGCAA
ATAAAACCTGGTTCCGCGACGAGGGTACAGACGCCGAAATCCGGCGGGTGATGGCCGGAGAGTACAAGGTGTCCTGGGTGGAAATTCTGCAAAG
AAAGAGATTCGGCAAACCTTACGGCGGATGGTACGTGAACTTCACCATCAAGTACCAGCCTAGAGATTATGGCCTGGACCCCAAGGTCAAGGGC
GGCATCGACATCGGCCTGAGCAGCCCTCTGGTCTGCGCCGTGACAAACAGCCTGGCCAGACTGACCATCAGAGATAACGACCTCGTGGCCTTCA
ACCGGAAGGCTATGGCTCGCCGCAGAACCCTGCTGAGACAGAATAGATACAAGAGATCTGGCCACGGCAGCGCCAACAAACTGAAGCCCATCGA
GGCCCTGACAGAGAAGAACGAGCTGTACAGAAAGGCCATCATGCGGCGGTGGGCCAGAGAAGCCGCTGATTTCTTCCGGCAGCACCGCGCCGCT
ACCGTGAACATGGAAGATCTGACCGGCATCAAGGACAGAGAGGACTACTTTAGCCAGATGCTGCGGTGCTACTGGAACTACAGCCAGCTGCAGA
CCATGCTGGAAAACAAGCTGAAAGAGTACGGAATCGCCGTGAAGTACATTGAGCCTAAGGATACATCTAAGACCTGTCACTCCTGCGGCCATGT
GAATGAGTACTTCGACTTCAATTACCGGAGCGCCCACAAGTTTCCAATGTTCAAGTGTGAAAAGTGCGGCGTTGAGTGCGGCGCCGATTACAAC
GCCGCTAGAAACATCGCCCAGGCC
SEQ ID NO: 56, Un2Cas12f1 (ME-B.20) codon-optimized coding sequence
ATGGAAGTGCAGAAAACAGTGATGAAAACCCTGAGCCTGAGAATCCTGCGGCCTCTGTACAGCCAAGAGATCGAGAAGGAAATCAAGGAGGAAA
AGGAAAGAAGAAAGCAGGCCGGCGGCACCGGCGAACTGGACGGAGGCTTTTACAAGAAGCTGGAAAAGAAGCATTCTGAGATGTTCAGCTTCGA
CAGACTGAACCTGCTGCTGAACCAGCTGCAGAGAGAGATCGCCAAGGTGTACAACCACGCCATCAGCGAGCTGTACATTGCCACCATCGCCCAG
GGCAACAAGAGCAACAAGCACTACATCTCTAGCATCGTGTACAATAGAGCCTACGGCTACTTCTACAACGCCTATATCGCTCTGGGCATCTGCA
GCAAGGTGGAAGCCAACTTCAGATCCAACGAGCTGCTCACCCAGCAGTCTGCCCTGCCCACCGCCAAAAGCGACAACTTCCCCATCGTCCTGCA
CAAGCAGAAGGGCGCTGAGGGCGAGGATGGCGGCTTCCGGATCAGCACCGAAGGATCTGATCTGATCTTCGAGATCCCTATCCCCTTCTACGAG
TACAACGGCGAGAACCGGAAGGAACCTTATAAGTGGGTGAAGAAGGGGGGACAAAAACCTGTGCTGAAGCTGATCCTGTCTACATTCCGGAGAC
AGAGAAACAAGGGCTGGGCCAAGGACGAGGGCACTGATGCCGAGATCCGGAAGGTTACAGAGGGCAAGTACCAGGTGTCTCAAATCGAGATCAA
CCGCGGCAAAAAACTGGGCGAGCACCAGAAGTGGTTCGCTAATTTTAGCATCGAGCAGCCTATCTACGAGAGAAAGCCCAATAGAAGCATCGTG
GGCGGCCTGGATGTGGGCATTAGAAGCCCTCTGGTTTGTGCCATCAACAACAGCTTTTCCAGATATAGCGTGGACAGCAATGACGTGTTCAAGT
TCTCCAAGCAGGTGTTCGCCTTCAGAAGGCGGCTGCTGTCCAAAAACAGCCTGAAACGTAAGGGCCACGGCGCTGCCCACAAGCTGGAACCCAT
CACCGAGATGACCGAAAAAAACGACAAGTTCCGGAAGAAGATCATCGAAAGATGGGCCAAGGAAGTGACCAACTTCTTCGTGAAGAACCAGGTC
GGAATAGTGCAGATCGAGGACCTGAGCACCATGAAGGACCGGGAAGATCACTTCTTCAACCAGTACCTGCGGGGCTTTTGGCCTTACTACCAGA
TGCAGACACTGATCGAGAACAAACTCAAAGAGTACGGAATCGAGGTGAAGCGGGTGCAAGCTAAATACACAAGCCAGCTGTGCAGCAATCCTAA
TTGCAGATACTGGAACAACTACTTTAATTTCGAGTACAGGAAAGTGAATAAGTTTCCAAAGTTCAAGTGTGAAAAGTGCAACCTGGAAATCAGC
GCCGACTATAACGCCGCTAGAAACCTGTCTACCCCAGACATCGAGAAGTTCGTGGCCAAGGCCACAAAGGGAATCAACCTGCCTGAGAAG
SEQ ID NO: 57, CiCas12f1 (ME-B.7) codon-optimized coding sequence
ATGAAAACCACCGAGAAGAACGTGCTGATGACCAAGTGCATCAAGGTTACACTGAACAGGTGTGTGAACTACAATATGAAAGAGATCATGAACA
TTATCCGGGAAATGCAGTACCTCAGCAGCAAGGCCTACAACCTGGCCACAAACTACCTGTACATCTGGGACACCAATTCTATGAACTTCAAGAA
CCTCTACGAGGAAAAGATCGTTGATAAAGACCTGCTGGGCAAGAGCAAGTCCGCCTGGATCGAGAACCGGATGAATGAAATCATGAAGGGCTTC
CTGACCAACAACGTGGCCCAGGCCAGACAGGACGTGATCAACAAGTATAACAAAAGCAAGAAAGATGGCCTGTTTATCGGCAAGGTCACCCTTC
CAAGCTACAAGATGAACGGCAAGGTGGTCATCCACAACAAGGCTTATAGATTCAGCAAGAATGAGGGCTACTTCGTGGAAATCGGCCTGTTCAA
CAAGGAAAAGAAGGAAGAGCTGAACTGCGACTGGATCAAGTTTAAGCTGGACAAAATCGACAGCAACAAGAAGGCCACCATCTACAAGATCCTG
AATGGCGATTACAAGCAGGGCAGCGCCCAGCTGCACATCAACAAGAAAGGAAAGATCGAGTTCATTATTTCTTATAGCTTCGAGCGGGAAAACT
CCATCAAGCTGGATAAGAACAGAACACTGGGAATCGACATCGGCATCGTGAACATCGCCGCCATGGCCATCTGGGATAACAATAAACAGGAGTG
GGAGCTGACCAGATACAGCCACAATCTGATCAGCGGCAACGAGGCCATCGCTCTGCGGCAGAAGTACTACAAACTGGGACTGAGAAACAAGGAA
CTGGAAAAGAACATCAACCGGGAACTGCACGAGCTCGAAGAGAAGGAATACAGAGGCCTGAGCACAAACATTATCTCTGGCCATAATCTGACCT
ACAAGCGTATCATGCTGAACAGCAAGAGAATCAGACTGTCTCAGTCCTGCAAGTGGTGCGGCAACAGCAAAGTGGGCCACGGTAGAAGAGTGCG
GTGCAAGCAAGTGGACAAGATAGGAAACAAGATCGAGAGATTCAAAGACACCTTCAACCACAAGTACAGCCGGTACATCGTGGACTTCGCCGTG
AAGAACAATTGCGGAATTATCCAGATGGAAAACCTGAAGAATTTCAACCCCAGCGAGAAGTTCCTGAAGGACTGGCCTTACTTTGACCTGCAGA
CAAAGATCGAATACAAGGCCAAGGAGTACGGCATCGAGGTGATCAAAGTGAATCCTAAGTACACCAGCAAGCGCTGCAGCAGATGTGGCTGTAT
CAACGAGCTGAACAGAGATTGCAAGAAGAACCAGTCTAAATTCAAGTGCGTGAACGACGAGTGCAACAACTATGAGAACGCTGACATCAACGCC
GCTAAGAACATCGCCCTGCCTTACATCGACAAGATCATCGAGCAATGTCTGGAGACAAACAAGGTGGTG
SEQ ID NO: 58, CpCas12f1 (ME-B.9) codon-optimized coding sequence
ATGAAGCTGAATAAATGCATCAAGGTGACCCTGGTCAAGTGCCTGAACTACGACTACAAAGAAATCAAACAGATCATCAGAGACTTCAACTACA
CCGCCTGCAAGGCCAGCAACAAAGCTATGAGAATGTGGTTCTTCCACACACAGGACATGATCGATAAGAAGAACAAGTACAAGGAATTTAACCA
GATCCAGTACGAGAAAGACACATACGGAAAGTCTTACAGAAACGTGATCGAGGGAGAGATGAAGAAAATCATGCCACTGGCCAATACCAGCAAC
GTTGGCACACTGCACCAGCAGCTCGTGCAGAACGACTGGTCCAGACTGAAGAAGGACATCCTGTCCTGTAAAGCCAACCTGCCCACCTACAAGC
TGTCCACCCCTTACTTCATCAAGAATGACAATTTTAAGCTGCGGAACCACAACGGCTACTTCGTGGACATTGCCTTCTTCAACAAGGAGGGCCT
GAAGCAGTACGGCTACAAGGCCGGCCATAAGTTCGAGTTTCAGATCGACAAACTGGACGGTAACAAGAAGTCCACCATCAACAAGATCATCAAC
GGCGAGTACAAGCAAGGCAGCGCCCAGCTGAGCATCAGCAATAAGGGCAAAATCGAACTGATCATCAGCTACAGCTTCGAAAAGGAGGAAGTGC
CCGTGCTGGACAAGAACAAAATCCTGGGAATCGACCTGGGCATCACCAACGTGGCCACAATGAGCGTGTACGACAGCATGCGGGAACAGTACGA
CTACTTCAGCTGGAAAACCAACGTGATCTCTGGCAAGGAGCTGATCGCTTTTAGACAGAAATACTACAACCTGAGGAGAGATATGAGCATCGCC
TCTAAAACAGCCGGACAGGGCAGATGCGGCCACGGCTACAAGACCAAGATGAAGAGCGTGAACAAGGTGCGGAATAAGATTGCAAACTTCGCCG
ATACATATAACCACAAGATTTCTAAGTACATCATCGAGTTCGCCATCAAGAACAACTGCGGCGTGATCCAAGTGGAAGATCTGAGCGGCGCCAC
CGCCGACACCCACAACAAGATGCTGAAGGATTGGTCTTATTACGACCTGCAGCAGAAGATCGAATACAAGGCTAAGGAGCAAGGCATCGAGGTG
ATCAAGGTCAACCCTAAGTATACCAGCAAGCGGTGCAGCAAATGTGGATGTATCCACGAGGATAATAGAGATTGCAGAAACAACCAGGCCAAGT
TCGAGTGCAAGGTGTGTGGCTACAATGAGAACGCTGACATCAACGCCAGCAAGAACATCGCCATTCCTGACATCGACAATATCATCAAGGGCAC
TGAGATCCTGCACAGCAAAGAAAACAAGGCTTCT
SEQ ID NO: 59, SvCas12f1 (ME-B.17) codon-optimized coding sequence
ATGACAACCAAGTGCGTGCAGGTCGCCATCGAGTACAGCAGTAACAACATCCTGAAGGAGGTGGACTTCTACAAGGAACTGCGGGACCTGCAAT
ACAACAGCTACCTGGCCTGCAACCGGGCCATCAGCTACATGTACGAGAACGACATGCAGAACTTCATCATAAAGGAAACTGATCTTCCTAGAAG
CGACGACAAGAAGCTGTACGGCAAGTCCTTCGCCGCTTGGATCGAGAACAGAATGAACGAGTACATGCCAGGCGCTCTGAGCAACAATGTGGCC
CAGACAAGACAGTTCGTGGTGAACAGATACAAGAATGACAAGAAAGCTGGCCTGCTGAAAGGCAACGTGTCCCTGACCACCTTCAAGCGGACCA
ATCCTATCATCATCCACAACAATGCCTACAACATCATCGAAACCCCTAAGGGCCTGGGAGCTGAGATCGGCTTTTTCAATCTGCCTAAGCAGAA
AGAGCTGGGAATCAAAAGAGTTAATTTTCTGTTCCCCAAGCTGGGCAGCAGCGAGAAGAGCATCATCCGGCGGCTGCTGGACAAATCTTATAAG
CAGGGCGCCATGCAGATCTCTTACAACCAGAAGAAGAAGAAATGGATGGCCACCATCAGCTTCAGCTTTAACCTGGAAGAAATCAAGACCAACG
AGAATCTCGTGATGGGCATCGACCTGGGCGTGTCTAAAGTGGCCACACTGAGCATCTACGACGCCAGCAAGTATGAGTATATTAAGATGAGCTT
TAAGGATACATGCATCGACGGCACCGAGCTGATGCACTACAGGCAGAAACTGGAAAGCAGACGCAAGGCCCTGAGCATCGCCAGCAAGTGGGCC
TCTGATAACAACCGTGGACACGGCTACAAGACCAAGATGGAGAAAGCCAACTACATGGGAAGAAAGTACAACAACTTCAGAGACACCTACAACC
ATAAGGTGTCTAGATACATCGTGGACGTGGCCATCAAGTATAGAGTGGGCCTGATCCAGATGGAAGATCTGTCCGGCTTCAGCGAGCAGCAGCA
GGAGAGCCTGCTGAAGAACTGGTCCTACTACGATCTGCAGCAAAAGATCAAGTACAAGGCAGAAGAGAACGGCATCAGAGTGTACTTCATCAAC
CCCAAGTACACATCACAGCGGTGCAGCAAGTGCGGCAACATCGATAAAGAAAATAGAAAGACCCAGGAGAGCTTCTCCTGTACAGTGTGTAATT
ACAAGGACAACGCCGACGTGAACGCCTCTAAGAACATTGCCATTCCTGACATCGAGAAGATCATCGAGGAACAGGTCAAGAAACAGTAC
SEQ ID NO: 60, AoCas12f1 (ME-A.7) codon-optimized coding sequence
ATGATCACAACCAGAAAGCTGAAGCTGGCAATCGTGTCCGACAACAAGAACGAGGCCTACAGCTTCATCCGGGACGAGACAAGAAACCAGAACA
GAGCTCTCAACGTGGCCTACAGCCACCTGTACTTCGAGTACATCGCCCAGGAGAAGCTGAAGCATTCTGATGCCGAGTACCAGGAGCACCTGGC
CAAGTACCAGGAGCTGGCCTCTAAGAAGTACCAGGAGTTCCTGAAGGTCAAAGAGAAAGCTAAATCTGACGAGACACTGCAGGCCAAGGTGGAC
AAAGCCAGAGAAGCCTATAACAAGGCCCAGGAGAAGGTGTACAAAATCGAGAAGGACTACAGCAAGAAGGCTCGAGAAATCTACCAACAGAGCG
TGGGCCTGGCCAAACAGACAAGAATCGATAAGCTGCTGAAGAATCAATTTAACCTGCACTACGACACCGTGGACAGAGTGGGCGGAACAGCCAT
AAGCCACTTCACCAACGACATGAAGAGCGGCGTGCTGCAAGGCAAAAGAAGCCTGCGGAATTACAAGAGCAGCAACCCTCTGATGATCAGAGCC
AGATCTATGAAGGTGTACGAGGAAAATAGCGATTACTACATCAAATGGATCAAGGACATCACATTTAAGATCATCATCAGCGCCGGCAGCAAGC
AGAGACAGAACATTGGAGAGCTGAAGTCCGTCCTCGTGAACATCATCGAGGGAAATTACAAAGCTTGTGATAGCAGCATCGGCGTGGATAAGGA
TCTGATCCTGAACCTGAGCATCGACATCCCCATCACCAAGGAAAACATCTTCATCCCTAACCGGGTGGTGGGAGTTGACCTGGGCCTGAAGATC
CCAGCCTACGTGTCCGTTAATGATACCCCTTACATCAAGCGGGCCATCGGCAATATAAACGACTTCCTGAAGGTGCGGACCCAGCTGCAATCTC
AGCGGCGCCGGCTGCAGAAGGCCCTGCAGAGCACCAACGGCGGCAAGGGTAGAAACAAGAAGATGCAGGGCCTGGAAAGACTGCAAGCTAAGGA
AAAGAACTTCGTGAACACCTACAACCACTTCCTGAGCAAGAACATCGTGGATTTTGCCGTGAAAAACAACGCTGGCATGATCCACATGGAAGAA
CTGAAGTTCGACAAGGTGAAGCACAAGTCTCTGCTGAGAAATTGGTCCTATTACCAGCTGCAGACCATGATCGAGTATAAGGCCAAGAGAGAGG
GCATCGAGGTGTATTACGTGGACGCCAGTTACACCTCCCAGACCTGCAGCAAGTGCGGCAACCTGGAAGAAGGCCAGAGGGAGACACAGGACAC
CTTCGTGTGCAAGAAGTGCGGCTACAGCGTGAACGCCGACTATAACGCCAGCCAGAATATTGCCAAGGCCAAAACCATCAAGGAGGAAAACCAG
CAG
SEQ ID NO: 61, Bc2Cas12f1 (ME-A.5) codon-optimized coding sequence
ATGATTCTGACCAGAAAGATCAAGCTGGTGATCGTGTCCGAGAACCGGGAGGAAGGCTACAACCTGATCAGAACAGAGATCCGGGAACAGCACA
AGGCACTGAACCTGGCTTATAACCACCTGTACTTCGAGCACAACGCCATCCAGAAACTGAAGCAGAATGATGAGGATTACAAGCAGAAAAGAAA
CAAGCTGCAGGAGCTGATCAACAAGAAATACGAGGAACATCAGAAGGCTAAGAATCTCGAGAAGAAGGAAGCCCTGAGAGAGGCCTATAACAAC
AAGAAGCAGGAGCTGTACAACTTCGAGAAGGAATACAATGAGAAGGCTAGACAGACATACCAGCAAGTGGTGGGCTTTACCCAACAGACCAGAG
TGCGGAACCTGATCAATAGAGAGTGCAACCTGATGAGCGACACCAAGGACGGCATCACCAGCAAGGTGACCCAGGACTATAAGAACGACTGTAA
AGCTGGACTGCTGATCGGCAAGCGGAGCCTGCGGAACTACAAGAAGGACAACCCCCTGCTTGTGCGTGGAAGAAGCCTTAAGTTCTACAAGGAA
GATGGAGATTACTTCATCAAGTGGAACAAGGGGACCATCTTCAAGTGCATCCTGCACATCAGAAAGAAGAACGTGGTGGAACTGCAGAGCGTGC
TGGAAAACGTGCTGCTGGGCGCCTACAAGGTGTGCGACTCTAGCATCGGATTTAACAACAAGGATATGATTCTGAATCTGAGCCTGAACATCCC
AGATAAGGAGACACAGGGCTACATCCCTGGCCGGGTGGTCGGCGTGGACCTGGGCCTGAAGATCCCTGCCTACCTGAGCCTGAGCGATAAGGTG
TACGTCCGGAAGGGCATCGGCAGCATCGACGACTTCCTGAGAGTTCGGACCCAGATGCAGAAGAGACGGCGGAGACTGCAGAAGTCTCTGGCCG
CTGTGAAGGGCGGCAAAGGCAGAGAAAAGAAGCTGAAAGCCCTGGACCACCTGAAGGGAAAGGAGGCCAACTTTGCCAAGACATATAATCACTT
CCTCTCCACCCAGATCGTGACATTCGCCGTGAAGAACCAGGCCGGCCAGATCAACATGGAATTCCTGGAATTCGACAAGATGAAGAACAAAAGC
CTGCTGAGGAACTGGTCCTACTACCAGCTGCAAATCATGGTCGAGTACAAGGCCAAGAGAGAGGGCATTATCATCAAGTACGTGGACGCCTACC
TGACCTCTCAGACCTGCAGCAAGTGTGATCACTACGAGGACGGCCAAAGAGAGAAACAAGAGAATTTCATGTGCAAGAATTGCGGCCTGGAAGT
GAACGCCGACTACAATGCCAGCCAGAACATCGCCAAGAGCACCAGCTACATCAGCGACTCTACAGAGTCCGAGTACCACAAGAAGAAACAGCAG
GTGCTGAAGGAAATCCTGGGCGAGAACGACATCATGAACGAACAGCTGTCTCTGTTCAACAATTGTGACGACATCGCC
SEQ ID NO: 62, CdCas12f1 (ME-A.3) codon-optimized coding sequence
ATGATCAGCACAAGAAAGATCAAGGTGAGGTGTGATGACAGCACCTTTTATACATTTTTCAGACAGGAGCAGAGAGAACAGAACAAGGCCCTGA
ACATCGGCATTGGAATCATCCACGCCAACGCCGTTCTGCATAATGTTGACTCTGGCGCCGAGAAGAAGCTGAAGAAATCCATCGAGGGACTGCA
GGGCAAGATCGACAAGCTCAACAAAGACCTGGAGAAAGAGAAGATCACTGATAAGAAGAAGGAAGAGGTGCTGAAGGCCATCGAAACCAACAAG
AAAATCCTGGACGGCGAGAAGAAGGTGTTCAAGGAAAGCGAGGAATACAGAAAGGGCATCGACGAGCTGTTCAAGAACACCTACCTGAAGTCTA
ATACCCTGGATCACGTGCTGGATAGCATGGTGAACATCCAGTACAAGCGGACCCTGTCCCTGGTGACCCAGCGGATCAAGAAAGACTACAGCAA
CGACTTCGTGGGCATCATCACCGGCCAGCAGAGCCTGCGGAACTACAGAAATGACAATCCTCTGATGATCAGCAACCAGCAGCTGAACTTCAAG
TACATCGATGATACATTCTACCTGGACATCATGTGCGGCTACCGGCTGGAAGTGGTGCTGGGGAAGCGGGACAACGAGAATGTGAACGAGCTGA
AGTCTACCCTGGAAAAGGTGATTTCTAAAGAGTATAAGGTGTGCGACAGCAGCATGCAGTTCAGCAAGAACAACAAGGATGTGATCCTGAACCT
GGTCATCGACATCCCACAGAATAGCAACGTGTACAAGCCTGTGGAAGGCAGAATTCTGGGCGTGGACCTCGGAGTGGCCGTGCCTATCTACATG
TGCCTGAACGACGACACCTACAAAAGAAAGGGCCTGGGCGACATCAACAACTTCCTGAGAGTGCGGCAGCAAATGCAAACAAGACGACGGAAAC
TGCAAAAGGACCTGACCCTGACAAACGGCGGAAAAGGCAGAAAGAAGAAAACACAACTGCTGGACAAGCTGCAGGAGAACGAGCGGAACTTCGT
GAAAACCTACAGCCACGCCCTGAGCAAGAGGGTGGTCGAGTTCGCCAAGAGCAACAAATGTGAATATATCAACATCGAGAAGCTGACCAAGGAC
GGCTTCGACAACATCATCCTGAGAAACTGGTCCTACTTCGAGCTGCAGAAAATGATCGAGTACAAGGCTGAGAGAGAGGGCATTACAGTGCGCT
ACGTGAACCCCGCCTACACCAGCCAGAAGTGTTCTAGATGCGGCGAAATCGACAAGGAGAACAGACAGACCCAGGCCAATTTTAAATGCACCAA
GTGCGGATTTGAACTCAACGCTGATCACAACGCCGCTATCAATATCGCCAGAAGCATCGAATTCGTG
SEQ ID NO: 63, Cs1Cas12f1 (ME-A.4) codon-optimized coding sequence
ATGAACACCGTCCGGAAGATCAAGCTGACCATCCTGGGCGATACCGAGACAAGAAACAAACAGTACAAGTGGATCAAGGATGAGCAGTATAACC
AGTACCGCGCCCTGAACCTGAGCATGACCTACATGGTCACCAACCTGATGCTGAAGAACAACGAGAGCGGCCTGGAAAATAGAAAGGAGAAAGA
CATCTTGAAGATCGAGAACAAGATCAAGAAGGACGAGGGCTCCCTGAAGAAAGAGCTGGCCAAGAAGAAGATCAATGAGGAGAAGATCGAAAAC
ATCAAGTCCAACATCGAAGAACTTAAGAGCGAGAAGGAGAAGCTGGAGAATGAGCTGAAGAATATTAAAGAGTACAGAAGCAACATCGATGAGG
AGTTCAAGAAGATGTACGTGGACGACCTGTACAACGTGCTGAACAAGATCAGCTTTCAGCACGAGGACATGAAATCTCTGGTGACCCAGCGGGT
CAAGAAGGACTTCAACAATGACGTGAAGGAAATCATGAGAGGAGATAGATCCGTGAGAAATTATAAGCGGAACTTCCCCATCCTGACAAGAGGC
CGGGACCTGAAGTTTCAGTACATCGAGAAGAGCGAAGATATTGAGATCAAGTGGATCGAGGGCATCAAGTTCAAGTGCATCCTGGGAAAACCTA
GCAAGTCTCTGGAACTGAAGCACGCCCTGCATAAGGTGATCAACAAGGAATACAAGGTGTGTGATTCCAGCCTGCAATTTGACAAGAACAACAA
CCTGATCCTGAACCTGACCCTAGACATCCCTCAGGATAACAAGTATGAGAAAATAACAAACAGAGTGGTTGGCGTGGACCTGGGCCTGAAGATC
CCAGCCTACGTGGCCCTGAACGACACAAAGTACATTAGGAAGGCCATCGGAAGCATCGACGATTTCCTGAAGGTGCGGACCCAGATGCAGAGCA
GAGTGCGCAAGCTGCAGAAAAGCCTGCAAGTGGTGCGGGGCGGCAAGGGCAGAAATAAGAAAATGAAAGCCCTGGAACGGTTCCGGGAAAAGGA
GAGGAATTTCGCCAGAAACTACAACCACTTCCTGAGCTACAACATCGTGAAGTTCGCCCTCGACAACAAGGCTGAGCAGATCAACCTGGAACTG
CTGGAAATGAAGAAGACCCAGAACAAAAGCATTCTGAGAAACTGGAGCTATTACCAGCTGCAAAACTTCATCGAGTACAAGGCCGAGAGAGTGG
GCATCAAAGTGAAATACATCGACCCTTACCACACATCTCAGACCTGTAGCGAGTGCGGCAACTACGAAGAAGGGCAGAGAGTGGAACAGGACAC
ATTCGTGTGCAAGCGGTGCTGGCACAAGATGAACGCCGACTACAACGCTGCTAGAAATATCGCCATGAGCTACAATTACATCTCTAAGAAGGAG
GAATCTGAGTACTACAAGAACAACAAGAACATGGTG
SEQ ID NO: 64, Cb3Cas12f1 (ME-A.10) codon-optimized coding sequence
ATGAACACCGTGCGGAAGATCAAGATCATCATCAACAACGAGAACAACGAGCTGAGAAAAGAACAGTATAAGTTCATCCGGGACAGTCAGTACG
CCCAGTACCAGGGCCTGAACAGATGCATGGGCTACCTGATGTCCGGCTTCTACGTGAACAACATGGACATCAAGTCCGAGGAGTTCAAGACCTG
GCAGAAGGGCGTGACAAACAGCGCCAACTTCTTCCAGGAGATCAGCTTCGGCAAGGGAATCGACAGCAAATCTTCTATCACCCAAAAGGTGAAG
AAAGACTTCTCCATCGCTCTGAAGAACGGCCTGGCCAAGGGCGAGCGGAACATCAACAACTACAAGAGAATCGCCCCTCTGATGACCAGAGGCA
GAAATCTGAAATTCAAGTACGACGACAATGAGCTTGATATCCTGATCAATTGGGTCAACAAGATCCAGTTCAAGTGCGTGCTGGGAGAGCACAA
GAACTCTCTGGAACTGCAACACACCCTGCACAAAGTGATCAACAATGAGTACAAGATCGGCCAGAGCAGCCTGTACTTCAACAAGAAGAACGAG
CTGATCCTGATCCTGACAATCGATATCCCCACCGCCAAAAGCAGCTACGAGCCTATCAAAGATAGAATCCTGGGTGTTGACCTGGGCATGGCCG
TGCCAGTGTACATGAGCATCAACGACAACTCCTATATCAAGAAGAGCCTGGGCAGCTACAGCGAGTTTGCCAAGGTGCGCAAGCAGTTTAAAGA
GAGAAGGAATAGACTGTACAAGCAGCTGGAAGCCTGCAAGGGCGGCAGAGGCAGAAAGGATAAGCTGAAGGCCATGAACCAGTTCAAAGAAAAA
GAGAAAAATTTCGCTAAGACCTACAACCACTTCCTGAGCAAGAACATCGTGGAGTTCGCCCTGAAGAACAAGTGTGAATTTATCCATCTGGAAA
AGATTGAGAGCAAGGGCCTCGAAAACAGCGTGCTGGCTAATTGGACCTACTACGACCTGCAGGAGAAGATCATTTACAAGGCCAAACGGGAAGG
CATTGGAATCAAGTTCGTGAACAGCAGCTATACAAGCCAAACATGCAGCAAGTGCAACTACGTGGACAAGGAGAACAGAAAGACCCAGGCCAAG
TTCATCTGTAAAAACTGCGGATTTAAGGCCAATGCTGATTACAACGCCTCTCAGAACATCAGCAAGTCTAAGGAGTTCATCAAG
SEQ ID NO: 65, Cb4Cas12f1 (ME-A.11) codon-optimized coding sequence
ATGAACATCGTGAAGAAAATCAAGCTGAGAATCATCGATAACGACAAGGAACTGTGCAAGAAGCAGTATCTGGGATTCACCGAGGAACAGAAGA
AAGAACTCATTGATAAGCAGTACAAATTTATCAGAGATTCTCAGTATCAGCAGTATCTGGGCTTCAACAGAGCCATGGGCTTTCTGATGAGCGG
CTACTACGCCAACAACATGGACATCAAATCCGACAACTTCAAGGAACACCAGAAAAAGCTGACCAATAGCCTGTACATCTTCGACGACATCAAG
TTCGGCGTCGGAATCGACAGCAAGTCTCTGATCGTGCAGCGGGTGAAGAAAGACTTCAGCACAGCCCTGAAGAACGGCCTCGCCAAAGGCGAAA
GATCTGTGACCAATTACAAGAGAACCTACCCTCTGCTTACAAGACACAGATCCATCAAATTCCTGTACGCCGAGAATGAGCTGGACATCTACCT
GGACTGGGTGAACAAAATCCGGTTCAGATGCGAGCTGGGCAATCATAAGAACAGCCTGGAACTGCAGCACACCCTGCGGAAGGTGATCACCGGC
GAGTACAAGATAAGCGACTCCTCCCTCGAGTTCAACAAGAAGAATGAGCTGATTCTGAACCTGAACCTGAACATCCCCGAGACAAAGGCTACAT
TCATCAAGGATCGGACCCTGGGCGTGGACCTGGGAATGGCCATCCCTGCCTACGTGAGCCTGTCTGACACCCCATACATCAGAAAGGGCTTTGG
CAGCTACGAGGAGTTCGCCAAGGTCAGAAACCAGTTTAAGGACAGGCGGAAGCGGCTGCTGAAACAACTGTCTCTGGTTGCTGGCGGCAAGGGC
AGAGCCAAGAAGCTGCACAGCATGGAGTTCCTGAAGAACAAGGAAAAGCAGTTCGCCAAGACCTACAACCACAGCCTGAGCAAGAAAATCATCG
ACTTCGCTCTGAAAAACAACTGTGAATACATCAACCTGGAGGACATCAAGTCTACAAGCCTGGAGGACAGAGTGCTGGGACAGTGGGGCTACTA
CCAGCTGCAAGAGCAGATCGAGTACAAGGCCAAGCTGGTGGGCATCAAGGTGCGGAAAGTGAAGGCCGCTTACACCAGCCAGACATGCAGCGAG
TGTGGCAACATCGACAAGGAGAACCGCAAGAATCAAAGCACCTTCAAGTGCACCAACGAGGATTGCAAACTGAACAAGAAAGGAATCAATGCAG
ATTGGAACGCCAGCATCAACATCGCCAGAAGCAAGGAGTTCATCAAG
SEQ ID NO: 66, BsCas12f1 (ME-A.12) codon-optimized coding sequence
ATGATCACCGTGCGGAAGGTCAAGCTGATTGTGAACTCTGAGGAGGCCGAGGAGATCAACAGAACATACAAGTTCATCAGAGATTCTATGTACG
CTCAGTACCAGGGCCTGAATAGATGTATGGGCTACCTGCTGTCTGGCTACTACGCCAACGGCATGGACATCAAGAGCGACGGCTTTAAGAACCA
CATGAAAACCATCAAGAACAGCCTGAACATCTTCGACGACATCAATTTCGGCATTGGAATCGACAGCAAGTCCGCCATTACACAGAAGGTGAAG
AAGGACTTCAGCACCAGCCTGAAGAATGGTCTTGCAAAGGGCGAGCGGGGCGCCACCAACTACAAAAGAAACTTTCCACTGATGACCAGAGGAA
GAGATGTGAAAATTAGCTACCTGGAAGATACAAACACCTTTGTGATCAAGTGGGTCAACAAGATCGAGTTTAAGGTGATCCTGGGCCAGAAAGA
TAACATCGAGCTGAGCCACACCCTGCACAAGATCATCAACAAGGAATACACCCTGGGACAATGTACCTTCGAGTTCGACAAGAACAACAAGCTG
CTCCTGGCCCTGAACATCAACATCCCCGATAATCTGATCAGCAAGAACAAGGAAATCATCCCTGGCAGAGTGCTGGGCGTGGACCTGGGCGTTA
AGGTGCCTGCCATGATCTGCCTGAACGACAATACCTTCATCAAGAAATCTATCGGCTCCTACAACGAGTTCTTCAAGGTGCGGAGCCAGTTCAA
GGCCAGACGGGAACGGCTGTACAAACAGCTAGAAAGCAGCAACGGCGGAAAGGGCCGGAAGCACAAACTGAAGGCCACAATGCAGTTTAGAGAC
AAAGAAAAGAACTTCGCCAGGACCTACAACCATTTCCTGAGCAAAAATATCATCGAGTTCGCTCAGAAGTATACCTGCGAGACAATCAACCTGG
AAGAACTGAACAAAAAGGGCTTCGATAACAACCTGCTCGGCAAGTGGGGCTACTACCAGCTGCAGAGCATGATCGAGTACAAGGCTGAGAGAGT
GGGCATCAAAGTGAAATACGTGGACCCTGCTTTCACATCCCAAACATGCAGCAAGTGCGGCTATGTGGACGAGGAAAACCGCATCACCCAGGAC
AAGTTCGAGTGCCAGAAGTGCGGCTTCACCCTGAACGCCGACCACAACGCCGCCATCAATATCGCCAGAAAG
SEQ ID NO: 67, Pt2Cas12f1 (ME-A.9) codon-optimized coding sequence
ATGATTGCCGTGAAGAAGCTGAAACTGACCATCGTGGAAGAAGAGGAGAAACGGAAGGAGCAGTACAAGTTTATCAGAGACAGCCAGTACGCCC
AGTACCAGGGCCTGAATCTGGCCATGGGCATCCTGACATCTGCTTATCTGGTGTCCGGCCGCGACATCAAGTCCGACCTGTTCAAAGACTCTCA
GAAGAGCCTGACCAACAGCAACGAGATCTTCAACGGCATCAACTTCGGAAAGGGAATCGATACCAAGAGCAGCATCACCCAGAAAGTGAAAAAG
GATTTCAGCACTAGCCTGAAGAACGGACTGGCCAAAGGCGAGAGAGGCTTTACCAACTACAAGCGGGACTTCCCACTGATGACCAGAGGAAGAG
ATCTGAAATTCTACGAGGAAGATAAGGAGTTCTACATCAAGTGGGTCAACAAGATCGTGTTCAAGATCCTGATCGGCAGAAAGGACAAGAACAA
GGTCGAGCTCATCCACACCCTGAACAAGGTGCTGAACAAGGAGTACAAGGTGAGCCAAAGCAGCCTGCAATTTGACAAGAATAACAAGCTGATC
CTGAACCTGACAATCGACATCCCTTACAAAAAGGTTGATGAGATCGTGAAGGACAGAGTGTGCGGCGTGGACATGGGCATCGCTATCCCCATCT
ACGTGGCCCTGAACGACGTGAGCTACGTGCGGGAAGGCATGGGCACCATCGATGAGTTCATGAAGCAGAGACTGCAGTTCCAGAGCAGACGGCG
GAGACTGCAACAGCAGCTGAAGAACGTGAACGGCGGCAAGGGCAGGAAGGACAAGCTGAAGGGCCTGGAATCTCTGAGAGAGAAGGAAAAATCT
TGGGTGAAAACCTACAATCACGCCCTGAGCAAGAGAGTIGTGGAGTTCGCCAAGAAAAACAAATGTGAATACATCCACCTGGAAAAGCTGACAA
AGGACGGCTTCGGCGACCGGCTGCTGCGGAATTGGTCCTACTACGAGCTGCAGGAGATGATCAAGTACAAGGCCGACAGAGTGGGTATCAAGGT
GAAGCACGTGAATCCTGCCTATACAAGCCAGACCTGTAGCGAGTGCGGCCATGCCGATAAGGAAAACAGAGAGACACAGGCCAAGTTTAAGTGC
CTGGAATGCGGCTTCGAGGCCAATGCCGACTACAACGCCGCTAGAAACATCGCTAAATCTGATAAGTTCGTGAAG
SEQ ID NO: 68, CrCas12f1 (ME-A.8) codon-optimized coding sequence
ATGATCGCCGTGCGGAAACTGAAAATCATGGTGCTGTGCGACGACGAGAGCAAGAAGAACGAGCAGTATAAGTTCCTGAGAGATAGCCAGTACG
CCCAGTACCTGGGCCTGAACCGGGCCATGAGCTTTCTGGCTAAGGAATACCTGTCTGGCGACAAAGAAAGATTCAAAGAGGCCAAGAAGAAGCT
GACCAACACATGCGAGTGCTACCAGAACATCAACTTCGGCACCGGCATCGACTCCAAGAGCCAGATCACCCAGAAAGTGAAGAAGGACCTTCAA
GCTGACATCAAGAATGGCCTGGCCAGAGGAGAGCGGAGCATCAGAAATTACCGGAGAACATTCCCACTGATTACAAGAGGCAGAGATCTGAAGT
TCAGCTACAACGGCGACGAGATCATCATTAAGTGGGTCAACAAAATCTACTTCAAGGTGCTGATCGGCAGAAAGGACAAGAACTACCTGGAACT
GATGCATACCCTGGAAAAGATCATCAACGGCGAGTACAAGGTGTGCACCAGCAGCATCCAGATCGACAAGAAACTGATCCTGAATCTGACACTC
GAAATCCCTGATAAGGTGAAGAAGGAGTTTCAGGAGAATAGAGTGCTGGGTGTTGACCTGGGCATCAAATTCCCCGCTTATGCTTGCGTGTCTG
ATAACACCTACGTGCGGAGATCTTTTGGCAGCATCGATGAATTCCTGAAGGTGCGCATCCAGTTCGACAAGAGACGGAAGCGGATCCAACAGCA
GCTGCAGAACGTGAAGGGAGGCAAGGGCAGAAAGGATAAGCTGCAGGCCCTGGACAGAATGCGGGACTGCGAGAGGAAGTGGGTGCGGAACTAC
AACCACGCCCTGTCTAAAAGAATCATCGACTTCGCCTTCAGAAACAAGTGCGGCATCATCCACCTGGAGAAGCTGGAAAAGGACGGCTTTAAGA
ACAAGCTGCTGCGGAACTGGTCCTACTACGAGCTGCAAGACATGATTGGATATAAGGCCGAGAGAGAGGGCATCGTGGTGAAGTACGTGGAACC
TGCCTACACCTCCCAGACCTGTAGCAAGTGTGGATACGTGGATAGAGAAAATAGACCTAGCCAGGAGCACTTCCTCTGCAAGGAATGTGGCTTC
GAGATCAATGCCGACCACAACGCCGCCATCAACATCGCCAGAAGCAACAAGGTCATCGTGGACAAG
SEQ ID NO: 69, ChCas12f1 (ME-A.2) codon-optimized coding sequence
ATGATCACAGTGCGGAAGCTGAAGCTGACAATCATCAATGACGACGAAACCAAGCGGAACGAGCAGTACAAGTTTATCAGAGATAGCCAGTACG
CCCAGTACCAGGGCCTGAACCTGGCTATGAGCGTGCTGACAAACGCCTACCTGTCAAGCAACAGAGATATCAAATCCGATCTCTTCAAGGAAAC
ACAGAAGAACCTGAAGAATAGCAGCCACATCTTCGACGACATCACCTTTGGAAAGGGAACAGACAACAAGAGCCTGATCAACCAGAAGGTGAAG
AAGGACTTCAACAGCGCCATCAAGAACGGCCTGGCTAGAGGCGAGAGAAACATCACCAACTACAAGAGGACCTTCCCCCTGATGACCAGAGGCA
CCGCCCTGAAGTTCAGCTACAAAGACGACTGCAGCGACGAGATCATCATCAAGTGGGTCAACAAGATCGTGTTCAAGGTGGTGATCGGCAGAAA
GGACAAAAATTACCTGGAACTGATGCACACCCTGAACAAGGTGATCAATGGAGAGTACAAGGTGGGCCAGAGCTCTATCTACTTCGATAAGTCC
AATAAGCTCATCCTGAACCTGACCCTGTACATCCCTGAGAAGAAAGATGATGACGCCATCAACGGCAGAACACTGGGCGTGGACCTGGGCATCA
AGTATCCTGCTTACGTGTGCCTGAATGACGACACCTTCATTAGACAGCATATCGGCGAGAGCCTGGAACTTTCTAAACAGAGAGAGCAGTTCAG
AAACCGGAGAAAGAGGCTGCAGCAACAACTGAAGAACGTGAAGGGCGGCAAGGGCCGCGAGAAAAAGCTGGCCGCCCTGGACAAAGTTGCCGTG
TGTGAACGGAACTTCGTGAAAACCTACAACCACACCATCAGCAAGCGGATCATCGATTTCGCCAAGAAGAACAAGTGTGAGTTCATCAATCTGG
AGCAGCTGACCAAGGATGGATTTGACAACATCATCCTGTCTAATTGGTCCTACTACGAGCTGCAAAACATGATTAAGTATAAGGCCGACCGGGA
AGGCATCAAGGTGCGGTACGTGAACCCAGCCTACACCAGCCAGAAGTGCAGCAAGTGCGGTTATATCGACAAAGAGAACAGACCTACACAGGAG
AAATTCAAGTGCATTAAGTGCGGCTTCGAGCTGAACGCTGATCACAACGCCGCCATCAACATTTCTAGACTGGAAGAG
SEQ ID NO: 70, Cs2Cas12f1 (ME-A.6) codon-optimized coding sequence
ATGATAACAGTGCGGAAGCTCAAACTGACCATCGTGGGCGACGAGCAAACAAGAAAGGAGCAGTACAAGATCATCAGAGATGAGCAGTACCAGC
AGTATAAGGCCCTGAACCTGTGCATGACCCTGCTGAATACCCACAACATCCTGAATAGCTACAACACCGGCAGCGAGAATAAGCTGAACAGCCA
GATTGAGAAGCTGGACAACAAGATCGAGAAGAACAAGATCGAGCTGAAGAAGGGCAACCTGAAGGAAAGCAAGATCGAAAAGCTGAACAAGAGC
ATCCTGGAACTGACCAAGGAAAAGGAAAAGCTGCAACAGGAGTACCTGTCCGCCAGCAAGTACAGAAGCGACATCGACGAGAAGCTGAAAGACA
TGTACATCAAAGACATGTATACAGTGGTGCAGAGCCAAGTGAACTTCAAAAGCAAAGATATGATGAGCCTGGTGACCCAGAGAGCCAAGAAGGA
TTTCAGCAACGCCCTGAAGAACGGCATGGCCCGGGGAGAGCGGAGCCTCATCAATTACAAGAGAGATTTTCCACTGATGACCAGAGGCGAGAGA
TGGCTGAAGTTTAAGTACAATGAGGAATCTGACGACATCTACATCGACTGGCTGCACGACATCAAGTTCAAGGTCATCCTGGGATATAAGAAGA
ACGAGAACTCTATCGAGCTGAGACACACACTGCATAAGGTGATCAACAAAGAGTACAAGATCTGCGACAGCTCCATGCAGTTCGACAGAAACAA
CAACCTGATCCTGAACCTGACCCTGGACATTCCTAACAAAGAGAGCAAGGGCTACGTGGAAGGCAGAACACTGGGCGTGGACCTGGGCATCAAA
TACCCCGCTTACGTGTGCCTGTCTGATGACACCTACAAGCGGAAGTCCATCGGCTGTGCCGAGGACTTCATCAGGGTTCGGGAACAGATCCGGG
GCAGAAGATACCGGCTGCAGAAGCAGCTGAGCATGGTCAAGGGAGGAAAGGGCCGCGATAAGAAACTTAGAGCTCTGGACAGAGTGCGGGAAGC
CGAACGCAACTTCGTGAAAACCTACAACCACATGATCAGCAAGAACATCATCAAGTTCGCCAAGGAGCACAACTGCGAGTACATCCACCTGGAA
AAGCTGACCAAGGACGGCTTTCCTGATATTATCCTGAGCAAGTGGTCCTACTACGAGCTGCAGAACATGATCGAATACAAGAGCGACAGAGAAG
GCATCAAAGTGCGGTACATCGATCCTGCCTACACCAGCCAGACATGCAGCAAGTGCGGCCACATCGACAAGGAAAATAGAATCAACCAGGAGAA
GTTCAAGTGTGTGAAATGTGGCTTCGAGCTGAACGCCGACCACAATGCTTCTATCAACATTTCTAGATCTAATAAATACCTGAAG
SEQ ID NO: 71, PhCas12f1 (ME-A.13) codon-optimized coding sequence
ATGAAAACCACCAGAAAGCTGAAGCTGACCATCATCGGCGACGAGGAAACAAGAAAGGAGCAGTACAAGATCATCAGAGAGGAACAGTACCAGC
AGTACAAGGCCCTGAACCTGTGCATGACCCTGCTGAACACACACAACATCCTGAACTCTTACAACACCGGCGCCGAGAACAAGCTGAATGCCCA
GATCGATTCTATCGACAAGAAGATCGAACAGGCCAAAAAAGAGCTGGAAAAAAAGGGCCTGAAAGAAAGCAAGGTGTCTAAGCTCAAGGAGACA
ATCGAGTTCCTGGAAAACGATAGAGAGAAGCTCAAAGACGAGTACCTGAACTCCAGCAAGTTTAGAAGCGACATTGATGAAAAGATGAAAGAGA
TGTACATCAAGGACATGTACACTGTGGTGCAGAACCAGGTGAACTTCCGGGCAAGAGATATGATGAGCCTGGTGACCCAGAGAGCCCGGAAGGA
CTTCAAGAACAGCCTGAAAAACGGCATGGCCAAGGGCGAGAGATCCCTGACAAACTATAAGCGGGACTTCCCTCTGATGACAAGGGGCGAACGG
TGGCTGAAATTCGAGTACGACAAAGATAGCGACGACATCCTGATCAACTGGATTCACGGTATCAAGTTCAAAGTTCTGCTGGGCTACAAAAAGA
ATGAGAACTCCATCGAGCTGAGACACACCCTGCATAAGGTCATCAACAAGGAGTACAAGATCTGCGACTCTAGCATGCAGTTCGATAGAAACAA
CAATCTGATCCTGAACCTCACCCTGGACATCCCTGACAAGCAGAACAACAACTACATCGAGAAGAGAACACTGGGAGTGGACCTGGGCATTAAG
TACCCAGCTTACGTGTGCCTGAATGACGACACCTACATTCGGAGCCACATCGGCGAGAGCCTGGAACTGCTGAAGCAGCGGGAACAATTTAAGG
ACAGGCGGAAGCGGCTGCAGCAGCAACTGAAGAACGTGAAGGGCGGAAAGGGCAGAAACAAGAAGCTGAGCGCCCTGAACAAACTGTCTGATAA
TGAGAGAAATTTCGCCAGAACCTACAACCACATGATCAGCAAGCGGATCGTGGAGTTCGCCAAGAAGCACAGATGTGAGTTCATCAACCTGGAA
AAGCTGACCAAGGACGGCTTCGACAACAATATCCTGAGCAACTGGAGCTACTACGAGCTGCAAAATATGATCGAGTATAAGGCCAAGCGCGAAG
GCATCGAGGTGCGGTACATCGACCCCGCCTACACCAGCCAGAAGTGCAGCAGATGCGGATATATCGACAAGGAGAACAGACAGACCCAGGAGAA
ATTCAAGTGTCTGAAGTGCGAATTTGAGATCAATGCTGATCACAACGCCGCCATTAACATCGCCAGAGCTTTGGAT
SEQ ID NO: 72, OpbCas12f1 codon-optimized coding sequence
ATGAGCGAGCAGGAGGCCGCTCAGGAGGGCACAAAGCTGCTGGCCAAAACCCTGACCTTCGGCCTGGGCAACCCTATGGGCTTCAAGTCCAAGG
GCTCCGTGCTGGTTGAGCTGACAGAGGACCAGCGCAAGGCCATCTACAACGGCCTGAGAGATGCCTCTACCGTGGTGGCCAGAATCATCAACCT
GCTGAACAGCAGGGAGTACATCCGGCAGATCATGAAAGTGCCTGAGGAACTGGTGGCACAGTTCAAGCCTAACTACAGCCTGGTGAAGGGACCT
CTTAAACGGCTGGGAATTGAGGAAGCCGAGCAGGTGGCCGGCAGCGTGTTGAGCCAGACCTTTGCCCTGGGAGTGAAACCTGACTTCCAGGGCG
AGCACGGCAAGGGCCTGCTGCTGAAGGGTGAAAGACAGATCCCCCTGCATAGAACCGACGGCACCCACCCTATCCCACAGCGAGCTACAGAGAC
AAGGCTGTTCCAAGTGGAAAAGAACTTCTACGTGGCCATGCAGGTTTTCGCCGAAACCTGGGCCAAGAAGCAGGAGCTGCCCAGCGGCTGGCTC
GCCTTCCCCATCAAGGTGAAGCCCAGAGATAAGACTATGGCCGGACAGCTGTTAAAGACAATCGGCGGCGAATGGAAGCTGAAGAATAGCCGGC
TGATGCGGAATCCTAGAACAGGAGGAAATAGATGGCTGGGCCAGATCGTGGTGGCTTTTGCCCCTGAGCCTTTCAAGAAGATGACCCGGTCTGT
CGTCATGGGAATCGACCTGGGCGTGAACGTGCCCGCCTGCCTGCACATTTCTGAAAACGGCAAACCTCTGCCTTGGGCCATGATGGTCGGCAGA
GGCAGAGACATGCTGAATACCAGAAACCTGATCAGATCTGAAATCGTGCACATCATCAAGGCCCTGAAAAGCAAGGACAGCCCACTGGACGGCA
AGGCCAGAGCCATCTATCGGGATAAGCTGCGGGACCTGAGAAAGAGAGAACGGAGAGTGATGAAGATGGCCAGCCAAACAGTGGCCGCCAGAAT
CGCCGATACCGCCAAGCGGCACGGCGCCGGCACCTGGCAGATGGAAGATCTGAGCCCTGACATCAAGACCGATCAGCCTTGGCTCGCCAGAAAC
TGGGCTCCCGGCATGCTGCTCGACGCCGTGCGGTGGCAGGCCAGACAATGTGGCGCCGAGCTGGTGATGGTGAACCCTGCCTACACCAGCCAGA
GATGCGCCAGATGCGGCCACATCGACCCTCAGAACCGGCCCAAGCAAACCGACTTTAAGTGCATGGCCTGTGGGCACGAGGACAACGCCGACAA
GAACGCTGCTAGAAACCTGTCCGTGGTGGGCATCGAGAAGCTGATCGCCGACTTCAAGGCTCCAAACGGCGCTGTGCAG

| | | SEQ | | SEQ | | SEQ | Direct | SEQ |
|---|---|---|---|---|---|---|---|---|
| | | ID | | ID | Repeat | ID | repeat | ID |
| Cas | Scaffold sequence | NO | tracrRNA sequence | NO | sequence | NC | sequence | NO |
| OsCas1 | AGGGACGACTTCCCGTCCCAAAATCG | 73 | AGGGACGACTTCCCGTCCCAAAATCG | 111 | GCGGCTTGAA | 145 | GTTGCAA | 179 |
| 2f1 | AGACAGTAGCCGTAAAACTTTGAGTT | | AGACAGTAGCCGTAAAACTTTGAGTT | | GG | | CCCGCGT | |
| (ME-B. | TCAGAGTGGGCGACACACTCGAAAAG | | TCAGAGTGGGCGACACACTCGAAAAG | | | | ATGGGCG | |
| 3) | GTTAAGATATGCACATAGTAATCCGT | | GTTAAGATATGCACATAGTAATCCGT | | | | CGGCTTG | |
| | GCATGAGCCGCGAAAGCGGCTTGAAG | | GCATGAGCCGC | | | | AAGG | |
| | G | | | | | | | |
| RhCas | ACGGTTGATTTAGCAACCGAAGTCTG | 74 | ACGGTTGATTTAGCAACCGAAGTCTG | 112 | TGGAATGTAA | 146 | GTTAAAA | 180 |
| 12f1 | AGGGCATGTAGAAAAAAGTATAGGTA | | AGGGCATGTAGAAAAAAGTATAGGTA | | AT | | GCTAACT | |
| (ME-A | TATACCAACATACTTGCATTGCCACT | | TATACCAACATACTTGCATTGCCACT | | | | ATAGTGG | |
| 1) | CGGAAAGGGTTAACCTTGGTCATTGT | | CGGAAAGGGTTAACCTTGGTCATTGT | | | | AATGTAA | |
| | GTTACCGACCAAGCATTCCAGAAATG | | GTTACCGACCAAGCATTCCA | | | | AT | |
| | GAATGTAAAT | | | | | | | |
| Ob2Ca | CTGGGACTTCACCCAAAATCGAGACA | 75 | CTGGGACTTCACCCAAAATCGAGACA | 113 | GTGGCTTGAA | 147 | GATGCAT | 181 |
| s12f1 | GTGGCCGTCAGCCTTCCCATCGGGAA | | GTGGCCGTCAGCCTTCCCATCGGGAA | | GG | | CTCACGC | |
| (ME-B. | GCGGGCAATACACTCGAAAAGGTTAA | | GCGGGCAATACACTCGAAAAGGTTAA | | | | GTGTCCG | |
| 4) | GATGCACATAGTAATCCGTGCATGAG | | GATGCACATAGTAATCCGTGCATGAG | | | | TGGCTTG | |
| | CCACGAAAGTGGCTTGAAGG | | CCAC | | | | AAGG | |
| Ob3Ca | AAGGGACGACTTCCCGTCCCAAAATC | 76 | AAGGGACGACTTCCCGTCCCAAAATC | 114 | TGTGACTTGA | 148 | GTTGCAA | 182 |
| s12f1 | GAGATAGTGGTCCTGATTCTTTGATT | | GAGATAGTGGTCCTGATTCTTTGATT | | AGG | | CCCGCTC | |
| (ME-B. | TCAAAGCGGACAATACACTCGATAAG | | TCAAAGCGGACAATACACTCGATAAG | | | | GCTGGTG | |
| 5) | GTTAAGATGCACATAGGAATCCGTGC | | GTTAAGATGCACATAGGAATCCGTGC | | | | TGACTTG | |
| | ATGGGTCACAGAAATGTGACTTGAAG | | ATGGGTCACA | | | | AAGG | |
| | G | | | | | | | |
| Cb1Ca | GATTCAGGGGCGACTTCCCGCCCTGA | 77 | GATTCAGGGGCGACTTCCCGCCCTGA | 115 | GGTGCGGCTT | 149 | GTTGCAA | 183 |
| s12f1 | AATCGAGAAAGTGGTCGTAAGCCGGA | | AATCGAGAAAGTGGTCGTAAGCCGGA | | GAAGG | | CACGCGC | |
| (ME-B. | AGCATTTCCGCAGACAATACACTCGA | | AGCATTTCCGCAGACAATACACTCGA | | | | GAAGGTG | |
| 14) | AAAGGTTAAGATATGCACATAGTAAT | | AAAGGTTAAGATATGCACATAGTAAT | | | | CGGCTTG | |
| | CCGTGCATGGGTCGCATTGAAAGGTG | | CCGTGCATGGGTCGCATT | | | | AAGG | |
| | CGGCTTGAAGG | | | | | | | |
| Cb2Ca | AATTTAGGACTTCCCTGAAATCGAGA | 78 | AATTTAGGACTTCCCTGAAATCGAGA | 116 | ATGACTTGAA | 150 | GTTGCAA | 184 |
| s12f1 | AAGTGGCCGTAAGACGCAGTTCCTTG | | AAGTGGCCGTAAGACGCAGTTCCTTG | | GG | | CACGCGC | |
| (ME-B. | CGCCGGCAATACACTCGAAAAGGTTA | | CGCCGGCAATACACTCGAAAAGGTTA | | | | GTAAGGA | |
| 1) | AGATGCACATAGTAATCCGTGCATGG | | AGATGCACATAGTAATCCGTGCATGG | | | | TGACTTG | |
| | GTCATGAAAATGACTTGAAGG | | GTCAT | | | | AAGG | |
| Cb5Ca | AATCGAGATAGCAGCCATTTGAAGAC | 79 | AATCGAGATAGCAGCCATTTGAAGAC | 117 | ACACTGGTTG | 151 | GTTGCAA | 185 |
| s12f1 | GGTCTTGCACTCGAAAAGGTCAAGAT | | GGTCTTGCACTCGAAAAGGTCAAGAT | | AAGG | | CTCGCAC | |
| (ME-B. | GCACACAATAATCCGTGCATGGTCAG | | GCACACAATAATCCGTGCATGGTCAG | | | | GTTGGCA | |
| 18) | TGTGAAAACACTGGTTGAAGG | | TGT | | | | CTGGTTG | |
| | | | | | | | AAGG | |
| Ob1Ca | TATTTAGGGCGACTTCACGTCCTCAA | 80 | TATTTAGGGCGACTTCACGTCCTCAA | 118 | GTGACTTGAA | 152 | GTTGCAA | 186 |
| s12f1 | ATCGAGAAAGTGAGCGTAAGACTTGG | | ATCGAGAAAGTGAGCGTAAGACTTGG | | GG | | TTTGTAT | |
| (ME-B. | CTTCTGTCAAGCGGTTAATACACTCG | | CTTCTGTCAAGCGGTTAATACACTCG | | | | ACGAGTG | |
| 15) | AGAAGGTTAATATGCACATAGTAATC | | AGAAGGTTAATATGCACATAGTAATC | | | | TGACTTG | |
| | CGTGCATGAGTCACTGAAAGTGACTT | | CGTGCATGAGTCACT | | | | AAGG | |
| | GAAGG | | | | | | | |
| EsCas1 | CCGGGCGGCTTGGCGTCCGTAAATCG | 81 | CCGGGCGGCTTGGCGTCCGTAAATCG | 119 | GGGTATAGTG | 153 | GTTGCAA | 187 |
| 2f1 | AGAAAGTACATTGTTAATATAGTGGA | | AGAAAGTACATTGTTAATATAGTGGA | | CAAG | | TCTACAT | |
| (ME-B. | TACACTCGATAAGGTTAACGCATACG | | TACACTCGATAAGGTTAACGCATACG | | | | GCACGGG | |
| 16) | ATATTAATCCCGTATGCCGTCTATAT | | ATATTAATCCCGTATGCCGTCTATAT | | | | TATAGTG | |
| | TTGAAAGGGTATAGTGCAAG | | TT | | | | CAAG | |
| Pt1Cas | AGTCGAGAAGTGCCGTAATAAGCATC | 82 | AGTCGAGAAGTGCCGTAATAAGCATC | 120 | ATGTATGATG | 154 | GTTTCTG | 188 |
| 12f1 | TAAAAATGCCTAACGGTAACACTCGA | | TAAAAATGCCTAACGGTAACACTCGA | | TGA | | AAGAAAC | |
| (ME-B. | TAAGGTAGTCCTGCTAGGCAGGCTGA | | TAAGGTAGTCCTGCTAGGCAGGCTGA | | | | TATGTAT | |
| 19) | AACCCTAGCCACAAAATCCGGCTAGG | | AACCCTAGCCACAAAATCCGGCTAGG | | | | GATGTGA | |
| | CATCATACGAAAATGTATGATGTGA | | CATCATAC | | | | AG | |
| RhgCa | TAAATCGAGAAGTGGCATAAATCCAT | 83 | TAAATCGAGAAGTGGCATAAATCCAT | 121 | AAGATGTGAG | 155 | ATTGCAA | 189 |
| s12f1 | ACTTGTGTGGTTGCAAAACACTCGAT | | ACTTGTGTGGTTGCAAAACACTCGAT | | | | CTGGTGC | |
| (ME-B. | AAGGTAAAAACGGTTAGCACCGTTTG | | AAGGTAAAAACGGTTAGCACCGTTTG | | | | ATGTGTA | |
| 2) | AAATTCCGAGTATAAAAGACCGCTCG | | AAATTCCGAGTATAAAAGACCGCTCG | | | | CAAGATG | |
| | GACGTCTTGAAAAAGATGTGAG | | GACGTCTT | | | | TGAG | |
| Bc1Cas | AAATGGAGAAGTAGCACATAAGAAAT | 84 | AAATGGAGAAGTAGCACATAAGAAAT | 122 | GATGTATTTA | 156 | GTTTAAC | 190 |
| 12f1 | TTACCAAGTGCCAACACTCCGTAAGG | | TTACCAAGTGCCAACACTCCGTAAGG | | AAT | | ACTAACA | |
| (ME-B. | TAGTATCAAATGTAAATAAACATTGA | | TAGTATCAAATGTAAATAAACATTGA | | | | TAAGATG | |
| 10) | TGCGTGGGCACTTTCATGCTCTGAAG | | TGCGTGGGCACTTTCATGCTCTGAAG | | | | TATTTAA | |
| | GGTGTAACACAAAAACCGTTACACAA | | GGTGTAACACAAAAACCGTTACACAA | | | | AT | |
| | TACATCGAAAGATGTATTTAAAT | | TACATC | | | | | |
| BfCas1 | TAAATGGAGAAGTGACACACGGTAAA | 85 | TAAATGGAGAAGTGACACACGGTAAA | 123 | GATGTATTGA | 157 | AGTTTAA | 191 |
| 2f1 | TGTACCAAGTGTAAACGCTCCATAAG | | TGTACCAAGTGTAAACGCTCCATAAG | | AAT | | ACCAAAC | |
| (ME-B. | GTAGTATCGAATGTTTAAAAACATTG | | GTAGTATCGAATGTTTAAAAACATTG | | | | AATAGAT | |
| 8) | ATACGTAGGCGTTATGAATGCCTTGA | | ATACGTAGGCGTTATGAATGCCTTGA | | | | GTATTGA | |
| | AGGGTGTAACACAAAGACCGTTGCAC | | AGGGTGTAACACAAAGACCGTTGCAC | | | | AAT | |
| | AATACATGAAAGATGTATTGAAAT | | AATACAT | | | | | |
| BtCas1 | AAATGGAGAAGTGATACACGGTAAAT | 86 | AAATGGAGAAGTGATACACGGTAAAT | 124 | GATGTATTGA | 158 | GTTAAAA | 192 |
| 2f1 | TTACCAAGTGTCAACGCACCGTAAGG | | TTACCAAGTGTCAACGCACCGTAAGG | | AAT | | CATAACA | |
| (ME-B. | TAGTATCGAATGTTAGAAAACATTGA | | TAGTATCGAATGTTAGAAAACATTGA | | | | ATAGATG | |
| 6) | TACATAGGCATTCTGAATGCTTTGAA | | TACATAGGCATTCTGAATGCTTTGAA | | | | TATTGAA | |
| | GGATGTAACACAAAGACCGTTACATA | | GGATGTAACACAAAGACCGTTACATA | | | | AT | |
| | ATACATTGAAAGATGTATTGAAAT | | ATACATT | | | | | |
| HsCas1 | GTGGTGGTTCGCGCAATGGGGCGAGT | 87 | GTGGTGGTTCGCGCAATGGGGCGAGT | 125 | GTGTGGGTTG | 159 | GTCACAC | 193 |
| 2f1 | TCACGTCCTTATGTTGAGAAGTGCCT | | TCACGTCCTTATGTTGAGAAGTGCCT | | AAAC | | CCTGTGC | |
| (ME-B. | GTAATTCAATGAATTATCATTGTTTG | | GTAATTCAATGAATTATCATTGTTTG | | | | GGGTGTG | |
| 12) | TGTAACGCTCAATAAGCCTGCACACA | | TGTAACGCTCAATAAGCCTGCACACA | | | | TGGGTTG | |
| | ATACCGCACGAAAGTGTGGGTTGAAA | | ATACCGCAC | | | | AAAC | |
| | C | | | | | | | |
| MsCas | GTTCTTTGAAATAAAGATATAGCTGC | 88 | GTTCTTTGAAATAAAGATATAGCTGC | 126 | ACTTCGAGG | 160 | GGTGTAG | 194 |
| 12f1 | CGGTAAAACGATAGCCCACGGGCAAT | | CGGTAAAACGATAGCCCACGGGCAAT | | | | GCGACCT | |
| (ME-B. | TGCGTGCGGCAGTTTAGGCCGACTCG | | TGCGTGCGGCAGTTTAGGCCGACTCG | | | | TTTTTTG | |
| 13) | AACGGCCTGAAGGTTGAGGTAAAGAC | | AACGGCCTGAAGGTTGAGGTAAAG | | | | CGGTGTA | |
| | TTCGAGG | | | | | | CTTCGAG | |
| | | | | | | | G | |
| ScCas1 | ATAAATTTCGTCCCGCGGTGATGGGT | 89 | ATAAATTTCGTCCCGCGGTGATGGGT | 127 | GTGGATTGAA | 161 | GCCGCAT | 195 |
| 2f1 | ATAGCCTTTGGGCAGAGTCCCAAAAT | | ATAGCCTTTGGGCAGAGTCCCAAAAT | | AC | | CTCGCAC | |
| (ME-B. | CGCGGATAAGACGCGGTTGCTTTCAA | | CGCGGATAAGACGCGGTTGCTTTCAA | | | | GCGTACG | |
| 11) | CAGCCGAAAATCGAGCTACGAAAGTG | | CAGCCGAAAATCGAGCTAC | | | | TGGATTG | |
| | GATTGAAAC | | | | | | AAAC | |
| Un2Ca | AATGTTATTCCATAATAACATTTGAT | 90 | AATGTTATTCCATAATAACATTTGAT | 128 | CGAATGAAGG | 162 | GTTGCAG | 196 |
| s12f1 | GCACACGATTCCTCCCTACAGTAGTT | | GCACACGATTCCTCCCTACAGTAGTT | | AATGCAAC | | AACCCGA | |
| (ME-B. | AGGTATAGCCGAAAGGTAGAGACTAA | | AGGTATAGCCGAAAGGTAGAGACTAA | | | | ATAGACG | |
| 20) | ATCTGTAGTTGGAGTGGGCCGCTTGC | | ATCTGTAGTTGGAGTGGGCCGCTTGC | | | | AATGAAG | |
| | ATCGGCCTAAAGTTGAGAAGTGTCAG | | ATCGGCCTAAAGTTGAGAAGTGTCAG | | | | GAATGCA | |
| | ACTCTGATAACCCTCAACGACGATAT | | ACTCTGATAACCCTCAACGACGATAT | | | | AC | |
| | TCTTTATTTCGGAAACGAATGAAGGA | | TCTTTATTTCG | | | | | |
| | ATGCAAC | | | | | | | |
| CiCas1 | AAAGGGTGATTTACCATCCTAAGTAG | 91 | AAAGGGTGATTTACCATCCTAAGTAG | 129 | ATAGGTTATA | 163 | TTAATGA | 197 |
| 2f1 | AGGAAACTCTTACAACCGCTCCAGTT | | AGGAAACTCTTACAACCGCTCCAGTT | | TTTAAAT | | TTAACAT | |
| (ME-B. | GAAATTTGCTGGTAAAAAGCTAGTAT | | GAAATTTGCTGGTAAAAAGCTAGTAT | | | | AGGTTAT | |
| 7) | AAAAAATGCTATAAGCATGGTGGGTA | | AAAAAATGCTATAAGCATGGTGGGTA | | | | ATTTAAA | |
| | CTATGATATAACCAATGAAAATAGGT | | CTATGATATAACCAAT | | | | T | |
| | TATATTTAAAT | | | | | | | |
| CpCas | GTATCTATAACATAAAACTAAGATTA | 92 | GTATCTATAACATAAAACTAAGATTA | 130 | GTTGTATTTA | 164 | TTGTTTA | 198 |
| 12f1 | TTAGTAAGTTTATAGAATAATATCTC | | TTAGTAAGTTTATAGAATAATATCTC | | AAT | | ATATTAA | |
| (ME-B. | GATAATGCTTCAAGTATTAATTCACT | | GATAATGCTTCAAGTATTAATTCACT | | | | CAAAGGT | |
| 9) | TGGTAAAGGTTGTGTAGGGGAGTGGC | | TGGTAAAGGTTGTGTAGGGGAGTGGC | | | | TGTATTT | |
| | TTTAAGTCAGAGTTCTACACCGATAC | | TTTAAGTCAGAGTTCTACACCGATAC | | | | AAAT | |
| | AACTGAAAGTTGTATTTAAAT | | AACT | | | | | |
| SvCas1 | AAGGTGAGAAGTGCTGTAGGAGTCCC | 93 | AAGGTGAGAAGTGCTGTAGGAGTCCC | 131 | GTTGTATTTA | 165 | GTTTAAG | 199 |
| 2f1 | TTGCAGTAACTCTCTACCAAACGCTC | | TTGCAGTAACTCTCTACCAAACGCTC | | AAT | | AATAACA | |
| (ME-B. | CTGTATTAAATAGGTAAAAGCCTTGT | | CTGTATTAAATAGGTAAAAGCCTTGT | | | | ATAGTTG | |
| 17) | GCAAAACATTAAAATGGGCGTGTACA | | GCAAAACATTAAAATGGGCGTGTACA | | | | TATTTAA | |
| | AGAATACAATGAAAGTTGTATTTAAA | | AGAATACAAT | | | | AT | |
| | T | | | | | | | |
| AoCas | AGGCTCAGGCGAATTTGCGTCTGTAG | 94 | AGGCTCAGGCGAATTTGCGTCTGTAG | 132 | GTGGATTGAA | 166 | GTCGCAA | 200 |
| 12f1 | AGGGAGGCAGGGTGTAACACGATAGC | | AGGGAGGCAGGGTGTAACACGATAGC | | AT | | CCTATAT | |
| (ME-A | CCGTATAGCACTCCCTAAAGGGTTAA | | CCGTATAGCACTCCCTAAAGGGTTAA | | | | GGATACG | |
| .7) | CCTTGGTCATTATGTTACCGACCAAG | | CCTTGGTCATTATGTTACCGACCAAG | | | | TGGATTG | |
| | CAATACCACAGAAAGTGGATTGAAAT | | CAATACCACA | | | | AAAT | |
| Bc2Cas | CGATTTAGCGTCTAAAGGGTGAGATT | 95 | CGATTTAGCGTCTAAAGGGTGAGATT | 133 | TATGGAATGT | 167 | GTTTGAT | 201 |
| 12f1 | GTAGCTCACCAAGGGTTAACTCTAGT | | GTAGCTCACCAAGGGTTAACTCTAGT | | AAAT | | ATCAACT | |
| (ME-A | CTTGTTATGTTACCGACTAGACATTC | | CTTGTTATGTTACCGACTAGACATTC | | | | ATATGGA | |
| .5) | TATATTGATATCGAATGAACATTCCG | | TATATTGATATCGAATGAACATTCCG | | | | ATGTAAA | |
| | TAGAAATATGGAATGTAAAT | | TA | | | | T | |
| CdCas | ATGTGAGGTCATGTGATATAGGCACT | 96 | ATGTGAGGTCATGTGATATAGGCACT | 134 | TGTTATTAAA | 168 | GTTGAAG | 202 |
| 12f1 | CGCAAAGATAGTTGCTAAAGGTAGCA | | CGCAAAGATAGTTGCTAAAGGTAGCA | | T | | AATAACA | |
| (ME-A | ATTATCATCGTCCTAGTGAATTGCTA | | ATTATCATCGTCCTAGTGAATTGCTA | | | | TGAGATG | |
| .3) | GGTAATAACAGAAATGTTATTAAAT | | GGTAATAACA | | | | TTTTTAA | |
| | | | | | | | AT | |
| Cs1Cas | AGATAATAAGAACAGGGCGATTTAAC | 97 | AGATAATAAGAACAGGGCGATTTAAC | 135 | TATGGAATGT | 169 | GTTTTAT | 203 |
| 12f1 | GTCCTAAGGCTGAGGGATATTTCCAC | | GTCCTAAGGCTGAGGGATATTTCCAC | | AAAT | | CTTAACT | |
| (ME-A | TCGGCAAGGGTTAATTTCGGATATTG | | TCGGCAAGGGTTAATTTCGGATATTG | | | | ATATGGA | |
| .4) | TGTTACCATCCGAACATTCCATGGAA | | TGTTACCATCCGAACATTCCATG | | | | ATGTAAA | |
| | ATATGGAATGTAAAT | | | | | | T | |
| Cb3Ca | AGTCTATTAATAAATAGGTATGTAAT | 98 | AGTCTATTAATAAATAGGTATGTAAT | 136 | TAGTATGTGA | 170 | GTTATAA | 204 |
| s12f1 | AGCATATAAACCGAAGGGTGAGAGAA | | AGCATATAAACCGAAGGGTGAGAGAA | | AT | | ATCTACT | |
| (ME-A | TAGACTTTCATGTATTAGGATTTACC | | TAGACTTTCATGTATTAGGATTTACC | | | | ATGTAGT | |
| .10) | AACAATACACTATTACTACTCACTAA | | AACAATACACTATTACTACTCACTAA | | | | ATGTGAA | |
| | GGGTAAGCCCAGGTGTTAAGTTACCG | | GGGTAAGCCCAGGTGTTAAGTTACCG | | | | T | |
| | CCTGGCATACTAGAAATAGTATGTGA | | CCTGGCATACTA | | | | | |
| | AT | | | | | | | |
| Cb4Ca | ACGGTATATTCAAATGCCGAAGAATG | 99 | ACGGTATATTCAAATGCCGAAGAATG | 137 | TATATGATGT | 171 | GTTATAA | 205 |
| s12f1 | AGAGATATTTGATTTAAAATAGCTAG | | AGAGATATTTGATTTAAAATAGCTAG | | GAAT | | GTTAACT | |
| (ME-A | GTTTAGGCCAACAGTTATAAAAATCT | | GTTTAGGCCAACAGTTATAAAAATCT | | | | ATAATTG | |
| .11) | ACTCGTTAAAGGTTAATCCAGATGTT | | ACTCGTTAAAGGTTAATCCAGATGTT | | | | ATGTGAA | |
| | ATGTTACTGTCTGGCATCATATAGAA | | ATGTTACTGTCTGGCATCATATA | | | | T | |
| | ATATATGATGTGAAT | | | | | | | |
| BsCas1 | TTGTATTGATGTTATATATAAATATA | 100 | TTGTATTGATGTTATATATAAATATA | 138 | TGGAATGTTA | 172 | GGTTTAA | 206 |
| 2f1 | TAGCAGTTACGGTAACTTAAAGTGCC | | TAGCAGTTACGGTAACTTAAAGTGCC | | AT | | TAGCACC | |
| (ME-A | GAAGGCTGAGGAGATGGATTAAATAT | | GAAGGCTGAGGAGATGGATTAAATAT | | | | ATAATGG | |
| .12) | ATAAGGTTTTGACCAACTATATACCA | | ATAAGGTTTTGACCAACTATATACCA | | | | AATGTTA | |
| | TCATCACTCGGTAAGGGTTAATCCTA | | TCATCACTCGGTAAGGGTTAATCCTA | | | | AT | |
| | ACAATGTGTGACCGTTAGGCGTTCCA | | ACAATGTGTGACCGTTAGGCGTTCCA | | | | | |
| | AAGAAATGGAATGTTAAT | | AA | | | | | |
| Pt2Cas | AGGGTGAGGGTATAGATAAAACGCAT | 101 | AGGGTGAGGGTATAGATAAAACGCAT | 139 | GTAGTGTGTA | 173 | GTTATAT | 207 |
| 12f1 | AAGGTAGTATGCCAAATATGTGCTAT | | AAGGTAGTATGCCAAATATGTGCTAT | | AAC | | AATGACT | |
| (ME-A | AACCACTCGCTAAGCCGAAAAAACCT | | AACCACTCGCTAAGCCGAAAAAACCT | | | | ATGTAGT | |
| .9) | TAGTTTATGATGGCAACTAAGCACAC | | TAGTTTATGATGGCAACTAAGCACAC | | | | GTGTAAA | |
| | TATGAAAGTAGTGTGTAAAC | | TAT | | | | C | |
| CrCas1 | TTATGTCCAGATTTAGTGGGGCAAGA | 102 | TTATGTCCAGATTTAGTGGGGCAAGA | 140 | GTGATATGTA | 174 | GTTATAT | 208 |
| 2f1 | GTGAGGGCGTAGGTTAAAAGTGTAAG | | GTGAGGGCGTAGGTTAAAAGTGTAAG | | AAG | | ATTAACT | |
| (ME-A | GCTAATAGCAACTACATTCTACGACA | | GCTAATAGCAACTACATTCTACGACA | | | | AAGTGAT | |
| .8) | CTCGCTAAACGGTAAAAACTCTAGCC | | CTCGCTAAACGGTAAAAACTCTAGCC | | | | ATGTAAA | |
| | TAATATTCTCAAGAGATTACGTGGCT | | TAATATTCTCAAGAGATTACGTGGCT | | | | G | |
| | AGACATATCACGAAAGTGATATGTAA | | AGACATATCAC | | | | | |
| | AG | | | | | | | |
| ChCas | TCTATAGGCGATTTAGCGTCTAAAGG | 103 | TCTATAGGCGATTTAGCGTCTAAAGG | 141 | TTGGAATGTA | 175 | ATTTGAT | 209 |
| 12f1 | TTGAGGGATAAGACAAAATGGTTAAG | | TTGAGGGATAAGACAAAATGGTTAAG | | AAT | | TAGAACC | |
| (ME-A | GTTTCGACCAACTAACTACTTATCCA | | GTTTCGACCAACTAACTACTTATCCA | | | | ATATTGG | |
| .2) | CTCGATAAACGGTAAAAACTCATACT | | CTCGATAAACGGTAAAAACTCATACT | | | | AATGTAA | |
| | AATATTCTTTATAGATAACGTGGTAT | | AATATTCTTTATAGATAACGTGGTAT | | | | AT | |
| | GACATTCCAAGAAATTGGAATGTAAA | | GACATTCCAA | | | | | |
| | T | | | | | | | |
| Cs2Cas | ATAGTCGATTCAGCGACTAAAGGTTG | 104 | ATAGTCGATTCAGCGACTAAAGGTTG | 142 | TAGTATGTGA | 176 | GTTTAAT | 210 |
| 12f1 | AGGATATAGGATAAATAGCTAGGATA | | AGGATATAGGATAAATAGCTAGGATA | | AT | | ATAAACT | |
| (ME-A | ACCTAAAGCTATCTATAATCACTCAA | | ACCTAAAGCTATCTATAATCACTCAA | | | | ATGTAGT | |
| .6) | TAAGGGTTAACTCTAGATGTTGTGTT | | TAAGGGTTAACTCTAGATGTTGTGTT | | | | ATGTGAA | |
| | ACCGTCTAGACATACATAGAAATAGT | | ACCGTCTAGACATACATA | | | | T | |
| | ATGTGAAT | | | | | | | |
| PhCas1 | ATAATAAATATATATAGGCGATTTAG | 105 | ATAATAAATATATATAGGCGATTTAG | 143 | TGGAATGTAA | 177 | ATTTGAT | 211 |
| 2f1 | CGTCTAAAGATTGAGGTGTAGGAACT | | CGTCTAAAGATTGAGGTGTAGGAACT | | AT | | TAGTACT | |
| (ME-A | AACGGTTAAGGTTTATACCAACTAAT | | AACGGTTAAGGTTTATACCAACTAAT | | | | ATAATGG | |
| .13) | TACCTATGCACTTGATAAACGGTAAA | | TACCTATGCACTTGATAAACGGTAAA | | | | AATGTAA | |
| | AACTTATACTAACATCCTTTATAGAT | | AACTTATACTAACATCCTTTATAGAT | | | | AT | |
| | AACGTGGTATGACATTCTAGAAATGG | | AACGTGGTATGACATTCTA | | | | | |
| | AATGTAAAT | | | | | | | |
| OpbCa | TATGGGGCTGGATTGCGACTTCGGGA | 106 | TATGGGGCTGGATTGCGACTTCGGGA | 144 | GTGAGTTGGG | 178 | CTTCCAA | 212 |
| s12f1 | GCGCAAACAGACCCAGAAGATGCCTT | | GCGCAAACAGACCCAGAAGATGCCTT | | GG | | TTTGCGC | |
| | CGGGCATCAACCGCCCTGCCAGGACG | | CGGGCATCAACCGCCCTGCCAGGACG | | | | GTGGGCG | |
| | GGGCGCAATTCACGAAAGTGAGTTGG | | GGGCGCAATTCAC | | | | TGAGTTG | |
| | GGG | | | | | | GGGG | |
| CnCas | ACAGGGCGATTTAACGTCCTAAGGCT | 107 | | | | | | |
| 12f1 | GAGAGAAGTTCCTTCTACTCGGCAAG | | | | | | | |
| | GGTTAATCTCGATTGTTGTGTTACCG | | | | | | | |
| | ATCGAGCGTTTCACAGAAATGTGAAA | | | | | | | |
| | TGTAAAT | | | | | | | |
| Un1Cas | ACCGCTTCACTTAGAGTGAAGGTGGG | 108 | | | | | | |
| 12f1 | CTGCTTGCATCAGCCTAATGTCGAGA | | | | | | | |
| | AGTGCTTTCTTCGGAAAGTAACCCTC | | | | | | | |
| | GAAACAAAGAAAGGAATGCAAC | | | | | | | |
| SpCas1 | CTCTGTTTCGCGCGCCAGGGCAGTTA | 109 | | | | | | |
| 2f1 | GGTGCCCTAAAAGAGCGAAGTGGCCG | | | | | | | |
| | AAAGGAAAGGCTAACGCTTCTCTAAC | | | | | | | |
| | GCTACGGCGACCTTGGCGAAATGCCA | | | | | | | |
| | TCAATACCACGCGAAAAACGCGTGGA | | | | | | | |
| | TTGAAAC | | | | | | | |
| AsCas1 | TCTATTCGTCGGTTCAGCGACGATAA | 110 | | | | | | |
| 2f1 | GCCGAGAAGTGCCAATAAAACTGTTA | | | | | | | |
| | AGTGGTTTGGTAACGCTCGGTAAGGT | | | | | | | |
| | AGCCAAAAGGCTGAAACTCCGTGCAC | | | | | | | |
| | AAAGACCGCACGGACGCTTCACATAT | | | | | | | |
| | AGCTCATAAACAAAGTTTGCGAGCTA | | | | | | | |
| | GCTTGTGGAGTGTGAAC | | | | | | | |

OsCas12f1 (ME-B.3)-D228A, SEQ ID NO: 221
MGKGVLAKVMKYELRYLDGCGDFSNMQEQVWALQRQTREILNRSIQIAFQWDCANSEHHRKTGEYLDLKTETGYKRLDGHIYNCLKGQYEDMAT
SNLNATIQKAWKKYNSSKKEILRGSMSIPSYKMNQPLTLDKNTVKLSEGERNPIVTLTLFSDKFKRAQGVSNVKFSMPLHDGTQRAIFANLMNG
TYQLGECQLVYKRPKWFLFVTYKFPPVEHPLDPDKILGVAMGEACALYASTFGEHGYLKIDGGEITKYAKKMEARIRSMQKQAAHCGEGRIGHG
TKTRVSVVYQAKDKVARFRDTINHRYSKALIDYALKNQCGTIQMEDLTGIKEDTGFPKFLRHWTYYDLQSKIEAKAAEHGIQVVKINPRHTSQR
CSRCGHIDKANRTSQADFCCTKCGFSANADFNASQNISIRNIDKIIAKAIGANRKQT
OsCas12f1 (ME-B.3)-D406A, SEQ ID NO: 222
MGKGVLAKVMKYELRYLDGCGDFSNMQEQVWALQRQTREILNRSIQIAFQWDCANSEHHRKTGEYLDLKTETGYKRLDGHIYNCLKGQYEDMAT
SNLNATIQKAWKKYNSSKKEILRGSMSIPSYKMNQPLTLDKNTVKLSEGERNPIVTLTLFSDKFKRAQGVSNVKFSMPLHDGTQRAIFANLMNG
TYQLGECQLVYKRPKWFLFVTYKFPPVEHPLDPDKILGVDMGEACALYASTFGEHGYLKIDGGEITKYAKKMEARIRSMQKQAAHCGEGRIGHG
TKTRVSVVYQAKDKVARFRDTINHRYSKALIDYALKNQCGTIQMEDLIGIKEDTGFPKFLRHWTYYDLQSKIEAKAAEHGIQVVKINPRHTSQR
CSRCGHIDKANRTSQADFCCTKCGFSANAAFNASQNISIRNIDKIIAKAIGANRKQT
RhCas12f1 (ME-A.1)-D210A, SEQ ID NO: 223
MITVRKLKILIDGESRNESYKFIRDSMYAQYLALNKAMSYLGTAYLSRDKEIFKEAIKSLNNSNPIFDNINFGKGIDTKSSVNQTVKKHIQADI
KNGLAKGERSIRNYKRDYPLMTRGRDLKFFYCDTNSTKVKVKWVNGIIFDVMLGKEYNKNDLELRSFLNRVINKEYKISQSSICFDKHNRLILN
LSVNITDNIPNEVVKGRIVGVALGMKIPAYVTLNDSEYIGKPIGDINDFLKVRKQFKERKERLQKQLAINKGGRGITNKMQLMDAFTNKEKNFA
NTYNHGVSKAIINFAKKYKAEQINVEFLALAGSEKEILSSTIRYWSYYQLQQMIEYKANREGIAVKYVDPYLTSQTCCKCGNYEVGQRINQELF
ECKLCGNKMNADRNASFNIARSTKYISSKEESDFYKQLK
RhCas12f1 (ME-A.1)-D388A, SEQ ID NO: 224
MITVRKLKILIDGESRNESYKFIRDSMYAQYLALNKAMSYLGTAYLSRDKEIFKEAIKSLNNSNPIFDNINFGKGIDTKSSVNQTVKKHIQADI
KNGLAKGERSIRNYKRDYPLMTRGRDLKFFYCDTNSTKVKVKWVNGIIFDVMLGKEYNKNDLELRSFLNRVINKEYKISQSSICFDKHNRLILN
LSVNITDNIPNEVVKGRIVGVDLGMKIPAYVTLNDSEYIGKPIGDINDFLKVRKQFKERKERLQKQLAINKGGRGITNKMQLMDAFTNKEKNFA
NTYNHGVSKAIINFAKKYKAEQINVEFLALAGSEKEILSSTIRYWSYYQLQQMIEYKANREGIAVKYVDPYLTSQTCCKCGNYEVGQRINQELF
ECKLCGNKMNAARNASFNIARSTKYISSKEESDFYKQLK
OsCas12f1 (ME-B.3)-D52R, SEQ ID NO: 225
MGKGVLAKVMKYELRYLDGCGDFSNMQEQVWALQRQTREILNRSIQIAFQWRCANSEHHRKTGEYLDLKTETGYKRLDGHIYNCLKGQYEDMAT
SNLNATIQKAWKKYNSSKKEILRGSMSIPSYKMNQPLTLDKNTVKLSEGERNPIVTLTLFSDKFKRAQGVSNVKFSMPLHDGTQRAIFANLMNG
TYQLGECQLVYKRPKWFLFVTYKFPPVEHPLDPDKILGVDMGEACALYASTFGEHGYLKIDGGEITKYAKKMEARIRSMQKQAAHCGEGRIGHG
TKTRVSVVYQAKDKVARFRDTINHRYSKALIDYALKNQCGTIQMEDLTGIKEDTGFPKFLRHWTYYDLQSKIEAKAAEHGIQVVKINPRHTSQR
CSRCGHIDKANRTSQADFCCTKCGFSANADFNASQNISIRNIDKIIAKAIGANRKQT
OsCas12f1 (ME-B.3)-D52RSEQ ID NO: 226
MGKGVLAKVMKYELRYLDGCGDFSNMQEQVWALQRQTREILNRSIQIAFQWRCANSEHHRKTGEYLDLKTETGYKRLDGHIYNCLKGQYEDMAT
SNLNATIQKAWKKYNSSKKEILRGSMSIPSYKMNQPLRLDKNTVKLSEGERNPIVTLTLFSDKFKRAQGVSNVKFSMPLHDGTQRAIFANLMNG
TYQLGECQLVYKRPKWFLFVTYKFPPVEHPLDPDKILGVDMGEACALYASTFGEHGYLKIDGGEITKYAKKMEARIRSMQKQAAHCGEGRIGHG
TKTRVSVVYQAKDKVARFRDTINHRYSKALIDYALKNQCGTIQMEDLTGIKEDTGFPKFLRHWTYYDLQSKIEAKAAEHGIQVVKINPRHTSQR
CSRCGHIDKANRTSQADFCCTKCGFSANADFNASQNISIRNIDKIIAKAIGANRKQT
RhCas12f1 (ME-A.1)-L270R, SEQ ID NO: 227
MITVRKLKILIDGESRNESYKFIRDSMYAQYLALNKAMSYLGTAYLSRDKEIFKEAIKSLNNSNPIFDNINFGKGIDTKSSVNQTVKKHIQADI
KNGLAKGERSIRNYKRDYPLMTRGRDLKFFYCDTNSTKVKVKWVNGIIFDVMLGKEYNKNDLELRSFLNRVINKEYKISQSSICFDKHNRLILN
LSVNITDNIPNEVVKGRIVGVDLGMKIPAYVTLNDSEYIGKPIGDINDFLKVRKQFKERKERLQKQLAINKGGRGITNKMQRMDAFTNKEKNFA
NTYNHGVSKAIINFAKKYKAEQINVEFLALAGSEKEILSSTIRYWSYYQLQQMIEYKANREGIAVKYVDPYLTSQTCCKCGNYEVGQRINQELF
ECKLCGNKMNADRNASFNIARSTKYISSKEESDFYKQLK
Sequences of enOsCas12f1-derived gene editing tools

| Name | Protein sequences |
|---|---|
| DD-enOs | MDYKDHDGDYKDHDIDYKDDDDKMAPKKKRKVGIHGVPAAGSISLIAALAVDYVIGMENAMPWNLPADLAWFKRNTLNKPVIMGRHT |
| Cas12f | WESIGRPLPGRKNIILSSQPSTDDRVTWVKSVDEAIAACGDVPEIMVIGGGRVIEQFLPKAQKLYLTHIDAEVEGDTHFPDYEPDDW |
| 1 (SEQ | ESVFSEFHDADAQNSHSYCFEILERRGSGSGSMPKKKRKVMGKGVLAKVMKYELRYLDGCGDFSNMQEQVWALQRQTREILNRSIQI |
| ID NO: | AFQWRCANSEHHRKTGEYLDLKTETGYKRLDGHIYNCLKGQYEDMATSNLNATIQKAWKKYNSSKKEILRGSMSIPSYKMNQPLRLD |
| 260) | KNTVKLSEGERNPIVTLTLFSDKFKRAQGVSNVKFSMPLHDGTQRAIFANLMNGTYQLGECQLVYKRPKWFLFVTYKFPPVEHPLDP |
| | DKILGVDMGEACALYASTFGEHGYLKIDGGEITKYAKKMEARIRSMQKQAAHCGEGRIGHGTKTRVSVVYQAKDKVARFRDTINHRY |
| | SKALIDYALKNQCGTIQMEDLTGIKEDTGFPKFLRHWTYYDLQSKIEAKAAEHGIQVVKINPRHTSQRCSRCGHIDKANRTSQADFC |
| | CTKCGFSANADFNASQNISIRNIDKIIAKAIGANRKQTKRPAATKKAGQAKKKKISLIAALAVDHVIGMETVMPWNLPADLAWFKRN |
| | TLNKPVIMGRHTWESIGRPLPGRKNIILSSQPSTDDRVTWVKSVDEAIAACGDVPEIMVIGGGRVYEQFLPKAQKLYLTHIDAEVEG |
| | DTHFPDYEPDDWESVFSEFHDADAQNSHSYCFEILERR |
| miniCRI | MNHDQEFDPPKVYPPVPAEKRKPIRVLSLFDGIATGLLVLKDLGIQVDRYIASEVCEDSITVGMVRHQGKIMYVGDVRSVTQKHIQE |
| SPRoff- | WGPFDLVIGGSPCNDLSIVNPARKGLYEGTGRLFFEFYRLLHDARPKEGDDRPFFWLFENVVAMGVSDKRDISRFLESNPVMIDAKE |
| v1 (SEQ | VSAAHRARYFWGNLPGMNRPLASTVNDKLELQECLEHGRIAKFSKVRTITTRSNSIKQGKDQHFPVFMNEKEDILWCTEMERVFGFP |
| ID NO: | VHYTDVSNMSRLARQRLLGRSWSVPVIRHLFAPLKEYFACVSSGNSNANSRGPSFSSGLVPLSLRGSHMGPMEIYKTVSAWKRQPVR |
| 261) | VLSLFRNIDKVLKSLGFLESGSGSGGGTLKYVEDVTNVVRRDVEKWGPFDLVYGSTQPLGSSCDRCPGWYMFQFHRILQYALPRQES |
| | QRPFFWIFMDNLLLTEDDQETTTRFLQTEAVTLQDVRGRDYQNAMRVWSNIPGLKSKHAPLTPKEEEYLQAQVRSRSKLDAPKVDLL |
| | VKNCLLPLREYFKYFSQNSLPLGGPSSGAPPPSGGSPAGSPTSTEEGTSESATPESGPGTSTEPSEGSAPGSPAGSPTSTEEGTSTE |
| | PSEGSAPGTSTEPSERGSGSGSMPKKKRKVMGKGVLAKVMKYELRYLDGCGDFSNMQEQVWALQRQTREILNRSIQIAFQWRCANSE |
| | HHRKTGEYLDLKTETGYKRLDGHIYNCLKGQYEDMATSNLNATIQKAWKKYNSSKKEILRGSMSIPSYKMNQPLRLDKNTVKLSEGE |
| | RNPIVTLTLFSDKFKRAQGVSNVKFSMPLHDGTQRAIFANLMNGTYQLGECQLVYKRPKWFLFVTYKFPPVEHPLDPDKILGVAMGE |
| | ACALYASTFGEHGYLKIDGGEITKYAKKMEARIRSMQKQAAHCGEGRIGHGTKTRVSVVYQAKDKVARFRDTINHRYSKALIDYALK |
| | NQCGTIQMEDLIGIKEDTGFPKFLRHWTYYDLQSKIEAKAAEHGIQVVKINPRHTSQRCSRCGHIDKANRTSQADFCCTKCGFSANA |
| | AFNASQNISIRNIDKIIAKAIGANRKQTKRPAATKKAGQAKKKKAYPYDVPDYASLGSGSPKKKRKVEDPKKKRKVDGIGSGSNGSS |
| | GSSELIKENMHMKLYMEGTVDNHHFKCTSEGEGKPYEGTQTMRIKVVEGGPLPFAFDILATSFLYGSKTFINHTQGIPDFFKQSFPE |
| | GFTWERVTTYEDGGVLTATQDTSLQDGCLIYNVKIRGVNFTSNGPVMQKKTLGWEAFTETLYPADGGLEGRNDMALKLVGGSHLIAN |
| | IKTTYRSKKPAKNLKMPGVYYVDYRLERIKEANNETYVEQHEVAVARYCDLPSKLGHKLNGGGGGMDAKSLTAWSRTLVTFKDVFVD |
| | FTREEWKLLDTAQQIVYRNVMLENYKNLVSLGYQLTKPDVILRLEKGEEP |
| miniCRI | MAYPYDVPDYASLGSGSPKKKRKVEDPKKKRKVDGIGSGSNGSSGSSELIKENMHMKLYMEGTVDNHHFKCTSEGEGKPYEGTQTMR |
| SPRoff- | IKVVEGGPLPFAFDILATSFLYGSKTFINHTQGIPDFFKQSFPEGFTWERVTTYEDGGVLTATQDTSLQDGCLIYNVKIRGVNFTSN |
| v2 (SEQ | GPVMQKKTLGWEAFTETLYPADGGLEGRNDMALKLVGGSHLIANIKTTYRSKKPAKNLKMPGVYYVDYRLERIKEANNETYVEQHEV |
| ID NO: | AVARYCDLPSKLGHKLNGGGGGMDAKSLTAWSRTLVTFKDVFVDFTREEWKLLDTAQQIVYRNVMLENYKNLVSLGYQLTKPDVILR |
| 262) | LEKGEEPRGSGSGSMPKKKRKVMGKGVLAKVMKYELRYLDGCGDFSNMQEQVWALQRQTREILNRSIQIAFQWRCANSEHHRKTGEY |
| | LDLKTETGYKRLDGHIYNCLKGQYEDMATSNLNATIQKAWKKYNSSKKEILRGSMSIPSYKMNQPLRLDKNTVKLSEGERNPIVTLT |
| | LFSDKFKRAQGVSNVKFSMPLHDGTQRAIFANLMNGTYQLGECQLVYKRPKWFLFVTYKFPPVEHPLDPDKILGVAMGEACALYAST |
| | FGEHGYLKIDGGEITKYAKKMEARIRSMQKQAAHCGEGRIGHGTKTRVSVVYQAKDKVARFRDTINHRYSKALIDYALKNQCGTIQM |
| | EDLTGIKEDTGFPKFLRHWTYYDLQSKIEAKAAEHGIQVVKINPRHTSQRCSRCGHIDKANRTSQADFCCTKCGFSANAAFNASQNI |
| | SIRNIDKIIAKAIGANRKQTKRPAATKKAGQAKKKKSSGMNHDQEFDPPKVYPPVPAEKRKPIRVLSLFDGIATGLLVLKDLGIQVD |
| | RYIASEVCEDSITVGMVRHQGKIMYVGDVRSVTQKHIQEWGPFDLVIGGSPCNDLSIVNPARKGLYEGTGRLFFEFYRLLHDARPKE |
| | GDDRPFFWLFENVVAMGVSDKRDISRFLESNPVMIDAKEVSAAHRARYFWGNLPGMNRPLASTVNDKLELQECLEHGRIAKFSKVRT |
| | ITTRSNSIKQGKDQHFPVFMNEKEDILWCTEMERVFGFPVHYTDVSNMSRLARQRLLGRSWSVPVIRHLFAPLKEYFACVSSGNSNA |
| | NSRGPSFSSGLVPLSLRGSHMGPMEIYKTVSAWKRQPVRVLSLFRNIDKVLKSLGFLESGSGSGGGTLKYVEDVTNVVRRDVEKWGP |
| | FDLVYGSTQPLGSSCDRCPGWYMFQFHRILQYALPRQESQRPFFWIFMDNLLLTEDDQETTTRFLQTEAVTLQDVRGRDYQNAMRVW |
| | SNIPGLKSKHAPLTPKEEEYLQAQVRSRSKLDAPKVDLLVKNCLLPLREYFKYFSQNSLPLGGPSSGAPPPSGGSPAGSPTSTEEGT |
| | SESATPESGPGTSTEPSEGSAPGSPAGSPTSTEEGTSTEPSEGSAPGTSTEPSE |
| miniCRI | MDYKDHDGDYKDHDIDYKDDDDKMPKKKRKVMGKGVLAKVMKYELRYLDGCGDFSNMQEQVWALQRQTREILNRSIQIAFQWRCANS |
| SPRoff- | EHHRKTGEYLDLKTETGYKRLDGHIYNCLKGQYEDMATSNLNATIQKAWKKYNSSKKEILRGSMSIPSYKMNQPLRLDKNTVKLSEG |
| v3 (SEQ | ERNPIVTLTLFSDKFKRAQGVSNVKFSMPLHDGTQRAIFANLMNGTYQLGECQLVYKRPKWFLFVTYKFPPVEHPLDPDKILGVAMG |
| ID NO: | EACALYASTFGEHGYLKIDGGEITKYAKKMEARIRSMQKQAAHCGEGRIGHGTKTRVSVVYQAKDKVARFRDTINHRYSKALIDYAL |
| 263) | KNQCGTIQMEDLTGIKEDTGFPKFLRHWTYYDLQSKIEAKAAEHGIQVVKINPRHTSQRCSRCGHIDKANRTSQADFCCTKCGFSAN |
| | AAFNASQNISIRNIDKIIAKAIGANRKQTKRPAATKKAGQAKKKKSSGMNHDQEFDPPKVYPPVPAEKRKPIRVLSLFDGIATGLLV |
| | LKDLGIQVDRYIASEVCEDSITVGMVRHQGKIMYVGDVRSVTQKHIQEWGPFDLVIGGSPCNDLSIVNPARKGLYEGTGRLFFEFYR |
| | LLHDARPKEGDDRPFFWLFENVVAMGVSDKRDISRFLESNPVMIDAKEVSAAHRARYFWGNLPGMNRPLASTVNDKLELQECLEHGR |
| | IAKFSKVRTITTRSNSIKQGKDQHFPVFMNEKEDILWCTEMERVFGFPVHYTDVSNMSRLARQRLLGRSWSVPVIRHLFAPLKEYFA |
| | CVSSGNSNANSRGPSFSSGLVPLSLRGSHMGPMEIYKTVSAWKRQPVRVLSLFRNIDKVLKSLGFLESGSGSGGGTLKYVEDVTNVV |
| | RRDVEKWGPFDLVYGSTQPLGSSCDRCPGWYMFQFHRILQYALPRQESQRPFFWIFMDNLLLTEDDQETTTRFLQTEAVTLQDVRGR |
| | DYQNAMRVWSNIPGLKSKHAPLTPKEEEYLQAQVRSRSKLDAPKVDLLVKNCLLPLREYFKYFSQNSLPLGGPSSGAPPPSGGSPAG |
| | SPTSTEEGTSESATPESGPGTSTEPSEGSAPGSPAGSPTSTEEGTSTEPSEGSAPGTSTEPSEAYPYDVPDYASLGSGSPKKKRKVE |
| | DPKKKRKVDGIGSGSNGSSGSSELIKENMHMKLYMEGTVDNHHFKCTSEGEGKPYEGTQTMRIKVVEGGPLPFAFDILATSFLYGSK |
| | TFINHTQGIPDFFKQSFPEGFTWERVTTYEDGGVLTATQDTSLQDGCLIYNVKIRGVNFTSNGPVMQKKTLGWEAFTETLYPADGGL |
| | EGRNDMALKLVGGSHLIANIKTTYRSKKPAKNLKMPGVYYVDYRLERIKEANNETYVEQHEVAVARYCDLPSKLGHKLNGGGGGMDA |
| | KSLTAWSRTLVTFKDVFVDFTREEWKLLDTAQQIVYRNVMLENYKNLVSLGYQLTKPDVILRLEKGEEP |
| miniCRI | MDYKDHDGDYKDHDIDYKDDDDKMPKKKRKVMGKGVLAKVMKYELRYLDGCGDFSNMQEQVWALQRQTREILNRSIQIAFQWRCANS |
| SPRoff- | EHHRKTGEYLDLKTETGYKRLDGHIYNCLKGQYEDMATSNLNATIQKAWKKYNSSKKEILRGSMSIPSYKMNQPLRLDKNTVKLSEG |
| v4 (SEQ | ERNPIVTLTLFSDKFKRAQGVSNVKFSMPLHDGTQRAIFANLMNGTYQLGECQLVYKRPKWFLFVTYKFPPVEHPLDPDKILGVAMG |
| ID NO: | EACALYASTFGEHGYLKIDGGEITKYAKKMEARIRSMQKQAAHCGEGRIGHGTKTRVSVVYQAKDKVARFRDTINHRYSKALIDYAL |
| 264) | KNQCGTIQMEDLTGIKEDTGFPKFLRHWTYYDLQSKIEAKAAEHGIQVVKINPRHTSQRCSRCGHIDKANRTSQADFCCTKCGFSAN |
| | AAFNASQNISIRNIDKIIAKAIGANRKQTKRPAATKKAGQAKKKKGSSGSSELIKENMHMKLYMEGTVDNHHFKCTSEGEGKPYEGT |
| | QTMRIKVVEGGPLPFAFDILATSFLYGSKTFINHTQGIPDFFKQSFPEGFTWERVTTYEDGGVLTATQDTSLQDGCLIYNVKIRGVN |
| | FTSNGPVMQKKTLGWEAFTETLYPADGGLEGRNDMALKLVGGSHLIANIKTTYRSKKPAKNLKMPGVYYVDYRLERIKEANNETYVE |
| | QHEVAVARYCDLPSKLGHKLNGGGGGMDAKSLTAWSRTLVTFKDVFVDFTREEWKLLDTAQQIVYRNVMLENYKNLVSLGYQLTKPD |
| | VILRLEKGEEPRGSGSGSMPKKKRKVMNHDQEFDPPKVYPPVPAEKRKPIRVLSLFDGIATGLLVLKDLGIQVDRYIASEVCEDSIT |
| | VGMVRHQGKIMYVGDVRSVTQKHIQEWGPFDLVIGGSPCNDLSIVNPARKGLYEGTGRLFFEFYRLLHDARPKEGDDRPFFWLFENV |
| | VAMGVSDKRDISRFLESNPVMIDAKEVSAAHRARYFWGNLPGMNRPLASTVNDKLELQECLEHGRIAKFSKVRTITTRSNSIKQGKD |
| | QHFPVFMNEKEDILWCTEMERVFGFPVHYTDVSNMSRLARQRLLGRSWSVPVIRHLFAPLKEYFACVSSGNSNANSRGPSFSSGLVP |
| | LSLRGSHMGPMEIYKTVSAWKRQPVRVLSLFRNIDKVLKSLGFLESGSGSGGGTLKYVEDVTNVVRRDVEKWGPFDLVYGSTQPLGS |
| | SCDRCPGWYMFQFHRILQYALPRQESQRPFFWIFMDNLLLTEDDQETTTRFLQTEAVTLQDVRGRDYQNAMRVWSNIPGLKSKHAPL |
| | TPKEEEYLQAQVRSRSKLDAPKVDLLVKNCLLPLREYFKYFSQNSLPLGGPSSGAPPPSGGSPAGSPTSTEEGTSESATPESGPGTS |
| | TEPSEGSAPGSPAGSPTSTEEGTSTEPSEGSAPGTSTEPSE |
| denOsCa | MDYKDHDGDYKDHDIDYKDDDDKMPKKKRKVMGKGVLAKVMKYELRYLDGCGDFSNMQEQVWALQRQTREILNRSIQIAFQWRCANS |
| s12f1-V | EHHRKTGEYLDLKTETGYKRLDGHIYNCLKGQYEDMATSNLNATIQKAWKKYNSSKKEILRGSMSIPSYKMNQPLRLDKNTVKLSEG |
| PR | ERNPIVTLTLFSDKFKRAQGVSNVKFSMPLHDGTQRAIFANLMNGTYQLGECQLVYKRPKWFLFVTYKFPPVEHPLDPDKILGVAMG |
| (SEQ ID | EACALYASTFGEHGYLKIDGGEITKYAKKMEARIRSMQKQAAHCGEGRIGHGTKTRVSVVYQAKDKVARFRDTINHRYSKALIDYAL |
| NO: | KNQCGTIQMEDLTGIKEDTGFPKFLRHWTYYDLQSKIEAKAAEHGIQVVKINPRHTSQRCSRCGHIDKANRTSQADFCCTKCGFSAN |
| 265) | AAFNASQNISIRNIDKIIAKAIGANRKQTKRPAATKKAGQAKKKKAYPYDVPDYASLGSGDGIGSGSNGSSLDALDDFDLDMLGSDA |
| | LDDFDLDMLGSDALDDFDLDMLGSDALDDFDLDMLGSGGSGSQYLPDTDDRHRIEEKRKRTYETFKSIMKKSPFSGPTDPRPPPRRI |
| | AVPSRSSASVPKPAPQPYPFTSSLSTINYDEFPTMVFPSGQISQASALAPAPPQVLPQAPAPAPAPAMVSALAQAPAPVPVLAPGPP |
| | QAVAPPAPKPTQAGEGTLSEALLQLQFDDEDLGALLGNSTDPAVFTDLASVDNSEFQQLLNQGIPVAPHTTEPMLMEYPEAITRLVT |
| | GAQRPPDPAPAPLGAPGLPNGLLSGDEDFSSIADMDFSALLSQISSGSGSGSRDSREGMFLPKPEAGSAISDVFEGREVCQPKRIRP |
| | FHPPGSPWANRPLPASLAPTPTGPVHEPVGSLTPAPVPQPLDPAPAVTPEASHLLEDPDEETSQAVKALREMADTVIPQKEEAAICG |
| | QMDLSHPPPRGHLDELTTTLESMTEDLNLDSPLTPELNEILDTFLNDECLLHAMHISTGLSIFDTSLFHMGGGSGEDPAAKRVKLDM |
| | GSGVSKGEEDNMAIIKEFMRFKVHMEGSVNGHEFEIEGEGEGRPYEGTQTAKLKVTKGGPLPFAWDILSPQFMYGSKAYVKHPADIP |
| | DYLKLSFPEGFKWERVMNFDDGGVVTVTQDSSLQDGEFIYKVKLRGTNFPSDGPVMQKKTMGWEASSERMYPEDGALKGEIKQRLKL |
| | KDGGHYDAEVKTTYKAKKPVQLPGAYNVNIKLDITSHNEDYTIVEQYERAEGRHSTGGMDELYK |

denOsCas12f1 (D52R + T132R + D228A + D406A), SEQ ID NO: 513
MGKGVLAKVMKYELRYLDGCGDFSNMQEQVWALQRQTREILNRSIQIAFQWRCANSEHHRKTGEYLDLKTETGYKRLDGHIYNCLKGQYEDMAT
SNLNATIQKAWKKYNSSKKEILRGSMSIPSYKMNQPLRLDKNTVKLSEGERNPIVTLTLFSDKFKRAQGVSNVKFSMPLHDGTQRAIFANLMNG
TYQLGECQLVYKRPKWFLFVTYKFPPVEHPLDPDKILGVAMGEACALYASTFGEHGYLKIDGGEITKYAKKMEARIRSMQKQAAHCGEGRIGHG
TKTRVSVVYQAKDKVARFRDTINHRYSKALIDYALKNQCGTIQMEDLTGIKEDTGFPKFLRHWTYYDLQSKIEAKAAEHGIQVVKINPRHTSQR
CSRCGHIDKANRTSQADFCCTKCGFSANAAFNASQNISIRNIDKIIAKAIGANRKQT
KRAB, SEQ ID NO: 514
RTLVTFKDVFVDFTREEWKLLDTAQQIVYRNVMLENYKNLVSLGYQLTKPDVILRLEKGEEP
denRhCas12f1 (ME-A.1)-D210A + L270R + D388A, SEQ ID NQ: 515
MITVRKLKILIDGESRNESYKFIRDSMYAQYLALNKAMSYLGTAYLSRDKEIFKEAIKSLNNSNPIFDNINFGKGIDTKSSVNQTVKKHIQADI
KNGLAKGERSIRNYKRDYPLMTRGRDLKFFYCDTNSTKVKVKWVNGIIFDVMLGKEYNKNDLELRSFLNRVINKEYKISQSSICFDKHNRLILN
LSVNITDNIPNEVVKGRIVGVALGMKIPAYVTLNDSEYIGKPIGDINDFLKVRKQFKERKERLQKQLAINKGGRGITNKMQRMDAFTNKEKNFA
NTYNHGVSKAIINFAKKYKAEQINVEFLALAGSEKEILSSTIRYWSYYQLQQMIEYKANREGIAVKYVDPYLTSQTCCKCGNYEVGQRINQELF
ECKLCGNKMNAARNASFNIARSTKYISSKEESDFYKQLK

1. (canceled)

2. A system comprising:
(1) a Cas12f polypeptide comprising an amino acid sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 1-34 (optionally any one of SEQ ID NOs: 1, 2, 4, 5, 15, 28, 29, 31, and 32), or a polynucleotide encoding the Cas12f polypeptide; and
(2) a guide nucleic acid or a polynucleotide encoding the guide nucleic acid, the guide nucleic acid comprising:
(i) a scaffold sequence capable of forming a complex with the Cas12f polypeptide; and
(ii) a guide sequence capable of hybridizing to a target sequence of a target DNA, thereby guiding the complex to the target DNA.

3.-4. (canceled)

5. The system of claim 2, wherein the Cas12f polypeptide has guide sequence-specific (on-target) dsDNA cleavage activity; optionally, wherein the Cas12f polypeptide substantially retains the guide sequence-specific (on-target) dsDNA cleavage activity of any one of SEQ ID NOs: 1-34 (optionally any one of SEQ ID NOs: 1, 2, 4, 5, 15, 28, 29, 31, and 32).

6. The system of claim 2, wherein the Cas12f polypeptide has an increased guide sequence-specific (on-target) dsDNA cleavage activity compared to that of any one of SEQ ID NOs: 1-34 (optionally any one of SEQ ID NOs: 1, 2, 4, 5, 15, 28, 29, 31, and 32) when both are used in combination with a same guide nucleic acid, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

7. The system of claim 6, wherein the Cas12f polypeptide comprises:
(i) an amino acid substitution at position 46, 49, 50, 52, 53, 54, 56, 57, 62, 63, 66, 70, 71, 72, 119, 120, 127, 132, 136, 141, 144, 146, 147, 148, 150, 264, 292, 293, 311, 313, 314, and/or 315 of SEQ ID NO: 1; or
(ii) an amino acid substitution at position 10, 11, 13, 14, 15, 17, 18, 19, 20, 27, 28, 31, 32, 40, 44, 47, 49, 51, 52, 55, 56, 59, 61, 63, 65, 68, 71, 84, 91, 94, 96, 99, 111, 112, 124, 125, 126, 127, 128, 129, 130, 131, 139, 140, 141, 146, 147, 150, 151, 156, 160, 163, 167, 170, 173, 178, 179, 180, 183, 192, 193, 194, 195, 196, 197, 198, 199, 200, 201, 202, 206, 215, 224, 225, 226, 227, 230, 235, 249, 254, 256, 257, 264, 265, 266, 269, 270, 272, 273, 276, 280, 283, 292, 295, 303, 309, 311, 313, 314, 316, 318, 319, 320, 321, 334, 337, 341, 344, 346, 349, 358, 363, 365, 366, 367, 368, 371, 372, 374, 375, 377, 380, 382, 393, 399, 403, 404, 406, 408, 409, 410, 411, 413, and/or 414 of SEQ ID NO: 2.

8. (canceled)

9. The system of claim 7, wherein the amino acid substitution is a substitution with a positively charged amino acid residue (such as, Lysine (Lys/K), Arginine (Arg/R), Histidine (His/H)), and optionally a substitution with Arginine (Arg/R).

10. The system of claim 6, wherein the Cas12f polypeptide comprises an amino acid substitution D52R and/or T132R relative to SEQ ID NO: 1;
optionally, wherein the Cas12f polypeptide comprises substitutions D52R and T132R relative to SEQ ID NO: 1; and/or
optionally, wherein the Cas12f polypeptide comprises the amino acid sequence of SEQ ID NO: 226, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 226.

11. The system of claim 6, wherein the Cas12f polypeptide comprises an amino acid substitution A56R, Y125R, S130R, T131R, I264R, L270R, and/or A273R relative to SEQ ID NO: 2;
optionally, wherein the Cas12f polypeptide comprises an amino acid substitution L270R relative to SEQ ID NO: 2; and/or
optionally, wherein the Cas12f polypeptide comprises the amino acid sequence of SEQ ID NO: 227, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 227.

12. (canceled)

13. The system of claim 2, wherein the Cas12f polypeptide is further engineered to substantially lack guide sequence-specific (on-target) dsDNA cleavage activity;
optionally, wherein the Cas12f polypeptide substantially lacks the guide sequence-specific (on-target) dsDNA cleavage activity of any one of SEQ ID NOs: 1-34 (optionally any one of SEQ ID NOs: 1, 2, 4, 5, 15, 28, 29, 31, and 32); and/or
optionally, wherein the Cas12f polypeptide has a decreased guide sequence-specific (on-target) dsDNA cleavage activity compared to that of any one of SEQ ID NOs: 1-34 (optionally any one of SEQ ID NOs: 1, 2, 4, 5, 15, 28, 29, 31, and 32) when both used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

14. The system of claim 13, wherein the Cas12f polypeptide comprises:
(i) an amino acid substitution at position 44, 79, 81, 82, 125, 131, 133, 138, 149, 151, 153, 228, 268, 270, 271, 274, 275, 277, 279, 282, 287, 291, 305, 308, 312, and/or 406 of SEQ ID NO: 1; or
(ii) an amino acid substitution at position 4, 7, 9, 23, 30, 33, 34, 35, 37, 38, 39, 41, 42, 46, 60, 62, 67, 69, 72, 75, 76, 77, 78, 80, 81, 82, 86, 90, 93, 97, 98, 101, 105, 107, 108, 114, 116, 121, 123, 135, 137, 143, 145, 148, 162, 165, 177, 185, 187, 189, 190, 207, 208, 209, 210, 212, 216, 217, 218, 219, 220, 231, 243, 278, 289, 290, 293, 296, 297, 302, 305, 307, 308, 310, 326, 327, 328, 329, 332, 336, 340, 347, 350, 356, 359, 362, 376, 378, 381, 388, 390, 391, 392, 395, and/or 396 of SEQ ID NO: 2.

15. (canceled)

16. The system of claim 14, wherein the amino acid substitution is a substitution with (1) a positively charged amino acid residue (such as, Lysine (Lys/K), Arginine (Arg/R), Histidine (His/H)), and optionally a substitution with Arginine (Arg/R); or (2) a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F)), and optionally a substitution with Alanine (Ala/A).

17. The system of claim 13, wherein the Cas12f polypeptide comprises an amino acid substitution D228A and/or D406A relative to SEQ ID NO: 1;
optionally, wherein the Cas12f polypeptide comprises the amino acid sequence of SEQ ID NO: 221 or 222, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 221 or 222.

18. The system of claim 17, wherein the Cas12f polypeptide comprises amino acid substitutions D52R, T132R, D228A, and D406A relative to SEQ ID NO: 1;
optionally, wherein the Cas12f polypeptide comprises the amino acid sequence of SEQ ID NO: 513, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 513.

19. The system of claim 13, wherein the Cas12f polypeptide comprises an amino acid substitution D210A and/or D388A relative to SEQ ID NO: 2;
optionally, wherein the Cas12f polypeptide comprises the amino acid sequence of SEQ ID NO: 223 or 224, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 223 or 224.

20. The system of claim 19, wherein the Cas12f polypeptide comprises amino acid substitutions D210A, L270R, and D388A relative to SEQ ID NO: 2;
optionally, wherein the Cas12f polypeptide comprises the amino acid sequence of SEQ ID NO: 515, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 515.

21. (canceled)

22. The system of claim 2, wherein the Cas12f polypeptide further comprises a functional domain fused to the Cas12f polypeptide;
optionally, wherein the functional domain is selected from the group consisting of a nuclear localization signal (NLS), a nuclear export signal (NES), a base editing domain, for example, a deaminase or a catalytic domain thereof, a base excising domain, an uracil glycosylase inhibitor (UGI) or a catalytic domain thereof, an uracil glycosylase (UNG) or a catalytic domain thereof, a methylpurine glycosylase (MPG) or a catalytic domain thereof, a methylase or a catalytic domain thereof, a demethylase or a catalytic domain thereof, an transcription activating domain (e.g., VP64 or VPR), an transcription inhibiting domain (e.g., KRAB moiety or SID moiety), a reverse transcriptase or a catalytic domain thereof, an exonuclease (e.g., T5E) or a catalytic domain thereof, a destabilized domain (e.g., destabilized domains (DD) of E. coli dihydrofolate reductase (ecDHFR)), a histone residue modification domain, a nuclease catalytic domain (e.g., FokI), a transcription modification factor, a light gating factor, a chemical inducible factor, a chromatin visualization factor, a targeting polypeptide for providing binding to a cell surface portion on a target cell or a target cell type, a reporter (e.g., fluorescent) polypeptide or a detection label (e.g., GST, HRP, CAT, GFP, HcRed, DsRed, CFP, YFP, BFP), a localization signal, a polypeptide targeting moiety, a DNA binding domain (e.g., MBP, Lex A DBD, Gal4 DBD), an epitope tag (e.g., His, myc, V5, FLAG, HA, VSV-G, Trx, etc), a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase, a functional domain exhibiting activity to modify a target DNA, selected from the group consisting of: methyltransferase activity, DNA repair activity, DNA damage activity, dismutase activity, alkylation activity, dealkylation activity, depurination activity, oxidation activity, deoxidation activity, pyrimidine dimer forming activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, and a catalytic domain thereof, and a functional fragment (e.g., a functional truncation) thereof, and any combination thereof.

23. The system of claim 22, wherein the Cas12f polypeptide further comprises a destabilized domain (e.g., destabilized domains (DD) of E. coli dihydrofolate reductase (ecDHFR)); optionally wherein the Cas12f polypeptide comprises the amino acid sequence of SEQ ID NO: 260, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 260.

24. The system of claim 22, wherein the Cas12f polypeptide further comprises a methylase or a catalytic domain thereof (e.g., DNA methyltransferase 3a (Dnmt3a) and DNA methyltransferase 3-like protein (Dnmt3L)) and a transcription inhibiting domain (e.g., KRAB moiety (e.g., SEQ ID NO: 514) or SID moiety); optionally wherein the Cas12f polypeptide comprises the amino acid sequence of any one of SEQ ID NOs: 261-264, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of any one of SEQ ID NOs: 261-264.

25. The system of claim 22, wherein the Cas12f polypeptide further comprises an transcription activating domain (e.g., VP64 or VPR); and optionally wherein the Cas12f polypeptide comprises the amino acid sequence of SEQ ID NO: 265, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 265.

26. The system of claim 2, wherein the guide nucleic acid is a guide RNA (gRNA), e.g., a single guide RNA (sgRNA).

27. The system of claim 2, wherein the scaffold sequence is 5′ to the guide sequence.

28. The system of claim 27, wherein the guide nucleic acid further comprises a polyU sequence having at least four consecutive U (uridine) 3′ to the guide sequence;
optionally, wherein the polyU sequence further comprises one A (adenosine) downstream of the at least four consecutive U; and/or
optionally, wherein the sequence encoding the polyU sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of SEQ ID NO:220; or a sequence having at most 1, 2, 3, 4, 5, 6, 7, 8, 9, or 10 nucleotide differences, whether consecutive or not, compared to the sequence of SEQ ID NO: 220.

29. The system of claim 27, wherein the scaffold sequence has substantially the same secondary structure as the secondary structure of any one of SEQ ID NOs: 73-106 (optionally any one of SEQ ID NOs: 73, 74, 76, 77, 87, 100, 101, 103, and 104).

30. The system of claim 27, wherein the scaffold sequence comprises a polynucleotide sequence having a sequence identity of at least about 60% (e.g., at least about 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 73-106 (optionally any one of SEQ ID NOs: 73, 74, 76, 77, 87, 100, 101, 103, and 104).

31. The system of claim 27, wherein the scaffold sequence leads to an increased guide sequence-specific (on-target) dsDNA cleavage activity compared to that led by any one of SEQ ID NOs: 73-106 (optionally any one of SEQ ID NOs: 73, 74, 76, 77, 87, 100, 101, 103, and 104) when both are used in otherwise identical guide nucleic acid in combination with a same Cas12f polypeptide, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

32. The system claim 29, wherein the scaffold sequence comprises a base pair substitution of a thermodynamically unstable base pair (e.g., a A-U base pair or a mismatched base pair) with a G-C base pair.

33. The system of claim 30, wherein the scaffold sequence comprises a base pair substitution of a thermodynamically unstable base pair (e.g., a A-U base pair or a mismatched base pair) with a G-C base pair relative to SEQ ID NO: 73 and comprises the polynucleotide sequence of any one of SEQ ID NOs: 234-236, 239-242, 244-247, and 250-251, or a polynucleotide sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of any one of SEQ ID NOs: 234-236, 239-242, 244-247, and 250-251; optionally, wherein the scaffold sequence comprises the polynucleotide sequence of SEQ ID NO: 244.

34. The system of claim 30, wherein the scaffold sequence comprises a base pair substitution of a thermodynamically unstable base pair (e.g., a A-U base pair or a mismatched base pair) with a G-C base pair relative to SEQ ID NO: 74 and comprises the polynucleotide sequence of SEQ ID NO: 257, or a polynucleotide sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the polynucleotide sequence of SEQ ID NO: 257.

35. The system of claim 2, wherein:
1) the Cas12f polypeptide comprises SEQ ID NO: 1 or a mutant thereof as defined in any preceding claim (e.g., SEQ ID NO: 226), and wherein the scaffold sequence comprises SEQ ID NO: 73 or a mutant thereof (e.g., SEQ ID NO: 244);
2) the Cas12f polypeptide comprises SEQ ID NO: 2 or a mutant thereof (e.g., SEQ ID NO: 227), and wherein the scaffold sequence comprises SEQ ID NO: 74 or a mutant thereof (e.g., SEQ ID NO: 257);
3) the Cas12f polypeptide comprises SEQ ID NO: 3 or a mutant thereof, and wherein the scaffold sequence comprises SEQ ID NO: 75 or a mutant thereof;
4) the Cas12f polypeptide comprises SEQ ID NO: 4 or a mutant thereof, and wherein the scaffold sequence comprises SEQ ID NO: 76 or a mutant thereof;
5) the Cas12f polypeptide comprises SEQ ID NO: 5 or a mutant thereof, and wherein the scaffold sequence comprises SEQ ID NO: 77 or a mutant thereof;
6) the Cas12f polypeptide comprises SEQ ID NO: 6 or a mutant thereof, and wherein the scaffold sequence comprises SEQ ID NO: 78 or a mutant thereof;
7) the Cas12f polypeptide comprises SEQ ID NO: 7 or a mutant thereof, and wherein the scaffold sequence comprises SEQ ID NO: 79 or a mutant thereof;
8) the Cas12f polypeptide comprises SEQ ID NO: 8 or a mutant thereof, and wherein the scaffold sequence comprises SEQ ID NO: 80 or a mutant thereof;
9) the Cas12f polypeptide comprises SEQ ID NO: 9 or a mutant thereof, and wherein the scaffold sequence comprises SEQ ID NO: 81 or a mutant thereof;
10) the Cas12f polypeptide comprises SEQ ID NO: 10 or a mutant thereof, and wherein the scaffold sequence comprises SEQ ID NO: 82 or a mutant thereof;
11) the Cas12f polypeptide comprises SEQ ID NO: 11 or a mutant thereof, and wherein the scaffold sequence comprises SEQ ID NO: 83 or a mutant thereof;
12) the Cas12f polypeptide comprises SEQ ID NO: 12 or a mutant thereof, and wherein the scaffold sequence comprises SEQ ID NO: 84 or a mutant thereof;
13) the Cas12f polypeptide comprises SEQ ID NO: 13 or a mutant thereof, and wherein the scaffold sequence comprises SEQ ID NO: 85 or a mutant thereof;
14) the Cas12f polypeptide comprises SEQ ID NO: 14 or a mutant thereof, and wherein the scaffold sequence comprises SEQ ID NO: 86 or a mutant thereof;
15) the Cas12f polypeptide comprises SEQ ID NO: 15 or a mutant thereof, and wherein the scaffold sequence comprises SEQ ID NO: 87 or a mutant thereof;
16) the Cas12f polypeptide comprises SEQ ID NO: 16 or a mutant thereof, and wherein the scaffold sequence comprises SEQ ID NO: 88 or a mutant thereof;
17) the Cas12f polypeptide comprises SEQ ID NO: 17 or a mutant thereof, and wherein the scaffold sequence comprises SEQ ID NO: 89 or a mutant thereof;
18) the Cas12f polypeptide comprises SEQ ID NO: 18 or a mutant thereof, and wherein the scaffold sequence comprises SEQ ID NO: 90 or a mutant thereof;
19) the Cas12f polypeptide comprises SEQ ID NO: 19 or a mutant thereof, and wherein the scaffold sequence comprises SEQ ID NO: 91 or a mutant thereof;
20) the Cas12f polypeptide comprises SEQ ID NO: 20 or a mutant thereof, and wherein the scaffold sequence comprises SEQ ID NO: 92 or a mutant thereof;
21) the Cas12f polypeptide comprises SEQ ID NO: 21 or a mutant thereof, and wherein the scaffold sequence comprises SEQ ID NO: 93 or a mutant thereof;
22) the Cas12f polypeptide comprises SEQ ID NO: 22 or a mutant thereof, and wherein the scaffold sequence comprises SEQ ID NO: 94 or a mutant thereof;
23) the Cas12f polypeptide comprises SEQ ID NO: 23 or a mutant thereof, and wherein the scaffold sequence comprises SEQ ID NO: 95 or a mutant thereof;
24) the Cas12f polypeptide comprises SEQ ID NO: 24 or a mutant thereof, and wherein the scaffold sequence comprises SEQ ID NO:96 or a mutant thereof;
25) the Cas12f polypeptide comprises SEQ ID NO: 25 or a mutant thereof, and wherein the scaffold sequence comprises SEQ ID NO: 97 or a mutant thereof;
26) the Cas12f polypeptide comprises SEQ ID NO: 26 or a mutant thereof, and wherein the scaffold sequence comprises SEQ ID NO: 98 or a mutant thereof;
27) the Cas12f polypeptide comprises SEQ ID NO: 27 or a mutant thereof, and wherein the scaffold sequence comprises SEQ ID NO: 99 or a mutant thereof;
28) the Cas12f polypeptide comprises SEQ ID NO: 28 or a mutant thereof, and wherein the scaffold sequence comprises SEQ ID NO: 100 or a mutant thereof;
29) the Cas12f polypeptide comprises SEQ ID NO: 29 or a mutant thereof, and wherein the scaffold sequence comprises SEQ ID NO: 101 or a mutant thereof;
30) the Cas12f polypeptide comprises SEQ ID NO: 30 or a mutant thereof, and wherein the scaffold sequence comprises SEQ ID NO: 102 or a mutant thereof;
31) the Cas12f polypeptide comprises SEQ ID NO: 31 or a mutant thereof, and wherein the scaffold sequence comprises SEQ ID NO: 103 or a mutant thereof;
32) the Cas12f polypeptide comprises SEQ ID NO: 32 or a mutant thereof, and wherein the scaffold sequence comprises SEQ ID NO: 104 or a mutant thereof;
33) the Cas12f polypeptide comprises SEQ ID NO: 33 or a mutant thereof, and wherein the scaffold sequence comprises SEQ ID NO: 105 or a mutant thereof; and/or
34) the Cas12f polypeptide comprises SEQ ID NO: 34 or a mutant thereof, and wherein the scaffold sequence comprises SEQ ID NO: 106 or a mutant thereof.

36.-68. (canceled)

69. The system of claim 2, wherein the target sequence comprises about or at least about 16 contiguous nucleotides of the target DNA, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or in a numerical range between any two of the preceding values, e.g., from about 16 to about 50, or from about 17 to about 22 contiguous nucleotides of the target DNA; optionally, wherein the target sequence comprises about 20 contiguous nucleotides of the target DNA.

70. The system of claim 2, wherein the reversely complementary sequence of the target sequence is immediately 3′ to a protospacer adjacent motif (PAM); optionally, wherein the PAM is 5′-TTN or 5′-CCN, wherein N is A, T, G, or C.

71. The system of claim 2, wherein the guide sequence is about or at least about 16 nucleotides in length, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any two of the preceding values, e.g., in a length of from about 16 to about 50 nucleotides, or from about 17 to about 22 nucleotides; optionally, wherein the spacer sequence is about 20 nucleotides in length.

72. The system of claim 2, wherein (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence.

73. The system of claim 2, wherein the system comprises two or more guide nuclei acids comprising two or more guide sequences capable of hybridizing to two or more target sequences of the same target DNA or different target DNAs, wherein the two or more guide sequences are the same or different, and wherein the two or more target sequences are the same or different.

74. The system of claim 2, wherein the target DNA is a dsDNA, such as, a eukaryotic dsDNA, e.g., a gene in a eukaryotic cell.

75. A polynucleotide encoding the Cas12f polypeptide of the system of claim 2 and the guide nucleic acid of the system of claim 2.

76. A delivery system comprising (1) the system of claim 2; and (2) a delivery vehicle.

77. A vector comprising the polynucleotide of claim 75; optionally wherein the vector is a plasmid vector, a recombinant AAV (rAAV) vector (vector genome), or a recombinant lentivirus vector.

78. A recombinant AAV particle comprising the rAAV vector genome of claim 77.

79. A ribonucleoprotein (RNP) comprising the Cas12f polypeptide of the system of claim 2 and the guide nucleic acid of the system of claim 2.

80. A lipid nanoparticle (LNP) comprising an RNA (e.g., mRNA) encoding the Cas12f polypeptide of the system of claim 2 and the guide nucleic acid of the system of claim 2.

81. A method for modifying a target DNA, comprising contacting the target DNA with the system of claim 2, wherein the spacer sequence is capable of hybridizing to a target sequence of the target DNA, wherein the target DNA is modified by the complex.

82.-83. (canceled)

84. A cell modified by the method of claim 81.

85.-88. (canceled)

89. The system of claim 27, wherein the gRNA comprises a tracrRNA linked to a crRNA comprising a guide sequence via a short linker, and optionally wherein the short linker is GAAA.

90. The system of claim 27, wherein the scaffold sequence comprises a tracrRNA linked to a repeat sequence via a short linker, and optionally wherein the short linker is GAAA.


SEQUENCE LISTING (515 sequences)

SEQ ID NO: 1 - protein, 433 aa, unidentified
MGKGVLAKVMKYELRYLDGCGDFSNMQEQVWALQRQTREILNRSIQIAFQWDCANSEHHRKTGEYLDLKTETGYKRLDGHIYNCLKGQYEDMATSNLNATIQKAWKKYNSSKKEILRGSMSIPSYKMNQPLTLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQT

SEQ ID NO: 2 - protein, 415 aa, unidentified
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVDLGMKIPAYVTLNDSEYIGKPIGDINDFLKVRKQFKERKERLQKQLAINKGGRGITNKMQLMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNADRNASFNIARSTKYISSKEESDFYKQLK

SEQ ID NO: 3 - protein, 432 aa, unidentified
MSKGSLAKVMKYELRYLDGAGSFEQMQERLWVLQRQTREILNRSTQISFHWDYTSREHFEQTGQYLDVFSETGYKRLDGYIYSRVKDSCGDMASGNINATLQKAWNKYGTSKLDVLRGQMSLPSYKKDQPLVIEKHNIRLSMDGQQALAEITLFSNKFKKENSLSSNVRFAFQLHDGTQRRILNSVLSGEYGLGQCQLVYDRPKWFLLLTYTFTPQNRQLDPDRILGVDLGECYALCASVFGEYGSLRIEGGEVTAYAKKLEARKRSLQKQAAVCGEGRKGHGTKTRVADAYQMQDRIANFRDTVNHRYSKALIDYALKNQCGTIQMEDLSGIRQDTGFPKFLQHWTYYDLQSKIENKAKEHDIRIVKINPRYTSQRCSKCGAIDSGSRTSQARFCCTKCGFTANADYNASQNISIKGIDLLIEKELGAKAE

SEQ ID NO: 4 - protein, 438 aa, unidentified
MGKGEISKVMKYELRYLDGSGSFEEMQQRVWALQRKTREIQNRTVQIAFHWDYINREHFIQTGNNLNVLQETGYKRLDGYIYDRLKGQSAEMSGANLNATIQTAWKKYNSAKPKVLSGTMSVPSFKRDQPLIINSNCVKFSRSESECLAELTLFSREYKKEHDLSSNVRFAIRLHDSTQRSILERVLSGAYRKGQCQLVYQRPKWFLFLTYSFFPMQHDLDPEKYLGVDLGECCALYASSVGEYGSLKLEGGEITAFAKQLEARKRSMQKQAAYCGEGRIGHGTKTRVADVYKMENRIANFRDTVNHRYSKALIDYAVKHQYGTIQMEDLSGIKNDTGFPKFLRHWTYFDLQEKIDAKAREHGIHVVKVNPQYTSQRCSKCGSIDSRNRKSQKEFCCLNCGYKVNADFNASQNLSIKGIDVIIQKYIGAKSKQTENNG

SEQ ID NO: 5 - protein, 428 aa, unidentified
MAKGTVTKVMKYELRYLSGFSDFHAMQQAVWGLQRQSREILNKTIQMAFHWDYISRENFNANGVYLDVKAETGYKTYDGYIYNSLKSAYADMAAANLNAAIQKAWKKYKDAKMEVLRGTMSTPSYRSDQPVLINKNCVKLFDGGVRLTLFSDRFKRENNLNGNLEFAVQLHDGTQRSIFANLLNGTYALGQCQLVYDKRKWFLLVTYIFTPEKHELDPEKILGVDLGQTYALYASSVCARGTFRIEGGEAAECAHRLEQRKRSLQQQARFCGEGRVGHGTKTRVAAVYSAGDKIASYRDSINHRYSKALVEYAVKNGYGTIQMEDLTGIQNDLDHPKRLQHWTYYDLQTKIENKAKEHGVGVVKVNPRYTSQRCSRCGHIERENRPTQKVFCCKACGFEGNADYNASQNLSMRNIDKIIEKELSAKGE

SEQ ID NO: 6 - protein, 430 aa, unidentified
MCALTKIMKYELRYLDGFPDFSAMQNAVWPLQRQTREILNRTIQEAYRWDYFSATKKKETGEYPDLQKETGYKRLDGYIYHVLSPDYPDFSSSGVNATIQKAWKKYKSSKADVWKGEMSLPSYKSDQPIVLHAKQIKLSGDTRAAAATLSLFSNKFKKEHEISGNVQFAITLHDNTQRTIYQKLRNGEYKLSESQLVYDKKKWFLYLAYSFNPAEHALDPEKILGVDMGEKFALYASSFGEYGHFKIEGSEVTEYAKALERRRRSLQQQARYCGEGRIGHGTKTRVGVVYREEDRIANFRSTINHRYSKALIEYAVKNGYGTIQMENLTGIKENLQFPRRLQHWTYYDLQSKIEAKAKEHGIAVVKVNPKHTSQRCSRCGHIAAENRPKQEVFQCVKCGYACNADFNASQNISIKDIEKLIQETIGANPK

SEQ ID NO: 7 - protein, 455 aa, unidentified
MAKKGNSQKKQIVKVMKYELKYEKGCADFNEMQNELWKLQRQTREVMNRTIQLCYHWSYVQAEYCKQHGCARRDVKPCDVYETNATSLDGYIYQLLKVEYPDFFMKNLNATLRKAHQKYDALLFDIQEGNSSIPSFKKDQPLIFEKKAICISKCLPDKRQITLSCFSDSYIDAHPTLDKITFTVRARSASEKSIFDHIISGKYALGTSQLVYEKKKWFFLLSYKFTPESVDVNPEKVLGVDLGVVNALCAGSVENPHDSLFIKGTEAIEQIRRLEARKRDLQKQARYPGDGRIGHGTKTRVSPVYQTRDAIARMQDTLNHRWSRALIDFACKKGYGTIQMEDLSGIKAMESEKPYLKHWTYFDLQSKIIYKAEEKGIRVVKVNPKCTSRRCSACGYISKENRKNQAEFLCVNCGYHHNADYNAAQNLSIPQIDRLIEKQLKEQESEESEAGTNPK

SEQ ID NO: 8 - protein, 437 aa, unidentified
MAEKTIVKVMKFELRYIDGAGEFSEMQKHLWELQKQTREVLNKTIQMGYALECKRFAHHDKTGQWLDDKELTGSKYKAVADYINAELKEDYNIFYSDCRNSTVRKAYKKFKDAKNKIFSGEMSLPSYRSNQPIIIHNRNVIIRGNAESALVGLKVFSDGFKALHGFPAAVNFKLCVKDGTQRAIIENVISEIYKISESQLIYDNKKWFLILAYRFTQKKNDLNPDKILGVDLGVKFAVYASSIGEYGSFRIKGGEVTEFIKRLEKRKKSLQNQATVCGDGRIGHGTKTRVADVYKARDKISNFQDTINHRYSRAIVDYARKNGYGTIQLEKLDNSIEKKGDYSPVLVHWTYYDLRTKMEYKAAEYGIKVIAVEPKYTSQRCSKCGYISSENRKTQESFECIKCGYKCNADFNASQNLSVRDIDRIIDEYLGANPELT

SEQ ID NO: 9 - protein, 433 aa, unidentified
MVCNKVVKIALICDQIDKDGKDVNYNDIYKLLWDLQKQTREAKNKVIRLCWEWSGYSSEYFKTHEEYPKDKEILGISLRSYLYNRIKGDYNLYSGNLSQSAKIAYIEYKNSLTDVLRGDKSIINYRENQPLDIKNKAIQLLYENDNFFVRVALINKDKRKELNFKDCSVRFKLLVKDDSTRTILERCFDEVYTITASKIMYNKKKKQWYINLGYKFTKEIDKTLDKDRILGVDLGVINPLVASVYGSYDRLIIGGGEIDKFRKRVEANKVQMLKQGKYCGDGRIGHGVNTRNKPAYNIEDKISRFRDTVNHKYSKAVVDYAVKNNCGTIQMEDLKGITQNKNERYLKNWTYFDLQTKIEYKAKALGIEVKYKNPKYTSQRCSKCGHIAEENRPEQKTFKCVKCGFKVNADYNASQNLAIKDIDKIIEQYYNKG

SEQ ID NO: 10 - protein, 424 aa, unidentified
MKYTKVMRYQIIKPLNAEWDELGMVLRDIQKETRAALNKTIQLCWEYQGFSADYKQIHGQYPKPKDVLGYTSMHGYAYDRLKNEFSKIASSNLSQTIKRAVDKWNSDLKEILRGDRSIPNFRKDCPIDIVKQSTKIQKCNDGYVLSLGLINREYKNELGRKNGVFDVLIKANDKTQQTILERIINGDYTYTASQIINHKNKWFINLTYQFETKETALDPNNVMGVDLGIVYPVYIAFNNSLHRYHIKGGEIERFRRQVEKRKRELLNQGKYCGDGRKGHGYATRTKSIESISDKIARFRDTCNHKYSRFIVDMALKHNCGIIQMEDLTGISKESTFLKNWTYYDLQQKIEYKAREAGIQVIKIEPQYTSQRCSKCGYIDKENRQEQATFKCIECGFKTNADYNAARNIAIPNIDKIIRKTLKMQ

SEQ ID NO: 11 - protein, 424 aa, unidentified
MATKVMRYQIIKPIDCNWDLFGKVLRDIQYDTRQIMNRTIQYCWEWQGYSSDYKIAKGEYPKTRETFGYSDMRGYAYDKLKSIYQRLNTANLTTSITRAVQRWKTDTKDVIRGDKSIACFRADVPIDLHNKSMNIEKSDDGYIVALSLASNIYKKELDRNSGQFSVLINEGNKSNRDVLDRCIAGQYKISASQILREKNKWFLNLSYSFEISKPDKSRDNILGIDVGIVHPVYMAVYNSPARRSISGGEIDNFRKQVQKRIKELQLQGKQCGEGRIGHGIKTRVKPIEFAKDKVANFRNTINHKYSKAIVEFAIKNGCGIIQMEDLKGINTDNVFLKNWTYYDLQQKVKYKAELEGIEVKLIDPQYTSQRCCKCGYIHRDNRPEQAKFKCIDCGFEVNADYNASLNIATPDIDKIILEFLKCET

SEQ ID NO: 12 - protein, 451 aa, unidentified
MGVTIKIMKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 13 - protein, 450 aa, unidentified
MSTVVKVMKYQIICPVNIEWKAFETYLRTLSYQVRTIGNRTIQKLWDFDNQSLNHFRENGVYPSAQQLYGCTQKTISGYIYDQLKEEYQDMNKANMSTTLQKTIKTWNSRKKEIRSGEMSIPSFRNNLPIDIHGNSIQITKEKSGDYIASLSLFSSNFIIENNLPNGKIQVKLSTRKQNSMKVILDRIIENTYAKGACMLHKHKNKWYLSIIYKPTVKEEHKFEEDLVMGIDMGKINVLYFAFNKGWIRGAISGEEIEAFRKKIEHRRISLLRQGKYCSGNRVGKGREKRIKPIDVLNNKIAKFRNATNHKYANYIVQQCLKYNCGTIQLENLQGISKEQTFLKNWTYFDLQEKIKQQAHQYGMKVVTIDPSYTSKRCSECGYIHKNNRKSQSTFECQQCNLKVHADYNAAKNISIYNIEKVIQKQLKLQEKLNSKKFTEQYIEQVENIN

SEQ ID NO: 14 - protein, 450 aa, unidentified
MSIAVKVMKYQIVCPVNIEWKTFEIYLRTLSYHFRTIGNRTIQKLWEYDNQSLKHFKDTGQYPSAQQLYGCTQKTISGYIYDQLKEEYQDINKANMSTTLQKTIKTWNSRKKEIWSGEMSIPSFRNNLPIDIHGNSIQIIKEKSGDYIASVSLFSSKFIKENDLPNGKILVKLSTRKQNSMKVILDRIIDSTYAKGACMLHKHKKKWYLSITYKSNIKEELKFDEDLIMGIDMGKINVLYFAFNKGLVRGAISGEEIEAFRKKIEHRRISLLRQGKYCSGNRIGKGRKKRIKPIEVLNDKIAKFRTATNHKYANYIVQQCLKYNCGTIQLEDLQGISKEQTFLKNWTYFDLQEKIKNQANQYGIKVVKIDPSYTSQRCSECGYIHKNNRQNQSTFECQQCSFKVHADYNAAKNISVYNIEKVIQRQLKLQEKLNLTKYKEQYIEQMENIN

SEQ ID NO: 15 - protein, 453 aa, unidentified
MRALENQKPLKSIKKPVCKISRTLSVPIQRPCGYVWNDFGHLLCIIRNDVAQAYNMAMSESYLYFSERENYKREHGKYPKVEQLAKRNVYKKLTENFPHIGTGILATIANKVESKLKKEYVEVMLKGTKSVSNYKKGTPIPIRAQGWKERTFKRKRKDKMTFHLLSKKAEQSKSLDFLKDEKGKIPCSFTVRIALKKLNNSQRAVYNRIWAGEYKAGAIDILQRKGKWFINISYHMLETKRLEKQLDKNVIVGVDLGIVNGVVCAVSNDAYDRLVLRKDIEGFRKQIWKRKHLAWKSTRRGGKGRKYYLRMSDSLKKKEHNFRNTLYHDWTRKVIDYALKHGAKVIQIEDLSGLVEAKKKMKKGVLKNWVISDFVEKLTYKAEEYGIEIVKVNPRYTSQRCHKCGHIEKDNRKEQSKFVCLKCGHSCNADFNAAKNIATKNIADIISASLPQT

SEQ ID NO: 16 - protein, 490 aa, unidentified
MTDEQARLQKVATFQIVKPVNMDWREFRKLLRDVRYRLWRLGNMAVSEAYLTFHKKYRMGQAQSDGAHKLSVLDKRLRQALIDEGVRVEELSRYSRKGAVSGYICGAFEKTKLSAIKSKSKWRDIINGRASLPVFRRDLAIPINCSDCQPRMIERTEAGEYQVDLRICLQDKELAPNGYPRVLLSTAKISDGQRAVLERLVSNKTNSLPGYRHRFFEIKEKRGKWFLSVSYDFPRAEAGKLHQDIIVGVDLGWSVPLYAALNKGYARIGWKKLEPLAKRIRHLQKQVKGRRLSMQRGGQADLAGPTARMGHGRRRNLQAIEKLEGKINDAYTTLNHQLSHCIIEFAKNNGAGVIQIEDLRGLADELRATFIGQNWRYHQLQEFIKYKAEEAGIKVVPPVNPFYTSRRCSVCGYLHKDFTFEYRQVNRKNGMSVMFECPECSKKAKEEGKEYKALNADYNAARNLATANIEEKIRLQCKEQGIEYTELPKS

SEQ ID NO: 17 - protein, 478 aa, unidentified
MKDYIRKTLSLRILRPYYGEEIEKEIAAAKKKSQAEGGDGALDNKFWDRLKAEHPEIISSREFYDLLDAIQRETTLYYNRAISKLYHSLIVEREQVSTAKALSAGPYHEFREKFNAYISLGLREKIQSNFRRKELARYQVALPTAKSDTFPIPIYKGFDKNGKGGFKVREIENGDFVIDLPLMAYHRVGGKAGREYIELDRPPAVLNVPVILSTSRRRANKTWFRDEGTDAEIRRVMAGEYKVSWVEILQRKRFGKPYGGWYVNFTIKYQPRDYGLDPKVKGGIDIGLSSPLVCAVTNSLARLTIRDNDLVAFNRKAMARRRTLLRQNRYKRSGHGSANKLKPIEALTEKNELYRKAIMRRWAREAADFFRQHRAATVNMEDLTGIKDREDYFSQMLRCYWNYSQLQTMLENKLKEYGIAVKYIEPKDTSKTCHSCGHVNEYFDFNYRSAHKFPMFKCEKCGVECGADYNAARNIAQA

SEQ ID NO: 18 - protein, 500 aa, unidentified
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 19 - protein, 493 aa, unidentified
MKTTEKNVLMTKCIKVTLNRCVNYNMKEIMNIIREMQYLSSKAYNLATNYLYIWDTNSMNFKNLYEEKIVDKDLLGKSKSAWIENRMNEIMKGFLTNNVAQARQDVINKYNKSKKDGLFIGKVTLPSYKMNGKVVIHNKAYRFSKNEGYFVEIGLFNKEKKEELNCDWIKFKLDKIDSNKKATIYKILNGDYKQGSAQLHINKKGKIEFIISYSFERENSIKLDKNRTLGIDIGIVNIAAMAIWDNNKQEWELTRYSHNLISGNEAIALRQKYYKLGLRNKELEKNINRELHELEEKEYRGLSTNIISGHNLTYKRIMLNSKRIRLSQSCKWCGNSKVGHGRRVRCKQVDKIGNKIERFKDTFNHKYSRYIVDFAVKNNCGIIQMENLKNFNPSEKFLKDWPYFDLQTKIEYKAKEYGIEVIKVNPKYTSKRCSRCGCINELNRDCKKNQSKFKCVNDECNNYENADINAAKNIALPYIDKIIEQCLETNKVV

SEQ ID NO: 20 - protein, 450 aa, unidentified
MKLNKCIKVTLVKCLNYDYKEIKQIIRDFNYTACKASNKAMRMWFFHTQDMIDKKNKYKEFNQIQYEKDTYGKSYRNVIEGEMKKIMPLANTSNVGTLHQQLVQNDWSRLKKDILSCKANLPTYKLSTPYFIKNDNFKLRNHNGYFVDIAFFNKEGLKQYGYKAGHKFEFQIDKLDGNKKSTINKIINGEYKQGSAQLSISNKGKIELIISYSFEKEEVPVLDKNKILGIDLGITNVATMSVYDSMREQYDYFSWKTNVISGKELIAFRQKYYNLRRDMSIASKTAGQGRCGHGYKTKMKSVNKVRNKIANFADTYNHKISKYIIEFAIKNNCGVIQVEDLSGATADTHNKMLKDWSYYDLQQKIEYKAKEQGIEVIKVNPKYTSKRCSKCGCIHEDNRDCRNNQAKFECKVCGYNENADINASKNIAIPDIDNIIKGTEILHSKENKAS

SEQ ID NO: 21 - protein, 437 aa, unidentified
MTTKCVQVAIEYSSNNILKEVDFYKELRDLQYNSYLACNRAISYMYENDMQNFIIKETDLPRSDDKKLYGKSFAAWIENRMNEYMPGALSNNVAQTRQFVVNRYKNDKKAGLLKGNVSLTTFKRTNPIIIHNNAYNIIETPKGLGAEIGFFNLPKQKELGIKRVNFLFPKLGSSEKSIIRRLLDKSYKQGAMQISYNQKKKKWMATISFSFNLEEIKTNENLVMGIDLGVSKVATLSIYDASKYEYIKMSFKDTCIDGTELMHYRQKLESRRKALSIASKWASDNNRGHGYKTKMEKANYMGRKYNNFRDTYNHKVSRYIVDVAIKYRVGLIQMEDLSGFSEQQQESLLKNWSYYDLQQKIKYKAEENGIRVYFINPKYTSQRCSKCGNIDKENRKTQESFSCTVCNYKDNADVNASKNIAIPDIEKIIEEQVKKQY

SEQ ID NO: 22 - protein, 471 aa, unidentified
MITTRKLKLAIVSDNKNEAYSFIRDETRNQNRALNVAYSHLYFEYIAQEKLKHSDAEYQEHLAKYQELASKKYQEFLKVKEKAKSDETLQAKVDKAREAYNKAQEKVYKIEKDYSKKAREIYQQSVGLAKQTRIDKLLKNQFNLHYDTVDRVGGTAISHFTNDMKSGVLQGKRSLRNYKSSNPLMIRARSMKVYEENSDYYIKWIKDITFKIIISAGSKQRQNIGELKSVLVNIIEGNYKACDSSIGVDKDLILNLSIDIPITKENIFIPNRVVGVDLGLKIPAYVSVNDTPYIKRAIGNINDFLKVRTQLQSQRRRLQKALQSTNGGKGRNKKMQGLERLQAKEKNFVNTYNHFLSKNIVDFAVKNNAGMIHMEELKFDKVKHKSLLRNWSYYQLQTMIEYKAKREGIEVYYVDASYTSQTCSKCGNLEEGQRETQDTFVCKKCGYSVNADYNASQNIAKAKTIKEENQQ

SEQ ID NO: 23 - protein, 496 aa, unidentified
MILTRKIKLVIVSENREEGYNLIRTEIREQHKALNLAYNHLYFEHNAIQKLKQNDEDYKQKRNKLQELINKKYEEHQKAKNLEKKEALREAYNNKKQELYNFEKEYNEKARQTYQQVVGFTQQTRVRNLINRECNLMSDTKDGITSKVTQDYKNDCKAGLLIGKRSLRNYKKDNPLLVRGRSLKFYKEDGDYFIKWNKGTIFKCILHIRKKNVVELQSVLENVLLGAYKVCDSSIGFNNKDMILNLSLNIPDKETQGYIPGRVVGVDLGLKIPAYLSLSDKVYVRKGIGSIDDFLRVRTQMQKRRRRLQKSLAAVKGGKGREKKLKALDHLKGKEANFAKTYNHFLSTQIVTFAVKNQAGQINMEFLEFDKMKNKSLLRNWSYYQLQIMVEYKAKREGIIIKYVDAYLTSQTCSKCDHYEDGQREKQENFMCKNCGLEVNADYNASQNIAKSTSYISDSTESEYHKKKQQVLKEILGENDIMNEQLSLFNNCDDIA

SEQ ID NO: 24 - protein, 461 aa, unidentified
MISTRKIKVRCDDSTFYTFFRQEQREQNKALNIGIGIIHANAVLHNVDSGAEKKLKKSIEGLQGKIDKLNKDLEKEKITDKKKEEVLKAIETNKKILDGEKKVFKESEEYRKGIDELFKNTYLKSNTLDHVLDSMVNIQYKRTLSLVTQRIKKDYSNDFVGIITGQQSLRNYRNDNPLMISNQQLNFKYIDDTFYLDIMCGYRLEVVLGKRDNENVNELKSTLEKVISKEYKVCDSSMQFSKNNKDVILNLVIDIPQNSNVYKPVEGRILGVDLGVAVPIYMCLNDDTYKRKGLGDINNFLRVRQQMQTRRRKLQKDLTLTNGGKGRKKKTQLLDKLQENERNFVKTYSHALSKRVVEFAKSNKCEYINIEKLTKDGFDNIILRNWSYFELQKMIEYKAEREGITVRYVNPAYTSQKCSRCGEIDKENRQTQANFKCTKCGFELNADHNAAINIARSIEFV

SEQ ID NO: 25 - protein, 482 aa, unidentified
MNTVRKIKLTILGDTETRNKQYKWIKDEQYNQYRALNLSMTYMVTNLMLKNNESGLENRKEKDILKIENKIKKDEGSLKKELAKKKINEEKIENIKSNIEELKSEKEKLENELKNIKEYRSNIDEEFKKMYVDDLYNVLNKISFQHEDMKSLVTQRVKKDFNNDVKEIMRGDRSVRNYKRNFPILTRGRDLKFQYIEKSEDIEIKWIEGIKFKCILGKPSKSLELKHALHKVINKEYKVCDSSLQFDKNNNLILNLTLDIPQDNKYEKITNRVVGVDLGLKIPAYVALNDTKYIRKAIGSIDDFLKVRTQMQSRVRKLQKSLQVVRGGKGRNKKMKALERFREKERNFARNYNHFLSYNIVKFALDNKAEQINLELLEMKKTQNKSILRNWSYYQLQNFIEYKAERVGIKVKYIDPYHTSQTCSECGNYEEGQRVEQDTFVCKRCWHKMNADYNAARNIAMSYNYISKKEESEYYKNNKNMV

SEQ ID NO: 26 - protein, 404 aa, unidentified
MNTVRKIKIIINNENNELRKEQYKFIRDSQYAQYQGLNRCMGYLMSGFYVNNMDIKSEEFKTWQKGVTNSANFFQEISFGKGIDSKSSITQKVKKDFSIALKNGLAKGERNINNYKRIAPLMTRGRNLKFKYDDNELDILINWVNKIQFKCVLGEHKNSLELQHTLHKVINNEYKIGQSSLYFNKKNELILILTIDIPTAKSSYEPIKDRILGVDLGMAVPVYMSINDNSYIKKSLGSYSEFAKVRKQFKERRNRLYKQLEACKGGRGRKDKLKAMNQFKEKEKNFAKTYNHFLSKNIVEFALKNKCEFIHLEKIESKGLENSVLANWTYYDLQEKIIYKAKREGIGIKFVNSSYTSQTCSKCNYVDKENRKTQAKFICKNCGFKANADYNASQNISKSKEFIK

SEQ ID NO: 27 - protein, 423 aa, unidentified
MNIVKKIKLRIIDNDKELCKKQYLGFTEEQKKELIDKQYKFIRDSQYQQYLGFNRAMGFLMSGYYANNMDIKSDNFKEHQKKLTNSLYIFDDIKFGVGIDSKSLIVQRVKKDFSTALKNGLAKGERSVTNYKRTYPLLTRHRSIKFLYAENELDIYLDWVNKIRFRCELGNHKNSLELQHTLRKVITGEYKISDSSLEFNKKNELILNLNLNIPETKATFIKDRTLGVDLGMAIPAYVSLSDTPYIRKGFGSYEEFAKVRNQFKDRRKRLLKQLSLVAGGKGRAKKLHSMEFLKNKEKQFAKTYNHSLSKKIIDFALKNNCEYINLEDIKSTSLEDRVLGQWGYYQLQEQIEYKAKLVGIKVRKVKAAYTSQTCSECGNIDKENRKNQSTFKCTNEDCKLNKKGINADWNASINIARSKEFIK

SEQ ID NO: 28 - protein, 400 aa, unidentified
MITVRKVKLIVNSEEAEEINRTYKFIRDSMYAQYQGLNRCMGYLLSGYYANGMDIKSDGFKNHMKTIKNSLNIFDDINFGIGIDSKSAITQKVKKDFSTSLKNGLAKGERGATNYKRNFPLMTRGRDVKISYLEDTNTFVIKWVNKIEFKVILGQKDNIELSHTLHKIINKEYTLGQCTFEFDKNNKLLLALNINIPDNLISKNKEIIPGRVLGVDLGVKVPAMICLNDNTFIKKSIGSYNEFFKVRSQFKARRERLYKQLESSNGGKGRKHKLKATMQFRDKEKNFARTYNHFLSKNIIEFAQKYTCETINLEELNKKGFDNNLLGKWGYYQLQSMIEYKAERVGIKVKYVDPAFTSQTCSKCGYVDEENRITQDKFECQKCGFTLNADHNAAINIARK

SEQ ID NO: 29 - protein, 401 aa, unidentified
MIAVKKLKLTIVEEEEKRKEQYKFIRDSQYAQYQGLNLAMGILTSAYLVSGRDIKSDLFKDSQKSLTNSNEIFNGINFGKGIDTKSSITQKVKKDFSTSLKNGLAKGERGFTNYKRDFPLMTRGRDLKFYEEDKEFYIKWVNKIVFKILIGRKDKNKVELIHTLNKVLNKEYKVSQSSLQFDKNNKLILNLTIDIPYKKVDEIVKDRVCGVDMGIAIPIYVALNDVSYVREGMGTIDEFMKQRLQFQSRRRRLQQQLKNVNGGKGRKDKLKGLESLREKEKSWVKTYNHALSKRVVEFAKKNKCEYIHLEKLTKDGFGDRLLRNWSYYELQEMIKYKADRVGIKVKHVNPAYTSQTCSECGHADKENRETQAKFKCLECGFEANADYNAARNIAKSDKFVK

SEQ ID NO: 30 - protein, 398 aa, unidentified
MIAVRKLKIMVLCDDESKKNEQYKFLRDSQYAQYLGLNRAMSFLAKEYLSGDKERFKEAKKKLTNTCECYQNINFGTGIDSKSQITQKVKKDLQADIKNGLARGERSIRNYRRTFPLITRGRDLKFSYNGDEIIIKWVNKIYFKVLIGRKDKNYLELMHTLEKIINGEYKVCTSSIQIDKKLILNLTLEIPDKVKKEFQENRVLGVDLGIKFPAYACVSDNTYVRRSFGSIDEFLKVRIQFDKRRKRIQQQLQNVKGGKGRKDKLQALDRMRDCERKWVRNYNHALSKRIIDFAFRNKCGIIHLEKLEKDGFKNKLLRNWSYYELQDMIGYKAEREGIVVKYVEPAYTSQTCSKCGYVDRENRPSQEHFLCKECGFEINADHNAAINIARSNKVIVDK

SEQ ID NO: 31 - protein, 402 aa, unidentified
MITVRKLKLTIINDDETKRNEQYKFIRDSQYAQYQGLNLAMSVLTNAYLSSNRDIKSDLFKETQKNLKNSSHIFDDITFGKGTDNKSLINQKVKKDFNSAIKNGLARGERNITNYKRTFPLMTRGTALKFSYKDDCSDEIIIKWVNKIVFKVVIGRKDKNYLELMHTLNKVINGEYKVGQSSIYFDKSNKLILNLTLYIPEKKDDDAINGRTLGVDLGIKYPAYVCLNDDTFIRQHIGESLELSKQREQFRNRRKRLQQQLKNVKGGKGREKKLAALDKVAVCERNFVKTYNHTISKRIIDFAKKNKCEFINLEQLTKDGFDNIILSNWSYYELQNMIKYKADREGIKVRYVNPAYTSQKCSKCGYIDKENRPTQEKFKCIKCGFELNADHNAAINISRLEE

SEQ ID NO: 32 - protein, 467 aa, unidentified
MITVRKLKLTIVGDEQTRKEQYKIIRDEQYQQYKALNLCMTLLNTHNILNSYNTGSENKLNSQIEKLDNKIEKNKIELKKGNLKESKIEKLNKSILELTKEKEKLQQEYLSASKYRSDIDEKLKDMYIKDMYTVVQSQVNFKSKDMMSLVTQRAKKDFSNALKNGMARGERSLINYKRDFPLMTRGERWLKFKYNEESDDIYIDWLHDIKFKVILGYKKNENSIELRHTLHKVINKEYKICDSSMQFDRNNNLILNLTLDIPNKESKGYVEGRTLGVDLGIKYPAYVCLSDDTYKRKSIGCAEDFIRVREQIRGRRYRLQKQLSMVKGGKGRDKKLRALDRVREAERNFVKTYNHMISKNIIKFAKEHNCEYIHLEKLTKDGFPDIILSKWSYYELQNMIEYKSDREGIKVRYIDPAYTSQTCSKCGHIDKENRINQEKFKCVKCGFELNADHNASINISRSNKYLK

SEQ ID NO: 33 - protein, 464 aa, unidentified
MKTTRKLKLTIIGDEETRKEQYKIIREEQYQQYKALNLCMTLLNTHNILNSYNTGAENKLNAQIDSIDKKIEQAKKELEKKGLKESKVSKLKETIEFLENDREKLKDEYLNSSKFRSDIDEKMKEMYIKDMYTVVQNQVNFRARDMMSLVTQRARKDFKNSLKNGMAKGERSLTNYKRDFPLMTRGERWLKFEYDKDSDDILINWIHGIKFKVLLGYKKNENSIELRHTLHKVINKEYKICDSSMQFDRNNNLILNLTLDIPDKQNNNYIEKRTLGVDLGIKYPAYVCLNDDTYIRSHIGESLELLKQREQFKDRRKRLQQQLKNVKGGKGRNKKLSALNKLSDNERNFARTYNHMISKRIVEFAKKHRCEFINLEKLTKDGFDNNILSNWSYYELQNMIEYKAKREGIEVRYIDPAYTSQKCSRCGYIDKENRQTQEKFKCLKCEFEINADHNAAINIARALD

SEQ ID NO: 34 - protein, 465 aa, unidentified
MSEQEAAQEGTKLLAKTLTFGLGNPMGFKSKGSVLVELTEDQRKAIYNGLRDASTVVARIINLLNSREYIRQIMKVPEELVAQFKPNYSLVKGPLKRLGIEEAEQVAGSVLSQTFALGVKPDFQGEHGKGLLLKGERQIPLHRTDGTHPIPQRATETRLFQVEKNFYVAMQVFAETWAKKQELPSGWLAFPIKVKPRDKTMAGQLLKTIGGEWKLKNSRLMRNPRTGGNRWLGQIVVAFAPEPFKKMTRSVVMGIDLGVNVPACLHISENGKPLPWAMMVGRGRDMLNTRNLIRSEIVHIIKALKSKDSPLDGKARAIYRDKLRDLRKRERRVMKMASQTVAARIADTAKRHGAGTWQMEDLSPDIKTDQPWLARNWAPGMLLDAVRWQARQCGAELVMVNPAYTSQRCARCGHIDPQNRPKQTDFKCMACGHEDNADKNAARNLSVVGIEKLIADFKAPNGAVQ

SEQ ID NO: 35 - protein, 497 aa, unidentified
MITVRKIKLTIMGDKDTRNSQYKWIRDEQYNQYRALNMGMTYLAVNDILYMNESGLEIRTIKDLKDCEKDIDKNKKEIEKLTARLEKEQNKKNSSSEKLDEIKYKISLVENKIEDYKLKIVELNKILEETQKERMDIQKEFKEKYVDDLYQVLDKIPFKHLDNKSLVTQRIKADIKSDKSNGLLKGERSIRNYKRNFPLMTRGRDLKFKYDDNDDIEIKWMEGIKFKVILGNRIKNSLELRHTLHKVIEGKYKICDSSLQFDKNNNLILNLTLDIPIDIVNKKVSGRVVGVDLGLKIPAYCALNDVEYIKKSIGRIDDFLKVRTQMQSRRRRLQIAIQSAKGGKGRVNKLQALERFAEKEKNFAKTYNHFLSSNIVKFAVSNQAEQINMELLSLKETQNKSILRNWSYYQLQTMIEYKAQREGIKVKYIDPYHTSQTCSKCGNYEEGQRESQADFICKKCGYKVNADYNAARNIAMSNKYITKKEESKYYKIKESMV

SEQ ID NO: 36 - protein, 529 aa, unidentified
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 37 - protein, 497 aa, unidentified
MGESVKAIKLKILDMFLDPECTKQDDNWRKDLSTMSRFCAEAGNMCLRDLYNYFSMPKEDRISSKDLYNAMYHKTKLLHPELPGKVANQIVNHAKDVWKRNAKLIYRNQISMPTYKITTAPIRLQNNIYKLIKNKNKYIIDVQLYSKEYSKDSGKGTHRYFLVAVRDSSTRMIFDRIMSKDHIDSSKSYTQGQLQIKKDHQGKWYCIIPYTFPTHETVLDPDKVMGVDLGVAKAVYWAFNSSYKRGCIDGGEIEHFRKMIRARRVSIQNQIKHSGDARKGHGRKRALKPIETLSEKEKNFRDTINHRYANRIVEAAIKQGCGTIQIENLEGIADTTGSKFLKNWPYYDLQTKIVNKAKEHGITVVAINPQYTSQRCSMCGYIEKTNRSSQAVFECKQCGYGSRTICINCRHVQVSGDVCEECGGIVKKENVNADYNAAKNISTPYIDQIIMEKCLELGIPYRSITCKECGHIQASGNTCEVCGSTNILKPKKIRKAK

SEQ ID NO: 38 - protein, 422 aa, unidentified
MIKVYRYEIVKPLDLDWKEFGTILRQLQQETRFALNKATQLAWEWMGFSSDYKDNHGEYPKSKDILGYTNVHGYAYHTIKTKAYRLNSGNLSQTIKRATDRFKAYQKEILRGDMSIPSYKRDIPLDLIKENISVNRMNHGDYIASLSLLSNPAKQEMNVKRKISVIIIVRGAGKTIMDRILSGEYQVSASQIIHDDRKNKWYLNISYDFEPQTRVLDLNKIMGIDLGVAVAVYMAFQHTPARYKLEGGEIENFRRQVESRRISMLRQGKYAGGARGGHGRDKRIKPIEQLRDKIANFRDTTNHRYSRYIVDMAIKEGCGTIQMEDLTNIRDIGSRFLQNWTYYDLQQKIIYKAEEAGIKVIKIDPQYTSQRCSECGNIDSGNRIGQAIFKCRACGYEANADYNAARNIAIPNIDKIIAESIK

SEQ ID NO: 39 - DNA, 1299 bp, synthetic construct
  source 1..1299: mol_type = other DNA
atgggaaagggcgtgctggccaaggtgatgaaatacgagctgagatacctggatggttgtggcgacttcagcaatatgcaggagcaggtgtgggccctgcagcggcagacacgggaaatcctgaacagatccatccaaatcgccttccaatgggactgcgccaacagcgagcaccacagaaagaccggcgagtacctggacctgaaaacggaaaccggctacaagagactggatggccacatctacaactgcctgaagggccagtacgaggacatggccacatctaacctgaacgccaccatccagaaggcttggaagaagtataactccagcaagaaggaaatcctgaggggcagcatgagcatccccagctacaagatgaaccagcctctgacactggacaagaataccgtgaaactgtctgagggcgagcggaacccaatcgtgaccctgacactgtttagcgacaagttcaagcgggcccagggcgtgtccaacgtgaagtttagcatgcctctgcacgacggcacccagagagccatcttcgccaacctgatgaacggcacctaccagctgggagagtgccagctggtgtacaaacggcctaagtggttcctgttcgtgacatacaagttcccccccgtggaacatcctctcgatcctgacaagattctgggcgtcgacatgggcgaggcctgcgcgctttatgcctctacattcggcgagcacggctacctgaagatcgatggaggcgagattacaaagtacgccaagaagatggaagctagaatccggagcatgcagaagcaggctgctcactgtggcgaaggcagaatcgggcacggcaccaaaacaagagtgtctgtggtgtaccaggccaaggacaaggtggccagattcagagataccatcaaccacagatactctaaggccctgatcgactacgccctgaagaaccagtgtggcaccatccagatggaagatctgaccggcatcaaggaagatacaggatttccaaagttcctgagacattggacctactacgacctgcagagcaagatcgaggctaaggccgccgagcacggcatccaagttgtcaagatcaaccctagacacaccagccagcgctgcagcagatgtggacacatcgacaaagccaatagaaccagccaagctgatttctgctgcaccaagtgcggcttcagcgccaatgccgactttaatgccagccagaacatcagcatcagaaacatcgacaagattatcgccaaggctatcggcgccaaccggaagcagacc

SEQ ID NO: 40 - DNA, 1245 bp, synthetic construct
  source 1..1245: mol_type = other DNA
atgattacagtgcggaagctgaagattctgatcgacggcgagagcaggaacgagagctacaagttcatccgggacagcatgtacgcccagtacctggccctgaacaaggccatgagctacctgggtaccgcctacctgagcagagataaggagatcttcaaggaggccatcaagtctctcaacaactctaatcctatcttcgacaacattaacttcggcaagggaatcgataccaagagcagcgtgaaccagacagtgaagaaacacatccaggccgacattaagaatggcctggccaagggcgaaagatccatccggaactacaagcgtgactaccccctgatgaccagaggcagagatctgaagtttttctactgcgacaccaatagcaccaaggtgaaggtgaaatgggtgaacggcatcatctttgacgtgatgctgggaaaggaatacaacaagaatgatctagagctgagatcttttctgaacagagtcatcaacaaggaatataagatctcccaaagcagcatctgcttcgacaagcacaacagactgatcctgaatctgagcgtgaacatcaccgacaacatccccaacgaggtggtgaagggcagaattgtgggcgtcgacctgggcatgaaaatcccagcttatgtgacactgaacgacagcgagtacatcggaaaacctatcggcgacatcaacgacttcctgaaggtgcggaagcagttcaaagaaagaaaggagcggctgcagaagcagctggccatcaacaagggcggcagaggcatcaccaacaagatgcagctgatggacgccttcaccaacaaggaaaagaacttcgctaatacctacaaccacggcgtttctaaggcaatcatcaactttgctaagaagtacaaggccgagcagatcaacgtggagttcctggctctggccggcagcgagaaggaaatcctgagctccacaatccgctactggtcctactatcaactgcaacagatgatcgagtacaaggccaaccgggaaggcatcgccgtgaagtacgtggacccttacctgacctcacagacctgctgcaagtgcggcaactacgaggtgggacagagaatcaaccaggagctgttcgagtgtaaactgtgtggcaataaaatgaatgccgatagaaacgccagcttcaacatcgccagaagcacaaagtacatcagctctaaagaggaaagcgacttctacaaacagctcaaa

SEQ ID NO: 41 - DNA, 1296 bp, synthetic construct
  source 1..1296: mol_type = other DNA
atgagcaagggcagccttgctaaggtgatgaaatacgagctgagatacctggatggcgccggctccttcgagcagatgcaggagagactgtgggtgctgcagaggcagacgcgggaaatcctgaacagaagcacccaaatcagctttcactgggactacacctctagagagcatttcgaacagaccggccagtacctggacgtgtttagcgagacaggctacaagagactggatggatatatctactcccgggtcaaggacagctgcggcgacatggccagcggcaacatcaacgccacactgcagaaggcttggaacaagtacggcacatctaaactggacgtgctgcggggccagatgtccctgccatcttataagaaggaccagcctctggtgatcgaaaagcacaacatcagactgagcatggacggccagcaggccctggctgagatcaccctgttcagcaacaaattcaagaaggaaaacagcctgtccagcaacgtgcggttcgcctttcagctgcacgacggcacccagcgcagaatcctgaacagcgtgctgtctggcgagtacggcctgggccaatgtcagctggtttacgacagacctaagtggttcctgctgctgacctacaccttcaccccccagaatagacagctggaccctgatagaatcctgggcgtggacctcggagagtgctacgccctctgtgcctctgtgttcggcgaatacggctctctgagaattgagggcggagaggtgaccgcctacgccaagaagctggaagcccggaagcggagcctgcagaaacaggccgctgtgtgcggcgagggaagaaagggacacggcaccaagacaagagtggccgacgcctaccagatgcaggaccggatcgccaattttcgggacaccgtcaaccacagatactccaaagccctgatcgactacgccctgaagaaccagtgcggcacaatccagatggaagatctgagcggcatccggcaagataccggattccccaagttcctgcaacactggacctactacgacctgcaaagcaagatcgagaacaaggccaaggaacacgacatcagaatcgtgaagatcaatcctagatataccagccagcggtgcagcaaatgtggcgccatcgatagcggcagcagaacatcgcaggctagattctgctgcaccaagtgcggcttcacagccaatgccgattacaacgctagccagaacatcagcattaagggcatcgacctgctgatcgagaaggaactgggagccaaggccgag

SEQ ID NO: 42 - DNA, 1314 bp, synthetic construct
  source 1..1314: mol_type = other DNA
atgggcaagggcgagatctctaaagtgatgaagtacgagctgagatacctggacggctctggcagcttcgaggaaatgcagcagagagtgtgggccctccagcgcaagaccagagagatccaaaacagaacagtgcagatcgccttccactgggactacatcaacagagaacatttcatccagaccggaaacaacctgaatgtgctgcaagaaaccggctacaaaaggctggacggctatatctatgatagactgaaaggacagagcgccgagatgagcggagctaatctgaatgctacaatccagacagcttggaagaagtacaacagcgctaaacctaaggtgctgagcggcaccatgagcgttccttcctttaagagagatcagcctctgatcatcaacagcaactgcgtgaaattctctagatctgagagcgagtgcctggccgagctgaccctgttcagccgggaatacaagaaagagcacgacctgtccagcaacgtgcggttcgccatcaggctgcacgatagcacccagcggagcattctggaaagagtcctgagtggcgcctacagaaagggccagtgccagctggtgtaccagcgccccaagtggttcctgtttctgacctactcttttttcccaatgcagcacgacctggatcctgagaaatatctgggcgtggacctcggcgaatgttgcgccctctacgccagctctgtgggcgagtacggaagcctgaagctggaaggcggggagatcaccgcctttgccaagcagctggaggccagaaagcggtccatgcagaaacaggccgcttactgcggcgagggcagaatcggacacggcacaaagacaagagtggccgatgtgtacaagatggaaaacagaatcgccaacttccgggacactgtgaaccacagatacagcaaggccctgatcgactacgccgtgaagcaccagtacggcacaatccagatggaagatctgagcggtattaagaacgacaccggcttccccaagttcctgcggcactggacctacttcgacctgcaggagaagatcgacgccaaggccagagagcacggcatccacgtggtgaaggtcaatcctcagtacaccagccagcggtgcagcaagtgcggcagcatcgacagcagaaaccggaagtcccagaaggaattctgctgtctgaactgtggctacaaggtgaacgccgacttcaacgcttcccaaaatctgagcatcaagggcatcgacgtgatcatccaaaagtacattggagccaagagcaagcagaccgagaacaacggc

SEQ ID NO: 43 - DNA, 1284 bp, synthetic construct
  source 1..1284: mol_type = other DNA
atggccaaaggcaccgtgaccaaggtgatgaaatacgagctgagatacctgtccggcttcagcgacttccacgccatgcagcaggccgtgtggggcctgcagagacagtctagagagattctgaacaagacaatccaaatggcctttcactgggattacatcagcagagaaaatttcaacgccaacggcgtgtacctggatgtgaaggccgaaaccggctacaagacctacgacggctacatctacaacagcctgaagagcgcctacgccgatatggccgctgcaaacctcaacgccgccatccagaaggcctggaagaagtacaaggacgccaagatggaagtcctgagaggcaccatgtcaacacctagctatagatccgatcagcctgtgctgatcaacaagaactgcgtgaagctgttcgacggcggagtgcggctgaccctgttctctgatagattcaagagagagaacaacctgaatggcaatttggagttcgccgtgcagctgcacgacggcacccagcggagcatcttcgccaatcttctgaacggcacctacgctctgggccagtgccagctggtgtatgacaagcggaagtggttcctgctggtgacctacatcttcaccccagaaaagcacgagctggaccctgagaagatcctgggcgtggacctgggccagacatatgctctgtacgccagcagcgtgtgcgccagaggcacattcagaatcgagggaggtgaggccgctgagtgcgcccacagactggaacagcgcaagagaagcctgcaacaacaagctagattttgcggagaaggcagagtgggccacggcacaaagaccagagtggccgctgtgtactctgctggcgacaaaatcgcctcttaccgggacagcatcaaccacagatacagcaaggccctggtcgagtacgccgtgaagaatggatacggcaccatccagatggaagatctgacaggcatccagaacgacctggatcaccctaagaggctgcagcactggacatactacgacctgcagaccaagattgagaacaaggccaaagagcacggagtgggcgttgtgaaggtgaaccccagatacaccagccagcggtgtagcagatgcggccatatcgagagagaaaacaggcccacacagaaagtcttttgttgtaaggcctgcggcttcgaggggaatgccgactacaacgccagccagaacctgtccatgcggaacatcgacaagatcatcgagaaggaactgagcgccaaaggagaa

SEQ ID NO: 44 - DNA, 1290 bp, synthetic construct
  source 1..1290: mol_type = other DNA
atgtgcgccctgacaaagatcatgaagtacgagctgagatacctggatggcttccctgatttttctgccatgcagaacgctgtgtggcccctgcagcggcagacccgggaaatcctgaacaggacaatccaggaggcctatcggtgggactacttcagcgccaccaagaagaaagaaaccggcgagtaccccgacctgcagaaggaaaccggctacaaaagactggacggctacatctaccacgtgctgagccccgactaccctgatttctctagcagtggagtcaatgctacaatccagaaggcctggaagaaatacaagtcctctaaggccgacgtgtggaagggcgaaatgagcctgcctagctacaagagcgaccagcctatcgtgctgcacgccaagcagatcaagctgagcggagataccagagccgccgctgctaccctgtctctgttcagcaacaagttcaagaaggagcacgagatcagcggcaacgtgcagttcgccatcacactgcacgacaacacccagagaaccatctaccagaagctgagaaacggcgagtacaagctgtccgagagccagctggtgtacgacaagaagaagtggttcctgtacctggcctacagcttcaaccctgccgaacatgccctggaccctgagaagatcctgggcgtggacatgggcgagaaattcgccctgtatgccagcagcttcggcgagtacggccatttcaagatcgagggaagcgaggtgaccgagtatgcgaaggctctggaaagaagaaggagatctctgcaacagcaggcccggtactgcggcgagggcagaatcggccacggcacaaagaccagagtgggcgtggtgtacagagaggaagatagaatcgccaacttcagatccaccattaaccacagatacagcaaggccctgatcgagtacgccgtgaagaacggatatggcaccattcagatggaaaacctcacaggtatcaaggagaacctgcaatttccaagacggctgcagcactggacctactacgacctgcagagcaagatcgaagccaaagccaaagaacacggcatcgccgtcgtgaaggtgaaccccaagcacaccagccagcgctgcagcagatgcggacacatcgctgccgagaaccggcctaaacaggaggtgttccaatgtgttaagtgcggctacgcttgtaatgccgattttaacgccagccaaaatatctccatcaaggacatcgaaaagctgatccaggagacaattggcgccaatcctaag

SEQ ID NO: 45 - DNA, 1365 bp, synthetic construct
  source 1..1365: mol_type = other DNA
atggccaagaagggtaattctcagaagaagcagatcgtgaaagttatgaaatacgagctgaagtatgaaaagggatgtgccgacttcaacgagatgcaaaacgagctgtggaagctgcaaagacagaccagagaagtgatgaatagaaccatccagctgtgctatcactggagctacgtgcaggcagagtactgcaaacaacacggctgtgccagacgggacgtcaagccttgcgacgtgtacgaaaccaacgccacctctctggacggctacatctaccagctgctgaaggtggaatacccagatttcttcatgaagaacctgaatgccacactgagaaaggctcaccagaagtacgacgccctgctgtttgacattcaggagggcaactctagcatccccagtttcaagaaggaccagcctctgatctttgagaagaaagccatctgcatcagcaagtgtctgcctgacaagcggcagattaccctgtcctgcttcagcgacagctacatcgacgcccacccaaccctggataagatcaccttcaccgtgcgggccagaagcgccagcgagaaaagcatcttcgaccacatcatctccggcaagtacgccctgggcacaagccagctcgtgtacgagaagaagaagtggttcttcctgctgagctacaagttcacacctgagagcgtggacgtgaaccccgagaaggtgctgggcgtggacctgggagtggtgaacgccctctgcgccggcagcgtggaaaaccctcatgattctctgttcatcaagggcacagaggctatcgagcagatccggcggctggaagctagaaagcgggacctgcagaagcaagccagataccccggcgacggcagaatcggccacggcaccaagaccagggtgagccctgtgtatcagacacgggatgccatcgccagaatgcaggacaccctgaaccacagatggtccagagccctgattgacttcgcctgcaagaaaggctacggcacaatccagatggaagatctgagcggcatcaaggccatggaatctgagaagccttacctgaagcactggacctacttcgatctgcagagcaagatcatctacaaggccgaagaaaagggcatcagagtggtcaaggtgaatcccaagtgtaccagcagacggtgcagcgcttgtggatatatctccaaagagaaccgcaagaaccaggctgagttcctgtgcgtcaactgcggctaccaccacaacgccgattacaacgccgctcagaacctgtctatccctcagatcgatagactgatcgagaagcagcttaaggaacaggagagcgaggaatccgaggccggaacaaaccctaag

SEQ ID NO: 46 - DNA, 1311 bp, synthetic construct
  source 1..1311: mol_type = other DNA
atggccgaaaagaccatcgtgaaagtgatgaaatttgagctgagatacatcgacggtgctggcgagttctccgagatgcagaaacatctgtgggagcttcagaagcagaccagagaagtgctgaacaaaaccattcagatgggctacgccctcgaatgcaagcggtttgcccaccacgacaagacaggacagtggctggatgacaaagagctgaccggatccaagtacaaggctgtcgctgactatatcaacgctgaactgaaggaagattacaacatcttctacagcgactgtagaaacagcacagtgcggaaggcctacaagaagttcaaggacgccaagaacaagatcttcagcggcgagatgagcctgccttcttatagaagcaaccagccaatcatcatccacaacagaaatgttatcatcagaggcaacgccgagagcgccctggtgggcctgaaggtgttcagcgacggatttaaggccctgcacggcttccctgccgccgtcaactttaagctgtgcgtgaaggacggcacccagcgggccatcatcgagaacgtgatcagcgagatctacaagatcagcgagtctcagctgatctatgataacaagaaatggttcctgatcctggcctacagattcacccagaagaagaacgacctgaatcccgacaagatcctgggagttgatctgggcgtgaagttcgccgtgtacgccagcagcatcggcgaatacggcagcttcagaattaagggaggcgaagtgaccgagttcatcaagagactggagaaaagaaagaagtccctgcagaatcaggccacagtgtgtggagacggccgcatcggccacggcactaaaacacgggtggccgatgtgtacaaggccagagacaagatcagcaatttccaggacaccatcaaccacagatactctagagctatcgtggactacgccagaaagaacggctacggcaccatccaactggaaaagctcgataatagcattgagaagaaaggcgattacagccctgtgctggtgcactggacctactacgacctgaggacaaagatggaatacaaggcagccgagtacggcatcaaagtgatcgccgtggaacccaagtacacctctcaaagatgcagcaagtgtggctacatctcttctgagaacagaaagacccaggagagcttcgagtgcatcaaatgcggctacaagtgcaacgccgacttcaacgcctcccaaaacctgagcgtgcgggacatcgacagaatcatcgatgagtacctgggcgccaaccctgaactgaca

SEQ ID NO: 47 - DNA, 1299 bp, synthetic construct
  source 1..1299: mol_type = other DNA
atggtttgtaacaaggtggtgaagatcgctctgatctgcgaccagatcgataaggatggaaaggacgtgaactacaatgacatctacaagctgctgtgggaccttcagaaacagacaagagaagccaagaacaaggtcatcagactgtgctgggagtggtccggctactctagcgagtatttcaaaacccacgaggaatatcctaaggataaagagatcctgggcatcagcctgagatcctacctgtacaatagaatcaagggcgactacaacctgtacagcggcaacttgtctcaatccgccaaaatcgcctacatcgagtacaagaacagcctcaccgacgtgctgcggggcgataaaagcatcatcaactacagggagaaccagccactggacatcaagaacaaggccatacagctgctgtacgagaacgacaactttttcgtgcgggtggccctgatcaacaaggacaagcggaaggagctgaacttcaaggactgcagcgtgcggtttaagctgctggtgaaggatgatagcacacggaccatcctggaaagatgcttcgacgaggtgtacacaatcaccgccagcaagatcatgtacaacaagaagaagaagcagtggtacatcaacctgggatacaaattcaccaaggaaatcgacaagacactggataaggatagaatcctcggcgtggacctgggcgtgatcaaccccctggtggctagcgtgtacggcagctacgacagactgatcattggaggcggagaaatcgacaagttcagaaagcgggtcgaggccaacaaagtgcagatgctgaaacagggcaagtactgcggcgacggcagaatcggccacggcgtgaacaccagaaacaaacctgcttataacatcgaggacaaaattagcagattccgggacaccgtgaatcataagtattctaaggctgtggtcgactacgccgtgaagaacaactgcggtacaatccagatggaagatctgaaaggcattacacagaacaagaatgagagatacctgaagaattggacctacttcgatctgcagaccaagatcgagtacaaggccaaggcactgggcatcgaagtgaagtacaagaatcctaagtacaccagccaaagatgttctaaatgcggccacatcgccgaggaaaaccgccccgagcagaaaaccttcaagtgcgtgaagtgtggatttaaggtgaacgccgactacaacgccagccagaatctggccatcaaggacatcgacaagatcatcgaacagtactacaacaagggc

SEQ ID NO: 48 - DNA, 1272 bp, synthetic construct
  source 1..1272: mol_type = other DNA
atgaagtacaccaaggtgatgagataccagatcatcaagccactgaatgccgagtgggacgagctgggaatggtgctgcgggacatccaaaaggaaaccagagccgccctgaacaagaccatccagctgtgctgggagtaccagggcttttccgccgattacaagcagatccacggccagtaccccaagcctaaagatgtgctgggctacacctctatgcacggctatgcctacgacaggctgaagaatgagttcagcaagatcgcttctagcaacctgagccagacgatcaaaagagccgtggacaagtggaacagcgacctgaaagagatcctgagaggcgatagaagcatccctaacttccggaaggactgccctatcgatattgtgaagcagagtaccaagatccagaaatgtaatgacggctacgtgctcagcctgggcctgatcaaccgggaatataagaacgagctgggaagaaagaacggagtgttcgacgtgctgatcaaagctaatgataaaacccagcaaacaatcctggaaagaatcatcaacggcgactacacctacaccgcctctcagatcattaatcacaagaacaagtggttcatcaacctgacataccagttcgaaaccaaggagacagccctggaccctaacaacgtgatgggcgtggacctgggaatcgtctaccctgtgtacatcgccttcaacaacagcctgcacagataccacatcaagggcggcgagattgagagattccgccggcaggtggaaaagcggaagagagaactgctgaaccagggcaagtactgcggcgacggcagaaagggccacggctacgccacaagaacaaagtccatcgagagcatctccgacaagatcgccagatttagagatacctgcaaccataagtacagccggttcatcgtggatatggccctgaagcacaactgcggcattatccagatggaagatctcaccggcatcagcaaggaatctacattcctgaagaactggacctactacgacctgcagcagaagatcgagtacaaggccagagaggccggaatccaggttatcaagatcgaaccccagtacacaagccaacggtgcagcaaatgtggatatatcgacaaggaaaacagacaagagcaggccaccttcaagtgtatcgagtgcggatttaagaccaacgccgactacaacgctgctaggaacatcgccatccccaatatcgataaaatcatcagaaagacactgaagatgcag

SEQ ID NO: 49 - DNA, 1272 bp, synthetic construct
  source 1..1272: mol_type = other DNA
atggctacaaaggtgatgagataccagattatcaagcctatcgattgcaattgggacctgttcggcaaggtgctgagagacatccagtatgatacaagacagatcatgaaccggaccatccagtactgctgggagtggcagggctacagcagcgactacaagatcgccaagggcgagtaccctaagacccgggaaaccttcggctacagcgatatgcggggctacgcctacgacaagctgaaatcaatctaccagaggctgaacacagccaatctgaccaccagcataacaagagccgtgcagcggtggaaaaccgataccaaggacgtgatcagaggcgacaaatccatcgcctgcttcagagccgacgtgccaatcgacctgcacaacaagagcatgaacattgagaagagcgacgacggctacatcgtggccctgagcctggccagcaacatctacaagaaggaactggacagaaattctggccagttcagcgtgctgatcaatgagggcaacaagtctaatcgggacgtcctggatagatgtatcgctggacagtataagattagcgcttctcagatcctgcgggaaaagaacaagtggttcctgaacctgtcctactcttttgagatcagcaagcccgataagtctagagacaacatcctgggaatcgacgttggaatcgtgcaccccgtgtacatggccgtgtacaacagccctgctagaaggagcatcagcggcggcgaaatcgacaacttccgcaagcaggtgcagaaaagaatcaaagagctgcagctgcaaggcaaacaatgtggcgagggcagaatcggccacggcatcaagacaagagtgaaacctatcgagtttgccaaagacaaggtcgccaacttccggaacaccatcaaccacaagtactccaaggctatcgtggaattcgccattaagaacggctgtggaatcatccaaatggaagatctgaaaggcatcaacaccgacaacgtgttcctcaagaactggacctactacgacctgcagcagaaggtgaagtacaaggccgagctggaaggaatcgaggtgaagctgatcgacccccagtacaccagccagcggtgctgcaagtgtggctatatccatagagataacagacctgagcaggccaagttcaagtgcatcgactgcggcttcgaggtgaacgccgattacaatgccagcctcaacatcgccacccctgacatcgacaagatcatcctggaatttctgaagtgcgagaca

SEQ ID NO: 50 - DNA, 1353 bp, synthetic construct
  source 1..1353: mol_type = other DNA
atgggcgtgacaatcaagatcatgaaataccagatcctgtgccctatgaacgtcgactggacaatcttcgagaaacatctgagaaatctgacctaccaggtgcggaccatcagcaacagaaccatccagcagctgtgggagttcgacgccctgagctttgattactttaaggaacgaggaacataccctacagtccaggacctgtacggctgcacccagaagaagatcgacggctacatctaccacacactgcagagcaagtatcccgacatccacaaaggcaacatgagcaccaccctgcagaagatcatcaaaacctggaagtctagaagaaatgagatcagaaagggcgagatgagcatccctagcttcagaaacagaattcccatcgacctgcacaacaacagcgtggacatcatcaaggaaaagaacggcgattatatcgccggcatctctctgttcagcagagatttccacaaggagaacggcgacgtgccaaagggcaagatcttcgtgaaactgggaacccagaaacagaaatccatgaaagtgatcctggatagactgatcaatcagacctacagcaaaggcgcttgtatgatccacaagtacaagaacaagtggtatctgagcatcacctacaagtttaacgctatcaaggaaaacaagttcgacaaggaactgatcatgggaatcgatatgggcggaatcaacaccgtgtacttcgcctttaacgagggcttcatccggagcaacatcaagtccgacgagatcaagatgttcaacgaacggatcagacagagaaggattaatctgcttaagcagtctaaatactgcagcaacagcagaacaggcaagggccggaccaagcgcctgcaacctatcgatgtgctgtccaataagatcgccaagttccggaactctacaaaccacaaatacgccaattacattgtgaagcagtgtctgaagcacaattgcggcagaatccagatggaactgctgaagggaatttctaagaacgacaaggttctgaaggactggacctacttcgacctgcaggagaagatcaagaaccaggccgagatctacggcatcgaagtgatcaaggtggtgcctgcctacaccagccagcggtgtagccaatgtggctacatctgcaaggagaacagatgcacacaggccatgttcgagtgcaagcagtgcggctacaagacccacgccgattataacgccgctaagaacatctccacctacgacatcgagaacatcatcaacaagcaactggccgtgcagagcaagctgcacagcaagaagtgcatggaagagtacatcgaggaactgggctacctggac

SEQ ID NO: 51 - DNA, 1350 bp, synthetic construct
  source 1..1350: mol_type = other DNA
atgtctaccgtggtcaaagtgatgaagtaccagatcatttgtcctgtgaacatcgagtggaaggcttttgagacatatctgcggaccctttcttatcaggttagaacaatcggcaaccgcactattcagaagctgtgggacttcgataaccagagcctgaatcactttcgcgagaatggcgtgtaccccagcgctcagcagctctacggttgcacccagaaaaccatcagtggatatatctacgaccagctgaaagaggagtatcaggacatgaacaaggcgaacatgtcgaccaccctgcagaaaaccatcaagacctggaactcgcgtaagaaggaaatccgcagcggcgagatgtctattcctagcttccggaacaacctccccatcgacatccatggcaattccatccagatcaccaaggagaagtccggggactacattgcctccctgtctctcttttcaagcaacttcatcatcgaaaacaacctgcccaacggcaagattcaagtcaagctgtccactcgcaagcagaactccatgaaggtgatcctggacagaatcatcgagaacacctacgcgaagggtgcctgcatgctgcacaagcacaaaaacaaatggtacttgagcatcatctacaagccgacagtaaaggaggaacataagttcgaggaagatctggtgatgggcatcgacatgggaaagatcaacgtgctgtacttcgccttcaacaagggctggatcagaggcgccatctccggggaggagattgaggccttcagaaagaaaattgagcacaggcggatctctctgctgagacagggcaaatactgcagcggaaaccgggtgggcaagggcagagagaagcggatcaagcctatcgacgtgctcaacaacaagattgccaagtttcgcaatgcaaccaaccacaagtacgccaactacatcgtgcagcagtgcctgaaatacaactgcggcaccatccagctggaaaacctgcaaggcatctccaaagaacagacgttcctgaagaactggacctatttcgacctgcaggagaaaatcaagcaacaggcccaccagtacggcatgaaggtggtgacaattgatccaagctacaccagtaagaggtgttctgaatgtggctacatccacaagaataaccgcaagagccagtccacattcgagtgccagcagtgtaatttgaaagtgcacgccgattacaacgccgctaagaacatcagcatctacaacatcgagaaggtcatccagaagcaactgaagctccaggaaaaactgaacagcaagaagttcaccgagcagtacatcgagcaggtggagaacatcaat

SEQ ID NO: 52 - DNA, 1350 bp, synthetic construct
  source 1..1350: mol_type = other DNA
atgtctatcgccgtgaaagtgatgaaatatcagatcgtttgtcctgtgaacatcgagtggaaaaccttcgagatctacctgagaaccctgtcttaccacttccggacaatcggcaatagaaccatccagaaactgtgggagtacgacaaccaaagcctgaagcacttcaaggacaccggccagtaccccagcgcccagcagctgtacggatgtacccagaaaaccatctctggctacatctacgaccagctgaaagaggaataccaggacatcaacaaggccaatatgagcacaacactgcagaaaaccattaagacctggaacagcagaaagaaagaaatctggtccggagagatgagcatccctagcttcagaaacaacctgcctatcgacatccacggcaacagcatccagatcatcaaggaaaagagcggcgactacatcgcttctgtgtctctgttcagcagcaagttcatcaaggaaaacgacctgcccaacggcaagatacttgtgaagctgagcacaagaaagcaaaatagcatgaaggtcatcctggacagaattatcgacagcacctacgccaaaggagcttgtatgctgcacaaacacaagaagaaatggtatctgtccataacatacaagtccaacatcaaggaagagctgaagttcgacgaggatctgatcatgggcatcgatatgggcaagatcaacgtcctgtacttcgccttcaacaagggcctggtgagaggcgccatctctggcgaagaaatcgaagcctttaggaagaagatcgagcacagaagaatctctctgctgcggcagggcaagtactgcagcggaaaccggatcggcaagggcagaaagaagcggattaagcctatcgaggtgctgaatgataagatcgctaaattcagaacagccaccaaccacaagtacgccaactacatcgtgcagcagtgcctgaagtacaactgcggcaccatccagctcgaggacctgcaaggcatcagcaaagaacagaccttcctgaagaactggacctacttcgacctgcaggagaagatcaagaatcaggccaatcaatacggcatcaaggtggtgaagatcgatcctagctacaccagccagcggtgcagcgagtgcggatatatccacaagaacaacagacagaaccagagtacctttgagtgccagcaatgcagctttaaggtgcacgccgactacaacgccgccaagaacatctccgtgtacaacatcgagaaggtgatccagcggcagctgaagctgcaggagaagctcaacctgacaaagtacaaggagcagtatatcgagcagatggaaaatattaac

SEQ ID NO: 53 - DNA, 1359 bp, synthetic construct
  source 1..1359: mol_type = other DNA
atgagagccctggaaaaccagaagcctctgaagtctatcaagaaacctgtgtgtaaaatcagcagaaccctgtctgtgcccattcagcggccttgtggctacgtgtggaacgatttcggccacctgctgtgcatcatcagaaacgacgtggcccaggcctataatatggccatgtctgagagctacctgtacttctccgagagagagaattacaagcgggaacacggcaaatatcctaaggtggaacaactggctaagcgcaacgtgtacaagaagttgacagagaacttcccccacatcggcaccggcatcctggccaccatcgccaacaaggtggaatctaaactgaagaaagagtacgtggaagtgatgctgaagggcacaaagtccgtgtccaactacaagaagggtacacctataccaatccgggctcagggctggaaggaacggacctttaagagaaagagaaaggacaagatgaccttccacctgctgagcaagaaggccgagcagagcaagagcctggatttcctgaaggacgagaagggcaagatcccctgcagcttcaccgtgcggatcgctctgaagaagctgaacaactcccagagagccgtgtacaacagaatctgggccggcgagtacaaggctggcgctatcgacatcctgcagcggaagggcaagtggttcatcaacatctcttatcacatgctggagacaaagcggctggaaaagcagctggacaagaatgtgatcgtgggagtggacctgggaatcgtcaacggcgtggtgtgcgccgtcagcaacgacgcctacgacagactcgtgctgcggaaggacatcgagggctttagaaagcagatctggaaaagaaagcacctggcctggaagagcaccaggcggggcggaaagggaagaaagtactacctgaggatgagcgacagcctcaagaagaaagagcacaacttcagaaataccctgtaccacgactggaccagaaaagtgatcgactacgccctgaagcacggcgccaaagttatccagatcgaagatctgagcggcctggtggaagccaagaagaaaatgaagaaaggcgtgctgaagaactgggtgatcagcgacttcgtcgagaagctgacatacaaagctgaggaatacggcattgagatcgtgaaggtgaaccccagatacaccagccagcgttgccataagtgtggacatatcgagaaggacaaccggaaagagcaaagcaagttcgtgtgcctgaagtgcggccacagctgcaacgccgattttaacgccgccaagaatatcgccacaaagaatatcgccgacatcatctccgccagcctgcctcagacc

SEQ ID NO: 54 - DNA, 1470 bp, synthetic construct
  source 1..1470: mol_type = other DNA
atgaccgacgagcaggccagactgcagaaagtggccacatttcagatcgtcaagcctgtgaacatggactggcgggagttcagaaagctgctgagagatgtgcggtacagactctggagattaggcaatatggccgtgtctgaggcttatctgacctttcacaagaagtacagaatgggccaggcccagagcgatggcgcccacaagctgagcgtcctggacaagcgcctgcggcaggctctgatcgacgagggcgttagagtggaagaactgagcagatacagccggaagggcgccgtgtccggctacatctgtggcgccttcgagaaaaccaagctgagcgccatcaagtccaagagcaagtggcgggacatcatcaatggcagagcatctctgcctgtgttcagacgggacctggccatccccatcaactgcagcgactgccagcctagaatgatcgagagaacagaggccggcgagtaccaggtggacctgaggatttgtctgcaagataaggagctggcccctaacggctaccccagggtgctgctgtctacagctaagatcagcgacggccagagagccgtgctggagagactcgtcagcaacaagaccaacagcctgcctggatacaggcacagattcttcgagatcaaggagaagcggggcaagtggttcctgtctgtgtcctacgacttccctagagctgaagccggcaaactgcaccaggacatcatcgtgggcgtggacctgggctggtccgtgccactgtacgccgctctgaacaagggctacgccagaatcggctggaagaagctggaaccactggccaagcggattcggcacctgcaaaagcaggtgaagggacggagactgagcatgcagcggggaggccaggccgacctggctggtcctaccgcccggatgggacacggcagaagaagaaacctgcaggccattgagaagctggaaggaaagatcaacgacgcctacaccaccctgaatcaccaactgagccactgcatcatcgagttcgccaagaacaacggcgctggcgtgatccagatcgaagatctgagaggcctggctgatgagctgcgggcgaccttcattggacagaactggcgataccaccagctgcaggagtttatcaaatataaggccgaggaagccggcatcaaggtggtgccccccgtgaatcctttctacaccagcagacgttgcagcgtgtgcggctacctgcataaggacttcaccttcgagtacagacaggttaaccggaagaacggcatgagcgtgatgttcgagtgccccgagtgcagcaagaaagccaaggaagagggaaaagagtacaaggcccttaatgccgattacaacgccgccagaaacctggccacagccaacatcgaggaaaagatcagactgcaatgtaaagaacagggcatcgaatatacagaactgcctaagtct

SEQ ID NO: 55 - DNA, 1434 bp, synthetic construct
  source 1..1434: mol_type = other DNA
atgaaggactacatcagaaagaccctgtccctgagaatcctgcggccctactacggcgaggaaatcgagaaagagatcgccgccgccaagaagaagtcccaggccgaggggggagacggcgctctggacaacaagttctgggaccggctgaaggccgagcaccctgagatcatctctagcagagagttctacgacctcctggatgccattcagcgggagacaaccctgtattacaatagagccatcagcaagctctaccacagcctgatcgtggaaagagagcaggtgtctaccgccaaggccctgagcgctggcccttaccacgagttcagagaaaagttcaacgcttatatcagcctgggccttagagagaagatccagagcaacttcagaagaaaggaactggccagataccaggtggccctgcctacagccaagagcgacaccttccctatccccatctacaagggcttcgacaagaacggaaaaggcggctttaaggttagagaaatcgagaacggcgactttgtgatcgacctgcccctgatggcctaccacagagtgggcggaaaggctggcagagagtacatcgaactggaccggcctccagccgtgctgaacgtgcctgtgatcctgtctacaagccggagaagggcaaataaaacctggttccgcgacgagggtacagacgccgaaatccggcgggtgatggccggagagtacaaggtgtcctgggtggaaattctgcaaagaaagagattcggcaaaccttacggcggatggtacgtgaacttcaccatcaagtaccagcctagagattatggcctggaccccaaggtcaagggcggcatcgacatcggcctgagcagccctctggtctgcgccgtgacaaacagcctggccagactgaccatcagagataacgacctcgtggccttcaaccggaaggctatggctcgccgcagaaccctgctgagacagaatagatacaagagatctggccacggcagcgccaacaaactgaagcccatcgaggccctgacagagaagaacgagctgtacagaaaggccatcatgcggcggtgggccagagaagccgctgatttcttccggcagcaccgcgccgctaccgtgaacatggaagatctgaccggcatcaaggacagagaggactactttagccagatgctgcggtgctactggaactacagccagctgcagaccatgctggaaaacaagctgaaagagtacggaatcgccgtgaagtacattgagcctaaggatacatctaagacctgtcactcctgcggccatgtgaatgagtacttcgacttcaattaccggagcgcccacaagtttccaatgttcaagtgtgaaaagtgcggcgttgagtgcggcgccgattacaacgccgctagaaacatcgcccaggcc

SEQ ID NO: 56 - DNA, 1500 bp, synthetic construct
  source 1..1500: mol_type = other DNA
atggaagtgcagaaaacagtgatgaaaaccctgagcctgagaatcctgcggcctctgtacagccaagagatcgagaaggaaatcaaggaggaaaaggaaagaagaaagcaggccggcggcaccggcgaactggacggaggcttttacaagaagctggaaaagaagcattctgagatgttcagcttcgacagactgaacctgctgctgaaccagctgcagagagagatcgccaaggtgtacaaccacgccatcagcgagctgtacattgccaccatcgcccagggcaacaagagcaacaagcactacatctctagcatcgtgtacaatagagcctacggctacttctacaacgcctatatcgctctgggcatctgcagcaaggtggaagccaacttcagatccaacgagctgctcacccagcagtctgccctgcccaccgccaaaagcgacaacttccccatcgtcctgcacaagcagaagggcgctgagggcgaggatggcggcttccggatcagcaccgaaggatctgatctgatcttcgagatccctatccccttctacgagtacaacggcgagaaccggaaggaaccttataagtgggtgaagaaggggggacaaaaacctgtgctgaagctgatcctgtctacattccggagacagagaaacaagggctgggccaaggacgagggcactgatgccgagatccggaaggttacagagggcaagtaccaggtgtctcaaatcgagatcaaccgcggcaaaaaactgggcgagcaccagaagtggttcgctaattttagcatcgagcagcctatctacgagagaaagcccaatagaagcatcgtgggcggcctggatgtgggcattagaagccctctggtttgtgccatcaacaacagcttttccagatatagcgtggacagcaatgacgtgttcaagttctccaagcaggtgttcgccttcagaaggcggctgctgtccaaaaacagcctgaaacgtaagggccacggcgctgcccacaagctggaacccatcaccgagatgaccgaaaaaaacgacaagttccggaagaagatcatcgaaagatgggccaaggaagtgaccaacttcttcgtgaagaaccaggtcggaatagtgcagatcgaggacctgagcaccatgaaggaccgggaagatcacttcttcaaccagtacctgcggggcttttggccttactaccagatgcagacactgatcgagaacaaactcaaagagtacggaatcgaggtgaagcgggtgcaagctaaatacacaagccagctgtgcagcaatcctaattgcagatactggaacaactactttaatttcgagtacaggaaagtgaataagtttccaaagttcaagtgtgaaaagtgcaacctggaaatcagcgccgactataacgccgctagaaacctgtctaccccagacatcgagaagttcgtggccaaggccacaaagggaatcaacctgcctgagaag

SEQ ID NO: 57 - DNA, 1479 bp, synthetic construct
  source 1..1479: mol_type = other DNA
atgaaaaccaccgagaagaacgtgctgatgaccaagtgcatcaaggttacactgaacaggtgtgtgaactacaatatgaaagagatcatgaacattatccgggaaatgcagtacctcagcagcaaggcctacaacctggccacaaactacctgtacatctgggacaccaattctatgaacttcaagaacctctacgaggaaaagatcgttgataaagacctgctgggcaagagcaagtccgcctggatcgagaaccggatgaatgaaatcatgaagggcttcctgaccaacaacgtggcccaggccagacaggacgtgatcaacaagtataacaaaagcaagaaagatggcctgtttatcggcaaggtcacccttccaagctacaagatgaacggcaaggtggtcatccacaacaaggcttatagattcagcaagaatgagggctacttcgtggaaatcggcctgttcaacaaggaaaagaaggaagagctgaactgcgactggatcaagtttaagctggacaaaatcgacagcaacaagaaggccaccatctacaagatcctgaatggcgattacaagcagggcagcgcccagctgcacatcaacaagaaaggaaagatcgagttcattatttcttatagcttcgagcgggaaaactccatcaagctggataagaacagaacactgggaatcgacatcggcatcgtgaacatcgccgccatggccatctgggataacaataaacaggagtgggagctgaccagatacagccacaatctgatcagcggcaacgaggccatcgctctgcggcagaagtactacaaactgggactgagaaacaaggaactggaaaagaacatcaaccgggaactgcacgagctcgaagagaaggaatacagaggcctgagcacaaacattatctctggccataatctgacctacaagcgtatcatgctgaacagcaagagaatcagactgtctcagtcctgcaagtggtgcggcaacagcaaagtgggccacggtagaagagtgcggtgcaagcaagtggacaagataggaaacaagatcgagagattcaaagacaccttcaaccacaagtacagccggtacatcgtggacttcgccgtgaagaacaattgcggaattatccagatggaaaacctgaagaatttcaaccccagcgagaagttcctgaaggactggccttactttgacctgcagacaaagatcgaatacaaggccaaggagtacggcatcgaggtgatcaaagtgaatcctaagtacaccagcaagcgctgcagcagatgtggctgtatcaacgagctgaacagagattgcaagaagaaccagtctaaattcaagtgcgtgaacgacgagtgcaacaactatgagaacgctgacatcaacgccgctaagaacatcgccctgccttacatcgacaagatcatcgagcaatgtctggagacaaacaaggtggtg

SEQ ID NO: 58 - DNA, 1350 bp, synthetic construct
  source 1..1350: mol_type = other DNA
atgaagctgaataaatgcatcaaggtgaccctggtcaagtgcctgaactacgactacaaagaaatcaaacagatcatcagagacttcaactacaccgcctgcaaggccagcaacaaagctatgagaatgtggttcttccacacacaggacatgatcgataagaagaacaagtacaaggaatttaaccagatccagtacgagaaagacacatacggaaagtcttacagaaacgtgatcgagggagagatgaagaaaatcatgccactggccaataccagcaacgttggcacactgcaccagcagctcgtgcagaacgactggtccagactgaagaaggacatcctgtcctgtaaagccaacctgcccacctacaagctgtccaccccttacttcatcaagaatgacaattttaagctgcggaaccacaacggctacttcgtggacattgccttcttcaacaaggagggcctgaagcagtacggctacaaggccggccataagttcgagtttcagatcgacaaactggacggtaacaagaagtccaccatcaacaagatcatcaacggcgagtacaagcaaggcagcgcccagctgagcatcagcaataagggcaaaatcgaactgatcatcagctacagcttcgaaaaggaggaagtgcccgtgctggacaagaacaaaatcctgggaatcgacctgggcatcaccaacgtggccacaatgagcgtgtacgacagcatgcgggaacagtacgactacttcagctggaaaaccaacgtgatctctggcaaggagctgatcgcttttagacagaaatactacaacctgaggagagatatgagcatcgcctctaaaacagccggacagggcagatgcggccacggctacaagaccaagatgaagagcgtgaacaaggtgcggaataagattgcaaacttcgccgatacatataaccacaagatttctaagtacatcatcgagttcgccatcaagaacaactgcggcgtgatccaagtggaagatctgagcggcgccaccgccgacacccacaacaagatgctgaaggattggtcttattacgacctgcagcagaagatcgaatacaaggctaaggagcaaggcatcgaggtgatcaaggtcaaccctaagtataccagcaagcggtgcagcaaatgtggatgtatccacgaggataatagagattgcagaaacaaccaggccaagttcgagtgcaaggtgtgtggctacaatgagaacgctgacatcaacgccagcaagaacatcgccattcctgacatcgacaatatcatcaagggcactgagatcctgcacagcaaagaaaacaaggcttct

SEQ ID NO: 59 - DNA, 1311 bp, synthetic construct
  source 1..1311: mol_type = other DNA
atgacaaccaagtgcgtgcaggtcgccatcgagtacagcagtaacaacatcctgaaggaggtggacttctacaaggaactgcgggacctgcaatacaacagctacctggcctgcaaccgggccatcagctacatgtacgagaacgacatgcagaacttcatcataaaggaaactgatcttcctagaagcgacgacaagaagctgtacggcaagtccttcgccgcttggatcgagaacagaatgaacgagtacatgccaggcgctctgagcaacaatgtggcccagacaagacagttcgtggtgaacagatacaagaatgacaagaaagctggcctgctgaaaggcaacgtgtccctgaccaccttcaagcggaccaatcctatcatcatccacaacaatgcctacaacatcatcgaaacccctaagggcctgggagctgagatcggctttttcaatctgcctaagcagaaagagctgggaatcaaaagagttaattttctgttccccaagctgggcagcagcgagaagagcatcatccggcggctgctggacaaatcttataagcagggcgccatgcagatctcttacaaccagaagaagaagaaatggatggccaccatcagcttcagctttaacctggaagaaatcaagaccaacgagaatctcgtgatgggcatcgacctgggcgtgtctaaagtggccacactgagcatctacgacgccagcaagtatgagtatattaagatgagctttaaggatacatgcatcgacggcaccgagctgatgcactacaggcagaaactggaaagcagacgcaaggccctgagcatcgccagcaagtgggcctctgataacaaccgtggacacggctacaagaccaagatggagaaagccaactacatgggaagaaagtacaacaacttcagagacacctacaaccataaggtgtctagatacatcgtggacgtggccatcaagtatagagtgggcctgatccagatggaagatctgtccggcttcagcgagcagcagcaggagagcctgctgaagaactggtcctactacgatctgcagcaaaagatcaagtacaaggcagaagagaacggcatcagagtgtacttcatcaaccccaagtacacatcacagcggtgcagcaagtgcggcaacatcgataaagaaaatagaaagacccaggagagcttctcctgtacagtgtgtaattacaaggacaacgccgacgtgaacgcctctaagaacattgccattcctgacatcgagaagatcatcgaggaacaggtcaagaaacagtac

SEQ ID NO: 60 - DNA, 1413 bp, synthetic construct
  source 1..1413: mol_type = other DNA
atgatcacaaccagaaagctgaagctggcaatcgtgtccgacaacaagaacgaggcctacagcttcatccgggacgagacaagaaaccagaacagagctctcaacgtggcctacagccacctgtacttcgagtacatcgcccaggagaagctgaagcattctgatgccgagtaccaggagcacctggccaagtaccaggagctggcctctaagaagtaccaggagttcctgaaggtcaaagagaaagctaaatctgacgagacactgcaggccaaggtggacaaagccagagaagcctataacaaggcccaggagaaggtgtacaaaatcgagaaggactacagcaagaaggctcgagaaatctaccaacagagcgtgggcctggccaaacagacaagaatcgataagctgctgaagaatcaatttaacctgcactacgacaccgtggacagagtgggcggaacagccataagccacttcaccaacgacatgaagagcggcgtgctgcaaggcaaaagaagcctgcggaattacaagagcagcaaccctctgatgatcagagccagatctatgaaggtgtacgaggaaaatagcgattactacatcaaatggatcaaggacatcacatttaagatcatcatcagcgccggcagcaagcagagacagaacattggagagctgaagtccgtcctcgtgaacatcatcgagggaaattacaaagcttgtgatagcagcatcggcgtggataaggatctgatcctgaacctgagcatcgacatccccatcaccaaggaaaacatcttcatccctaaccgggtggtgggagttgacctgggcctgaagatcccagcctacgtgtccgttaatgataccccttacatcaagcgggccatcggcaatataaacgacttcctgaaggtgcggacccagctgcaatctcagcggcgccggctgcagaaggccctgcagagcaccaacggcggcaagggtagaaacaagaagatgcagggcctggaaagactgcaagctaaggaaaagaacttcgtgaacacctacaaccacttcctgagcaagaacatcgtggattttgccgtgaaaaacaacgctggcatgatccacatggaagaactgaagttcgacaaggtgaagcacaagtctctgctgagaaattggtcctattaccagctgcagaccatgatcgagtataaggccaagagagagggcatcgaggtgtattacgtggacgccagttacacctcccagacctgcagcaagtgcggcaacctggaagaaggccagagggagacacaggacaccttcgtgtgcaagaagtgcggctacagcgtgaacgccgactataacgccagccagaatattgccaaggccaaaaccatcaaggaggaaaaccagcag

SEQ ID NO: 61 - DNA, 1488 bp, synthetic construct
  source 1..1488: mol_type = other DNA
atgattctgaccagaaagatcaagctggtgatcgtgtccgagaaccgggaggaaggctacaacctgatcagaacagagatccgggaacagcacaaggcactgaacctggcttataaccacctgtacttcgagcacaacgccatccagaaactgaagcagaatgatgaggattacaagcagaaaagaaacaagctgcaggagctgatcaacaagaaatacgaggaacatcagaaggctaagaatctcgagaagaaggaagccctgagagaggcctataacaacaagaagcaggagctgtacaacttcgagaaggaatacaatgagaaggctagacagacataccagcaagtggtgggctttacccaacagaccagagtgcggaacctgatcaatagagagtgcaacctgatgagcgacaccaaggacggcatcaccagcaaggtgacccaggactataagaacgactgtaaagctggactgctgatcggcaagcggagcctgcggaactacaagaaggacaaccccctgcttgtgcgtggaagaagccttaagttctacaaggaagatggagattacttcatcaagtggaacaaggggaccatcttcaagtgcatcctgcacatcagaaagaagaacgtggtggaactgcagagcgtgctggaaaacgtgctgctgggcgcctacaaggtgtgcgactctagcatcggatttaacaacaaggatatgattctgaatctgagcctgaacatcccagataaggagacacagggctacatccctggccgggtggtcggcgtggacctgggcctgaagatccctgcctacctgagcctgagcgataaggtgtacgtccggaagggcatcggcagcatcgacgacttcctgagagttcggacccagatgcagaagagacggcggagactgcagaagtctctggccgctgtgaagggcggcaaaggcagagaaaagaagctgaaagccctggaccacctgaagggaaaggaggccaactttgccaagacatataatcacttcctctccacccagatcgtgacattcgccgtgaagaaccaggccggccagatcaacatggaattcctggaattcgacaagatgaagaacaaaagcctgctgaggaactggtcctactaccagctgcaaatcatggtcgagtacaaggccaagagagagggcattatcatcaagtacgtggacgcctacctgacctctcagacctgcagcaagtgtgatcactacgaggacggccaaagagagaaacaagagaatttcatgtgcaagaattgcggcctggaagtgaacgccgactacaatgccagccagaacatcgccaagagcaccagctacatcagcgactctacagagtccgagtaccacaagaagaaacagcaggtgctgaaggaaatcctgggcgagaacgacatcatgaacgaacagctgtctctgttcaacaattgtgacgacatcgcc

SEQ ID NO: 62 - DNA, 1383 bp, synthetic construct
  source 1..1383: mol_type = other DNA
atgatcagcacaagaaagatcaaggtgaggtgtgatgacagcaccttttatacatttttcagacaggagcagagagaacagaacaaggccctgaacatcggcattggaatcatccacgccaacgccgttctgcataatgttgactctggcgccgagaagaagctgaagaaatccatcgagggactgcagggcaagatcgacaagctcaacaaagacctggagaaagagaagatcactgataagaagaaggaagaggtgctgaaggccatcgaaaccaacaagaaaatcctggacggcgagaagaaggtgttcaaggaaagcgaggaatacagaaagggcatcgacgagctgttcaagaacacctacctgaagtctaataccctggatcacgtgctggatagcatggtgaacatccagtacaagcggaccctgtccctggtgacccagcggatcaagaaagactacagcaacgacttcgtgggcatcatcaccggccagcagagcctgcggaactacagaaatgacaatcctctgatgatcagcaaccagcagctgaacttcaagtacatcgatgatacattctacctggacatcatgtgcggctaccggctggaagtggtgctggggaagcgggacaacgagaatgtgaacgagctgaagtctaccctggaaaaggtgatttctaaagagtataaggtgtgcgacagcagcatgcagttcagcaagaacaacaaggatgtgatcctgaacctggtcatcgacatcccacagaatagcaacgtgtacaagcctgtggaaggcagaattctgggcgtggacctcggagtggccgtgcctatctacatgtgcctgaacgacgacacctacaaaagaaagggcctgggcgacatcaacaacttcctgagagtgcggcagcaaatgcaaacaagacgacggaaactgcaaaaggacctgaccctgacaaacggcggaaaaggcagaaagaagaaaacacaactgctggacaagctgcaggagaacgagcggaacttcgtgaaaacctacagccacgccctgagcaagagggtggtcgagttcgccaagagcaacaaatgtgaatatatcaacatcgagaagctgaccaaggacggcttcgacaacatcatcctgagaaactggtcctacttcgagctgcagaaaatgatcgagtacaaggctgagagagagggcattacagtgcgctacgtgaaccccgcctacaccagccagaagtgttctagatgcggcgaaatcgacaaggagaacagacagacccaggccaattttaaatgcaccaagtgcggatttgaactcaacgctgatcacaacgccgctatcaatatcgccagaagcatcgaattcgtg

SEQ ID NO: 63 - DNA, 1446 bp, synthetic construct
  source 1..1446: mol_type = other DNA
atgaacaccgtccggaagatcaagctgaccatcctgggcgataccgagacaagaaacaaacagtacaagtggatcaaggatgagcagtataaccagtaccgcgccctgaacctgagcatgacctacatggtcaccaacctgatgctgaagaacaacgagagcggcctggaaaatagaaaggagaaagacatcttgaagatcgagaacaagatcaagaaggacgagggctccctgaagaaagagctggccaagaagaagatcaatgaggagaagatcgaaaacatcaagtccaacatcgaagaacttaagagcgagaaggagaagctggagaatgagctgaagaatattaaagagtacagaagcaacatcgatgaggagttcaagaagatgtacgtggacgacctgtacaacgtgctgaacaagatcagctttcagcacgaggacatgaaatctctggtgacccagcgggtcaagaaggacttcaacaatgacgtgaaggaaatcatgagaggagatagatccgtgagaaattataagcggaacttccccatcctgacaagaggccgggacctgaagtttcagtacatcgagaagagcgaagatattgagatcaagtggatcgagggcatcaagttcaagtgcatcctgggaaaacctagcaagtctctggaactgaagcacgccctgcataaggtgatcaacaaggaatacaaggtgtgtgattccagcctgcaatttgacaagaacaacaacctgatcctgaacctgaccctagacatccctcaggataacaagtatgagaaaataacaaacagagtggttggcgtggacctgggcctgaagatcccagcctacgtggccctgaacgacacaaagtacattaggaaggccatcggaagcatcgacgatttcctgaaggtgcggacccagatgcagagcagagtgcgcaagctgcagaaaagcctgcaagtggtgcggggcggcaagggcagaaataagaaaatgaaagccctggaacggttccgggaaaaggagaggaatttcgccagaaactacaaccacttcctgagctacaacatcgtgaagttcgccctcgacaacaaggctgagcagatcaacctggaactgctggaaatgaagaagacccagaacaaaagcattctgagaaactggagctattaccagctgcaaaacttcatcgagtacaaggccgagagagtgggcatcaaagtgaaatacatcgacccttaccacacatctcagacctgtagcgagtgcggcaactacgaagaagggcagagagtggaacaggacacattcgtgtgcaagcggtgctggcacaagatgaacgccgactacaacgctgctagaaatatcgccatgagctacaattacatctctaagaaggaggaatctgagtactacaagaacaacaagaacatggtg

SEQ ID NO: 64 - DNA, 1212 bp, synthetic construct
  source 1..1212: mol_type = other DNA
atgaacaccgtgcggaagatcaagatcatcatcaacaacgagaacaacgagctgagaaaagaacagtataagttcatccgggacagtcagtacgcccagtaccagggcctgaacagatgcatgggctacctgatgtccggcttctacgtgaacaacatggacatcaagtccgaggagttcaagacctggcagaagggcgtgacaaacagcgccaacttcttccaggagatcagcttcggcaagggaatcgacagcaaatcttctatcacccaaaaggtgaagaaagacttctccatcgctctgaagaacggcctggccaagggcgagcggaacatcaacaactacaagagaatcgcccctctgatgaccagaggcagaaatctgaaattcaagtacgacgacaatgagcttgatatcctgatcaattgggtcaacaagatccagttcaagtgcgtgctgggagagcacaagaactctctggaactgcaacacaccctgcacaaagtgatcaacaatgagtacaagatcggccagagcagcctgtacttcaacaagaagaacgagctgatcctgatcctgacaatcgatatccccaccgccaaaagcagctacgagcctatcaaagatagaatcctgggtgttgacctgggcatggccgtgccagtgtacatgagcatcaacgacaactcctatatcaagaagagcctgggcagctacagcgagtttgccaaggtgcgcaagcagtttaaagagagaaggaatagactgtacaagcagctggaagcctgcaagggcggcagaggcagaaaggataagctgaaggccatgaaccagttcaaagaaaaagagaaaaatttcgctaagacctacaaccacttcctgagcaagaacatcgtggagttcgccctgaagaacaagtgtgaatttatccatctggaaaagattgagagcaagggcctcgaaaacagcgtgctggctaattggacctactacgacctgcaggagaagatcatttacaaggccaaacgggaaggcattggaatcaagttcgtgaacagcagctatacaagccaaacatgcagcaagtgcaactacgtggacaaggagaacagaaagacccaggccaagttcatctgtaaaaactgcggatttaaggccaatgctgattacaacgcctctcagaacatcagcaagtctaaggagttcatcaag

SEQ ID NO: 65 - DNA, 1269 bp, synthetic construct
  source 1..1269: mol_type = other DNA
atgaacatcgtgaagaaaatcaagctgagaatcatcgataacgacaaggaactgtgcaagaagcagtatctgggattcaccgaggaacagaagaaagaactcattgataagcagtacaaatttatcagagattctcagtatcagcagtatctgggcttcaacagagccatgggctttctgatgagcggctactacgccaacaacatggacatcaaatccgacaacttcaaggaacaccagaaaaagctgaccaatagcctgtacatcttcgacgacatcaagttcggcgtcggaatcgacagcaagtctctgatcgtgcagcgggtgaagaaagacttcagcacagccctgaagaacggcctcgccaaaggcgaaagatctgtgaccaattacaagagaacctaccctctgcttacaagacacagatccatcaaattcctgtacgccgagaatgagctggacatctacctggactgggtgaacaaaatccggttcagatgcgagctgggcaatcataagaacagcctggaactgcagcacaccctgcggaaggtgatcaccggcgagtacaagataagcgactcctccctcgagttcaacaagaagaatgagctgattctgaacctgaacctgaacatccccgagacaaaggctacattcatcaaggatcggaccctgggcgtggacctgggaatggccatccctgcctacgtgagcctgtctgacaccccatacatcagaaagggctttggcagctacgaggagttcgccaaggtcagaaaccagtttaaggacaggcggaagcggctgctgaaacaactgtctctggttgctggcggcaagggcagagccaagaagctgcacagcatggagttcctgaagaacaaggaaaagcagttcgccaagacctacaaccacagcctgagcaagaaaatcatcgacttcgctctgaaaaacaactgtgaatacatcaacctggaggacatcaagtctacaagcctggaggacagagtgctgggacagtggggctactaccagctgcaagagcagatcgagtacaaggccaagctggtgggcatcaaggtgcggaaagtgaaggccgcttacaccagccagacatgcagcgagtgtggcaacatcgacaaggagaaccgcaagaatcaaagcaccttcaagtgcaccaacgaggattgcaaactgaacaagaaaggaatcaatgcagattggaacgccagcatcaacatcgccagaagcaaggagttcatcaag

SEQ ID NO: 66 - DNA, 1200 bp, synthetic construct
  source 1..1200: mol_type = other DNA
atgatcaccgtgcggaaggtcaagctgattgtgaactctgaggaggccgaggagatcaacagaacatacaagttcatcagagattctatgtacgctcagtaccagggcctgaatagatgtatgggctacctgctgtctggctactacgccaacggcatggacatcaagagcgacggctttaagaaccacatgaaaaccatcaagaacagcctgaacatcttcgacgacatcaatttcggcattggaatcgacagcaagtccgccattacacagaaggtgaagaaggacttcagcaccagcctgaagaatggtcttgcaaagggcgagcggggcgccaccaactacaaaagaaactttccactgatgaccagaggaagagatgtgaaaattagctacctggaagatacaaacacctttgtgatcaagtgggtcaacaagatcgagtttaaggtgatcctgggccagaaagataacatcgagctgagccacaccctgcacaagatcatcaacaaggaatacaccctgggacaatgtaccttcgagttcgacaagaacaacaagctgctcctggccctgaacatcaacatccccgataatctgatcagcaagaacaaggaaatcatccctggcagagtgctgggcgtggacctgggcgttaaggtgcctgccatgatctgcctgaacgacaataccttcatcaagaaatctatcggctcctacaacgagttcttcaaggtgcggagccagttcaaggccagacgggaacggctgtacaaacagctagaaagcagcaacggcggaaagggccggaagcacaaactgaaggccacaatgcagtttagagacaaagaaaagaacttcgccaggacctacaaccatttcctgagcaaaaatatcatcgagttcgctcagaagtatacctgcgagacaatcaacctggaagaactgaacaaaaagggcttcgataacaacctgctcggcaagtggggctactaccagctgcagagcatgatcgagtacaaggctgagagagtgggcatcaaagtgaaatacgtggaccctgctttcacatcccaaacatgcagcaagtgcggctatgtggacgaggaaaaccgcatcacccaggacaagttcgagtgccagaagtgcggcttcaccctgaacgccgaccacaacgccgccatcaatatcgccagaaag

SEQ ID NO: 67 - DNA, 1203 bp, synthetic construct
  source 1..1203: mol_type = other DNA
atgattgccgtgaagaagctgaaactgaccatcgtggaagaagaggagaaacggaaggagcagtacaagtttatcagagacagccagtacgcccagtaccagggcctgaatctggccatgggcatcctgacatctgcttatctggtgtccggccgcgacatcaagtccgacctgttcaaagactctcagaagagcctgaccaacagcaacgagatcttcaacggcatcaacttcggaaagggaatcgataccaagagcagcatcacccagaaagtgaaaaaggatttcagcactagcctgaagaacggactggccaaaggcgagagaggctttaccaactacaagcgggacttcccactgatgaccagaggaagagatctgaaattctacgaggaagataaggagttctacatcaagtgggtcaacaagatcgtgttcaagatcctgatcggcagaaaggacaagaacaaggtcgagctcatccacaccctgaacaaggtgctgaacaaggagtacaaggtgagccaaagcagcctgcaatttgacaagaataacaagctgatcctgaacctgacaatcgacatcccttacaaaaaggttgatgagatcgtgaaggacagagtgtgcggcgtggacatgggcatcgctatccccatctacgtggccctgaacgacgtgagctacgtgcgggaaggcatgggcaccatcgatgagttcatgaagcagagactgcagttccagagcagacggcggagactgcaacagcagctgaagaacgtgaacggcggcaagggcaggaaggacaagctgaagggcctggaatctctgagagagaaggaaaaatcttgggtgaaaacctacaatcacgccctgagcaagagagttgtggagttcgccaagaaaaacaaatgtgaatacatccacctggaaaagctgacaaaggacggcttcggcgaccggctgctgcggaattggtcctactacgagctgcaggagatgatcaagtacaaggccgacagagtgggtatcaaggtgaagcacgtgaatcctgcctatacaagccagacctgtagcgagtgcggccatgccgataaggaaaacagagagacacaggccaagtttaagtgcctggaatgcggcttcgaggccaatgccgactacaacgccgctagaaacatcgctaaatctgataagttcgtgaag

SEQ ID NO: 68 - DNA, 1194 bp, synthetic construct
  source 1..1194: mol_type = other DNA
atgatcgccgtgcggaaactgaaaatcatggtgctgtgcgacgacgagagcaagaagaacgagcagtataagttcctgagagatagccagtacgcccagtacctgggcctgaaccgggccatgagctttctggctaaggaatacctgtctggcgacaaagaaagattcaaagaggccaagaagaagctgaccaacacatgcgagtgctaccagaacatcaacttcggcaccggcatcgactccaagagccagatcacccagaaagtgaagaaggaccttcaagctgacatcaagaatggcctggccagaggagagcggagcatcagaaattaccggagaacattcccactgattacaagaggcagagatctgaagttcagctacaacggcgacgagatcatcattaagtgggtcaacaaaatctacttcaaggtgctgatcggcagaaaggacaagaactacctggaactgatgcataccctggaaaagatcatcaacggcgagtacaaggtgtgcaccagcagcatccagatcgacaagaaactgatcctgaatctgacactcgaaatccctgataaggtgaagaaggagtttcaggagaatagagtgctgggtgttgacctgggcatcaaattccccgcttatgcttgcgtgtctgataacacctacgtgcggagatcttttggcagcatcgatgaattcctgaaggtgcgcatccagttcgacaagagacggaagcggatccaacagcagctgcagaacgtgaagggaggcaagggcagaaaggataagctgcaggccctggacagaatgcgggactgcgagaggaagtgggtgcggaactacaaccacgccctgtctaaaagaatcatcgacttcgccttcagaaacaagtgcggcatcatccacctggagaagctggaaaaggacggctttaagaacaagctgctgcggaactggtcctactacgagctgcaagacatgattggatataaggccgagagagagggcatcgtggtgaagtacgtggaacctgcctacacctcccagacctgtagcaagtgtggatacgtggatagagaaaatagacctagccaggagcacttcctctgcaaggaatgtggcttcgagatcaatgccgaccacaacgccgccatcaacatcgccagaagcaacaaggtcatcgtggacaag

SEQ ID NO: 69 - DNA, 1206 bp, synthetic construct
  source 1..1206: mol_type = other DNA
atgatcacagtgcggaagctgaagctgacaatcatcaatgacgacgaaaccaagcggaacgagcagtacaagtttatcagagatagccagtacgcccagtaccagggcctgaacctggctatgagcgtgctgacaaacgcctacctgtcaagcaacagagatatcaaatccgatctcttcaaggaaacacagaagaacctgaagaatagcagccacatcttcgacgacatcacctttggaaagggaacagacaacaagagcctgatcaaccagaaggtgaagaaggacttcaacagcgccatcaagaacggcctggctagaggcgagagaaacatcaccaactacaagaggaccttccccctgatgaccagaggcaccgccctgaagttcagctacaaagacgactgcagcgacgagatcatcatcaagtgggtcaacaagatcgtgttcaaggtggtgatcggcagaaaggacaaaaattacctggaactgatgcacaccctgaacaaggtgatcaatggagagtacaaggtgggccagagctctatctacttcgataagtccaataagctcatcctgaacctgaccctgtacatccctgagaagaaagatgatgacgccatcaacggcagaacactgggcgtggacctgggcatcaagtatcctgcttacgtgtgcctgaatgacgacaccttcattagacagcatatcggcgagagcctggaactttctaaacagagagagcagttcagaaaccggagaaagaggctgcagcaacaactgaagaacgtgaagggcggcaagggccgcgagaaaaagctggccgccctggacaaagttgccgtgtgtgaacggaacttcgtgaaaacctacaaccacaccatcagcaagcggatcatcgatttcgccaagaagaacaagtgtgagttcatcaatctggagcagctgaccaaggatggatttgacaacatcatcctgtctaattggtcctactacgagctgcaaaacatgattaagtataaggccgaccgggaaggcatcaaggtgcggtacgtgaacccagcctacaccagccagaagtgcagcaagtgcggttatatcgacaaagagaacagacctacacaggagaaattcaagtgcattaagtgcggcttcgagctgaacgctgatcacaacgccgccatcaacatttctagactggaagag

SEQ ID NO: 70 - DNA, 1401 bp, synthetic construct
  source 1..1401: mol_type = other DNA
atgataacagtgcggaagctcaaactgaccatcgtgggcgacgagcaaacaagaaaggagcagtacaagatcatcagagatgagcagtaccagcagtataaggccctgaacctgtgcatgaccctgctgaatacccacaacatcctgaatagctacaacaccggcagcgagaataagctgaacagccagattgagaagctggacaacaagatcgagaagaacaagatcgagctgaagaagggcaacctgaaggaaagcaagatcgaaaagctgaacaagagcatcctggaactgaccaaggaaaaggaaaagctgcaacaggagtacctgtccgccagcaagtacagaagcgacatcgacgagaagctgaaagacatgtacatcaaagacatgtatacagtggtgcagagccaagtgaacttcaaaagcaaagatatgatgagcctggtgacccagagagccaagaaggatttcagcaacgccctgaagaacggcatggcccggggagagcggagcctcatcaattacaagagagattttccactgatgaccagaggcgagagatggctgaagtttaagtacaatgaggaatctgacgacatctacatcgactggctgcacgacatcaagttcaaggtcatcctgggatataagaagaacgagaactctatcgagctgagacacacactgcataaggtgatcaacaaagagtacaagatctgcgacagctccatgcagttcgacagaaacaacaacctgatcctgaacctgaccctggacattcctaacaaagagagcaagggctacgtggaaggcagaacactgggcgtggacctgggcatcaaataccccgcttacgtgtgcctgtctgatgacacctacaagcggaagtccatcggctgtgccgaggacttcatcagggttcgggaacagatccggggcagaagataccggctgcagaagcagctgagcatggtcaagggaggaaagggccgcgataagaaacttagagctctggacagagtgcgggaagccgaacgcaacttcgtgaaaacctacaaccacatgatcagcaagaacatcatcaagttcgccaaggagcacaactgcgagtacatccacctggaaaagctgaccaaggacggctttcctgatattatcctgagcaagtggtcctactacgagctgcagaacatgatcgaatacaagagcgacagagaaggcatcaaagtgcggtacatcgatcctgcctacaccagccagacatgcagcaagtgcggccacatcgacaaggaaaatagaatcaaccaggagaagttcaagtgtgtgaaatgtggcttcgagctgaacgccgaccacaatgcttctatcaacatttctagatctaataaatacctgaag

SEQ ID NO: 71 - DNA, 1392 bp, synthetic construct
  source 1..1392: mol_type = other DNA
atgaaaaccaccagaaagctgaagctgaccatcatcggcgacgaggaaacaagaaaggagcagtacaagatcatcagagaggaacagtaccagcagtacaaggccctgaacctgtgcatgaccctgctgaacacacacaacatcctgaactcttacaacaccggcgccgagaacaagctgaatgcccagatcgattctatcgacaagaagatcgaacaggccaaaaaagagctggaaaaaaagggcctgaaagaaagcaaggtgtctaagctcaaggagacaatcgagttcctggaaaacgatagagagaagctcaaagacgagtacctgaactccagcaagtttagaagcgacattgatgaaaagatgaaagagatgtacatcaaggacatgtacactgtggtgcagaaccaggtgaacttccgggcaagagatatgatgagcctggtgacccagagagcccggaaggacttcaagaacagcctgaaaaacggcatggccaagggcgagagatccctgacaaactataagcgggacttccctctgatgacaaggggcgaacggtggctgaaattcgagtacgacaaagatagcgacgacatcctgatcaactggattcacggtatcaagttcaaagttctgctgggctacaaaaagaatgagaactccatcgagctgagacacaccctgcataaggtcatcaacaaggagtacaagatctgcgactctagcatgcagttcgatagaaacaacaatctgatcctgaacctcaccctggacatccctgacaagcagaacaacaactacatcgagaagagaacactgggagtggacctgggcattaagtacccagcttacgtgtgcctgaatgacgacacctacattcggagccacatcggcgagagcctggaactgctgaagcagcgggaacaatttaaggacaggcggaagcggctgcagcagcaactgaagaacgtgaagggcggaaagggcagaaacaagaagctgagcgccctgaacaaactgtctgataatgagagaaatttcgccagaacctacaaccacatgatcagcaagcggatcgtggagttcgccaagaagcacagatgtgagttcatcaacctggaaaagctgaccaaggacggcttcgacaacaatatcctgagcaactggagctactacgagctgcaaaatatgatcgagtataaggccaagcgcgaaggcatcgaggtgcggtacatcgaccccgcctacaccagccagaagtgcagcagatgcggatatatcgacaaggagaacagacagacccaggagaaattcaagtgtctgaagtgcgaatttgagatcaatgctgatcacaacgccgccattaacatcgccagagctttggat

SEQ ID NO: 72 - DNA, 1395 bp, synthetic construct
  source 1..1395: mol_type = other DNA
atgagcgagcaggaggccgctcaggagggcacaaagctgctggccaaaaccctgaccttcggcctgggcaaccctatgggcttcaagtccaagggctccgtgctggttgagctgacagaggaccagcgcaaggccatctacaacggcctgagagatgcctctaccgtggtggccagaatcatcaacctgctgaacagcagggagtacatccggcagatcatgaaagtgcctgaggaactggtggcacagttcaagcctaactacagcctggtgaagggacctcttaaacggctgggaattgaggaagccgagcaggtggccggcagcgtgttgagccagacctttgccctgggagtgaaacctgacttccagggcgagcacggcaagggcctgctgctgaagggtgaaagacagatccccctgcatagaaccgacggcacccaccctatcccacagcgagctacagagacaaggctgttccaagtggaaaagaacttctacgtggccatgcaggttttcgccgaaacctgggccaagaagcaggagctgcccagcggctggctcgccttccccatcaaggtgaagcccagagataagactatggccggacagctgttaaagacaatcggcggcgaatggaagctgaagaatagccggctgatgcggaatcctagaacaggaggaaatagatggctgggccagatcgtggtggcttttgcccctgagcctttcaagaagatgacccggtctgtcgtcatgggaatcgacctgggcgtgaacgtgcccgcctgcctgcacatttctgaaaacggcaaacctctgccttgggccatgatggtcggcagaggcagagacatgctgaataccagaaacctgatcagatctgaaatcgtgcacatcatcaaggccctgaaaagcaaggacagcccactggacggcaaggccagagccatctatcgggataagctgcgggacctgagaaagagagaacggagagtgatgaagatggccagccaaacagtggccgccagaatcgccgataccgccaagcggcacggcgccggcacctggcagatggaagatctgagccctgacatcaagaccgatcagccttggctcgccagaaactgggctcccggcatgctgctcgacgccgtgcggtggcaggccagacaatgtggcgccgagctggtgatggtgaaccctgcctacaccagccagagatgcgccagatgcggccacatcgaccctcagaaccggcccaagcaaaccgactttaagtgcatggcctgtgggcacgaggacaacgccgacaagaacgctgctagaaacctgtccgtggtgggcatcgagaagctgatcgccgacttcaaggctccaaacggcgctgtgcag

SEQ ID NO: 73 - RNA, 131 nt, synthetic construct
  source 1..131: mol_type = other RNA
agggacgacttcccgtcccaaaatcgagacagtagccgtaaaactttgagtttcagagtgggcgacacactcgaaaaggttaagatatgcacatagtaatccgtgcatgagccgcgaaagcggcttgaagg

SEQ ID NO: 74 - RNA, 140 nt, synthetic construct
  source 1..140: mol_type = other RNA
acggttgatttagcaaccgaagtctgagggcatgtagaaaaaagtataggtatataccaacatacttgcattgccactcggaaagggttaaccttggtcattgtgttaccgaccaagcattccagaaatggaatgtaaat

SEQ ID NO: 75 - RNA, 124 nt, synthetic construct
  source 1..124: mol_type = other RNA
ctgggacttcacccaaaatcgagacagtggccgtcagccttcccatcgggaagcgggcaatacactcgaaaaggttaagatgcacatagtaatccgtgcatgagccacgaaagtggcttgaagg

SEQ ID NO: 76 - RNA, 131 nt, synthetic construct
  source 1..131: mol_type = other RNA
aagggacgacttcccgtcccaaaatcgagatagtggtcctgattctttgatttcaaagcggacaatacactcgataaggttaagatgcacataggaatccgtgcatgggtcacagaaatgtgacttgaagg

SEQ ID NO: 77 - RNA, 141 nt, synthetic construct
  source 1..141: mol_type = other RNA
gattcaggggcgacttcccgccctgaaatcgagaaagtggtcgtaagccggaagcatttccgcagacaatacactcgaaaaggttaagatatgcacatagtaatccgtgcatgggtcgcattgaaaggtgcggcttgaagg

SEQ ID NO: 78 - RNA, 125 nt, synthetic construct
  source 1..125: mol_type = other RNA
aatttaggacttccctgaaatcgagaaagtggccgtaagacgcagttccttgcgccggcaatacactcgaaaaggttaagatgcacatagtaatccgtgcatgggtcatgaaaatgacttgaagg

SEQ ID NO: 79 - RNA, 99 nt, synthetic construct
  source 1..99: mol_type = other RNA
aatcgagatagcagccatttgaagacggtcttgcactcgaaaaggtcaagatgcacacaataatccgtgcatggtcagtgtgaaaacactggttgaagg

SEQ ID NO: 80 - RNA, 135 nt, synthetic construct
  source 1..135: mol_type = other RNA
tatttagggcgacttcacgtcctcaaatcgagaaagtgagcgtaagacttggcttctgtcaagcggttaatacactcgagaaggttaatatgcacatagtaatccgtgcatgagtcactgaaagtgacttgaagg

SEQ ID NO: 81 - RNA, 124 nt, synthetic construct
  source 1..124: mol_type = other RNA
ccgggcggcttggcgtccgtaaatcgagaaagtacattgttaatatagtggatacactcgataaggttaacgcatacgatattaatcccgtatgccgtctatatttgaaagggtatagtgcaag

SEQ ID NO: 82 - RNA, 129 nt, synthetic construct
  source 1..129: mol_type = other RNA
agtcgagaagtgccgtaataagcatctaaaaatgcctaacggtaacactcgataaggtagtcctgctaggcaggctgaaaccctagccacaaaatccggctaggcatcatacgaaaatgtatgatgtga

SEQ ID NO: 83 - RNA, 126 nt, synthetic construct
  source 1..126: mol_type = other RNA
taaatcgagaagtggcataaatccatacttgtgtggttgcaaaacactcgataaggtaaaaacggttagcaccgtttgaaattccgagtataaaagaccgctcggacgtcttgaaaaagatgtgag

SEQ ID NO: 84 - RNA, 153 nt, synthetic construct
  source 1..153: mol_type = other RNA
aaatggagaagtagcacataagaaatttaccaagtgccaacactccgtaaggtagtatcaaatgtaaataaacattgatgcgtgggcactttcatgctctgaagggtgtaacacaaaaaccgttacacaatacatcgaaagatgtatttaaat

SEQ ID NO: 85 - RNA, 154 nt, synthetic construct
  source 1..154: mol_type = other RNA
taaatggagaagtgacacacggtaaatgtaccaagtgtaaacgctccataaggtagtatcgaatgtttaaaaacattgatacgtaggcgttatgaatgccttgaagggtgtaacacaaagaccgttgcacaatacatgaaagatgtattgaaat

SEQ ID NO: 86 - RNA, 154 nt, synthetic construct
  source 1..154: mol_type = other RNA
aaatggagaagtgatacacggtaaatttaccaagtgtcaacgcaccgtaaggtagtatcgaatgttagaaaacattgatacataggcattctgaatgctttgaaggatgtaacacaaagaccgttacataatacattgaaagatgtattgaaat

SEQ ID NO: 87 - RNA, 131 nt, synthetic construct
  source 1..131: mol_type = other RNA
gtggtggttcgcgcaatggggcgagttcacgtccttatgttgagaagtgcctgtaattcaatgaattatcattgtttgtgtaacgctcaataagcctgcacacaataccgcacgaaagtgtgggttgaaac

SEQ ID NO: 88 - RNA, 111 nt, synthetic construct
  source 1..111: mol_type = other RNA
gttctttgaaataaagatatagctgccggtaaaacgatagcccacgggcaattgcgtgcggcagtttaggccgactcgaacggcctgaaggttgaggtaaagacttcgagg

SEQ ID NO: 89 - RNA, 113 nt, synthetic construct
  source 1..113: mol_type = other RNA
ataaatttcgtcccgcggtgatgggtatagcctttgggcagagtcccaaaatcgcggataagacgcggttgctttcaacagccgaaaatcgagctacgaaagtggattgaaac

SEQ ID NO: 90 - RNA, 189 nt, synthetic construct
  source 1..189: mol_type = other RNA
aatgttattccataataacatttgatgcacacgattcctccctacagtagttaggtatagccgaaaggtagagactaaatctgtagttggagtgggccgcttgcatcggcctaaagttgagaagtgtcagactctgataaccctcaacgacgatattctttatttcggaaacgaatgaaggaatgcaac

SEQ ID NO: 91 - RNA, 141 nt, synthetic construct
  source 1..141: mol_type = other RNA
aaagggtgatttaccatcctaagtagaggaaactcttacaaccgctccagttgaaatttgctggtaaaaagctagtataaaaaatgctataagcatggtgggtactatgatataaccaatgaaaataggttatatttaaat

SEQ ID NO: 92 - RNA, 151 nt, synthetic construct
  source 1..151: mol_type = other RNA
gtatctataacataaaactaagattattagtaagtttatagaataatatctcgataatgcttcaagtattaattcacttggtaaaggttgtgtaggggagtggctttaagtcagagttctacaccgatacaactgaaagttgtatttaaat

SEQ ID NO: 93 - RNA, 131 nt, synthetic construct
  source 1..131: mol_type = other RNA
aaggtgagaagtgctgtaggagtcccttgcagtaactctctaccaaacgctcctgtattaaataggtaaaagccttgtgcaaaacattaaaatgggcgtgtacaagaatacaatgaaagttgtatttaaat

SEQ ID NO: 94 - RNA, 130 nt, synthetic construct
  source 1..130: mol_type = other RNA
aggctcaggcgaatttgcgtctgtagagggaggcagggtgtaacacgatagcccgtatagcactccctaaagggttaaccttggtcattatgttaccgaccaagcaataccacagaaagtggattgaaat

SEQ ID NO: 95 - RNA, 124 nt, synthetic construct
  source 1..124: mol_type = other RNA
cgatttagcgtctaaagggtgagattgtagctcaccaagggttaactctagtcttgttatgttaccgactagacattctatattgatatcgaatgaacattccgtagaaatatggaatgtaaat

SEQ ID NO: 96 - RNA, 103 nt, synthetic construct
  source 1..103: mol_type = other RNA
atgtgaggtcatgtgatataggcactcgcaaagatagttgctaaaggtagcaattatcatcgtcctagtgaattgctaggtaataacagaaatgttattaaat

SEQ ID NO: 97 - RNA, 119 nt, synthetic construct
  source 1..119: mol_type = other RNA
agataataagaacagggcgatttaacgtcctaaggctgagggatatttccactcggcaagggttaatttcggatattgtgttaccatccgaacattccatggaaatatggaatgtaaat

SEQ ID NO: 98 - RNA, 158 nt, synthetic construct
  source 1..158: mol_type = other RNA
agtctattaataaataggtatgtaatagcatataaaccgaagggtgagagaatagactttcatgtattaggatttaccaacaatacactattactactcactaagggtaagcccaggtgttaagttaccgcctggcatactagaaatagtatgtgaat

SEQ ID NO: 99 - RNA, 145 nt, synthetic construct
  source 1..145: mol_type = other RNA
acggtatattcaaatgccgaagaatgagagatatttgatttaaaatagctaggtttaggccaacagttataaaaatctactcgttaaaggttaatccagatgttatgttactgtctggcatcatatagaaatatatgatgtgaat

SEQ ID NO: 100 - RNA, 174 nt, synthetic construct
  source 1..174: mol_type = other RNA
ttgtattgatgttatatataaatatatagcagttacggtaacttaaagtgccgaaggctgaggagatggattaaatatataaggttttgaccaactatataccatcatcactcggtaagggttaatcctaacaatgtgtgaccgttaggcgttccaaagaaatggaatgttaat

SEQ ID NO: 101 - RNA, 124 nt, synthetic construct
  source 1..124: mol_type = other RNA
agggtgagggtatagataaaacgcataaggtagtatgccaaatatgtgctataaccactcgctaagccgaaaaaaccttagtttatgatggcaactaagcacactatgaaagtagtgtgtaaac

SEQ ID NO: 102 - RNA, 158 nt, synthetic construct
  source 1..158: mol_type = other RNA
ttatgtccagatttagtggggcaagagtgagggcgtaggttaaaagtgtaaggctaatagcaactacattctacgacactcgctaaacggtaaaaactctagcctaatattctcaagagattacgtggctagacatatcacgaaagtgatatgtaaag

SEQ ID NO: 103 - RNA, 157 nt, synthetic construct
  source 1..157: mol_type = other RNA
tctataggcgatttagcgtctaaaggttgagggataagacaaaatggttaaggtttcgaccaactaactacttatccactcgataaacggtaaaaactcatactaatattctttatagataacgtggtatgacattccaagaaattggaatgtaaat

SEQ ID NO: 104 - RNA, 138 nt, synthetic construct
  source 1..138: mol_type = other RNA
atagtcgattcagcgactaaaggttgaggatataggataaatagctaggataacctaaagctatctataatcactcaataagggttaactctagatgttgtgttaccgtctagacatacatagaaatagtatgtgaat

SEQ ID NO: 105 - RNA, 165 nt, synthetic construct
  source 1..165: mol_type = other RNA
ataataaatatatataggcgatttagcgtctaaagattgaggtgtaggaactaacggttaaggtttataccaactaattacctatgcacttgataaacggtaaaaacttatactaacatcctttatagataacgtggtatgacattctagaaatggaatgtaaat

SEQ ID NO: 106 - RNA, 107 nt, synthetic construct
  source 1..107: mol_type = other RNA
tatggggctggattgcgacttcgggagcgcaaacagacccagaagatgccttcgggcatcaaccgccctgccaggacggggcgcaattcacgaaagtgagttggggg

SEQ ID NO: 107 - RNA, 111 nt, synthetic construct
  source 1..111: mol_type = other RNA
acagggcgatttaacgtcctaaggctgagagaagttccttctactcggcaagggttaatctcgattgttgtgttaccgatcgagcgtttcacagaaatgtgaaatgtaaat

SEQ ID NO: 108 - RNA, 100 nt, synthetic construct
  source 1..100: mol_type = other RNA
accgcttcacttagagtgaaggtgggctgcttgcatcagcctaatgtcgagaagtgctttcttcggaaagtaaccctcgaaacaaagaaaggaatgcaac

SEQ ID NO: 109 - RNA, 137 nt, synthetic construct
  source 1..137: mol_type = other RNA
ctctgtttcgcgcgccagggcagttaggtgccctaaaagagcgaagtggccgaaaggaaaggctaacgcttctctaacgctacggcgaccttggcgaaatgccatcaataccacgcgaaaaacgcgtggattgaaac

SEQ ID NO: 110 - RNA, 173 nt, synthetic construct
  source 1..173: mol_type = other RNA
tctattcgtcggttcagcgacgataagccgagaagtgccaataaaactgttaagtggtttggtaacgctcggtaaggtagccaaaaggctgaaactccgtgcacaaagaccgcacggacgcttcacatatagctcataaacaaagtttgcgagctagcttgtggagtgtgaac

SEQ ID NO: 111 - RNA, 115 nt, synthetic construct
  source 1..115: mol_type = other RNA
agggacgacttcccgtcccaaaatcgagacagtagccgtaaaactttgagtttcagagtgggcgacacactcgaaaaggttaagatatgcacatagtaatccgtgcatgagccgc

SEQ ID NO: 112 - RNA, 124 nt, synthetic construct
  source 1..124: mol_type = other RNA
acggttgatttagcaaccgaagtctgagggcatgtagaaaaaagtataggtatataccaacatacttgcattgccactcggaaagggttaaccttggtcattgtgttaccgaccaagcattcca

SEQ ID NO: 113 - RNA, 108 nt, synthetic construct
  source 1..108: mol_type = other RNA
ctgggacttcacccaaaatcgagacagtggccgtcagccttcccatcgggaagcgggcaatacactcgaaaaggttaagatgcacatagtaatccgtgcatgagccac

SEQ ID NO: 114 - RNA, 114 nt, synthetic construct
  source 1..114: mol_type = other RNA
aagggacgacttcccgtcccaaaatcgagatagtggtcctgattctttgatttcaaagcggacaatacactcgataaggttaagatgcacataggaatccgtgcatgggtcaca

SEQ ID NO: 115 - RNA, 122 nt, synthetic construct
  source 1..122: mol_type = other RNA
gattcaggggcgacttcccgccctgaaatcgagaaagtggtcgtaagccggaagcatttccgcagacaatacactcgaaaaggttaagatatgcacatagtaatccgtgcatgggtcgcatt

SEQ ID NO: 116 - RNA, 109 nt, synthetic construct
  source 1..109: mol_type = other RNA
aatttaggacttccctgaaatcgagaaagtggccgtaagacgcagttccttgcgccggcaatacactcgaaaaggttaagatgcacatagtaatccgtgcatgggtcat

SEQ ID NO: 117 - RNA, 81 nt, synthetic construct
  source 1..81: mol_type = other RNA
aatcgagatagcagccatttgaagacggtcttgcactcgaaaaggtcaagatgcacacaataatccgtgcatggtcagtgt

SEQ ID NO: 118 - RNA, 119 nt, synthetic construct
  source 1..119: mol_type = other RNA
tatttagggcgacttcacgtcctcaaatcgagaaagtgagcgtaagacttggcttctgtcaagcggttaatacactcgagaaggttaatatgcacatagtaatccgtgcatgagtcact

SEQ ID NO: 119 - RNA, 106 nt, synthetic construct
  source 1..106: mol_type = other RNA
ccgggcggcttggcgtccgtaaatcgagaaagtacattgttaatatagtggatacactcgataaggttaacgcatacgatattaatcccgtatgccgtctatattt

SEQ ID NO: 120 - RNA, 112 nt, synthetic construct
  source 1..112: mol_type = other RNA
agtcgagaagtgccgtaataagcatctaaaaatgcctaacggtaacactcgataaggtagtcctgctaggcaggctgaaaccctagccacaaaatccggctaggcatcatac

SEQ ID NO: 121 - RNA, 112 nt, synthetic construct
  source 1..112: mol_type = other RNA
taaatcgagaagtggcataaatccatacttgtgtggttgcaaaacactcgataaggtaaaaacggttagcaccgtttgaaattccgagtataaaagaccgctcggacgtctt

SEQ ID NO: 122 - RNA, 136 nt, synthetic construct
  source 1..136: mol_type = other RNA
aaatggagaagtagcacataagaaatttaccaagtgccaacactccgtaaggtagtatcaaatgtaaataaacattgatgcgtgggcactttcatgctctgaagggtgtaacacaaaaaccgttacacaatacatc

SEQ ID NO: 123 - RNA, 137 nt, synthetic construct
  source 1..137: mol_type = other RNA
taaatggagaagtgacacacggtaaatgtaccaagtgtaaacgctccataaggtagtatcgaatgtttaaaaacattgatacgtaggcgttatgaatgccttgaagggtgtaacacaaagaccgttgcacaatacat

SEQ ID NO: 124 - RNA, 137 nt, synthetic construct
  source 1..137: mol_type = other RNA
aaatggagaagtgatacacggtaaatttaccaagtgtcaacgcaccgtaaggtagtatcgaatgttagaaaacattgatacataggcattctgaatgctttgaaggatgtaacacaaagaccgttacataatacatt

SEQ ID NO: 125 - RNA, 113 nt, synthetic construct
  source 1..113: mol_type = other RNA
gtggtggttcgcgcaatggggcgagttcacgtccttatgttgagaagtgcctgtaattcaatgaattatcattgtttgtgtaacgctcaataagcctgcacacaataccgcac

SEQ ID NO: 126 - RNA, 102 nt, synthetic construct
  source 1..102: mol_type = other RNA
gttctttgaaataaagatatagctgccggtaaaacgatagcccacgggcaattgcgtgcggcagtttaggccgactcgaacggcctgaaggttgaggtaaag

SEQ ID NO: 127 - RNA, 97 nt, synthetic construct
  source 1..97: mol_type = other RNA
ataaatttcgtcccgcggtgatgggtatagcctttgggcagagtcccaaaatcgcggataagacgcggttgctttcaacagccgaaaatcgagctac

SEQ ID NO: 128 - RNA, 167 nt, synthetic construct
  source 1..167: mol_type = other RNA
aatgttattccataataacatttgatgcacacgattcctccctacagtagttaggtatagccgaaaggtagagactaaatctgtagttggagtgggccgcttgcatcggcctaaagttgagaagtgtcagactctgataaccctcaacgacgatattctttatttcg

SEQ ID NO: 129 - RNA, 120 nt, synthetic construct
  source 1..120: mol_type = other RNA
aaagggtgatttaccatcctaagtagaggaaactcttacaaccgctccagttgaaatttgctggtaaaaagctagtataaaaaatgctataagcatggtgggtactatgatataaccaat

SEQ ID NO: 130 - RNA, 134 nt, synthetic construct
  source 1..134: mol_type = other RNA
gtatctataacataaaactaagattattagtaagtttatagaataatatctcgataatgcttcaagtattaattcacttggtaaaggttgtgtaggggagtggctttaagtcagagttctacaccgatacaact

SEQ ID NO: 131 - RNA, 114 nt, synthetic construct
  source 1..114: mol_type = other RNA
aaggtgagaagtgctgtaggagtcccttgcagtaactctctaccaaacgctcctgtattaaataggtaaaagccttgtgcaaaacattaaaatgggcgtgtacaagaatacaat

SEQ ID NO: 132 - RNA, 114 nt, synthetic construct
  source 1..114: mol_type = other RNA
aggctcaggcgaatttgcgtctgtagagggaggcagggtgtaacacgatagcccgtatagcactccctaaagggttaaccttggtcattatgttaccgaccaagcaataccaca

SEQ ID NO: 133 - RNA, 106 nt, synthetic construct
  source 1..106: mol_type = other RNA
cgatttagcgtctaaagggtgagattgtagctcaccaagggttaactctagtcttgttatgttaccgactagacattctatattgatatcgaatgaacattccgta

SEQ ID NO: 134 - RNA, 88 nt, synthetic construct
  source 1..88: mol_type = other RNA
atgtgaggtcatgtgatataggcactcgcaaagatagttgctaaaggtagcaattatcatcgtcctagtgaattgctaggtaataaca

SEQ ID NO: 135 - RNA, 101 nt, synthetic construct
  source 1..101: mol_type = other RNA
agataataagaacagggcgatttaacgtcctaaggctgagggatatttccactcggcaagggttaatttcggatattgtgttaccatccgaacattccatg

SEQ ID NO: 136 - RNA, 142 nt, synthetic construct
  source 1..142: mol_type = other RNA
agtctattaataaataggtatgtaatagcatataaaccgaagggtgagagaatagactttcatgtattaggatttaccaacaatacactattactactcactaagggtaagcccaggtgttaagttaccgcctggcatacta

SEQ ID NO: 137 - RNA, 127 nt, synthetic construct
  source 1..127: mol_type = other RNA
acggtatattcaaatgccgaagaatgagagatatttgatttaaaatagctaggtttaggccaacagttataaaaatctactcgttaaaggttaatccagatgttatgttactgtctggcatcatata

SEQ ID NO: 138 - RNA, 158 nt, synthetic construct
  source 1..158: mol_type = other RNA
ttgtattgatgttatatataaatatatagcagttacggtaacttaaagtgccgaaggctgaggagatggattaaatatataaggttttgaccaactatataccatcatcactcggtaagggttaatcctaacaatgtgtgaccgttaggcgttccaaa

SEQ ID NO: 139 - RNA, 107 nt, synthetic construct
  source 1..107: mol_type = other RNA
agggtgagggtatagataaaacgcataaggtagtatgccaaatatgtgctataaccactcgctaagccgaaaaaaccttagtttatgatggcaactaagcacactat

SEQ ID NO: 140 - RNA, 141 nt, synthetic construct
  source 1..141: mol_type = other RNA
ttatgtccagatttagtggggcaagagtgagggcgtaggttaaaagtgtaaggctaatagcaactacattctacgacactcgctaaacggtaaaaactctagcctaatattctcaagagattacgtggctagacatatcac

SEQ ID NO: 141 - RNA, 140 nt, synthetic construct
  source 1..140: mol_type = other RNA
tctataggcgatttagcgtctaaaggttgagggataagacaaaatggttaaggtttcgaccaactaactacttatccactcgataaacggtaaaaactcatactaatattctttatagataacgtggtatgacattccaa

SEQ ID NO: 142 - RNA, 122 nt, synthetic construct
  source 1..122: mol_type = other RNA
atagtcgattcagcgactaaaggttgaggatataggataaatagctaggataacctaaagctatctataatcactcaataagggttaactctagatgttgtgttaccgtctagacatacata

SEQ ID NO: 143 - RNA, 149 nt, synthetic construct
  source 1..149: mol_type = other RNA
ataataaatatatataggcgatttagcgtctaaagattgaggtgtaggaactaacggttaaggtttataccaactaattacctatgcacttgataaacggtaaaaacttatactaacatcctttatagataacgtggtatgacattcta

SEQ ID NO: 144 - RNA, 91 nt, synthetic construct
  source 1..91: mol_type = other RNA
tatggggctggattgcgacttcgggagcgcaaacagacccagaagatgccttcgggcatcaaccgccctgccaggacggggcgcaattcac

SEQ ID NO: 145 - RNA, 12 nt, synthetic construct
  source 1..12: mol_type = other RNA
gcggcttgaagg

SEQ ID NO: 146 - RNA, 12 nt, synthetic construct
  source 1..12: mol_type = other RNA
tggaatgtaaat

SEQ ID NO: 147 - RNA, 12 nt, synthetic construct
  source 1..12: mol_type = other RNA
gtggcttgaagg

SEQ ID NO: 148 - RNA, 13 nt, synthetic construct
  source 1..13: mol_type = other RNA
tgtgacttgaagg

SEQ ID NO: 149 - RNA, 15 nt, synthetic construct
  source 1..15: mol_type = other RNA
ggtgcggcttgaagg

SEQ ID NO: 150 - RNA, 12 nt, synthetic construct
  source 1..12: mol_type = other RNA
atgacttgaagg

SEQ ID NO: 151 - RNA, 14 nt, synthetic construct
  source 1..14: mol_type = other RNA
acactggttgaagg

SEQ ID NO: 152 - RNA, 12 nt, synthetic construct
  source 1..12: mol_type = other RNA
gtgacttgaagg

SEQ ID NO: 153 - RNA, 14 nt, synthetic construct
  source 1..14: mol_type = other RNA
gggtatagtgcaag

SEQ ID NO: 154 - RNA, 13 nt, synthetic construct
  source 1..13: mol_type = other RNA
atgtatgatgtga

SEQ ID NO: 155 - RNA, 10 nt, synthetic construct
  source 1..10: mol_type = other RNA
aagatgtgag

SEQ ID NO: 156 - RNA, 13 nt, synthetic construct
  source 1..13: mol_type = other RNA
gatgtatttaaat

SEQ ID NO: 157 - RNA, 13 nt, synthetic construct
  source 1..13: mol_type = other RNA
gatgtattgaaat

SEQ ID NO: 158 - RNA, 13 nt, synthetic construct
  source 1..13: mol_type = other RNA
gatgtattgaaat

SEQ ID NO: 159 - RNA, 14 nt, synthetic construct
  source 1..14: mol_type = other RNA
gtgtgggttgaaac

SEQ ID NO: 160 - skipped

SEQ ID NO: 161 - RNA, 12 nt, synthetic construct
  source 1..12: mol_type = other RNA
gtggattgaaac

SEQ ID NO: 162 - RNA, 18 nt, synthetic construct
  source 1..18: mol_type = other RNA
cgaatgaaggaatgcaac

SEQ ID NO: 163 - RNA, 17 nt, synthetic construct
  source 1..17: mol_type = other RNA
ataggttatatttaaat

SEQ ID NO: 164 - RNA, 13 nt, synthetic construct
  source 1..13: mol_type = other RNA
gttgtatttaaat

SEQ ID NO: 165 - RNA, 13 nt, synthetic construct
  source 1..13: mol_type = other RNA
gttgtatttaaat

SEQ ID NO: 166 - RNA, 12 nt, synthetic construct
  source 1..12: mol_type = other RNA
gtggattgaaat

SEQ ID NO: 167 - RNA, 14 nt, synthetic construct
  source 1..14: mol_type = other RNA
tatggaatgtaaat

SEQ ID NO: 168 - RNA, 11 nt, synthetic construct
  source 1..11: mol_type = other RNA
tgttattaaat

SEQ ID NO: 169 - RNA, 14 nt, synthetic construct
  source 1..14: mol_type = other RNA
tatggaatgtaaat

SEQ ID NO: 170 - RNA, 12 nt, synthetic construct
  source 1..12: mol_type = other RNA
tagtatgtgaat

SEQ ID NO: 171 - RNA, 14 nt, synthetic construct
  source 1..14: mol_type = other RNA
tatatgatgtgaat

SEQ ID NO: 172 - RNA, 12 nt, synthetic construct
  source 1..12: mol_type = other RNA
tggaatgttaat

SEQ ID NO: 173 - RNA, 13 nt, synthetic construct
  source 1..13: mol_type = other RNA
gtagtgtgtaaac

SEQ ID NO: 174 - RNA, 13 nt, synthetic construct
  source 1..13: mol_type = other RNA
gtgatatgtaaag

SEQ ID NO: 175 - RNA, 13 nt, synthetic construct
  source 1..13: mol_type = other RNA
ttggaatgtaaat

SEQ ID NO: 176 - RNA, 12 nt, synthetic construct
  source 1..12: mol_type = other RNA
tagtatgtgaat

SEQ ID NO: 177 - RNA, 12 nt, synthetic construct
  source 1..12: mol_type = other RNA
tggaatgtaaat

SEQ ID NO: 178 - RNA, 12 nt, synthetic construct
  source 1..12: mol_type = other RNA
gtgagttggggg

SEQ ID NO: 179 - RNA, 32 nt, synthetic construct
  source 1..32: mol_type = other RNA
gttgcaacccgcgtatgggcgcggcttgaagg

SEQ ID NO: 180 - RNA, 30 nt, synthetic construct
  source 1..30: mol_type = other RNA
gttaaaagctaactatagtggaatgtaaat

SEQ ID NO: 181 - RNA, 32 nt, synthetic construct
  source 1..32: mol_type = other RNA
gatgcatctcacgcgtgtccgtggcttgaagg

SEQ ID NO: 182 - RNA, 32 nt, synthetic construct
  source 1..32: mol_type = other RNA
gttgcaacccgctcgctggtgtgacttgaagg

SEQ ID NO: 183 - RNA, 32 nt, synthetic construct
  source 1..32: mol_type = other RNA
gttgcaacacgcgcgaaggtgcggcttgaagg

SEQ ID NO: 184 - RNA, 32 nt, synthetic construct
  source 1..32: mol_type = other RNA
gttgcaacacgcgcgtaaggatgacttgaagg

SEQ ID NO: 185 - RNA, 32 nt, synthetic construct
  source 1..32: mol_type = other RNA
gttgcaactcgcacgttggcactggttgaagg

SEQ ID NO: 186 - RNA, 32 nt, synthetic construct
  source 1..32: mol_type = other RNA
gttgcaatttgtatacgagtgtgacttgaagg

SEQ ID NO: 187 - RNA, 32 nt, synthetic construct
  source 1..32: mol_type = other RNA
gttgcaatctacatgcacgggtatagtgcaag

SEQ ID NO: 188 - RNA, 30 nt, synthetic construct
  source 1..30: mol_type = other RNA
gtttctgaagaaactatgtatgatgtgaag

SEQ ID NO: 189 - RNA, 32 nt, synthetic construct
  source 1..32: mol_type = other RNA
attgcaactggtgcatgtgtacaagatgtgag

SEQ ID NO: 190 - RNA, 30 nt, synthetic construct
  source 1..30: mol_type = other RNA
gtttaacactaacataagatgtatttaaat

SEQ ID NO: 191 - RNA, 31 nt, synthetic construct
  source 1..31: mol_type = other RNA
agtttaaaccaaacaatagatgtattgaaat

SEQ ID NO: 192 - RNA, 30 nt, synthetic construct
  source 1..30: mol_type = other RNA
gttaaaacataacaatagatgtattgaaat

SEQ ID NO: 193 - RNA, 32 nt, synthetic construct
  source 1..32: mol_type = other RNA
gtcacaccctgtgcgggtgtgtgggttgaaac

SEQ ID NO: 194 - RNA, 36 nt, synthetic construct
  source 1..36: mol_type = other RNA
ggtgtaggcgacctttttttgcggtgtacttcgagg

SEQ ID NO: 195 - RNA, 32 nt, synthetic construct
  source 1..32: mol_type = other RNA
gccgcatctcgcacgcgtacgtggattgaaac

SEQ ID NO: 196 - RNA, 37 nt, synthetic construct
  source 1..37: mol_type = other RNA
gttgcagaacccgaatagacgaatgaaggaatgcaac

SEQ ID NO: 197 - RNA, 29 nt, synthetic construct
  source 1..29: mol_type = other RNA
ttaatgattaacataggttatatttaaat

SEQ ID NO: 198 - RNA, 32 nt, synthetic construct
  source 1..32: mol_type = other RNA
ttgtttaatattaacaaaggttgtatttaaat

SEQ ID NO: 199 - RNA, 30 nt, synthetic construct
  source 1..30: mol_type = other RNA
gtttaagaataacaatagttgtatttaaat

SEQ ID NO: 200 - RNA, 32 nt, synthetic construct
  source 1..32: mol_type = other RNA
gtcgcaacctatatggatacgtggattgaaat

SEQ ID NO: 201 - RNA, 29 nt, synthetic construct
  source 1..29: mol_type = other RNA
gtttgatatcaactatatggaatgtaaat

SEQ ID NO: 202 - RNA, 30 nt, synthetic construct
  source 1..30: mol_type = other RNA
gttgaagaataacatgagatgtttttaaat

SEQ ID NO: 203 - RNA, 29 nt, synthetic construct
  source 1..29: mol_type = other RNA
gttttatcttaactatatggaatgtaaat

SEQ ID NO: 204 - RNA, 29 nt, synthetic construct
  source 1..29: mol_type = other RNA
gttataaatctactatgtagtatgtgaat

SEQ ID NO: 205 - RNA, 29 nt, synthetic construct
  source 1..29: mol_type = other RNA
gttataagttaactataattgatgtgaat

SEQ ID NO: 206 - RNA, 30 nt, synthetic construct
  source 1..30: mol_type = other RNA
ggtttaatagcaccataatggaatgttaat

SEQ ID NO: 207 - RNA, 29 nt, synthetic construct
  source 1..29: mol_type = other RNA
gttatataatgactatgtagtgtgtaaac

SEQ ID NO: 208 - RNA, 29 nt, synthetic construct
  source 1..29: mol_type = other RNA
gttatatattaactaagtgatatgtaaag

SEQ ID NO: 209 - RNA, 30 nt, synthetic construct
  source 1..30: mol_type = other RNA
atttgattagaaccatattggaatgtaaat

SEQ ID NO: 210 - RNA, 29 nt, synthetic construct
  source 1..29: mol_type = other RNA
gtttaatataaactatgtagtatgtgaat

SEQ ID NO: 211 - RNA, 30 nt, synthetic construct
  source 1..30: mol_type = other RNA
atttgattagtactataatggaatgtaaat

SEQ ID NO: 212 - RNA, 32 nt, synthetic construct
  source 1..32: mol_type = other RNA
cttccaatttgcgcgtgggcgtgagttggggg

SEQ ID NO: 213 - DNA, 27 bp, synthetic construct
  source 1..27: mol_type = other DNA
tttcccattacagtaggagcatacggg

SEQ ID NO: 214 - DNA, 20 bp, synthetic construct
  source 1..20: mol_type = other DNA
ccattacagtaggagcatac

SEQ ID NO: 215 - RNA, 18 nt, synthetic construct
  source 1..18: mol_type = other RNA
gggtcttcgagaagacct

SEQ ID NO: 216 - protein, 7 aa, synthetic construct
PKKKRKV

SEQ ID NO: 217 - DNA, 21 bp, synthetic construct
  source 1..21: mol_type = other DNA
cctaagaagaagagaaaggtg

SEQ ID NO: 218 - protein, 16 aa, synthetic construct
KRPAATKKAGQAKKKK

SEQ ID NO: 219 - DNA, 48 bp, synthetic construct
  source 1..48: mol_type = other DNA
aaaaggccggcggccacgaagaaggccggccaggcaaagaagaagaag

SEQ ID NO: 220 - RNA, 12 nt, synthetic construct
  source 1..12: mol_type = other RNA
ttttattttttt

SEQ ID NO: 221 - protein, 433 aa, synthetic construct
MGKGVLAKVMKYELRYLDGCGDFSNMQEQVWALQRQTREILNRSIQIAFQWDCANSEHHRKTGEYLDLKTETGYKRLDGHIYNCLKGQYEDMATSNLNATIQKAWKKYNSSKKEILRGSMSIPSYKMNQPLTLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQT

SEQ ID NO: 222 - protein, 433 aa, synthetic construct
MGKGVLAKVMKYELRYLDGCGDFSNMQEQVWALQRQTREILNRSIQIAFQWDCANSEHHRKTGEYLDLKTETGYKRLDGHIYNCLKGQYEDMATSNLNATIQKAWKKYNSSKKEILRGSMSIPSYKMNQPLTLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQT

SEQ ID NO: 223 - protein, 415 aa, synthetic construct
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVALGMKIPAYVTLNDSEYIGKPIGDINDFLKVRKQFKERKERLQKQLAINKGGRGITNKMQLMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNADRNASFNIARSTKYISSKEESDFYKQLK

SEQ ID NO: 224 - protein, 415 aa, synthetic construct
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVDLGMKIPAYVTLNDSEYIGKPIGDINDFLKVRKQFKERKERLQKQLAINKGGRGITNKMQLMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNAARNASFNIARSTKYISSKEESDFYKQLK

SEQ ID NO: 225 - protein, 433 aa, synthetic construct
MGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLTLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQT

SEQ ID NO: 226 - protein, 433 aa, synthetic construct
MGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQT

SEQ ID NO: 227 - protein, 415 aa, synthetic construct
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVDLGMKIPAYVTLNDSEYIGKPIGDINDFLKVRKQFKERKERLQKQLAINKGGRGITNKMQRMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNADRNASFNIARSTKYISSKEESDFYKQLK

SEQ ID NO: 228 - RNA, 125 nt, synthetic construct
  source 1..125: mol_type = other RNA
agggacttcccccaaaatcgagacagtagccgtaaaactttgagtttcagagtgggcgacacactcgaaaaggttaagatatgcacatagtaatccgtgcatgagccgcgaaagcggcttgaagg

SEQ ID NO: 229 - RNA, 109 nt, synthetic construct
  source 1..109: mol_type = other RNA
atcgagacagtagccgtaaaactttgagtttcagagtgggcgacacactcgaaaaggttaagatatgcacatagtaatccgtgcatgagccgcgaaagcggcttgaagg

SEQ ID NO: 230 - RNA, 113 nt, synthetic construct
  source 1..113: mol_type = other RNA
agggacgacttcccgtcccaaaatcgagacagtagccagtttggcgacacactcgaaaaggttaagatatgcacatagtaatccgtgcatgagccgcgaaagcggcttgaagg

SEQ ID NO: 231 - RNA, 95 nt, synthetic construct
  source 1..95: mol_type = other RNA
agggacgacttcccgtcccaaaatcgagagtttctcgaaaaggttaagatatgcacatagtaatccgtgcatgagccgcgaaagcggcttgaagg

SEQ ID NO: 232 - RNA, 123 nt, synthetic construct
  source 1..123: mol_type = other RNA
agggacgacttcccgtcccaaaatcgagacagtagccgtaaaactttgagtttcagagtgggcgacacactcgaaaaggttaagatatgcacatagtaatccgtgcatgaggaaacttgaagg

SEQ ID NO: 233 - RNA, 117 nt, synthetic construct
  source 1..117: mol_type = other RNA
agggacgacttcccgtcccaaaatcgagacagtagccgtaaaactttgagtttcagagtgggcgacacactcgaaaaggttaagatatgcacatagtaatccgtgcatgaaagaagg

SEQ ID NO: 234 - RNA, 131 nt, synthetic construct
  source 1..131: mol_type = other RNA
agggccgacttcccggcccaaaatcgagacagtagccgtaaaactttgagtttcagagtgggcgacacactcgaaaaggttaagatatgcacatagtaatccgtgcatgagccgcgaaagcggcttgaagg

SEQ ID NO: 235 - RNA, 131 nt, synthetic construct
  source 1..131: mol_type = other RNA
agggacgacttcccgtcccaaaatcgagacagtcgccgtaaaactttgagtttcagagtgggcgacacactcgaaaaggttaagatatgcacatagtaatccgtgcatgagccgcgaaagcggcttgaagg

SEQ ID NO: 236 - RNA, 131 nt, synthetic construct
  source 1..131: mol_type = other RNA
agggacgacttcccgtcccaaaatcgagacagtagccgtaaaactctgagtttcagagtgggcgacacactcgaaaaggttaagatatgcacatagtaatccgtgcatgagccgcgaaagcggcttgaagg

SEQ ID NO: 237 - RNA, 113 nt, synthetic construct
  source 1..113: mol_type = other RNA
agggacgacttcccgtcccaaaatcgagacagtcgccagtttggcgacacactcgaaaaggttaagatatgcacatagtaatccgtgcatgagccgcgaaagcggcttgaagg

SEQ ID NO: 238 - RNA, 131 nt, synthetic construct
  source 1..131: mol_type = other RNA
agggacgacttcccgtcccaaaatcgagacagtagccgtaaaactttgagtttcagagtgggcgacacactcgaaaaggttaagatacgcacatagtaatccgtgcgtgagccgcgaaagcggcttgaagg

SEQ ID NO: 239 - RNA, 131 nt, synthetic construct
  source 1..131: mol_type = other RNA
agggccgacttcccggcccaaaatcgagacagtcgccgtaaaactctgagtttcagagtgggcgacacactcgaaaaggttaagatatgcacatagtaatccgtgcatgagccgcgaaagcggcttgaagg

SEQ ID NO: 240 - RNA, 131 nt, synthetic construct
  source 1..131: mol_type = other RNA
agggccgacttcccggcccaaaatcgagacagtagccgtaaaactttgagtttcagagtgggctacacactcgaaaaggttaagatatgcacatagtaatccgtgcatgagccgcgaaagcggcttgaagg

SEQ ID NO: 241 - RNA, 131 nt, synthetic construct
  source 1..131: mol_type = other RNA
agggccgacttcccggcccaaaagcgagacagtagccgtaaaactttgagtttcagagtgggcgacacactcgcaaaggttaagatatgcacatagtaatccgtgcatgagccgcgaaagcggcttgaagg

SEQ ID NO: 242 - RNA, 131 nt, synthetic construct
  source 1..131: mol_type = other RNA
agggccgacttcccggcccaaaatcgcgacagtagccgtaaaactttgagtttcagagtgggcgacacacgcgaaaaggttaagatatgcacatagtaatccgtgcatgagccgcgaaagcggcttgaagg

SEQ ID NO: 243 - RNA, 131 nt, synthetic construct
  source 1..131: mol_type = other RNA
agggccgacttcccggcccaaaatcgagacagtagccgtaaagctttgagtttcagagcgggcgacacactcgaaaaggttaagatatgcacatagtaatccgtgcatgagccgcgaaagcggcttgaagg

SEQ ID NO: 244 - RNA, 131 nt, synthetic construct
  source 1..131: mol_type = other RNA
agggccgacttcccggcccaaaatcgagacagtagccgtaaaacgttgagtttcagcgtgggcgacacactcgaaaaggttaagatatgcacatagtaatccgtgcatgagccgcgaaagcggcttgaagg

SEQ ID NO: 245 - RNA, 131 nt, synthetic construct
  source 1..131: mol_type = other RNA
agggccgacttcccggcccaaaatcgagacagtagccgtaaaacttcgagtttcggagtgggcgacacactcgaaaaggttaagatatgcacatagtaatccgtgcatgagccgcgaaagcggcttgaagg

SEQ ID NO: 246 - RNA, 131 nt, synthetic construct
  source 1..131: mol_type = other RNA
agggccgacttcccggcccaaaatcgagacagtagccgtaaaactttgcgttgcagagtgggcgacacactcgaaaaggttaagatatgcacatagtaatccgtgcatgagccgcgaaagcggcttgaagg

SEQ ID NO: 247 - RNA, 131 nt, synthetic construct
  source 1..131: mol_type = other RNA
agggccgacttcccggcccaaaatcgagacagtagccgtaaaactttgagtttcagagtgggcgacacactcgaaaaggttaagatgtgcacatagtaatccgtgcacgagccgcgaaagcggcttgaagg

SEQ ID NO: 248 - RNA, 131 nt, synthetic construct
  source 1..131: mol_type = other RNA
agggccgacttcccggcccaaaatcgagacagtagccgtaaaactttgagtttcagagtgggcgacacactcgaaaaggttaagatatgcacatagtaatccgtgcatgagccgcgaaagcggctcgaagg

SEQ ID NO: 249 - RNA, 131 nt, synthetic construct
  source 1..131: mol_type = other RNA
agggccgacttcccggcccaaaatcgagacagtagccgtaaaactttgagtttcagagtgggcgacacactcgaaaaggttaagatatgcacatagtaatccgtgcatgcgccgcgaaagcggcgtgaagg

SEQ ID NO: 250 - RNA, 131 nt, synthetic construct
  source 1..131: mol_type = other RNA
agggccgacttcccggcccaaaatcgagacagtcgccgtaaaactttgagtttcagagtgggcgacacactcgaaaaggttaagatatgcacatagtaatccgtgcatgagccgcgaaagcggcttgaagg

SEQ ID NO: 251 - RNA, 131 nt, synthetic construct
  source 1..131: mol_type = other RNA
agggccgacttcccggcccaaaatcgagacagtagccgtaaaactctgagtttcagagtgggcgacacactcgaaaaggttaagatatgcacatagtaatccgtgcatgagccgcgaaagcggcttgaagg

SEQ ID NO: 252 - RNA, 121 nt, synthetic construct
  source 1..121: mol_type = other RNA
aagtctgagggcatgtagaaaaaagtataggtatataccaacatacttgcattgccactcggaaagggttaaccttggtcattgtgttaccgaccaagcattccagaaatggaatgtaaat

SEQ ID NO: 253 - RNA, 132 nt, synthetic construct
  source 1..132: mol_type = other RNA
acggttgatttagcaaccgaagtgggcatgtagaaaaaagtataggtatataccaacatacttgcattgccacaaagggttaaccttggtcattgtgttaccgaccaagcattccagaaatggaatgtaaat

SEQ ID NO: 254 - RNA, 133 nt, synthetic construct
  source 1..133: mol_type = other RNA
acggttgatttagcaaccgaagtctggcatgtagaaaaaagtataggtatataccaacatacttgcattgccggaaagggttaaccttggtcattgtgttaccgaccaagcattccagaaatggaatgtaaat

SEQ ID NO: 255 - RNA, 133 nt, synthetic construct
  source 1..133: mol_type = other RNA
acggttgatttagcaaccgaagtctgaggggtagaaaaaagtataggtatataccaacatacttgcccactcggaaagggttaaccttggtcattgtgttaccgaccaagcattccagaaatggaatgtaaat

SEQ ID NO: 256 - RNA, 128 nt, synthetic construct
  source 1..128: mol_type = other RNA
acggttgatttagcaaccgaagtctgagggcatggtataggtatataccaacataccattgccactcggaaagggttaaccttggtcattgtgttaccgaccaagcattccagaaatggaatgtaaat

SEQ ID NO: 257 - RNA, 140 nt, synthetic construct
  source 1..140: mol_type = other RNA
acggctgatttagcagccgaagtctgagggcatgtagaaaaaagtataggtatataccaacatacttgcattgccactcggaaagggttaaccttggtcattgtgttaccgaccaagcattccagaaatggaatgtaaat

SEQ ID NO: 258 - RNA, 140 nt, synthetic construct
  source 1..140: mol_type = other RNA
acggtcgatttagcgaccgaagtctgagggcatgtagaaaaaagtataggtatataccaacatacttgcattgccactcggaaagggttaaccttggtcattgtgttaccgaccaagcattccagaaatggaatgtaaat

SEQ ID NO: 259 - RNA, 140 nt, synthetic construct
  source 1..140: mol_type = other RNA
acggccgatttagcggccgaagtctgagggcatgtagaaaaaagtataggtatataccaacatacttgcattgccactcggaaagggttaaccttggtcattgtgttaccgaccaagcattccagaaatggaatgtaaat

SEQ ID NO: 260 - protein, 821 aa, synthetic construct
MDYKDHDGDYKDHDIDYKDDDDKMAPKKKRKVGIHGVPAAGSISLIAALAVDYVIGMENAMPWNLPADLAWFKRNTLNKPVIMGRHTWESIGRPLPGRKNIILSSQPSTDDRVTWVKSVDEAIAACGDVPEIMVIGGGRVIEQFLPKAQKLYLTHIDAEVEGDTHFPDYEPDDWESVFSEFHDADAQNSHSYCFEILERRGSGSGSMPKKKRKVMGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVDMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANADFNASQNISIRNIDKIIAKAIGANRKQTKRPAATKKAGQAKKKKISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 261 - protein, 1442 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSERGSGSGSMPKKKRKVMGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQTKRPAATKKAGQAKKKKAYPYDVPDYASLGSGSPKKKRKVEDPKKKRKVDGIGSGSNGSSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEP

SEQ ID NO: 262 - protein, 1446 aa, synthetic construct
MAYPYDVPDYASLGSGSPKKKRKVEDPKKKRKVDGIGSGSNGSSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEPRGSGSGSMPKKKRKVMGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQTKRPAATKKAGQAKKKKSSGMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSE

SEQ ID NO: 263 - protein, 1461 aa, synthetic construct
MDYKDHDGDYKDHDIDYKDDDDKMPKKKRKVMGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQTKRPAATKKAGQAKKKKSSGMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEAYPYDVPDYASLGSGSPKKKRKVEDPKKKRKVDGIGSGSNGSSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEP

SEQ ID NO: 264 - protein, 1433 aa, synthetic construct
MDYKDHDGDYKDHDIDYKDDDDKMPKKKRKVMGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQTKRPAATKKAGQAKKKKGSSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEPRGSGSGSMPKKKRKVMNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSE

SEQ ID NO: 265 - protein, 1282 aa, synthetic construct
MDYKDHDGDYKDHDIDYKDDDDKMPKKKRKVMGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQTKRPAATKKAGQAKKKKAYPYDVPDYASLGSGDGIGSGSNGSSLDALDDFDLDMLGSDALDDFDLDMLGSDALDDFDLDMLGSDALDDFDLDMLGSGGSGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFHMGGGSGEDPAAKRVKLDMGSGVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFDDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 266 - DNA, 20 bp, synthetic construct
  source 1..20: mol_type = other DNA
ccggtggtcactctgtatgc

SEQ ID NO: 267 - DNA, 20 bp, synthetic construct
  source 1..20: mol_type = other DNA
cgtctttgactctaaggccc

SEQ ID NO: 268 - DNA, 20 bp, synthetic construct
  source 1..20: mol_type = other DNA
ctctgccccaggctgcagct

SEQ ID NO: 269 - DNA, 20 bp, synthetic construct
  source 1..20: mol_type = other DNA
caggtcatcacagttggggc

SEQ ID NO: 270 - DNA, 20 bp, synthetic construct
  source 1..20: mol_type = other DNA
tccaggagtgggaagcggcg

SEQ ID NO: 271 - DNA, 20 bp, synthetic construct
  source 1..20: mol_type = other DNA
ctcgggctctggcaggtgac

SEQ ID NO: 272 - DNA, 20 bp, synthetic construct
  source 1..20: mol_type = other DNA
actctaaggcccaagggggc

SEQ ID NO: 273 - DNA, 20 bp, synthetic construct
  source 1..20: mol_type = other DNA
ggggtgagggtgtctacgcc

SEQ ID NO: 274 - DNA, 20 bp, synthetic construct
  source 1..20: mol_type = other DNA
cattccagacctggggcatg

SEQ ID NO: 275 - DNA, 20 bp, synthetic construct
  source 1..20: mol_type = other DNA
ttcggaaaagccagctggtc

SEQ ID NO: 276 - DNA, 20 bp, synthetic construct
  source 1..20: mol_type = other DNA
cccagagcatcccgtggaac

SEQ ID NO: 277 - DNA, 20 bp, synthetic construct
  source 1..20: mol_type = other DNA
ttcctcccaggcctggagtt

SEQ ID NO: 278 - DNA, 20 bp, synthetic construct
  source 1..20: mol_type = other DNA
gggaccaactttggccgctg

SEQ ID NO: 279 - DNA, 20 bp, synthetic construct
  source 1..20: mol_type = other DNA
gccgctgtgtggacctcttt

SEQ ID NO: 280 - DNA, 20 bp, synthetic construct
  source 1..20: mol_type = other DNA
ccccaggggaggacatcatt

SEQ ID NO: 281 - DNA, 20 bp, synthetic construct
  source 1..20: mol_type = other DNA
tgtcacagagtgggacatca

SEQ ID NO: 282 - DNA, 20 bp, synthetic construct
  source 1..20: mol_type = other DNA
gcagagaagtggatcagtct

SEQ ID NO: 283 - DNA, 20 bp, synthetic construct
  source 1..20: mol_type = other DNA
caggttggcagctgttttgc

SEQ ID NO: 284 - DNA, 20 bp, synthetic construct
  source 1..20: mol_type = other DNA
caggactgtatggtcagcac

SEQ ID NO: 285 - DNA, 20 bp, synthetic construct
  source 1..20: mol_type = other DNA
tccctacacccgcaccttgg

SEQ ID NO: 286 - DNA, 20 bp, synthetic construct
  source 1..20: mol_type = other DNA
cctctcgcagtcagagcgca

SEQ ID NO: 287 - DNA, 20 bp, synthetic construct
  source 1..20: mol_type = other DNA
ggctgcccgccggggatacc

SEQ ID NO: 288 - DNA, 20 bp, synthetic construct
  source 1..20: mol_type = other DNA
aaaagggtggctcaccagct

SEQ ID NO: 289 - DNA, 20 bp, synthetic construct
  source 1..20: mol_type = other DNA
tgtcgactacatcgaggagg

SEQ ID NO: 290 - DNA, 20 bp, synthetic construct
  source 1..20: mol_type = other DNA
gagcatcccgtggaacctgg

SEQ ID NO: 291 - DNA, 20 bp, synthetic construct
  source 1..20: mol_type = other DNA
caaatgtcgccttggaaaga

SEQ ID NO: 292 - DNA, 20 bp, synthetic construct
  source 1..20: mol_type = other DNA
tcagacggccgtgcttacct

SEQ ID NO: 293 - DNA, 20 bp, synthetic construct
  source 1..20: mol_type = other DNA
cctggcaggggtggtcagcg

SEQ ID NO: 294 - DNA, 20 bp, synthetic construct
  source 1..20: mol_type = other DNA
ggcctggagtttattcggaa

SEQ ID NO: 295 - DNA, 20 bp, synthetic construct
  source 1..20: mol_type = other DNA
cccgccaggggcagcagcac

SEQ ID NO: 296 - DNA, 20 bp, synthetic construct
  source 1..20: mol_type = other DNA
gccctcgccaggcgctggca

SEQ ID NO: 297 - DNA, 20 bp, synthetic construct
  source 1..20: mol_type = other DNA
gcacctacctcgggagctga

SEQ ID NO: 298 - DNA, 20 bp, synthetic construct
  source 1..20: mol_type = other DNA
cctcctcacctttccaggtc

SEQ ID NO: 299 - DNA, 20 bp, synthetic construct
  source 1..20: mol_type = other DNA
agaccagccggtgaccctgg

SEQ ID NO: 300 - DNA, 20 bp, synthetic construct
  source 1..20: mol_type = other DNA
gggtcaccggctggtcttgg

SEQ ID NO: 301 - DNA, 20 bp, synthetic construct
  source 1..20: mol_type = other DNA
aagtccccagggtcaccggc

SEQ ID NO: 302 - DNA, 20 bp, synthetic construct
  source 1..20: mol_type = other DNA
ggggaggacatcattggtgc

SEQ ID NO: 303 - DNA, 20 bp, synthetic construct
  source 1..20: mol_type = other DNA
ctctgtgacacaaagcaggt

SEQ ID NO: 304 - DNA, 20 bp, synthetic construct
  source 1..20: mol_type = other DNA
acctggtggccgccctgccc

SEQ ID NO: 305 - DNA, 20 bp, synthetic construct
  source 1..20: mol_type = other DNA
tgggtgctggggggcagggc

SEQ ID NO: 306 - DNA, 20 bp, synthetic construct
  source 1..20: mol_type = other DNA
ccctgccatcctgcttacct

SEQ ID NO: 307 - DNA, 20 bp, synthetic construct
  source 1..20: mol_type = other DNA
ggccctttttgcaggttggc

SEQ ID NO: 308 - DNA, 20 bp, synthetic construct
  source 1..20: mol_type = other DNA
gatgaggagctgctgagctg

SEQ ID NO: 309 - DNA, 20 bp, synthetic construct
  source 1..20: mol_type = other DNA
ctcctggagaaactggagca

SEQ ID NO: 310 - DNA, 20 bp, synthetic construct
  source 1..20: mol_type = other DNA
tttccgtctttgactctaag

SEQ ID NO: 311 - DNA, 20 bp, synthetic construct
  source 1..20: mol_type = other DNA
agggggcaagctggtctgcc

SEQ ID NO: 312 - DNA, 20 bp, synthetic construct
  source 1..20: mol_type = other DNA
caacgcttttgggggtgagg

SEQ ID NO: 313 - DNA, 20 bp, synthetic construct
  source 1..20: mol_type = other DNA
aaagcgttgtgggcccggca

SEQ ID NO: 314 - DNA, 20 bp, synthetic construct
  source 1..20: mol_type = other DNA
ggccaactgcagcgtccaca

SEQ ID NO: 315 - DNA, 20 bp, synthetic construct
  source 1..20: mol_type = other DNA
tgctggcctcagctggtgga

SEQ ID NO: 316 - DNA, 20 bp, synthetic construct
  source 1..20: mol_type = other DNA
gcctcctacctgtgaggacg

SEQ ID NO: 317 - DNA, 20 bp, synthetic construct
  source 1..20: mol_type = other DNA
gggcaagcccagcctcctac

SEQ ID NO: 318 - DNA, 20 bp, synthetic construct
  source 1..20: mol_type = other DNA
ggctgcagctcccactggga

SEQ ID NO: 319 - DNA, 20 bp, synthetic construct
  source 1..20: mol_type = other DNA
ctgggaggtggaggaccttg

SEQ ID NO: 320 - DNA, 20 bp, synthetic construct
  source 1..20: mol_type = other DNA
caagccgcctgtgctgaggc

SEQ ID NO: 321 - DNA, 20 bp, synthetic construct
  source 1..20: mol_type = other DNA
cgcactggttgggctgacct

SEQ ID NO: 322 - DNA, 20 bp, synthetic construct
  source 1..20: mol_type = other DNA
ggtctggaatgcaaagtcaa

SEQ ID NO: 323 - DNA, 20 bp, synthetic construct
  source 1..20: mol_type = other DNA
ggacgtgggaggtcccaggg

SEQ ID NO: 324 - DNA, 20 bp, synthetic construct
  source 1..20: mol_type = other DNA
tgaacacatgcacggccaca

SEQ ID NO: 325 - DNA, 20 bp, synthetic construct
  source 1..20: mol_type = other DNA
gctccagatttctaatacca

SEQ ID NO: 326 - DNA, 20 bp, synthetic construct
  source 1..20: mol_type = other DNA
tgcctccagacacactgcta

SEQ ID NO: 327 - DNA, 20 bp, synthetic construct
  source 1..20: mol_type = other DNA
acaccttataggaaaaccag

SEQ ID NO: 328 - DNA, 20 bp, synthetic construct
  source 1..20: mol_type = other DNA
tcatcgtctgctcctcctct

SEQ ID NO: 329 - DNA, 20 bp, synthetic construct
  source 1..20: mol_type = other DNA
tagatgctgtccgaggcagt

SEQ ID NO: 330 - DNA, 20 bp, synthetic construct
  source 1..20: mol_type = other DNA
agaaaggctgctgatgacac

SEQ ID NO: 331 - DNA, 20 bp, synthetic construct
  source 1..20: mol_type = other DNA
tttggcaacttacccagagg

SEQ ID NO: 332 - DNA, 20 bp, synthetic construct
  source 1..20: mol_type = other DNA
ctcctcagttgtgagcccat

SEQ ID NO: 333 - DNA, 20 bp, synthetic construct
  source 1..20: mol_type = other DNA
tacaaattcctcctcagttg

SEQ ID NO: 334 - DNA, 20 bp, synthetic construct
  source 1..20: mol_type = other DNA
cagtaagatttggtgtctat

SEQ ID NO: 335 - DNA, 20 bp, synthetic construct
  source 1..20: mol_type = other DNA
tctcataggtggtattcaca

SEQ ID NO: 336 - DNA, 20 bp, synthetic construct
  source 1..20: mol_type = other DNA
acagccaacgactccggccc

SEQ ID NO: 337 - DNA, 20 bp, synthetic construct
  source 1..20: mol_type = other DNA
tggtattagaaatctggagc

SEQ ID NO: 338 - DNA, 20 bp, synthetic construct
  source 1..20: mol_type = other DNA
ttaacttctcacgtgtcttc

SEQ ID NO: 339 - DNA, 20 bp, synthetic construct
  source 1..20: mol_type = other DNA
accatcagaggacacttgga

SEQ ID NO: 340 - DNA, 20 bp, synthetic construct
  source 1..20: mol_type = other DNA
gcaacttacccagaggcaaa

SEQ ID NO: 341 - DNA, 20 bp, synthetic construct
  source 1..20: mol_type = other DNA
tagaagggatatacaaagtg

SEQ ID NO: 342 - DNA, 20 bp, synthetic construct
  source 1..20: mol_type = other DNA
tatatcccttctacaaattc

SEQ ID NO: 343 - DNA, 20 bp, synthetic construct
  source 1..20: mol_type = other DNA
gtgtctatttccactttgta

SEQ ID NO: 344 - DNA, 20 bp, synthetic construct
  source 1..20: mol_type = other DNA
gactttcacaccttatagga

SEQ ID NO: 345 - DNA, 20 bp, synthetic construct
  source 1..20: mol_type = other DNA
gactcactggttttcctata

SEQ ID NO: 346 - DNA, 20 bp, synthetic construct
  source 1..20: mol_type = other DNA
ctttgtatatcccttctaca

SEQ ID NO: 347 - DNA, 20 bp, synthetic construct
  source 1..20: mol_type = other DNA
gtaagatttggtgtctattt

SEQ ID NO: 348 - DNA, 20 bp, synthetic construct
  source 1..20: mol_type = other DNA
gcaaggcagaggaggagcag

SEQ ID NO: 349 - DNA, 20 bp, synthetic construct
  source 1..20: mol_type = other DNA
agtgtcctctgatggtcaaa

SEQ ID NO: 350 - DNA, 20 bp, synthetic construct
  source 1..20: mol_type = other DNA
gggcaccggtgaatccaagt

SEQ ID NO: 351 - DNA, 20 bp, synthetic construct
  source 1..20: mol_type = other DNA
ggtgtcatcagcagcctttc

SEQ ID NO: 352 - DNA, 20 bp, synthetic construct
  source 1..20: mol_type = other DNA
gaggcaaatggctcccaggt

SEQ ID NO: 353 - DNA, 20 bp, synthetic construct
  source 1..20: mol_type = other DNA
tgcagctctccagactcact

SEQ ID NO: 354 - DNA, 20 bp, synthetic construct
  source 1..20: mol_type = other DNA
tgagcatgcagaggtgagta

SEQ ID NO: 355 - DNA, 20 bp, synthetic construct
  source 1..20: mol_type = other DNA
gtccaacttctgggctgttc

SEQ ID NO: 356 - DNA, 20 bp, synthetic construct
  source 1..20: mol_type = other DNA
ggaggcccgaccggggccgg

SEQ ID NO: 357 - DNA, 20 bp, synthetic construct
  source 1..20: mol_type = other DNA
tgctgtcttgggtgcattgg

SEQ ID NO: 358 - DNA, 20 bp, synthetic construct
  source 1..20: mol_type = other DNA
tgtcctcagtggtcccaggc

SEQ ID NO: 359 - DNA, 20 bp, synthetic construct
  source 1..20: mol_type = other DNA
cagattatgcggatcaaacc

SEQ ID NO: 360 - DNA, 20 bp, synthetic construct
  source 1..20: mol_type = other DNA
cagaaaatcagttcgaggaa

SEQ ID NO: 361 - DNA, 20 bp, synthetic construct
  source 1..20: mol_type = other DNA
cctttcctcgaactgatttt

SEQ ID NO: 362 - DNA, 20 bp, synthetic construct
  source 1..20: mol_type = other DNA
gtttttgcccctttcccttt

SEQ ID NO: 363 - DNA, 20 bp, synthetic construct
  source 1..20: mol_type = other DNA
ttgcgctttcgtttttgccc

SEQ ID NO: 364 - DNA, 20 bp, synthetic construct
  source 1..20: mol_type = other DNA
cttttgcctttttgcagtcc

SEQ ID NO: 365 - DNA, 20 bp, synthetic construct
  source 1..20: mol_type = other DNA
tccgctctgagcaaggccca

SEQ ID NO: 366 - DNA, 20 bp, synthetic construct
  source 1..20: mol_type = other DNA
ttgtgctgtaggaagctcat

SEQ ID NO: 367 - DNA, 20 bp, synthetic construct
  source 1..20: mol_type = other DNA
cccctttccctttcctcgaa

SEQ ID NO: 368 - DNA, 20 bp, synthetic construct
  source 1..20: mol_type = other DNA
cctttttgcagtccctgtgg

SEQ ID NO: 369 - DNA, 20 bp, synthetic construct
  source 1..20: mol_type = other DNA
cagtccctgtgggccttgct

SEQ ID NO: 370 - DNA, 20 bp, synthetic construct
  source 1..20: mol_type = other DNA
tttgtacaagatccgcagac

SEQ ID NO: 371 - DNA, 20 bp, synthetic construct
  source 1..20: mol_type = other DNA
tacaagatccgcagacgtgt

SEQ ID NO: 372 - DNA, 20 bp, synthetic construct
  source 1..20: mol_type = other DNA
caggaacatttacacgtctg

SEQ ID NO: 373 - DNA, 20 bp, synthetic construct
  source 1..20: mol_type = other DNA
gccccagctaccacctcctc

SEQ ID NO: 374 - DNA, 20 bp, synthetic construct
  source 1..20: mol_type = other DNA
gctaccacctcctccccggc

SEQ ID NO: 375 - DNA, 20 bp, synthetic construct
  source 1..20: mol_type = other DNA
gaagttggacgaaaagtttc

SEQ ID NO: 376 - DNA, 20 bp, synthetic construct
  source 1..20: mol_type = other DNA
ggccctggcccgggcctcgg

SEQ ID NO: 377 - DNA, 20 bp, synthetic construct
  source 1..20: mol_type = other DNA
cagcccgagccggagaggga

SEQ ID NO: 378 - DNA, 20 bp, synthetic construct
  source 1..20: mol_type = other DNA
agacagcagaaagttcatgg

SEQ ID NO: 379 - DNA, 20 bp, synthetic construct
  source 1..20: mol_type = other DNA
ggctgcacccatggcagaag

SEQ ID NO: 380 - DNA, 20 bp, synthetic construct
  source 1..20: mol_type = other DNA
tggcagaaggaggagggcag

SEQ ID NO: 381 - DNA, 20 bp, synthetic construct
  source 1..20: mol_type = other DNA
ctgaggagtccaacatcacc

SEQ ID NO: 382 - DNA, 20 bp, synthetic construct
  source 1..20: mol_type = other DNA
cctgcatggtgatgttggac

SEQ ID NO: 383 - DNA, 20 bp, synthetic construct
  source 1..20: mol_type = other DNA
aagatgcccacctgcatggt

SEQ ID NO: 384 - DNA, 20 bp, synthetic construct
  source 1..20: mol_type = other DNA
cttcccaaagatgcccacct

SEQ ID NO: 385 - DNA, 20 bp, synthetic construct
  source 1..20: mol_type = other DNA
cacataggccattcagaaac

SEQ ID NO: 386 - DNA, 20 bp, synthetic construct
  source 1..20: mol_type = other DNA
gagggagacacaagttgata

SEQ ID NO: 387 - DNA, 20 bp, synthetic construct
  source 1..20: mol_type = other DNA
agaacacatacccctgggcc

SEQ ID NO: 388 - DNA, 20 bp, synthetic construct
  source 1..20: mol_type = other DNA
ccctggctacctcccctacc

SEQ ID NO: 389 - DNA, 20 bp, synthetic construct
  source 1..20: mol_type = other DNA
agaacacatacccctgggcc

SEQ ID NO: 390 - DNA, 20 bp, synthetic construct
  source 1..20: mol_type = other DNA
ggggaggacatcattggtgc

SEQ ID NO: 391 - DNA, 20 bp, synthetic construct
  source 1..20: mol_type = other DNA
ttgtgcagtgccaggtgaaa

SEQ ID NO: 392 - DNA, 19 bp, synthetic construct
  source 1..19: mol_type = other DNA
ctcctcagaaccaagcgtc

SEQ ID NO: 393 - DNA, 20 bp, synthetic construct
  source 1..20: mol_type = other DNA
cctcactcctgctcggtgaa

SEQ ID NO: 394 - DNA, 20 bp, synthetic construct
  source 1..20: mol_type = other DNA
tcaaggggctgctgtgagga

SEQ ID NO: 395 - DNA, 20 bp, synthetic construct
  source 1..20: mol_type = other DNA
ccttcagctaaaataaagga

SEQ ID NO: 396 - DNA, 20 bp, synthetic construct
  source 1..20: mol_type = other DNA
gctcagcaggcacctgcctc

SEQ ID NO: 397 - DNA, 20 bp, synthetic construct
  source 1..20: mol_type = other DNA
gtcaggttggctgctgggct

SEQ ID NO: 398 - DNA, 20 bp, synthetic construct
  source 1..20: mol_type = other DNA
taggcatgagccgctgcacc

SEQ ID NO: 399 - DNA, 20 bp, synthetic construct
  source 1..20: mol_type = other DNA
tgcggatcaaacctcaccaa

SEQ ID NO: 400 - DNA, 20 bp, synthetic construct
  source 1..20: mol_type = other DNA
cacatctgagctggctttcc

SEQ ID NO: 401 - DNA, 20 bp, synthetic construct
  source 1..20: mol_type = other DNA
taagggagaaaataggtccc

SEQ ID NO: 402 - DNA, 20 bp, synthetic construct
  source 1..20: mol_type = other DNA
gttgtgctgtaggaagctca

SEQ ID NO: 403 - DNA, 20 bp, synthetic construct
  source 1..20: mol_type = other DNA
ctcctcagttgtgagcccat

SEQ ID NO: 404 - DNA, 20 bp, synthetic construct
  source 1..20: mol_type = other DNA
acagccaacgactccggccc

SEQ ID NO: 405 - DNA, 20 bp, synthetic construct
  source 1..20: mol_type = other DNA
tacatcttcacccaccaggg

SEQ ID NO: 406 - DNA, 20 bp, synthetic construct
  source 1..20: mol_type = other DNA
tgtggacagcatgtatatgt

SEQ ID NO: 407 - DNA, 20 bp, synthetic construct
  source 1..20: mol_type = other DNA
cagcagcccccgcatcgcat

SEQ ID NO: 408 - DNA, 20 bp, synthetic construct
  source 1..20: mol_type = other DNA
ccctggctacctcccctacc

SEQ ID NO: 409 - DNA, 20 bp, synthetic construct
  source 1..20: mol_type = other DNA
gagggagacacaagttgata

SEQ ID NO: 410 - DNA, 20 bp, synthetic construct
  source 1..20: mol_type = other DNA
ctgtgtcagtttgtgccacc

SEQ ID NO: 411 - DNA, 20 bp, synthetic construct
  source 1..20: mol_type = other DNA
aagggagaaaataggtcccc

SEQ ID NO: 412 - DNA, 20 bp, synthetic construct
  source 1..20: mol_type = other DNA
cgtccaacttctgggctgtt

SEQ ID NO: 413 - DNA, 20 bp, synthetic construct
  source 1..20: mol_type = other DNA
ctctgccccaggctgcagct

SEQ ID NO: 414 - DNA, 20 bp, synthetic construct
  source 1..20: mol_type = other DNA
tgcctccagacacactgcta

SEQ ID NO: 415 - DNA, 20 bp, synthetic construct
  source 1..20: mol_type = other DNA
gtccaacttctgggctgttc

SEQ ID NO: 416 - DNA, 20 bp, synthetic construct
  source 1..20: mol_type = other DNA
tactttgtcctccggttctg

SEQ ID NO: 417 - DNA, 20 bp, synthetic construct
  source 1..20: mol_type = other DNA
actttagtgactagccgcca

SEQ ID NO: 418 - DNA, 20 bp, synthetic construct
  source 1..20: mol_type = other DNA
ggttctctctatagccattg

SEQ ID NO: 419 - DNA, 20 bp, synthetic construct
  source 1..20: mol_type = other DNA
ctgatctggacaaaagcaaa

SEQ ID NO: 420 - DNA, 20 bp, synthetic construct
  source 1..20: mol_type = other DNA
ctggaaggcacttggcatct

SEQ ID NO: 421 - DNA, 20 bp, synthetic construct
  source 1..20: mol_type = other DNA
ccttggcatggtggaggtag

SEQ ID NO: 422 - DNA, 20 bp, synthetic construct
  source 1..20: mol_type = other DNA
cctctgccccaggctgcagc

SEQ ID NO: 423 - DNA, 20 bp, synthetic construct
  source 1..20: mol_type = other DNA
ctgaacacatgcacggccac

SEQ ID NO: 424 - DNA, 20 bp, synthetic construct
  source 1..20: mol_type = other DNA
ctgctgtcttgggtgcattg

SEQ ID NO: 425 - DNA, 20 bp, synthetic construct
  source 1..20: mol_type = other DNA
cagtaagatttggtgtctat

SEQ ID NO: 426 - DNA, 20 bp, synthetic construct
  source 1..20: mol_type = other DNA
tctcataggtggtattcaca

SEQ ID NO: 427 - DNA, 20 bp, synthetic construct
  source 1..20: mol_type = other DNA
atggtcctaggtggcttcac

SEQ ID NO: 428 - DNA, 20 bp, synthetic construct
  source 1..20: mol_type = other DNA
tgggtgccaaggtcctccac

SEQ ID NO: 429 - DNA, 20 bp, synthetic construct
  source 1..20: mol_type = other DNA
gattcaccggtgccctgggt

SEQ ID NO: 430 - DNA, 20 bp, synthetic construct
  source 1..20: mol_type = other DNA
ggtgcattggagccttgcct

SEQ ID NO: 431 - DNA, 20 bp, synthetic construct
  source 1..20: mol_type = other DNA
aatagatcagagaggccagg

SEQ ID NO: 432 - DNA, 20 bp, synthetic construct
  source 1..20: mol_type = other DNA
gtgacccagccagccatacc

SEQ ID NO: 433 - DNA, 20 bp, synthetic construct
  source 1..20: mol_type = other DNA
gtgccaccaccataccgcca

SEQ ID NO: 434 - DNA, 20 bp, synthetic construct
  source 1..20: mol_type = other DNA
gatccgcataatctggaaag

SEQ ID NO: 435 - DNA, 20 bp, synthetic construct
  source 1..20: mol_type = other DNA
tagatgctgtccgaggcagt

SEQ ID NO: 436 - DNA, 20 bp, synthetic construct
  source 1..20: mol_type = other DNA
agaaaggctgctgatgacac

SEQ ID NO: 437 - DNA, 20 bp, synthetic construct
  source 1..20: mol_type = other DNA
cggaactgcatgctcaccac

SEQ ID NO: 438 - DNA, 20 bp, synthetic construct
  source 1..20: mol_type = other DNA
ggctgacctcgtggcctcag

SEQ ID NO: 439 - DNA, 20 bp, synthetic construct
  source 1..20: mol_type = other DNA
ccaaagaaccctcccacagg

SEQ ID NO: 440 - DNA, 20 bp, synthetic construct
  source 1..20: mol_type = other DNA
tgctgtaggaagctcatctc

SEQ ID NO: 441 - DNA, 20 bp, synthetic construct
  source 1..20: mol_type = other DNA
atgatgtcctcccctggggc

SEQ ID NO: 442 - DNA, 20 bp, synthetic construct
  source 1..20: mol_type = other DNA
aatcttactggaaggcactt

SEQ ID NO: 443 - DNA, 20 bp, synthetic construct
  source 1..20: mol_type = other DNA
cggctcctccgaagcgagaa

SEQ ID NO: 444 - DNA, 20 bp, synthetic construct
  source 1..20: mol_type = other DNA
ctttgccccaggggaggaca

SEQ ID NO: 445 - DNA, 20 bp, synthetic construct
  source 1..20: mol_type = other DNA
ctgcatgctcatggaatggg

SEQ ID NO: 446 - DNA, 20 bp, synthetic construct
  source 1..20: mol_type = other DNA
tggcatggtggaggtagagc

SEQ ID NO: 447 - DNA, 20 bp, synthetic construct
  source 1..20: mol_type = other DNA
tggggactttggggaccaac

SEQ ID NO: 448 - DNA, 20 bp, synthetic construct
  source 1..20: mol_type = other DNA
tcgaaggtctgtatactcac

SEQ ID NO: 449 - DNA, 20 bp, synthetic construct
  source 1..20: mol_type = other DNA
tggtggacatcttccaggag

SEQ ID NO: 450 - DNA, 20 bp, synthetic construct
  source 1..20: mol_type = other DNA
gctggtcttgggcattggtg

SEQ ID NO: 451 - DNA, 20 bp, synthetic construct
  source 1..20: mol_type = other DNA
gtgaatccaagtgtcctctg

SEQ ID NO: 452 - DNA, 20 bp, synthetic construct
  source 1..20: mol_type = other DNA
cttaccttggcatggtggag

SEQ ID NO: 453 - DNA, 20 bp, synthetic construct
  source 1..20: mol_type = other DNA
gactttcacaccttatagga

SEQ ID NO: 454 - DNA, 20 bp, synthetic construct
  source 1..20: mol_type = other DNA
ctttgtatatcccttctaca

SEQ ID NO: 455 - DNA, 20 bp, synthetic construct
  source 1..20: mol_type = other DNA
gcaaggcagaggaggagcag

SEQ ID NO: 456 - DNA, 20 bp, synthetic construct
  source 1..20: mol_type = other DNA
cccctggggcaaagaggtcc

SEQ ID NO: 457 - DNA, 20 bp, synthetic construct
  source 1..20: mol_type = other DNA
cctcagttgtgagcccatgc

SEQ ID NO: 458 - DNA, 20 bp, synthetic construct
  source 1..20: mol_type = other DNA
ccgaagcgagaacagcccag

SEQ ID NO: 459 - DNA, 20 bp, synthetic construct
  source 1..20: mol_type = other DNA
ctggggcaaagaggtccaca

SEQ ID NO: 460 - DNA, 20 bp, synthetic construct
  source 1..20: mol_type = other DNA
ttctacaaattcctcctcag

SEQ ID NO: 461 - DNA, 20 bp, synthetic construct
  source 1..20: mol_type = other DNA
ggcccgagctagcacttctc

SEQ ID NO: 462 - DNA, 20 bp, synthetic construct
  source 1..20: mol_type = other DNA
tggaggttgcctggcaccta

SEQ ID NO: 463 - DNA, 20 bp, synthetic construct
  source 1..20: mol_type = other DNA
aggcagtcctgccatcaatg

SEQ ID NO: 464 - DNA, 20 bp, synthetic construct
  source 1..20: mol_type = other DNA
aagcgagaacagcccagaag

SEQ ID NO: 465 - DNA, 20 bp, synthetic construct
  source 1..20: mol_type = other DNA
ggggaggacatcattggtgc

SEQ ID NO: 466 - DNA, 20 bp, synthetic construct
  source 1..20: mol_type = other DNA
cctgcatggtgatgttggac

SEQ ID NO: 467 - DNA, 20 bp, synthetic construct
  source 1..20: mol_type = other DNA
aagatgcccacctgcatggt

SEQ ID NO: 468 - DNA, 20 bp, synthetic construct
  source 1..20: mol_type = other DNA
ggggactttggggaccaact

SEQ ID NO: 469 - DNA, 20 bp, synthetic construct
  source 1..20: mol_type = other DNA
tctacaaattcctcctcagt

SEQ ID NO: 470 - DNA, 20 bp, synthetic construct
  source 1..20: mol_type = other DNA
ggtggacatcttccaggagt

SEQ ID NO: 471 - DNA, 20 bp, synthetic construct
  source 1..20: mol_type = other DNA
tggggcaaagaggtccacac

SEQ ID NO: 472 - DNA, 20 bp, synthetic construct
  source 1..20: mol_type = other DNA
gcatcgcatcaggggcacac

SEQ ID NO: 473 - DNA, 20 bp, synthetic construct
  source 1..20: mol_type = other DNA
ctggtcctcagggaaccagg

SEQ ID NO: 474 - DNA, 20 bp, synthetic construct
  source 1..20: mol_type = other DNA
tttgcccctcacttggtaga

SEQ ID NO: 475 - DNA, 20 bp, synthetic construct
  source 1..20: mol_type = other DNA
catcgcatcaggggcacaca

SEQ ID NO: 476 - DNA, 20 bp, synthetic construct
  source 1..20: mol_type = other DNA
ccaggttgggggtcagtacc

SEQ ID NO: 477 - DNA, 20 bp, synthetic construct
  source 1..20: mol_type = other DNA
agtgccttccagtaagattt

SEQ ID NO: 478 - DNA, 20 bp, synthetic construct
  source 1..20: mol_type = other DNA
tccaatcgagaccctggtgg

SEQ ID NO: 479 - DNA, 20 bp, synthetic construct
  source 1..20: mol_type = other DNA
caactcggccagggtgagct

SEQ ID NO: 480 - DNA, 20 bp, synthetic construct
  source 1..20: mol_type = other DNA
tccagtaagatttggtgtct

SEQ ID NO: 481 - DNA, 20 bp, synthetic construct
  source 1..20: mol_type = other DNA
caggctgcagctcccactgg

SEQ ID NO: 482 - DNA, 20 bp, synthetic construct
  source 1..20: mol_type = other DNA
atgcagctctccagactcac

SEQ ID NO: 483 - DNA, 20 bp, synthetic construct
  source 1..20: mol_type = other DNA
tcctccttctgccatgggtg

SEQ ID NO: 484 - DNA, 20 bp, synthetic construct
  source 1..20: mol_type = other DNA
cctgtgctgaggccacgagg

SEQ ID NO: 485 - DNA, 20 bp, synthetic construct
  source 1..20: mol_type = other DNA
tgcatgtgttcagaaaggct

SEQ ID NO: 486 - DNA, 20 bp, synthetic construct
  source 1..20: mol_type = other DNA
attggctttgatttccctag

SEQ ID NO: 487 - DNA, 20 bp, synthetic construct
  source 1..20: mol_type = other DNA
cctagggtccagcttcaaat

SEQ ID NO: 488 - DNA, 20 bp, synthetic construct
  source 1..20: mol_type = other DNA
ccaccagttcttaggcaact

SEQ ID NO: 489 - DNA, 20 bp, synthetic construct
  source 1..20: mol_type = other DNA
tctctcagcaaacacattac

SEQ ID NO: 490 - DNA, 20 bp, synthetic construct
  source 1..20: mol_type = other DNA
atttccctagggtccagctt

SEQ ID NO: 491 - DNA, 20 bp, synthetic construct
  source 1..20: mol_type = other DNA
aagctggaccctagggaaat

SEQ ID NO: 492 - DNA, 20 bp, synthetic construct
  source 1..20: mol_type = other DNA
ctgagagagaaacagttgcc

SEQ ID NO: 493 - DNA, 20 bp, synthetic construct
  source 1..20: mol_type = other DNA
ctctcctagaccatttccca

SEQ ID NO: 494 - DNA, 20 bp, synthetic construct
  source 1..20: mol_type = other DNA
atgaaacgttcttgtcttag

SEQ ID NO: 495 - DNA, 20 bp, synthetic construct
  source 1..20: mol_type = other DNA
gtataaaatacagagctaag

SEQ ID NO: 496 - DNA, 20 bp, synthetic construct
  source 1..20: mol_type = other DNA
ccagttcttaggcaactgtt

SEQ ID NO: 497 - DNA, 20 bp, synthetic construct
  source 1..20: mol_type = other DNA
ccaatcactttactctccta

SEQ ID NO: 498 - DNA, 20 bp, synthetic construct
  source 1..20: mol_type = other DNA
ctagggcagagaacaggatt

SEQ ID NO: 499 - DNA, 20 bp, synthetic construct
  source 1..20: mol_type = other DNA
tggcattgtcatacgtgtat

SEQ ID NO: 500 - DNA, 20 bp, synthetic construct
  source 1..20: mol_type = other DNA
aatcaatatagggccacaca

SEQ ID NO: 501 - DNA, 20 bp, synthetic construct
  source 1..20: mol_type = other DNA
tgtattcaagctcaaggcct

SEQ ID NO: 502 - DNA, 20 bp, synthetic construct
  source 1..20: mol_type = other DNA
tgctacttactgggaatttg

SEQ ID NO: 503 - DNA, 151 bp, synthetic construct
  source 1..151: mol_type = other DNA
agggacgacttcccgtcccaaaatcgagacagtagccgtaaaactttgagtttcagagtgggcgacacactcgaaaaggttaagatatgcacatagtaatccgtgcatgagccgcgaaagcggcttgaaggtgctgtcttgggtgcattgg

SEQ ID NO: 504 - DNA, 151 bp, synthetic construct
  source 1..151: mol_type = other DNA
agggacgacttcccgtcccaaaatcgagacagtagccgtaaaactttgagtttcagagtgggcgacacactcgaaaaggttaagatatgcacatagtaatccgtgcatgagccgcgaaagcggcttgaaggcagtaagatttggtgtctat

SEQ ID NO: 505 - DNA, 151 bp, synthetic construct
  source 1..151: mol_type = other DNA
agggccgacttcccggcccaaaatcgagacagtagccgtaaaacgttgagtttcagcgtgggcgacacactcgaaaaggttaagatatgcacatagtaatccgtgcatgagccgcgaaagcggcttgaaggtgctgtcttgggtgcattgg

SEQ ID NO: 506 - DNA, 151 bp, synthetic construct
  source 1..151: mol_type = other DNA
agggccgacttcccggcccaaaatcgagacagtagccgtaaaacgttgagtttcagcgtgggcgacacactcgaaaaggttaagatatgcacatagtaatccgtgcatgagccgcgaaagcggcttgaaggcagtaagatttggtgtctat

SEQ ID NO: 507 - DNA, 160 bp, synthetic construct
  source 1..160: mol_type = other DNA
acggttgatttagcaaccgaagtctgagggcatgtagaaaaaagtataggtatataccaacatacttgcattgccactcggaaagggttaaccttggtcattgtgttaccgaccaagcattccagaaatggaatgtaaatcctgcatggtgatgttggac

SEQ ID NO: 508 - DNA, 160 bp, synthetic construct
  source 1..160: mol_type = other DNA
acggttgatttagcaaccgaagtctgagggcatgtagaaaaaagtataggtatataccaacatacttgcattgccactcggaaagggttaaccttggtcattgtgttaccgaccaagcattccagaaatggaatgtaaataagatgcccacctgcatggt

SEQ ID NO: 509 - DNA, 160 bp, synthetic construct
  source 1..160: mol_type = other DNA
acggctgatttagcagccgaagtctgagggcatgtagaaaaaagtataggtatataccaacatacttgcattgccactcggaaagggttaaccttggtcattgtgttaccgaccaagcattccagaaatggaatgtaaatcctgcatggtgatgttggac

SEQ ID NO: 510 - DNA, 160 bp, synthetic construct
  source 1..160: mol_type = other DNA
acggctgatttagcagccgaagtctgagggcatgtagaaaaaagtataggtatataccaacatacttgcattgccactcggaaagggttaaccttggtcattgtgttaccgaccaagcattccagaaatggaatgtaaataagatgcccacctgcatggt

SEQ ID NO: 511 - DNA, 20 bp, synthetic construct
  source 1..20: mol_type = other DNA
ttgtgctggacggtgacgta

SEQ ID NO: 512 - DNA, 20 bp, synthetic construct
  source 1..20: mol_type = other DNA
cctagggtccagcttcaaat

SEQ ID NO: 513 - protein, 433 aa, synthetic construct
MGKGVLAKVMKYELRYLDGCGDFSNMQEQVWALQRQTREILNRSIQIAFQWRCANSEHHRKTGEYLDLKTETGYKRLDGHIYNCLKGQYEDMATSNLNATIQKAWKKYNSSKKEILRGSMSIPSYKMNQPLRLDKNTVKLSEGERNPIVTLTLFSDKFKRAQGVSNVKFSMPLHDGTQRAIFANLMNGTYQLGECQLVYKRPKWFLFVTYKFPPVEHPLDPDKILGVAMGEACALYASTFGEHGYLKIDGGEITKYAKKMEARIRSMQKQAAHCGEGRIGHGTKTRVSVVYQAKDKVARFRDTINHRYSKALIDYALKNQCGTIQMEDLTGIKEDTGFPKFLRHWTYYDLQSKIEAKAAEHGIQVVKINPRHTSQRCSRCGHIDKANRTSQADFCCTKCGFSANAAFNASQNISIRNIDKIIAKAIGANRKQT

SEQ ID NO: 514 - protein, 62 aa, synthetic construct
RTLVTFKDVFVDFTREEWKLLDTAQQIVYRNVMLENYKNLVSLGYQLTKPDVILRLEKGEEP

SEQ ID NO: 515 - protein, 415 aa, synthetic construct
MITVRKLKILIDGESRNESYKFIRDSMYAQYLALNKAMSYLGTAYLSRDKEIFKEAIKSLNNSNPIFDNINFGKGIDTKSSVNQTVKKHIQADIKNGLAKGERSIRNYKRDYPLMTRGRDLKFFYCDTNSTKVKVKWVNGIIFDVMLGKEYNKNDLELRSFLNRVINKEYKISQSSICFDKHNRLILNLSVNITDNIPNEVVKGRIVGVALGMKIPAYVTLNDSEYIGKPIGDINDFLKVRKQFKERKERLQKQLAINKGGRGITNKMQRMDAFTNKEKNFANTYNHGVSKAIINFAKKYKAEQINVEFLALAGSEKEILSSTIRYWSYYQLQQMIEYKANREGIAVKYVDPYLTSQTCCKCGNYEVGQRINQELFECKLCGNKMNAARNASFNIARSTKYISSKEESDFYKQLK